(19) United States
(12) Patent Application Publication
(10) Pub. No.: US 2008/0318957 A1
(43) Pub. Date: Dec. 25, 2008

(54) POLYBASIC BACTERIAL EFFLUX PUMP INHIBITORS AND THERAPEUTIC USES THEREOF

(75) Inventors: TOMASZ GLINKA, Cupertino, CA (US); Olga Lomovskaya, Mountain View, CA (US); Keith Bostian, Atherton, CA (US); David M. Wallace, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: Mpex Pharmaceuticals, Inc., San Deigo, CA (US)

(21) Appl. No.: 12/116,172

(22) Filed: May 6, 2008

Related U.S. Application Data
(60) Provisional application No. 60/917,616, filed on May 11, 2007.

(51) Int. Cl. A61K 31/47; C07D 215/38; A61K 31/5375; A61P 31/04; C07D 265/30

(52) U.S. Cl. 514/237.8; 546/162; 544/168; 514/311

(57) ABSTRACT

Disclosed are compounds having polybasic functionalities. The compounds inhibit bacterial efflux pump inhibitors and are used in combination with an anti-bacterial agent to treat or prevent bacterial infections. These combinations can be effective against bacterial infections that have developed resistance to anti-bacterial agents through an efflux pump mechanism.

### RELATED APPLICATIONS

This application claims the benefit of U.S. Provisional Application No. 60/917,616, filed May 11, 2007, which is incorporated herein by reference in its entirety.

### BACKGROUND OF THE INVENTION

1. Field of the Invention

This invention relates to the field of antimicrobial agents and more specifically it relates to Efflux Pump Inhibitor (EPI) compounds to be co-administered with antimicrobial agents for the treatment of infections caused by drug resistant pathogens. The invention includes novel compounds useful as efflux pump inhibitors, compositions and devices comprising such efflux pump inhibitors, and therapeutic use of such compounds.

2. Description of the Related Art

Antibiotics have been effective tools in the treatment of infectious diseases during the last half-century. From the development of antibiotic therapy to the late 1980s there was almost complete control over bacterial infections in developed countries. However, in response to the pressure of antibiotic usage, multiple resistance mechanisms have become widespread and are threatening the clinical utility of antibacterial therapy. The increase in antibiotic resistant strains has been particularly common in major hospitals and care centers. The consequences of the increase in resistant strains include higher morbidity and mortality, longer patient hospitalization, and an increase in treatment costs.

Bacteria have developed several different mechanisms to overcome the action of antibiotics. These mechanisms of resistance can be specific for a molecule or a family of antibiotics, or can be non-specific and be involved in resistance to unrelated antibiotics. Several mechanisms of resistance can exist in a single bacterial strain, and those mechanisms may act independently or they may act synergistically to overcome the action of an antibiotic or a combination of antibiotics. Specific mechanisms include degradation of the drug, inactivation of the drug by enzymatic modification, and alteration of the drug target. There are, however, more general mechanisms of drug resistance, in which access of the antibiotic to the target is prevented or reduced by decreasing the transport of the antibiotic into the cell or by increasing the efflux of the drug from the cell to the outside medium. Both mechanisms can lower the concentration of drug at the target site and allow bacterial survival in the presence of one or more antibiotics that would otherwise inhibit or kill the bacterial cells. Some bacteria utilize both mechanisms, combining a low permeability of the cell wall (including membranes) with an active efflux of antibiotics.

In recent years interest in efflux-mediated resistance in bacteria has been triggered by the growing amount of data implicating efflux pumps in clinical isolates. The phenomenon of antibiotic efflux was first discovered in 1980, in the context of the mechanism of tetracycline resistance in enterobacteria. Since then, it has been shown that efflux of antibiotics can be mediated by more than one pump in a single organism and that almost all antibiotics are subject to resistance by this mechanism.

Some efflux pumps selectively extrude specific antibiotics. Examples of such pumps include the Tet or CmlA transporters, which can extrude tetracycline or chloramphenicol, respectively. Other efflux pumps, so-called multi-drug resistance (MDR) pumps, extrude a variety of structurally diverse compounds. In the latter case, a single efflux system may confer resistance to multiple antibiotics with different modes of action. In this respect, bacterial MDR pumps are similar to mammalian MDR transporters. In fact, one such pump, P-glycoprotein, the first discovered MDR pump, confers multiple drug resistance on cancer cells and is considered to be one of the major reasons tumor resistance to anti-cancer therapy. A typical example of bacterial MDR pump is MexAB-OprM from Pseudomonas aeruginosa. This pump has been shown to affect the susceptibility of the organism to almost all antibiotic classes which fluoroquinolones, β-lactams, macrolides, phenicols, tetracyclines, and oxazolidinones.

Efflux pumps in gram-positive bacteria excrete their substrates across a single cytoplasmic membrane. This is also the case for some pumps in gram-negative bacteria, and as a result their substrates are effluxed into the periplasmic space. Other efflux pumps from gram-negative bacteria efflux their substrates directly into the external medium, bypassing the periplasm and the outer membrane. These pumps are organized in complex three component structures, which traverse both inner and outer membranes. They consist of a transporter located in the cytoplasmic membrane, an outer membrane channel and a periplasmic ‘linker’ protein, which brings the other two components into contact. It is clearly advantageous for gram-negative bacteria to efflux drugs by bypassing the periplasm and outer membrane. In gram-negative bacteria the outer membrane significantly slows down the entry of both lipophilic and hydrophilic agents. The former, such as erythromycin and fusidic acid, are hindered by the lipopolysaccharide components of the outer leaflet of the outer membrane bilayer. Hydrophilic agents cross the outer membrane through water-filled porins whose size prevents rapid diffusion, even for small compounds such as fluoroquinolones and some β-lactams. Thus, direct efflux creates the possibility for two different mechanisms to work synergistically to provide the cell with a potent defense mechanism. Furthermore, direct efflux into the medium leads to decreased amounts of drugs not only in the cytoplasmic but also in the periplasmic space. This could explain the apparently paradoxical finding that efflux pumps protect gram-negative bacteria from β-lactam antibiotics whose target penicillin-binding proteins are found in the periplasm.

Many MDR pumps are encoded by the genes, which are normal constituents of bacterial chromosomes. In this case increased antibiotic resistance is a consequence of over-expression of these genes. Thus bacteria have the potential to develop multi-drug resistance without the acquisition of multiple specific resistance determinants. In some cases, the simultaneous operation of efflux pumps and other resistance mechanisms in the same cell results in synergistic effects.

While some genes encoding efflux pumps are not expressed in wild type cells and require induction or regulatory mutations for expression to occur, other efflux genes are expressed constitutively. As a result wild type cells have basal level of efflux activity. This basal activity of multi-drug efflux pumps in wild type cells contribute to intrinsic antibiotic resistance, or more properly, decreased antibiotic susceptibility. This intrinsic resistance may be low enough for the bacteria to still be clinically susceptible to therapy. However, the bacteria might be even more susceptible if efflux pumps were rendered non-functional, allowing lower doses of antibiotics to be effective. To illustrate, P. aeruginosa laboratory-derived mutant strain PAM1626, which does not produce any measurable amounts of efflux pump is 8 to 10 fold more susceptible to levofloxacin and meropenem than the parent strain P. aeruginosa PAM1020, which produces the basal level of MexAB-OprM efflux pump. Were it not for efflux pumps, the spectrum of activity of many so-called ‘gram-positive’antibiotics could be expanded to previously non-susceptible gram-negative species. This can be applied to ‘narrow-spectrum’ β-lactams, macrolides, lincosamides, streptogramins, rifamycins, fusidic acid, and oxazolidinones—all of which have a potent antibacterial effect against engineered mutants lacking efflux pumps.

It is clear that in many cases, a dramatic effect on the susceptibility of problematic pathogens would be greatly enhanced if efflux-mediated resistance were to be nullified. Two approaches to combat the adverse effects of efflux on the efficacy of antimicrobial agents can be envisioned: identification of derivatives of known antibiotics that are not effluxed and development of therapeutic agents that inhibit transport activity of efflux pumps and could be used in combination with existing antibiotics to increase their potency.

There are several examples when the first approach has been successfully reduced to practice. These examples include new fluoroquinolones, which are not affected by multidrug resistance pumps in Staphylococcus aureus or Streptococcus pneumoniae or new tetracycline and macrolide derivatives, which are not recognized by the corresponding antibiotic-specific pumps. However, this approach appears to be much less successful in the case of multidrug resistance pumps from gram-negative bacteria. In gram-negative bacteria, particular restrictions are imposed on the structure of successful drugs: they must be amphiphilic in order to cross both membranes. It is this very property that makes antibiotics good substrates of multi-drug resistance efflux pumps from gram-negative bacteria. In the case of these bacteria the efflux pump inhibitory approach becomes the major strategy in improving the clinical effectiveness of existing antibacterial therapy.

The efflux pump inhibitory approach was first validated in the case of mammalian P-glycoprotein. And the first inhibitors have been found among compounds with previously described and quite variable pharmacological activities. For example, P-glycoprotein-mediated resistance, can be reversed by calcium channel blockers such as verpamyl and azidopine, immunosuppressive agents cyclosporin A and FK506 as well as antifungal agents such as rapamycin and FK520 (Raymond et al, 1994). It is important that efflux pump inhibitory activity was by no means connected to other activities of these compounds. In fact, the most advanced inhibitor of P-glycoprotein is a structural derivative of cyclosporin A and is devoid if immunosuppressive activity.

### SUMMARY OF THE INVENTION

Some embodiments disclosed herein include bacterial efflux pump inhibitors having polybasic functionality. Other embodiments disclosed herein include pharmaceutical compositions and methods of treatment using these compounds. One embodiment disclosed herein includes a compound having the structure of formula I, II or III:

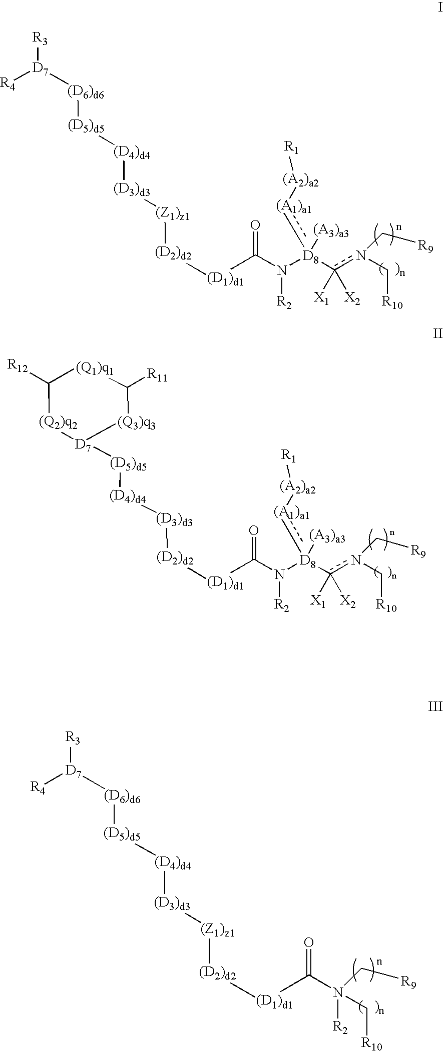

or a pharmaceutically acceptable salt or pro-drug thereof wherein;
each bond represented by a dashed and solid line represents a bond selected from the group consisting of a single bond and a double bond;
each R₁ is independently selected from C₁-C₆ alkyl, C₃-C₇ carbocyclyl, heterocyclyl, aryl and heteroaryl, each optionally substituted with up to 3 substituents independently selected from the group consisting of a halide, alkyl, carbocyclyl, —(CH₂)ₙaryl, —OR₂, —OR₁₀, —S(R₂)₂, —SO₂NHR₁₀, —(CH₂)ₙSH, —CF₃, —OCF₃, —N(R₂)₂, NO₂, —CN, —CO₂alkyl, —CO₂aryl and —C(O)aryl;
each R₂ is independently selected from H and C₁-C₆ alkyl;
R₃ is selected from —(CH₂)ₙCHR₅R₆, —(CH₂)ₙNR₅R₆, and (CH₂)ₘC(═O)NR₅R₆;
each R₄ is independently selected from —NHR₂, —(CH₂)ₙCHR₅R₆, —(CH₂)ₙNR₅R₆, —(CH₂)ₘC(═O)NR₅R₆, and —C(═NR₅)NR₅R₅;
each R₅ is independently selected from H and —(CH₂)ₘNH₂;
each R₆ is independently selected from —(CH₂)ₙNHR₇, (CH₂)ₙNHC(═NH)NH₂, —(CH₂)ₙNHC(R₂)═NH, —(CH₂)ₙC(═NH)NH₂, and (CH₂)ₙN⁺(CH₃)₃;
each R₇ is independently selected from H, alkyl, —C(═O)CH(R₁₃)(NH₂), —C(═O)A₂CH₂NH₂, Alanine, Arginine, Asparagine, Aspartic acid, Glutamic acid, Glutamine, Cysteine, Glycine, Histidine, Isoleucine, Leucine, Lysine, Methionine, Phenylalanine, Proline, Serine, Threonine, Tryptophan, Tyrosine, and Valine;
R₈ is selected from H, alkyl, aryl, SH and OH;
R₉ is selected from H, C₁-C₆ alkyl, C₃-C₁₀ carbocyclyl, heterocyclyl, aryl, heteroaryl, and —NHC(O)-aryl, each optionally substituted with up to 3 substituents independently selected from the group consisting of a halide, alkyl, carbocyclyl, —(CH₂)ₙR₁, —(CH═CH)ₙR₁, —OR₂, —OR₁, ═O, —S(R₂)₂, —SR₁, —SO₂NR₁R₂, —(CH₂)ₙSH, —CF₃, —OCF₃, —N(R₂)₂, —NO₂, —CN, —(C═X)R₁, —(C═X)R₂, —CO₂alkyl, —CO₂aryl, heteroaryl optionally substituted with C₁-C₆ alkyl, and aryl optionally substituted with C₁-C₆ alkyl;

R₁₀ is selected from C₁-C₆ alkyl, C₃-C₁₀ carbocyclyl, heterocyclyl, aryl, heteroaryl, and —NHC(O)-aryl, each optionally substituted with up to 3 substituents independently selected from the group consisting of a halide, alkyl, carbocyclyl, —(CH₂)ₙR₁, —OR₂, —OR₁, ═O, —S(R₂)₂, —SR₁, —SO₂NR₁R₂, —(CH₂)ₙSH, —CF₃, —OCF₃, —N(R₂)₂, —NO₂, —CN, —(C═X)R₁, —(C═X)R₂, —CO₂alkyl, and —CO₂aryl;
R₉ and R₁₀ are optionally linked to form a ring;
R₁₁ is selected from H, —(CH₂)ₙNHR₂ and —(CH₂)ₙCHR₅R₆;
R₁₂ is selected from —(CH₂)ₙNHR₂ and —(CH₂)ₙCHR₅R₆;
R₁₃ is selected from —(CH₂)ₙCHR₅(CH₂)ₙNH₂, —(CH₂)ₘNR₅(CH₂)ₙNH₂ and —(CH₂)ₘC(═O)NR₅(CH₂)ₙNH₂;
A₁ is —[C(R₂R₈)]ₘ or ═CR₂[C(R₂R₈)]ₘ—, wherein if A₁ is ═CR₂[C(R₂R₈)]ₘ—, then a3 is 0;
A₂ is —(CH₂)ₘ—, —C(═X)—, —O(CH₂)ₙ—, —S(CH₂)ₙ—, —CH═CH—, —C(═N—OR₂)—, or —NR₂—;
A₃ is H, C₁-C₆ alkyl, a lone electron pair when D₈ is N, or A₃ is —CH₂-bonded to A₁, A₂ or R₁ to form a ring;
a1, a2 and a3 are independently equal to 0 or 1;
D₁ is selected from —CH₂—, —N(NHR₇)—, —CH(NHR₇)—, —CH[(CH₂)ₘNHR₇]—, —CH(R₂)—, and —CH(CH₂SH)—;
D₂, D₃, D₄, D₅ and D₆ are independently selected from the group consisting of —(CH₂)ₘ—, —CH(R₂)—, —CH(NHR₇)—, —N(R₅)—, —O—, —S—, —C(═X)—, —S(═O)— and —SO₂—, wherein any two atoms of D₂, D₃, D₄, D₅ and D₆ are optionally linked to form a three, four, five or six membered saturated ring;
D₇ is selected from N, ═C< where the carbon forms a double bond with an adjacent carbon in one of D₁-D₆, CH and CR₄;
D₈ is selected from C and N;
d1, d2, d3, d4, d5 and d6 are independently equal to 0 or 1;
Q₁ is selected from —CH₂—, —N(R₂)N(R₂)—, and —N(R₂)—;
Q₂ and Q₃ are independently selected from the group consisting of —CH₂— and —N(R₂)—;
with the proviso that no more than one of Q₁, Q₂, and Q₃ comprises a nitrogen;
q1, q2, and q3 are independently equal to 0 or 1;
X₁ and X₂ are each hydrogen or taken together are ═O or ═S,
or X₁ is hydrogen and X₂ is —O— or —S— bonded to R₁₀ to form a 5- or 6-membered heterocyclyl,
or X₁ is absent and X₂ is —O— or —S— bonded to R₁₀ to form a 5- or 6-membered heterocyclyl or heteroaryl, wherein when X₁ is absent, the bond to nitrogen represented by a dashed and solid line is a double bond;
each X is independently O or S;
Z₁ is an aryl, heteroaryl, carbocyclyl, or heterocyclyl;
z1 is 0 or 1;
if z1 is 0 then at least two from the group consisting of d1, d2, d3, d4, d5 and
d6 are equal to 1, if z1 is 1 then at least one from the group consisting of d1, d2, d3, d4, d5 and d6 is equal to 1;
each n is independently an integer of 0 to 4; and
each m is independently an integer of 1 to 3.

Another embodiment disclosed herein includes a compound having the structure of formula IV:

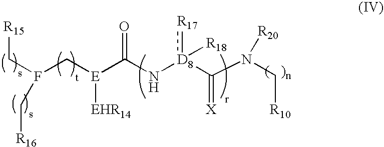

or a pharmaceutically acceptable salt or prodrug thereof, wherein:
D₈ is selected from C and N;
each E is independently CH or N;
F is selected from the group consisting of:

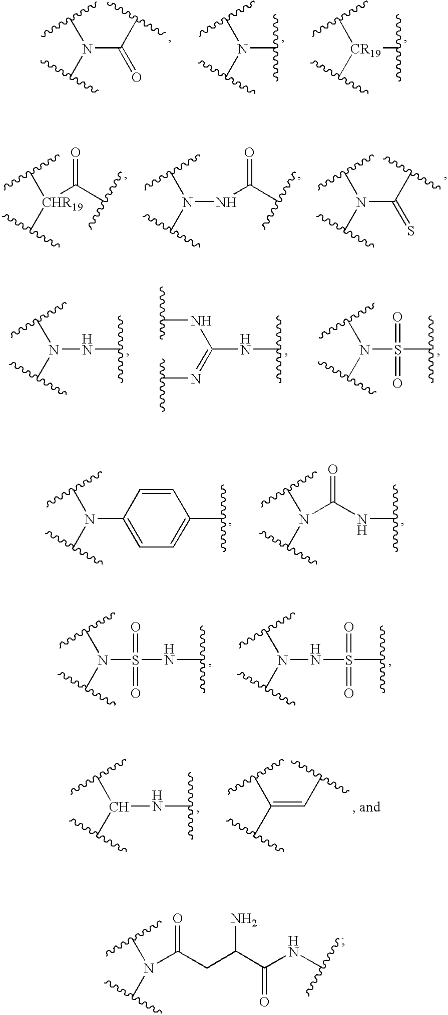

X is O or S;
R₁₀ is selected from carbocyclyl, heterocyclyl, aryl, heteroaryl, —NHC(O)-aryl, and aralkyl, each optionally substituted with up to 3 substituents independently selected from the group consisting of a halide, alkyl, —CF₃, —OCF₃, —NO₂, —CN, —OH, ═O, carbocyclyl, heterocyclyl, aryl optionally substituted with halide or —OH, heteroaryl optionally substituted with alkyl, —O-aryl optionally substituted with —O—C₁-C₆ alkyl, —O-heteroaryl, —O-heterocyclyl, —SO₂NH-heteroaryl, —O—C₁-C₆ alkyl, —SO₂NEt₂, SMe, di(C₁-C₆)alkylamino, —CH₂-heterocyclyl optionally substituted with alkyl, —CH₂-aryl, —C(O)aryl, and —CH═CH-aryl;
R₁₄ is selected from H, —C(O)—CH(Me)(NH₂), —C(O)—CH(CH₂OH)(NH₂), and —(CH₂)ₜNH₂;
R₁₅ and R₁₆ are independently selected from —NH₂, —NHC(═NH)NH₂, —N⁺(CH₃)₃, —NHCH₂CH₂NH₂, —N(CH₂CH₂NH₂)₂, —C(O)N(CH₂CH₂NH₂)₂, —CH(CH₂NH₂)₂, and —CH₂(NH₂)(CH₂NH₂),
or R₁₅ and R₁₆ together with F form a heterocyclyl substituted with at least two substituents independently selected from —(CH₂)ₙNH₂, —(CH₂)ₙNHC(═NH)NH₂—(CH₂)ₙN⁺(CH₃)₃, —(CH₂)ₙNHCH₂CH₂NH₂, —(CH₂)ₙN(CH₂CH₂NH₂)₂, —(CH₂)ₙC(O)N(CH₂CH₂NH₂)₂, and —(CH₂)SCH(CH₂NH₂)₂;
R₁₇ is selected from alkyl, aralkyl, heteroaralkyl, carbocyclyl-alkyl, heterocyclyl-alkyl, aryl, and carbocyclyl, each optionally substituted with up to 3 substituents independently selected from the group consisting of —CF₃, —OH, —OCF₃, halide, —CN, alkyl, —O-aralkyl, aryl, —S(CH₃)₂, —C(O)aryl, —S-aralkyl optionally substituted with —OMe, ═O, and ═N—OH;
R₁₈ is H, alkyl, or absent,
or R₁₇ together with R₁₈ form a carbocyclyl optionally substituted with aryl or heteroaryl;
R₁₉ is H, —CH₂NH₂, or —CH₂CH₂NH₂;
R₂₀ is H or alkyl;
each t is independently an integer from 1 to 4;
each s is independently an integer from 0 to 3;
r is 0 or 1; and
n is an integer from 0 to 4.

Other embodiments disclosed herein include methods of inhibiting a bacterial efflux pump by administering to a subject infected with a bacteria a compound according to any of the above formulas.

Another embodiment disclosed herein includes a method of treating or preventing a bacterial infection by co-administering to a subject infected with a bacteria or subject to infection with a bacteria, a compound according to any of the above formulas and another anti-bacterial agent.

Another embodiment disclosed herein includes a pharmaceutical composition that has a compound according to any of the above formulas and a pharmaceutically acceptable carrier, diluent, or excipient.

It is to be understood that both the foregoing general description and the following detailed description are exemplary and explanatory only and are not restrictive of the invention, as claimed.

### DETAILED DESCRIPTION OF THE PREFERRED EMBODIMENT

Compositions and methods for inhibiting intrinsic drug resistance and/or preventing acquired drug resistance in microbes would be of tremendous benefit. Certain embodiments provide such compositions and methods.

Some embodiments relate to a method for treating a microbial infection whose causative microbe employs an efflux pump resistance mechanism, comprising contacting the microbial cell with an efflux pump inhibitor in combination with an antimicrobial agent. The efflux pump inhibitors of preferred embodiments can comprise polybasic structures, as disclosed herein.

Some embodiments include a method for prophylactic treatment of a mammal. In this method, an efflux pump inhibitor is administered to a mammal at risk of a microbial infection, e.g., a bacterial infection. In some embodiments, an antimicrobial agent is administered in combination with or coadministered with the efflux pump inhibitor.

Some embodiments also feature a method of enhancing the antimicrobial activity of an antimicrobial agent against a microbe, in which such a microbe is contacted with a efflux pump inhibitor, and an antibacterial agent.

In some embodiments, pharmaceutical compositions are provided that are effective for treatment of an infection of an animal, e.g., a mammal, by a microbe, such as a bacterium or a fungus. The composition includes a pharmaceutically acceptable carrier and an efflux pump inhibitor as described herein. Some embodiments provide antimicrobial formulations that include an antimicrobial agent, an efflux pump inhibitor, and a carrier. In some embodiments, the antimicrobial agent is an antibacterial agent.

In some embodiments, the efflux pump inhibitor is administered to the lungs as an aerosol. In some such embodiments, a co-administered antimicrobial agent may be administered in conjunction with the efflux pump inhibitor by any known means.

### DEFINITIONS

In this specification and in the claims, the following terms have the meanings as defined. As used herein, “alkyl” means a branched, or straight chain chemical group containing only carbon and hydrogen, such as methyl, isopropyl, isobutyl, sec-butyl and pentyl. Alkyl groups can either be unsubstituted or substituted with one or more substituents, e.g., halogen, alkoxy, acyloxy, amino, amido, cyano, nitro, hydroxyl, mercapto, carboxy, carbonyl, benzyloxy, aryl, heteroaryl, or other functionality that may be suitably blocked, if necessary for purposes of the invention, with a protecting group. Alkyl groups can be saturated or unsaturated (e.g., containing —C═C— or —C≡C— subunits), at one or several positions. Typically, alkyl groups will comprise 1 to 8 carbon atoms, preferably 1 to 6, and more preferably 1 to 4 carbon atoms.

As used herein, “carbocyclyl” means a cyclic ring system containing only carbon atoms in the ring system backbone, such as cyclopropyl, cyclobutyl, cyclopentyl, cyclohexyl, and cyclohexenyl. Carbocyclyls may include multiple fused rings. Carbocyclyls may have any degree of saturation provided that at least one ring in the ring system is not aromatic. Carbocyclyl groups can either be unsubstituted or substituted with one or more substituents, e.g., halogen, alkoxy, acyloxy, amino, amido, cyano, nitro, hydroxyl, mercapto, carboxy, carbonyl, benzyloxy, aryl, heteroaryl, or other functionality that may be suitably blocked, if necessary for purposes of the invention, with a protecting group. Typically, carbocyclyl groups will comprise 3 to 10 carbon atoms, preferably 3 to 6.

As used herein, “lower alkyl” means a subset of alkyl, and thus is a hydrocarbon substituent, which is linear, or branched. Preferred lower alkyls are of 1 to about 4 carbons, and may be branched or linear. Examples of lower alkyl include butyl, propyl, isopropyl, ethyl, and methyl. Likewise, radicals using the terminology “lower” refer to radicals preferably with 1 to about 4 carbons in the alkyl portion of the radical.

As used herein, “amido” means a H—CON— or alkyl-CON—, aryl-CON— or heterocyclyl-CON group wherein the alkyl, cycloalkyl, aryl or heterocyclyl group is as herein described.

As used herein, “aryl” means an aromatic radical having a single-ring (e.g., phenyl) or multiple condensed rings (e.g., naphthyl or anthryl) with only carbon atoms present in the ring backbone. Aryl groups can either be unsubstituted or substituted with one or more substitutents, e.g., amino, cyano, hydroxyl, lower alkyl, haloalkyl, alkoxy, nitro, halo, mercapto, and other substituents. A preferred carbocyclic aryl is phenyl.

As used herein, the term “heteroaryl” means an aromatic radical having one or more heteroatom(s) (e.g., N, O, or S) in the ring backbone and may include a single ring (e.g., pyridine) or multiple condensed rings (e.g., quinoline). Heteroaryl groups can either be unsubstituted or substituted with one or more substituents, e.g., amino, cyano, nitro, hydroxyl, alkyl, cycloalkyl, haloalkyl, alkoxy, aryl, halo, and mercapto. Examples of heteroaryl include thienyl, pyrridyl, furyl, oxazolyl, oxadiazolyl, pyrollyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl and others.

In these definitions it is clearly contemplated that substitution on the aryl and heteroaryl rings is within the scope of certain embodiments. Where substitution occurs, the radical is called substituted aryl or substituted heteroaryl. Preferably one to three and more preferably one or two substituents occur on the aryl ring. Though many substituents will be useful, preferred substituents include those commonly found in aryl compounds, such as alkyl, cycloalkyl, hydroxy, alkoxy, cyano, halo, haloalkyl, mercapto and the like.

As used herein, “amide” includes both RNR′CO— (in the case of R=alkyl, alkaminocarbonyl-) and RCONR′— (in the case of R=alkyl, alkyl carbonylamino-).

As used herein, the term “ester” includes both ROCO— (in the case of R=alkyl, alkoxycarbonyl-) and RCOO— (in the case of R=alkyl, alkylcarbonyloxy-).

As used herein, “acyl” means an H—CO— or alkyl-CO—, aryl-CO— or heterocyclyl-CO— group wherein the alkyl, cycloalkyl, aryl or heterocyclcyl group is as herein described. Preferred acyls contain a lower alkyl. Exemplary alkyl acyl groups include formyl, acetyl, propanoyl, 2-methylpropanoyl, t-butylacetyl, butanoyl and palmitoyl.

As used herein, “halo or halide” is a chloro, bromo, fluoro or iodo atom radical. Chloro, bromo and fluoro are preferred halides. The term “halo” also contemplates terms sometimes referred to as “halogen”, or “halide”.

As used herein, “haloalkyl” means a hydrocarbon substituent, which is linear or branched or cyclic alkyl, alkenyl or alkynyl substituted with chloro, bromo, fluoro or iodo atom(s). Most preferred of these are fluoroalkyls, wherein one or more of the hydrogen atoms have been substituted by fluoro. Preferred haloalkyls are of 1 to about 3 carbons in length, More preferred haloalkyls are 1 to about 2 carbons, and most preferred are 1 carbon in length. The skilled artisan will recognize then that as used herein, “haloalkylene” means a diradical variant of haloalkyl, such diradicals may act as spacers between radicals, other atoms, or between the parent ring and another functional group.

As used herein, “heterocyclyl” means a cyclic ring system comprising at least one heteroatom in the ring system backbone. Heterocyclyls may include multiple fused rings. Heterocyclyls may have any degree of saturation provided that at least one ring in the ring system is not aromatic. Heterocyclyls may be substituted or unsubstituted, and are attached to other groups via any available valence, preferably any available carbon or nitrogen. More preferred heterocycles are of 5 or 6 members. In six membered monocyclic heterocycles, the heteroatom(s) are selected from one up to three of O, N or S, and wherein when the heterocycle is five membered, preferably it has one or two heteroatoms selected from O, N, or S.

As used herein quaternary ammonium refers to a positively charged nitrogen atom linked to four aliphatic carbon atoms or a positively charged nitrogen of the heteroaryl ring linked to an aliphatic carbon as in N-pridinium, N-thiazolium, N-imidazolium, N-triazolium and like.

As used herein, “substituted amino” means an amino radical which is substituted by one or two alkyl, cycloalkyl, aryl, or heterocyclyl groups, wherein the alkyl, aryl or heterocyclyl are defined as above.

As used herein, “substituted thiol” means RS— group wherein R is an alkyl, an aryl, or a heterocyclyl group, wherein the alkyl, cycloalkyl, aryl or heterocyclyl are defined as above.

As used herein, “sulfonyl” means an alkylSO₂, arylSO₂ or heterocyclyl-SO₂ group wherein the alkyl, cycloalkyl, aryl or heterocyclyl are defined as above.

As used herein, “sulfamido” means an alkyl-N—S(O)₂N—, aryl-NS(O)₂N— or heterocyclyl-NS(O)₂N— group wherein the alkyl, cycloalkyl, aryl or heterocyclcyl group is as herein described.

As used herein, “sulfonamido” means an alkyl-S(O)₂N—, aryl-S(O)₂N— or heterocyclyl-S(O)₂N— group wherein the alkyl, cycloalkyl, aryl or heterocyclcyl group is as herein described.

As used herein, “ureido” means an alkyl-NCON—, aryl-NCON— or heterocyclyl-NCON— group wherein the alkyl, cycloalkyl, aryl or heterocyclcyl group is as herein described

As used herein, when two groups are indicated to be “linked” or “bonded” to form a “ring,” it is to be understood that a bond is formed between the two groups and may involve replacement of a hydrogen atom on one or both groups with the bond, thereby forming a carbocyclyl, heterocyclyl, aryl, or heteroaryl ring. The skilled artisan will recognize that such rings can and are readily formed by routine chemical reactions, and it is within the purview of the skilled artisan to both envision such rings and the methods of their formations. Preferred are rings having from 3-7 members, more preferably 5 or 6 members. As used herein the term “ring” or “rings” when formed by the combination of two radicals refers to heterocyclic, carbocyclic, aryl, or heteroaryl rings.

The skilled artisan will recognize that some structures described herein may be resonance forms or tautomers of compounds that may be fairly represented by other chemical structures, even when kinetically, the artisan recognizes that such structures are only a very small portion of a sample of such compound(s). Such compounds are clearly contemplated within the scope of this invention, though such resonance forms or tautomers are not represented herein.

The term “administration” or “administering” refers to a method of giving a dosage of an antimicrobial pharmaceutical composition to a vertebrate or invertebrate, including a mammal, a bird, a fish, or an amphibian, where the method is, e.g. intrarespiratory, topical, oral, intravenous, intraperitoneal, or intramuscular. The preferred method of administration can vary depending on various factors, e.g. the components of the pharmaceutical composition, the site of the potential or actual bacterial infection, the microbe involved, and the severity of an actual microbial infection.

A “diagnostic” as used herein is a compound, method, system, or device that assists in the identification and characterization of a health or disease state. The diagnostic can be used in standard assays as is known in the art.

The term “efflux pump” refers to a protein assembly that exports substrate molecules from the cytoplasm or periplasm of a cell, in an energy dependent fashion. Thus an efflux pump will typically be located in the cytoplasmic membrane of the cell (spanning the cytoplasmic membrane). In Gram-negative bacteria the pump may span the periplasmic space and there may also be portion of the efflux pump, which spans the outer membrane.

An “efflux pump inhibitor” (“EPI”) is a compound that specifically interferes with the ability of an efflux pump to export its normal substrate, or other compounds such as an antibiotic. The inhibitor may have intrinsic antimicrobial (e.g., antibacterial) activity of its own, but at least a significant portion of the relevant activity is due to the efflux pump inhibiting activity.

The term “mammal” is used in its usual biological sense. Thus, it specifically includes humans, cattle, horses, dogs, and cats, but also includes many other species.

The term “microbial infection” refers to the invasion of the host organism, whether the organism is a vertebrate, invertebrate, fish, plant, bird, or mammal, by pathogenic microbes. This includes the excessive growth of microbes that are normally present in or on the body of a mammal or other organism. More generally, a microbial infection can be any situation in which the presence of a microbial population(s) is damaging to a host mammal. Thus, a mammal is “suffering” from a microbial infection when excessive numbers of a microbial population are present in or on a mammal's body, or when the effects of the presence of a microbial population(s) is damaging the cells or other tissue of a mammal. Specifically, this description applies to a bacterial infection. Note that the compounds of preferred embodiments are also useful in treating microbial growth or contamination of cell cultures or other media, or inanimate surfaces or objects, and nothing herein should limit the preferred embodiments only to treatment of higher organisms, except when explicitly so specified in the claims.

The term “multidrug resistance pump” refers to an efflux pump that is not highly specific to a particular antibiotic. The term thus includes broad substrate pumps (efflux a number of compounds with varying structural characteristics). These pumps are different from pumps, which are highly specific for tetracyclines. Tetracycline efflux pumps are involved in specific resistance to tetracycline in bacteria. However, they do not confer resistance to other antibiotics. The genes for the tetracycline pump components are found in plasmids in Gram-negative as well as in Gram-positive bacteria.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g. in the Merck Index, Merck & Company, Rahway, N.J. Considerations for the inclusion of various components in pharmaceutical compositions are described, e.g. in Gilman et al. (Eds.) (1990); Goodman and Gilman's: The Pharmacological Basis of Therapeutics, 8th Ed., Pergamon Press.

The term “pharmaceutically acceptable salt” refers to salts that retain the biological effectiveness and properties of the compounds of the preferred embodiments and, which are not biologically or otherwise undesirable. In many cases, the compounds of the preferred embodiments are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in World Patent Publication 87/05297, Johnston et al., published Sep. 11, 1987 (incorporated by reference herein).

“Solvate” refers to the compound formed by the interaction of a solvent and an EPI, a metabolite, or salt thereof. Suitable solvates are pharmaceutically acceptable solvates including hydrates.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

In the context of the response of a microbe, such as a bacterium, to an antimicrobial agent, the term “susceptibility” refers to the sensitivity of the microbe for the presence of the antimicrobial agent. So, to increase the susceptibility means that the microbe will be inhibited by a lower concentration of the antimicrobial agent in the medium surrounding the microbial cells. This is equivalent to saying that the microbe is more sensitive to the antimicrobial agent. In most cases the minimum inhibitory concentration (MIC) of that antimicrobial agent will have been reduced.

By “therapeutically effective amount” or “pharmaceutically effective amount” is meant an amount of an efflux pump inhibitor, or amounts individually of an efflux pump inhibitor and an antimicrobial agent, as disclosed in the preferred embodiments, which have a therapeutic effect, which generally refers to the inhibition to some extent of the normal metabolism of microbial cells causing or contributing to a microbial infection. The doses of efflux pump inhibitor and antimicrobial agent, which are useful in combination as a treatment, are therapeutically effective amounts. Thus, as used herein, a therapeutically effective amount means those amounts of efflux pump inhibitor and antimicrobial agent which, when used in combination, produce the desired therapeutic effect as judged by clinical trial results and/or model animal infection studies. In particular embodiments, the efflux pump inhibitor and antimicrobial agent are combined in pre-determined proportions and thus a therapeutically effective amount would be an amount of the combination. This amount and the amount of the efflux pump inhibitor and antimicrobial agent individually can be routinely determined by one of skill in the art, and will vary, depending on several factors, such as the particular microbial strain involved and the particular efflux pump inhibitor and antimicrobial agent used. This amount can further depend upon the patient's height, weight, sex, age and medical history. For prophylactic treatments, a therapeutically effective amount is that amount which would be effective if a microbial infection existed.

A therapeutic effect relieves, to some extent, one or more of the symptoms of the infection, and includes curing an infection. “Curing” means that the symptoms of active infection are eliminated, including the elimination of excessive members of viable microbe of those involved in the infection. However, certain long-term or permanent effects of the infection may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment” refers to treating a patient who is not yet infected, but who is susceptible to, or otherwise at risk of, a particular infection. The term “therapeutic treatment” refers to administering treatment to a patient already suffering from an infection. Thus, in preferred embodiments, treating is the administration to a mammal (either for therapeutic or prophylactic purposes) of therapeutically effective amounts of an efflux pump inhibitor and an antibacterial (or antimicrobial) agent in combination (either simultaneously or serially).

### Compounds

Some embodiments include compounds containing within the Box A fragment at least two basic nitrogen functionalities basic enough to be protonated to an appreciable degree at physiological pH of 7.4. One embodiment includes a compound having the structure of formula (I):

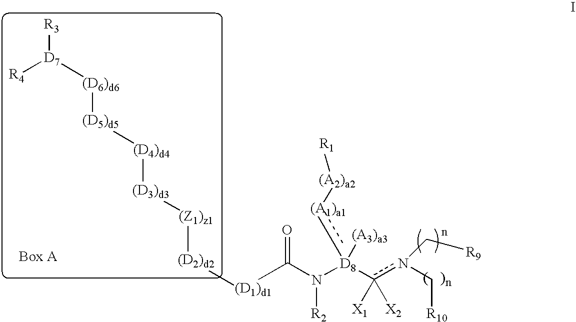

or a pharmaceutically acceptable salt or pro-drug thereof wherein;
each bond represented by a dashed and solid line represents a bond selected from the group consisting of a single bond and a double bond;
each R₁ is independently selected from C₁-C₆ alkyl, C₃-C₇ carbocyclyl, heterocyclyl, aryl and heteroaryl, each optionally substituted with up to 3 substituents independently selected from the group consisting of a halide, alkyl, carbocyclyl, —(CH₂)ₙaryl, —OR₂, —OR₁₀, —S(R₂)₂, —SO₂NHR₁₀, —(CH₂)ₙSH, —CF₃, —OCF₃, —N(R₂)₂, —NO₂, —CN, —CO₂alkyl, —CO₂aryl and —C(O)aryl;
each R₂ is independently selected from H and C₁-C₆ alkyl;
R₃ is selected from —(CH₂)ₙCHR₅R₆, —(CH₂)ₙNR₅R₆, and —(CH₂)ₘC(═O)NR₅R₆;
each R₄ is independently selected from —NHR₂, —(CH₂)ₙCHR₅R₆, —(CH₂)ₙNR₅R₆, —(CH₂)ₘC(═O)NR₅R₆, and —C(═NR₅)NR₅R₅;
each R₅ is independently selected from H and —(CH₂)ₘNH₂;
each R₆ is independently selected from —(CH₂)ₙNHR₇, —(CH₂)ₙNHC(═NH)NH₂, —(CH₂)ₙNHC(R₂)═NH, —(CH₂)ₙC(═NH)NH₂, and —(CH₂)ₙN⁺(CH₃)₃;
each R₇ is independently selected from H, alkyl, —C(═O)CH(R₁₃)(NH₂), —C(═O)A₂CH₂NH₂, Alanine, Arginine, Asparagine, Aspartic acid, Glutamic acid, Glutamine, Cysteine, Glycine, Histidine, Isoleucine, Leucine, Lysine, Methionine, Phenylalanine, Proline, Serine, Threonine, Tryptophan, Tyrosine, and Valine;
R₈ is selected from H, alkyl, aryl, SH and OH;
R₉ is selected from H, C₁-C₆ alkyl, C₃-C₁₀ carbocyclyl, heterocyclyl, aryl, heteroaryl, and —NHC(O)-aryl, each optionally substituted with up to 3 substituents independently selected from the group consisting of a halide, alkyl, carbocyclyl, —(CH₂)ₙR₁, —(CH═CH)ₙR₁, —OR₂, —OR₁, ═O, —S(R₂)₂, —SR₁, —SO₂NR₁R₂, —(CH₂)ₙSH, —CF₃, —OCF₃, —N(R₂)₂, —NO₂, —CN, —(C═X)R₁, —(C═X)R₂, —CO₂alkyl, —CO₂aryl, heteroaryl optionally substituted with C₁-C₆ alkyl, and aryl optionally substituted with C₁-C₆ alkyl;
R₁₀ is selected from C₁-C₆ alkyl, C₃-C₁₀ carbocyclyl, heterocyclyl, aryl, heteroaryl, and —NHC(O)-aryl, each optionally substituted with up to 3 substituents independently selected from the group consisting of a halide, alkyl, carbocyclyl, —(CH₂)ₙR₁, —OR₂, —OR₁, ═O, —S(R₂)₂, —SR₁, —SO₂NR₁R₂, —(CH₂)ₙSH, —CF₃, —OCF₃, —N(R₂)₂, —NO₂, —CN, —(C═X)R₁, —(C═X)R₂, —CO₂alkyl, and —CO₂aryl;
R₉ and R₁₀ are optionally linked to form a ring;
R₁₃ is selected from —(CH₂)ₙCHR₅(CH₂)ₙNH₂, —(CH₂)ₘNR₅(CH₂)ₙNH₂ and —(CH₂)ₘC(═O)NR₅(CH₂)ₙNH₂;
A₁ is —[C(R₂R₈)]ₘ— or ═CR₂[C(R₂R₈)]ₘ—, wherein if A₁ is ═CR₂[C(R₂R₈)]ₘ—, then a3 is 0;
A₂ is —(CH₂)ₘ—, —C(═X)—, —O(CH₂)ₙ—, —S(CH₂)ₙ—, —CH═CH—, —C(═N—OR₂)—, or —NR₂—;
A₃ is H, C₁-C₆ alkyl, a lone electron pair when D₈ is N, or A₃ is —CH₂-bonded to A₁, A₂ or R₁ to form a ring;
a1, a2 and a3 are independently equal to 0 or 1;
D₁ is selected from —CH₂—, —N(NHR₇)—, —CH(NHR₇)—, —CH[(CH₂)ₘNHR₇]—, —CH(R₂)—, and —CH(CH₂SH)—;
D₂, D₃, D₄, D₅ and D₆ are independently selected from the group consisting of —(CH₂)ₘ—, —CH(R₂)—, —CH(NHR₇)—, —N(R₅)—, —O—, —S—, —C(═X)—, —S(═O)— and —SO₂—, wherein any two atoms of D₂, D₃, D₄, D₅ and D₆ are optionally linked to form a three, four, five or six membered saturated ring;
D₇ is selected from N, ═C< where the carbon forms a double bond with an adjacent carbon in one of D₁-D₆, CH and CR₄;
D₈ is selected from C and N;
d1, d2, d3, d4, d5 and d6 are independently equal to 0 or 1;
X₁ and X₂ are each hydrogen or taken together are ═O or ═S,
or X₁ is hydrogen and X₂ is —O— or —S— bonded to R₁₀ to form a 5- or 6-membered heterocyclyl,
or X₁ is absent and X₂ is —O— or —S— bonded to R₁₀ to form a 5- or 6-membered heterocyclyl or heteroaryl, wherein when X₁ is absent, the bond to nitrogen represented by a dashed and solid line is a double bond;
each X is independently O or S;
Z₁ is an aryl, heteroaryl, carbocyclyl, or heterocyclyl;
z1 is 0 or 1;
if z1 is 0 then at least two from the group consisting of d1, d2, d3, d4, d5 and d6 are equal to 1, if z1 is 1 then at least one from the group consisting of d1, d2, d3, d4, d5 and d6 is equal to 1;
each n is independently an integer of 0 to 4; and
each m is independently an integer of 1 to 3.

In another embodiment, the compounds have the structure of formula (II)

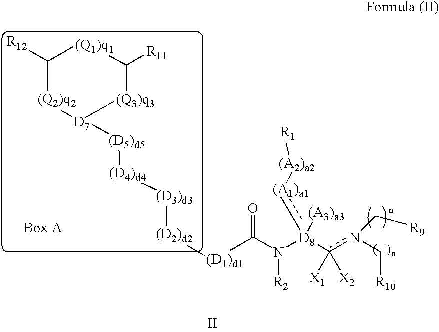

or a pharmaceutically acceptable salt or pro-drug thereof wherein;
each bond represented by a dashed and solid line represents a bond selected from the group consisting of a single bond and a double bond;
each R₁ is independently selected from C₁-C₆ alkyl, C₃-C₇ carbocyclyl, heterocyclyl, aryl and heteroaryl, each optionally substituted with up to 3 substituents independently selected from the group consisting of a halide, alkyl, carbocyclyl, —(CH₂)ₙaryl, —OR₂, —OR₁₀, —S(R₂)₂, —SO₂NHR₁₀, —(CH₂)ₙSH, —CF₃, —OCF₃, —N(R₂)₂, —NO₂, —CN, —CO₂alkyl, —CO₂aryl and —C(O)aryl;
each R₂ is independently selected from H and C₁-C₆ alkyl;
each R₄ is independently selected from —NHR₂, —(CH₂)ₙCHR₅R₆, —(CH₂)ₙNR₅R₆, —(CH₂)ₘC(═O)NR₅R₆, and —C(═NR₅)NR₅R₅;
each R₅ is independently selected from H and —(CH₂)ₘNH₂;
each R₆ is independently selected from —(CH₂)ₙNHR₇, —(CH₂)ₙNHC(═NH)NH₂, —(CH₂)ₙNHC(R₂)═NH, —(CH₂)ₙC(═NH)NH₂, and —(CH₂)ₙN⁺(CH₃)₃;
each R₇ is independently selected from H, alkyl, —C(═O)CH(R₁₃)(NH₂), —C(═O)A₂CH₂NH₂, Alanine, Arginine, Asparagine, Aspartic acid, Glutamic acid, Glutamine, Cysteine, Glycine, Histidine, Isoleucine, Leucine, Lysine, Methionine, Phenylalanine, Proline, Serine, Threonine, Tryptophan, Tyrosine, and Valine;
R₈ is selected from H, alkyl, aryl, SH and OH;
R₉ is selected from H, C₁-C₆ alkyl, C₃-C₁₀ carbocyclyl, heterocyclyl, aryl, heteroaryl, and —NHC(O)-aryl, each optionally substituted with up to 3 substituents independently selected from the group consisting of a halide, alkyl, carbocyclyl, —(CH₂)ₙR₁, —(CH═CH)ₙR₁, —OR₂, —OR₁, ═O, —S(R₂)₂, —SR₁, —SO₂NR₁R₂, —(CH₂)ₙSH, —CF₃, —OCF₃, —N(R₂)₂, —NO₂, —CN, —(C═X)R₁, —(C═X)R₂, —CO₂alkyl, —CO₂aryl, heteroaryl optionally substituted with C₁-C₆ alkyl, and aryl optionally substituted with C₁-C₆ alkyl;
R₁₀ is selected from C₁-C₆ alkyl, C₃-C₁₀ carbocyclyl, heterocyclyl, aryl, heteroaryl, and —NHC(O)-aryl, each optionally substituted with up to 3 substituents independently selected from the group consisting of a halide, alkyl, carbocyclyl, —(CH₂)ₙR₁, —OR₂, —OR₁, ═O, —S(R₂)₂, —SR₁, —SO₂NR₁R₂, —(CH₂)ₙSH, —CF₃, —OCF₃, —N(R₂)₂, —NO₂, —CN, —(C═X)R₁, —(C═X)R₂, —CO₂alkyl, and —CO₂aryl;
R₉ and R₁₀ are optionally linked to form a ring;
R₁₁ is selected from H, —(CH₂)ₙNHR₂ and —(CH₂)ₙCHR₅R₆;
R₁₂ is selected from —(CH₂)ₙNHR₂ and —(CH₂)ₙCHR₅R₆;
R₁₃ is selected from —(CH₂)ₙCHR₅(CH₂)ₙNH₂, —(CH₂)ₘNR₅(CH₂)ₙNH₂ and —(CH₂)ₘC(═O)NR₅(CH₂)ₙNH₂;
A₁ is —[C(R₂R₈)]ₘ— or ═CR₂[C(R₂R₈)]ₘ—, wherein if A₁ is ═CR₂[C(R₂R₈)]ₘ—, then a3 is 0;
A₂ is —(CH₂)ₘ—, —C(═X)—, —O(CH₂)ₙ—, —S(CH₂)ₙ—, —CH═CH—, —C(═N—OR₂)—, or —NR₂—;
A₃ is H, C₁-C₆ alkyl, a lone electron pair when D₈ is N, or A₃ is —CH₂-bonded to A₁, A₂ or R₁ to form a ring;
a1, a2 and a3 are independently equal to 0 or 1;
D₁ is selected from —CH₂—, —N(NHR₇)—, —CH(NHR₇)—, —CH[(CH₂)ₘNHR₇]—, —CH(R₂)—, and —CH(CH₂SH)—;
D₂, D₃, D₄, D₅ and D₆ are independently selected from the group consisting of —(CH₂)ₘ—, —CH(R₂)—, —CH(NHR₇)—, —N(R₅)—, —O—, —S—, —C(═X)—, —S(═O)— and —SO₂—, wherein any two atoms of D₂, D₃, D₄, D₅ and D₆ are optionally linked to form a three, four, five or six membered saturated ring;
D₇ is selected from N, =C< where the carbon forms a double bond with an adjacent carbon in one of D₁-D₆, CH and CR₄;
D₈ is selected from C and N;
d1, d2, d3, d4, d5 and d6 are independently equal to 0 or 1;
Q₁ is selected from —CH₂—, —N(R₂)N(R₂)—, and —N(R₂)—;
Q₂ and Q₃ are independently selected from the group consisting of —CH₂— and —N(R₂)—;
with the proviso that no more than one of Q₁, Q₂, and Q₃ comprises a nitrogen;
q1, q2, and q3 are independently equal to 0 or 1;
X₁ and X₂ are each hydrogen or taken together are ═O or ═S,
or X₁ is hydrogen and X₂ is —O— or —S— bonded to R₁₀ to form a 5- or 6-membered heterocyclyl,
or X₁ is absent and X₂ is —O— or —S— bonded to R₁₀ to form a 5- or 6-membered heterocyclyl or heteroaryl, wherein when X₁ is absent, the bond to nitrogen represented by a dashed and solid line is a double bond;
each X is independently O or S;
Z₁ is an aryl, heteroaryl, carbocyclyl, or heterocyclyl;
z1 is 0 or 1;
if z1 is 0 then at least two from the group consisting of d1, d2, d3, d4, d5 and d6 are equal to 1, if z1 is 1 then at least one from the group consisting of d1, d2, d3, d4, d5 and d6 is equal to 1;
each n is independently an integer of 0 to 4; and
each m is independently an integer of 1 to 3.

In another embodiment, the compounds have the structure of formula (III):

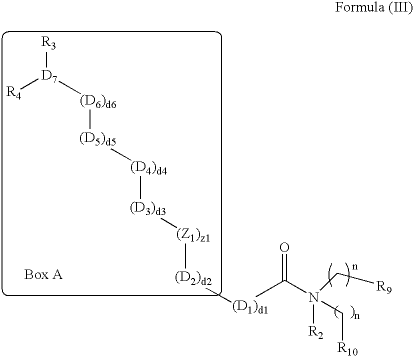

or a pharmaceutically acceptable salt or pro-drug thereof wherein;
each bond represented by a dashed and solid line represents a bond selected from the group consisting of a single bond and a double bond;
each R₁ is independently selected from C₁-C₆ alkyl, C₃-C₇ carbocyclyl, heterocyclyl, aryl and heteroaryl, each optionally substituted with up to 3 substituents independently selected from the group consisting of a halide, alkyl, carbocyclyl, —(CH₂)ₙaryl, —OR₂, —OR₁₀, —S(R₂)₂, —SO₂NHR₁₀, —(CH₂)ₙSH, —CF₃, —OCF₃, —N(R₂)₂, —NO₂, —CN, —CO₂alkyl, —CO₂aryl and —C(O)aryl;
each R₂ is independently selected from H and C₁-C₆ alkyl;
R₃ is selected from —(CH₂)ₙCHR₅R₆, —(CH₂)ₙNR₅R₆, and —(CH₂)ₘC(═O)NR₅R₆;
each R₄ is independently selected from —NHR₂, —(CH₂)ₙCHR₅R₆, —(CH₂)ₙNR₅R₆, —(CH₂)ₘC(═O)NR₅R₆, and —C(═NR₅)NR₅R₅;
each R₅ is independently selected from H and —(CH₂)ₘNH₂;
each R₆ is independently selected from —(CH₂)ₙNHR₇, —(CH₂)ₙNHC(═NH)NH₂, —(CH₂)ₙNHC(R₂)═NH, —(CH₂)ₙC(═NH)NH₂, and —(CH₂)ₙN⁺(CH₃)₃;
each R₇ is independently selected from H, alkyl, —C(═O)CH(R₁₃)(NH₂), —C(═O)A₂CH₂NH₂, Alanine, Arginine, Asparagine, Aspartic acid, Glutamic acid, Glutamine, Cysteine, Glycine, Histidine, Isoleucine, Leucine, Lysine, Methionine, Phenylalanine, Proline, Serine, Threonine, Tryptophan, Tyrosine, and Valine;
R₈ is selected from H, alkyl, aryl, SH and OH;
R₉ is selected from H, C₁-C₆ alkyl, C₃-C₁₀ carbocyclyl, heterocyclyl, aryl, heteroaryl, and —NHC(O)-aryl, each optionally substituted with up to 3 substituents independently selected from the group consisting of a halide, alkyl, carbocyclyl, —(CH₂)ₙR₁, —(CH═CH)ₙR₁, —OR₂, —OR₁, ═O, —S(R₂)₂, —SR₁, —SO₂NR₁R₂, —(CH₂)ₙSH, —CF₃, —OCF₃, —N(R₂)₂, —NO₂, —CN, —(C═X)R₁, —(C═X)R₂, —CO₂alkyl, —CO₂aryl, heteroaryl optionally substituted with C₁-C₆ alkyl, and aryl optionally substituted with C₁-C₆ alkyl;
R₁₀ is selected from C₁-C₆ alkyl, C₃-C₁₀ carbocyclyl, heterocyclyl, aryl, heteroaryl, and —NHC(O)-aryl, each optionally substituted with up to 3 substituents independently selected from the group consisting of a halide, alkyl, carbocyclyl, —(CH₂)ₙR₁, —OR₂, —OR₁, ═O, —S(R₂)₂, —SR₁, —SO₂NR₁R₂, —(CH₂)ₙSH, —CF₃, —OCF₃, —N(R₂)₂, —NO₂, —CN, —(C═X)R₁, —(C═X)R₂, —CO₂alkyl, and —CO₂aryl;
R₉ and R₁₀ are optionally linked to form a ring;
R₁₁ is selected from H, —(CH₂)ₙNHR₂ and —(CH₂)ₙCHR₅R₆;
R₁₂ is selected from —(CH₂)ₙNHR₂ and —(CH₂)ₙCHR₅R₆;
R₁₃ is selected from —(CH₂)ₙCHR₅(CH₂)ₙNH₂, —(CH₂)ₘNR₅(CH₂)ₙNH₂ and —(CH₂)ₘC(═O)NR₅(CH₂)ₙNH₂;
D₁ is selected from —CH₂—, —N(NHR₇)—, —CH(NHR₇)—, —CH[(CH₂)ₘNHR₇]—, —CH(R₂)—, and —CH(CH₂SH)—;
D₂, D₃, D₄, D₅ and D₆ are independently selected from the group consisting of —(CH₂)ₘ—, —CH(R₂)—, —CH(NHR₇)—, —N(R₅)—, —O—, —S—, —C(═X)—, —S(═O)— and —SO₂—, wherein any two atoms of D₂, D₃, D₄, D₅ and D₆ are optionally linked to form a three, four, five or six membered saturated ring;
D₇ is selected from N, ═C< where the carbon forms a double bond with an adjacent carbon in one of D₁-D₆, CH and CR₄;
D₈ is selected from C and N;
d1, d2, d3, d4, d5 and d6 are independently equal to 0 or 1;
each X is independently O or S;
Z₁ is an aryl, heteroaryl, carbocyclyl, or heterocyclyl;
z1 is 0 or 1;
if z1 is 0 then at least two from the group consisting of d1, d2, d3, d4, d5 and d6 are equal to 1, if z1 is 1 then at least one from the group consisting of d1, d2, d3, d4, d5 and d6 is equal to 1;
each n is independently an integer of 0 to 4; and
each m is independently an integer of 1 to 3.

In another embodiment, the compounds have the structure of formula (IV):

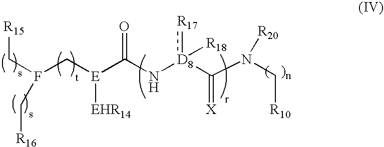

or a pharmaceutically acceptable salt or prodrug thereof, wherein:
D₈ is selected from C and N;
each E is independently CH or N;
F is selected from the group consisting of:

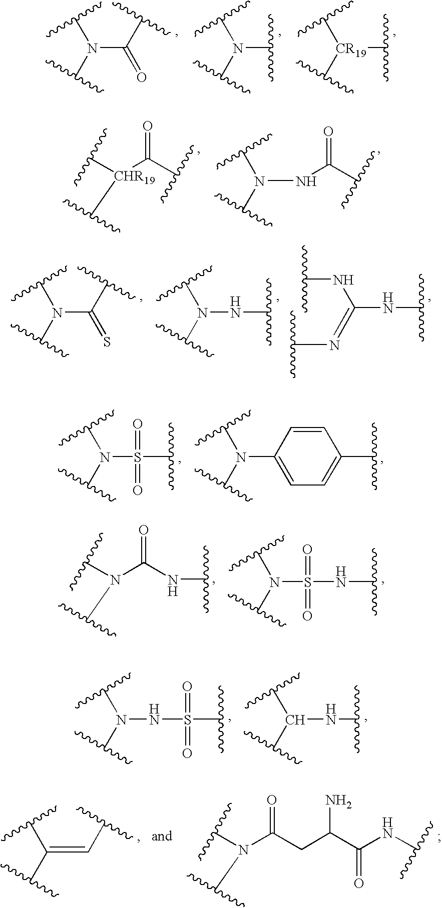

X is O or S;
R₁₀ is selected from carbocyclyl, heterocyclyl, aryl, heteroaryl, —NHC(O)-aryl, and aralkyl, each optionally substituted with up to 3 substituents independently selected from the group consisting of a halide, alkyl, —CF₃, —OCF₃, —NO₂, —CN, —OH, ═O, carbocyclyl, heterocyclyl, aryl optionally substituted with halide or —OH, heteroaryl optionally substituted with alkyl, —O-aryl optionally substituted with —O—C₁-C₆ alkyl, —O-heteroaryl, —O-heterocyclyl, —SO₂NH-heteroaryl, —O—C₁-C₆ alkyl, —SO₂NEt₂, SMe, di(C₁-C₆)alkylamino, —CH₂-heterocyclyl optionally substituted with alkyl, —CH₂-aryl, —C(O)aryl, and —CH═CH-aryl;
R₁₄ is selected from H, —C(O)—CH(Me)(NH₂), —C(O)—CH(CH₂OH)(NH₂), and —(CH₂)ₜNH₂;
R₁₅ and R₁₆ are independently selected from —NH₂, —NHC(═NH)NH₂, —N⁺(CH₃)₃, —NHCH₂CH₂NH₂, —N(CH₂CH₂NH₂)₂, —C(O)N(CH₂CH₂NH₂)₂, —CH(CH₂NH₂)₂, and —CH₂(NH₂)(CH₂NH₂), or R₁₅ and R₁₆ together with F form a heterocyclyl substituted with at least two substituents independently selected from —(CH₂)ₙNH₂, —(CH₂)ₙNHC(═NH)NH₂—(CH₂)ₙN⁺(CH₃)₃, —(CH₂)ₙNHCH₂CH₂NH₂, —(CH₂)ₙN(CH₂CH₂NH₂)₂, —(CH₂)ₙC(O)N(CH₂CH₂NH₂)₂, and —(CH₂)SCH(CH₂NH₂)₂;
R₁₇ is selected from alkyl, aralkyl, heteroaralkyl, carbocyclyl-alkyl, heterocyclyl-alkyl, aryl, and carbocyclyl, each optionally substituted with up to 3 substituents independently selected from the group consisting of —CF₃, —OH, —OCF₃, halide, —CN, alkyl, —O-aralkyl, aryl, —S(CH₃)₂, —C(O)aryl, —S-aralkyl optionally substituted with —OMe, ═O, and ═N—OH;
R₁₈ is H, alkyl, or absent,
or R₁₇ together with R₁₈ form a carbocyclyl optionally substituted with aryl or heteroaryl;
R₁₉ is H, —CH₂NH₂, or —CH₂CH₂NH₂;
R₂₀ is H or alkyl;
each t is independently an integer from 1 to 4;
each s is independently an integer from 0 to 3;
r is 0 or 1; and
n is an integer from 0 to 4.

Some embodiments of the compounds of formulas (I) — (VI) are shown below. Although the structures are shown with defined configurations at selected stereocenters, the shown stereochemistries are not meant to be limiting and all possible stereoisomers of the shown structures are contemplated. Compounds of any absolute and relative configurations at the stereocenters as well as mixtures of enantiomers and diastereoisomers of any given structure are also contemplated.

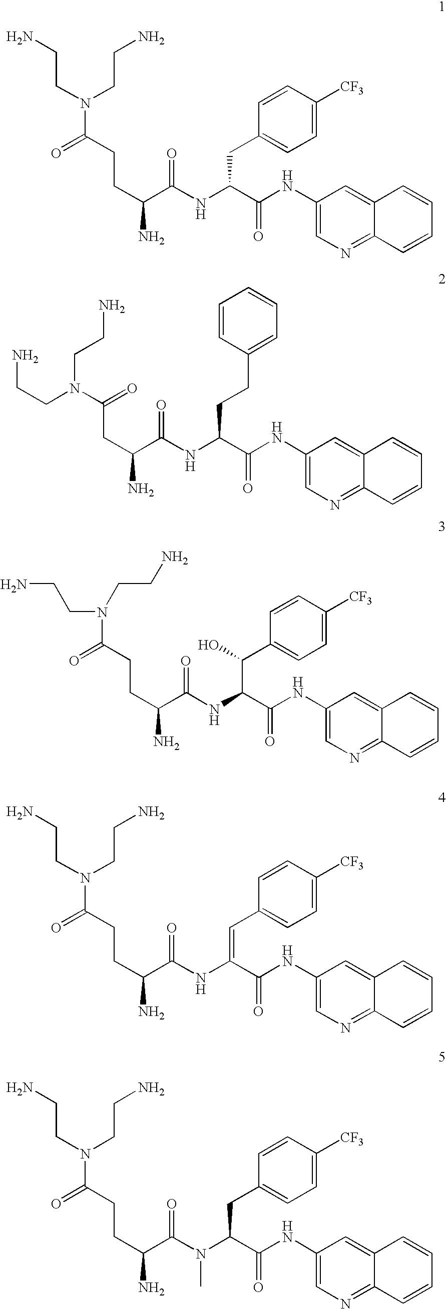

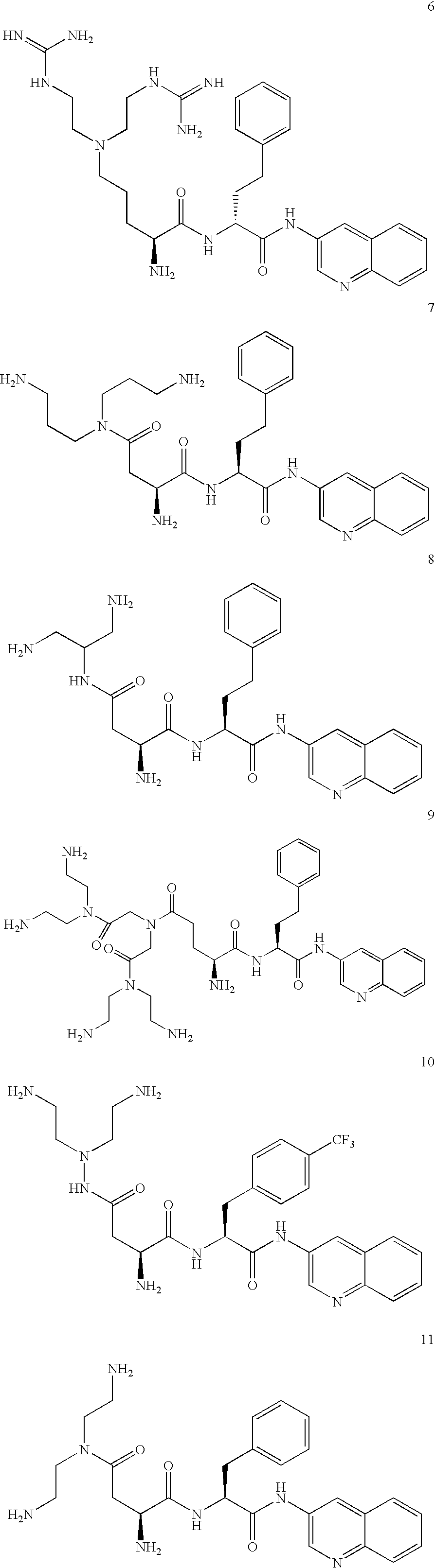

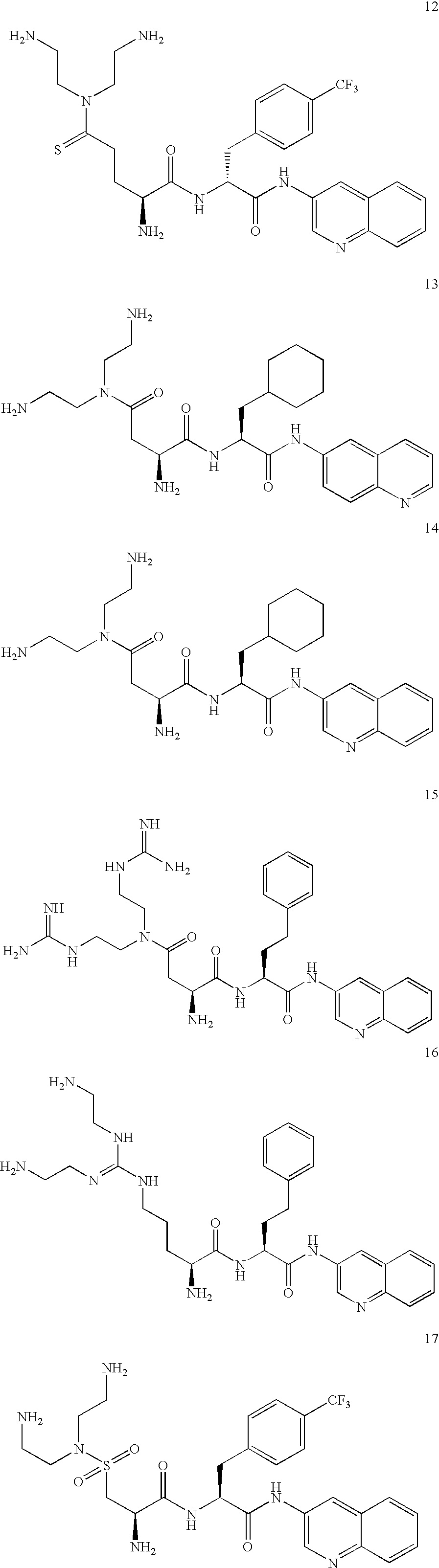

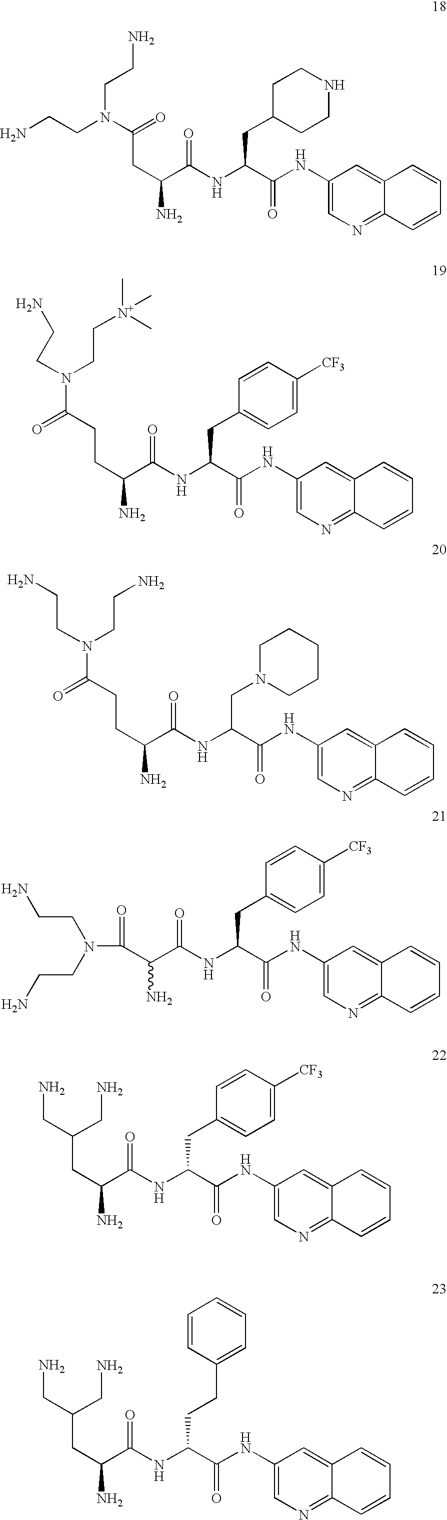

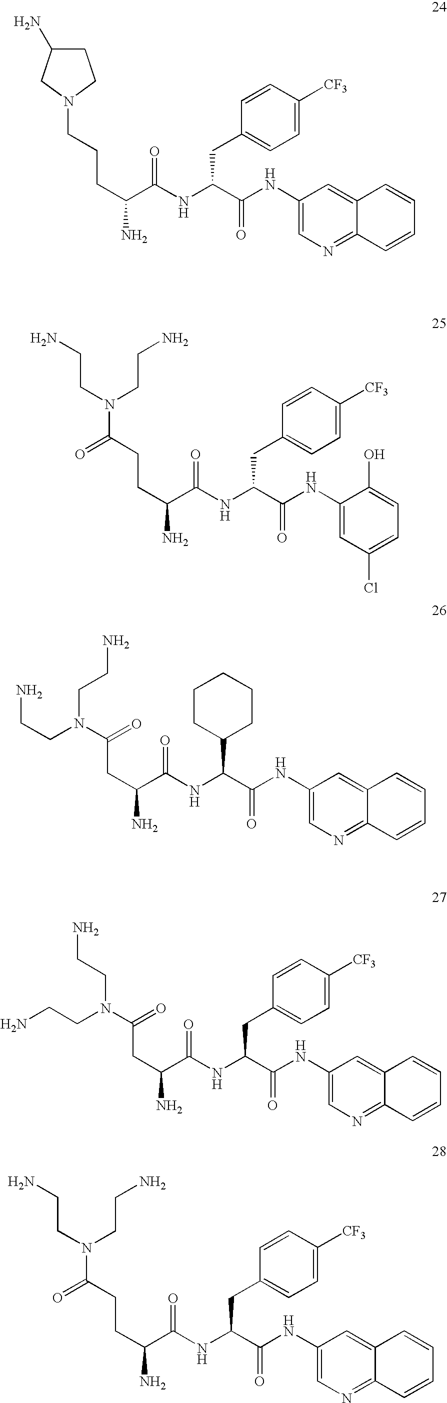

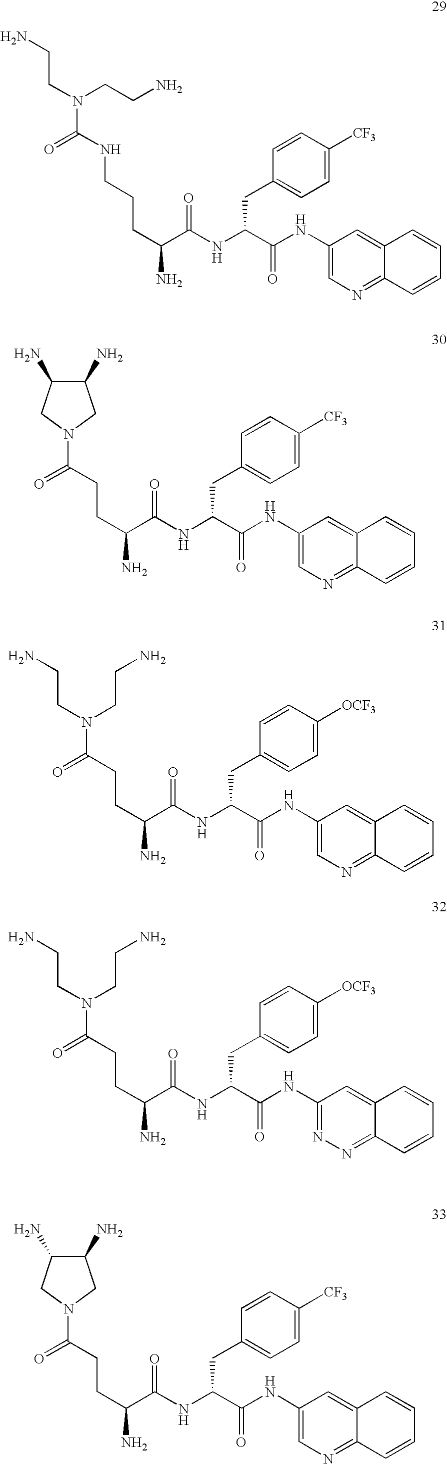

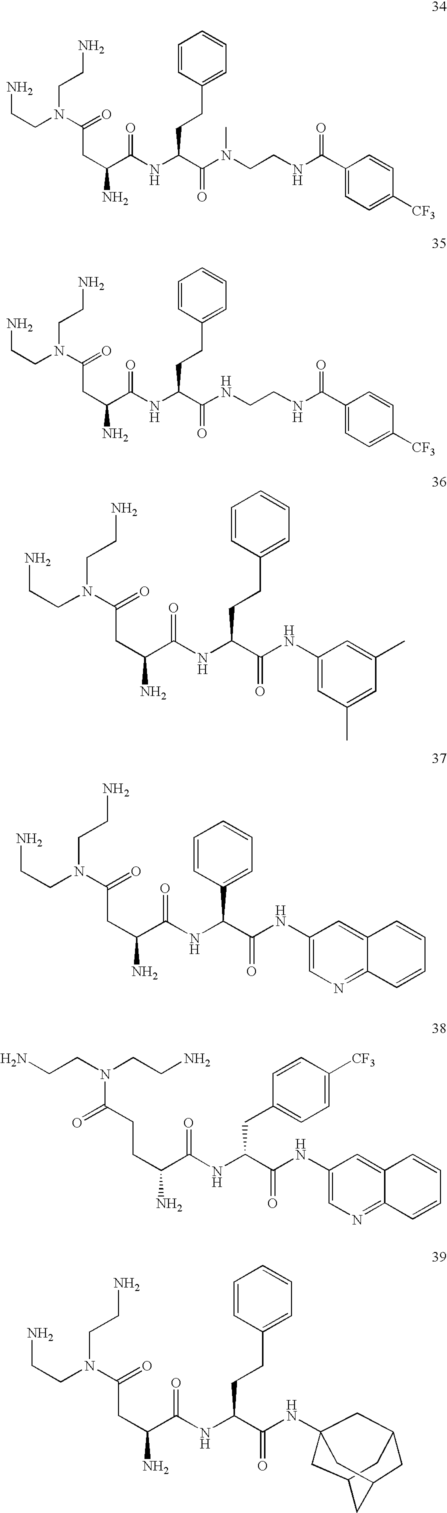

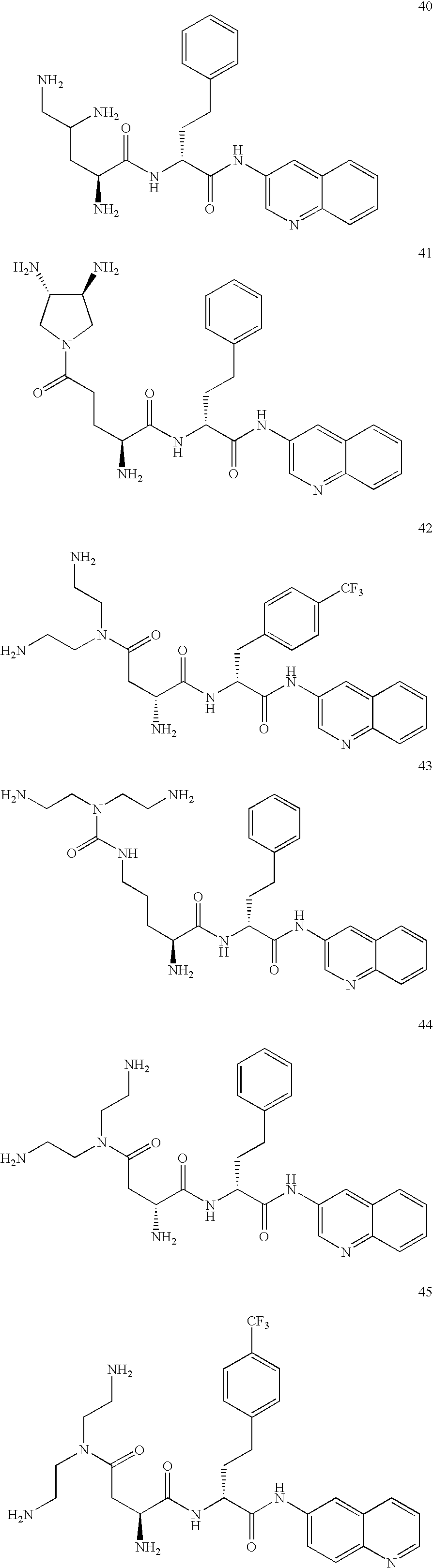

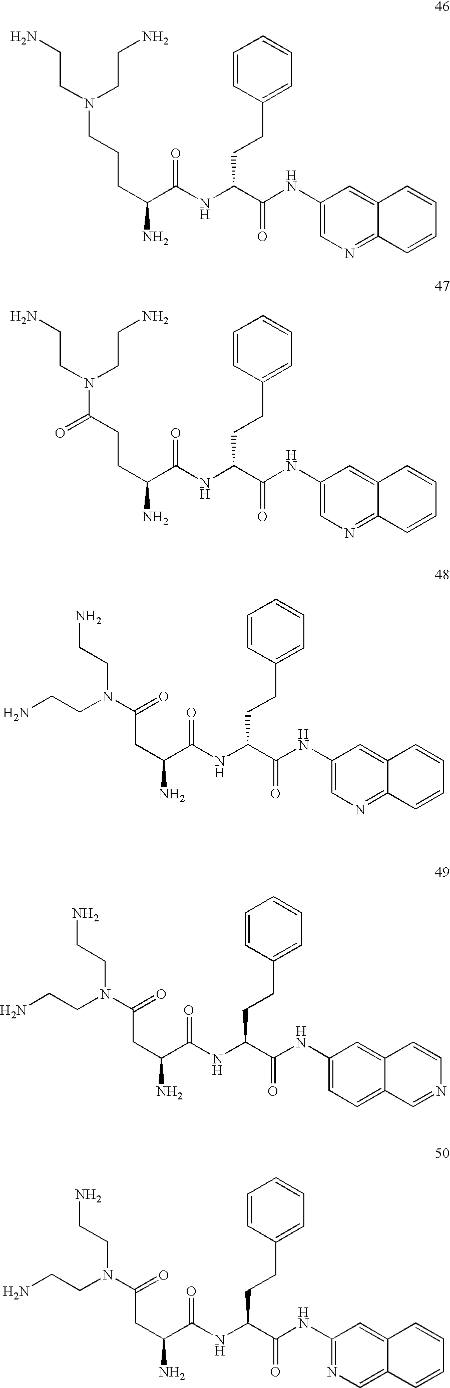

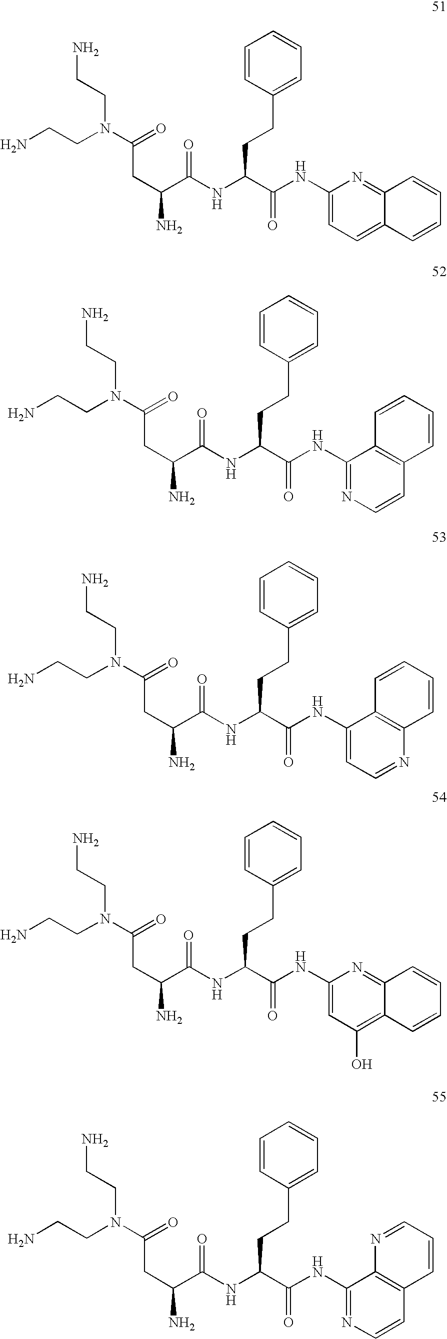

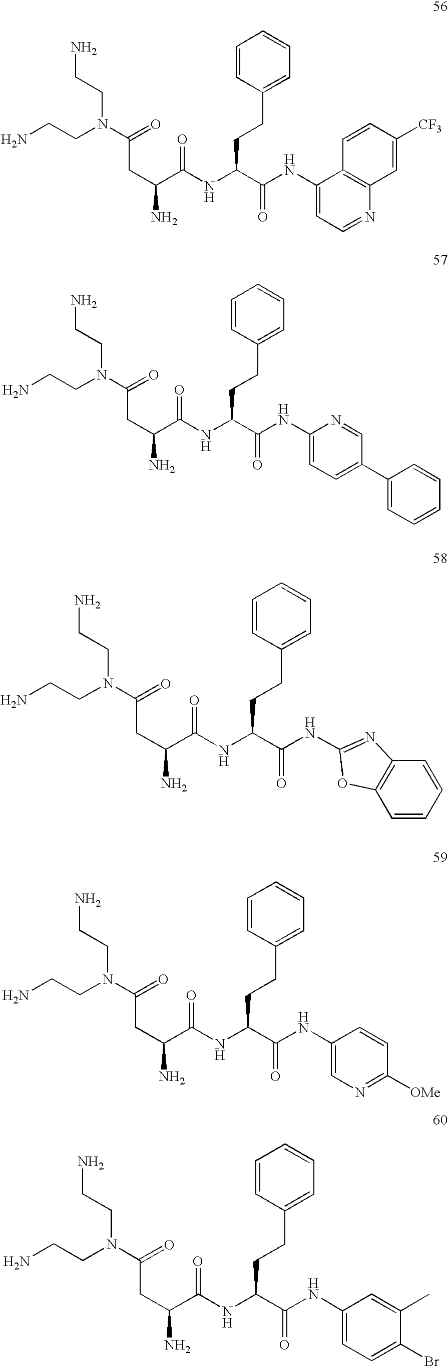

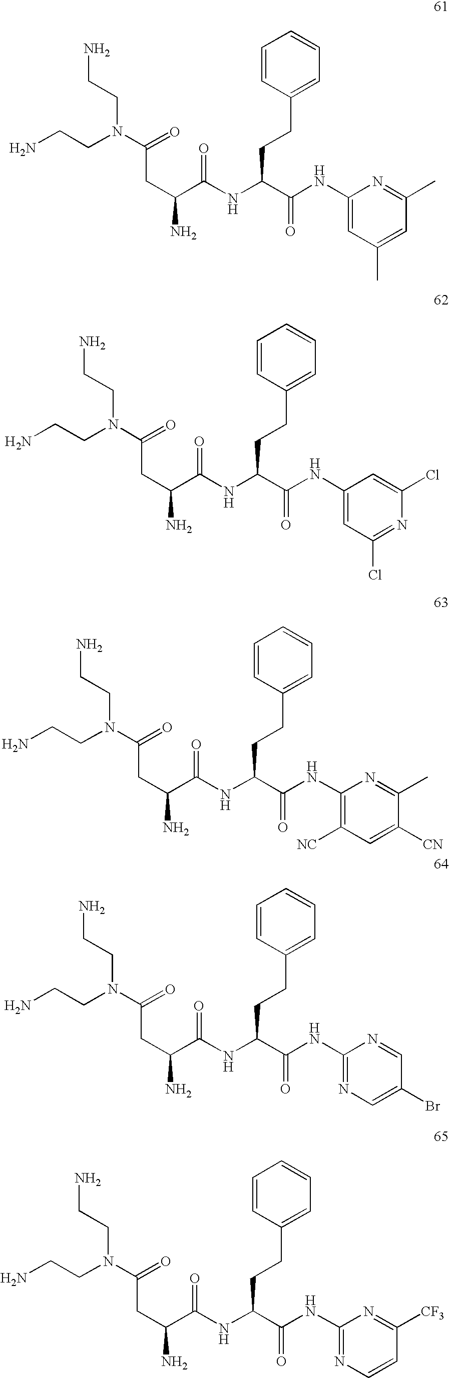

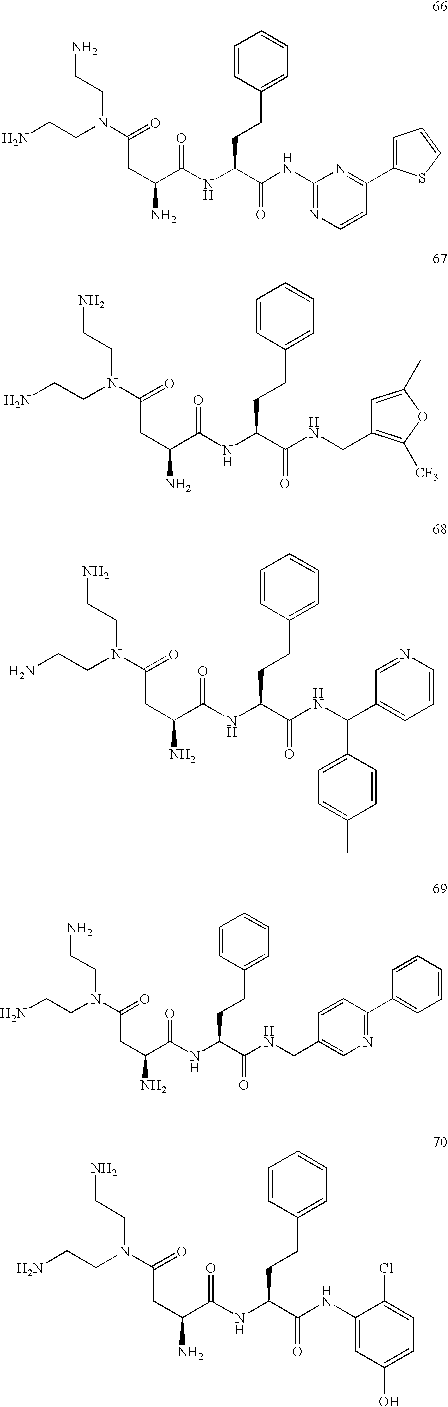

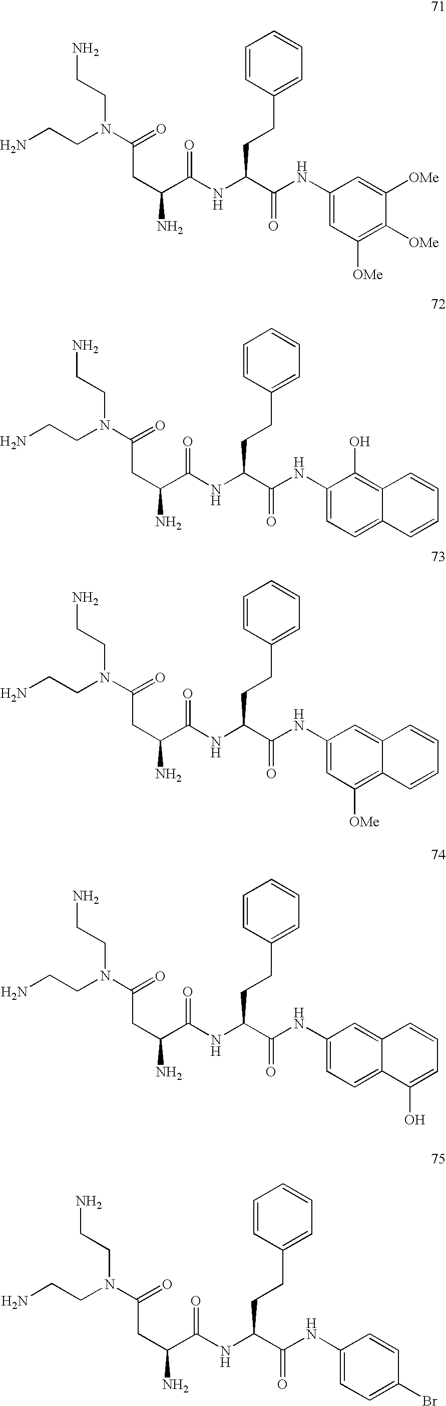

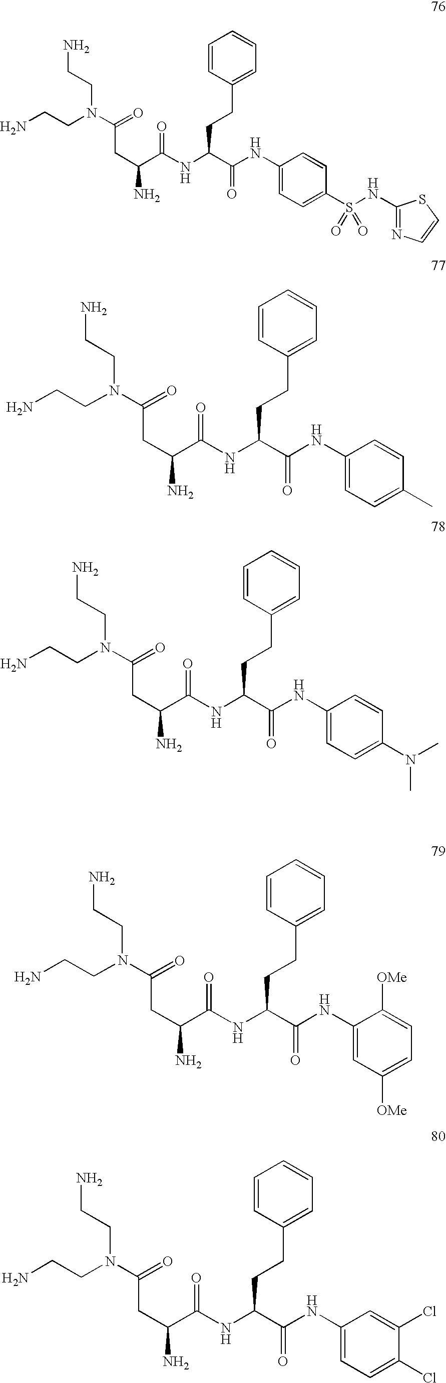

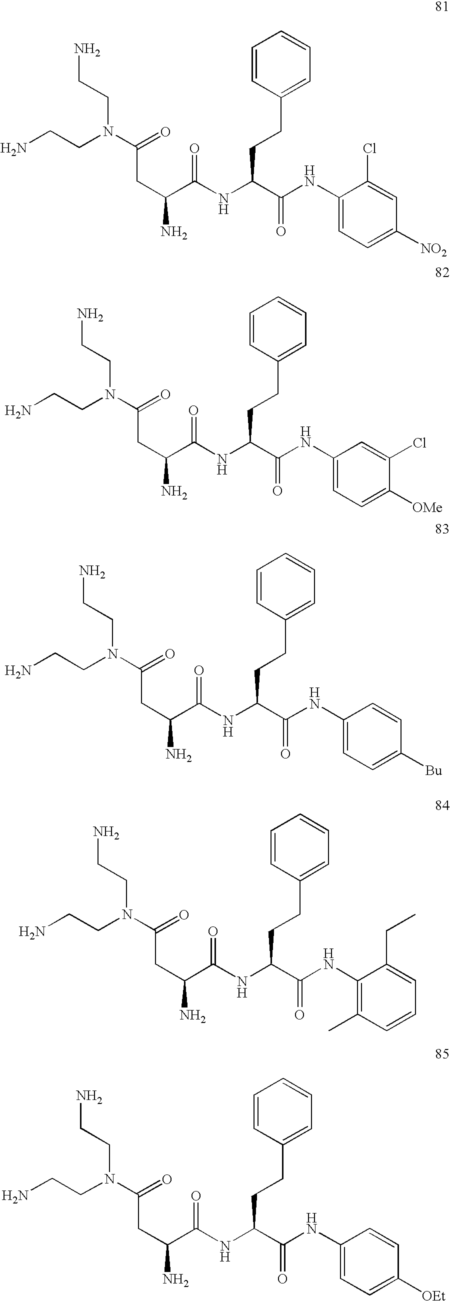

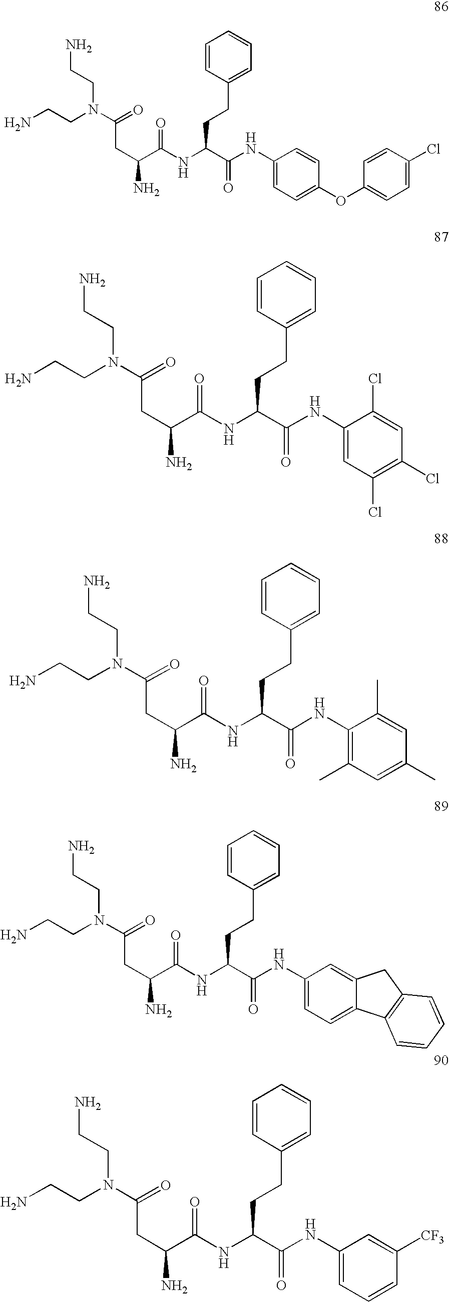

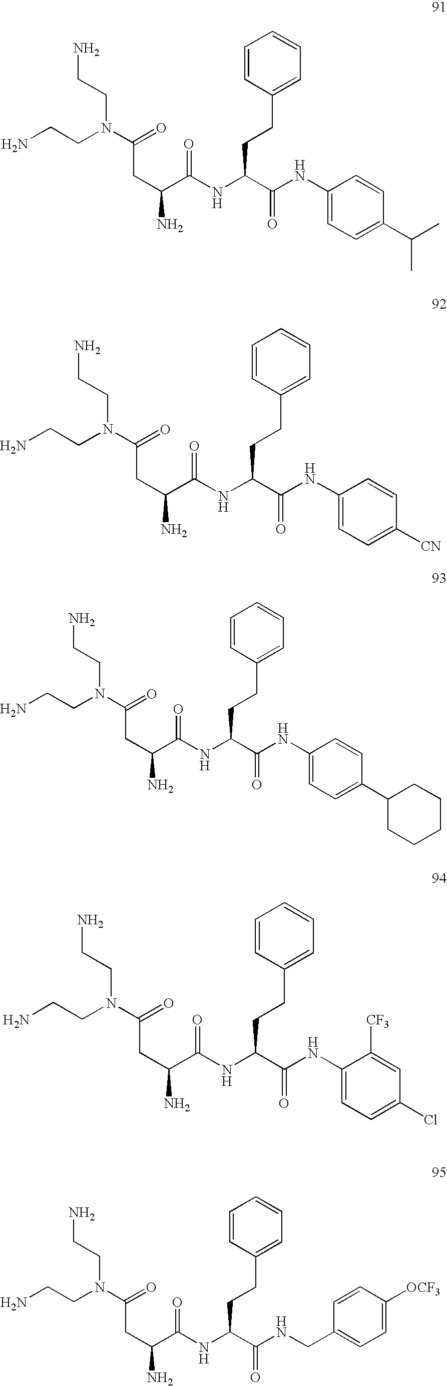

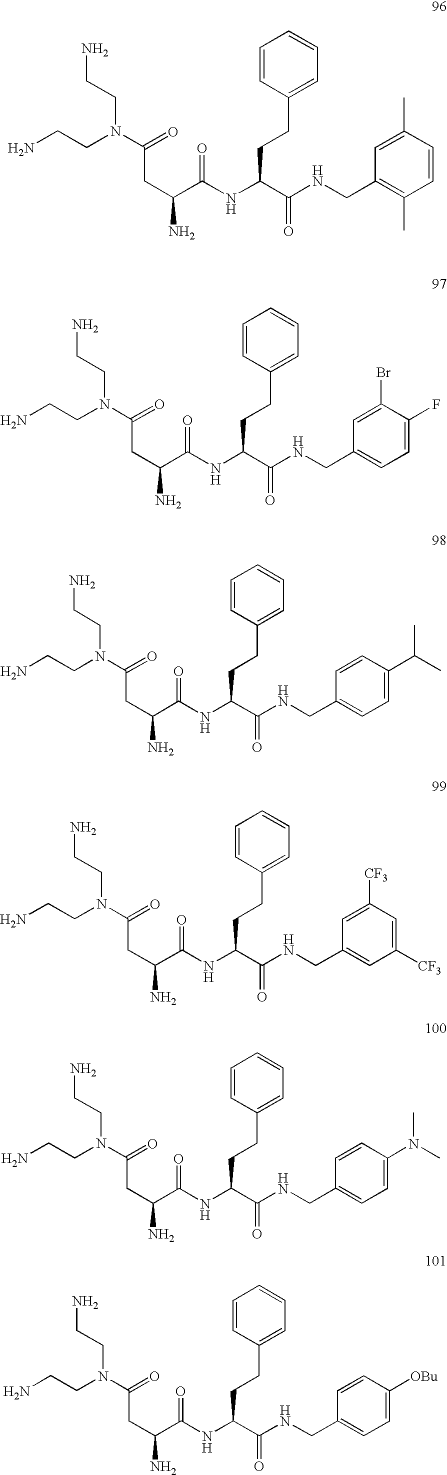

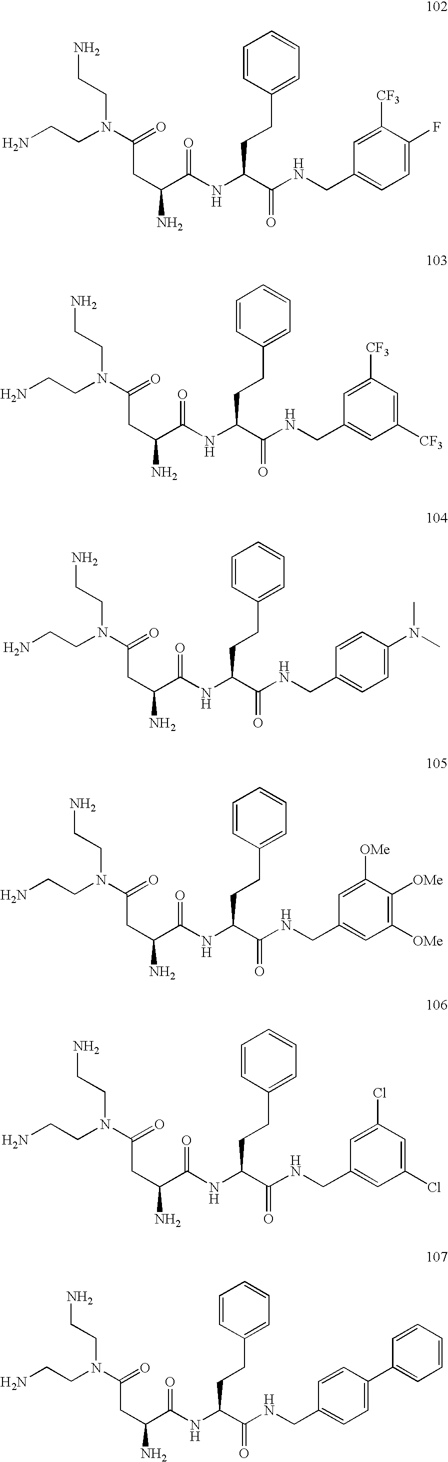

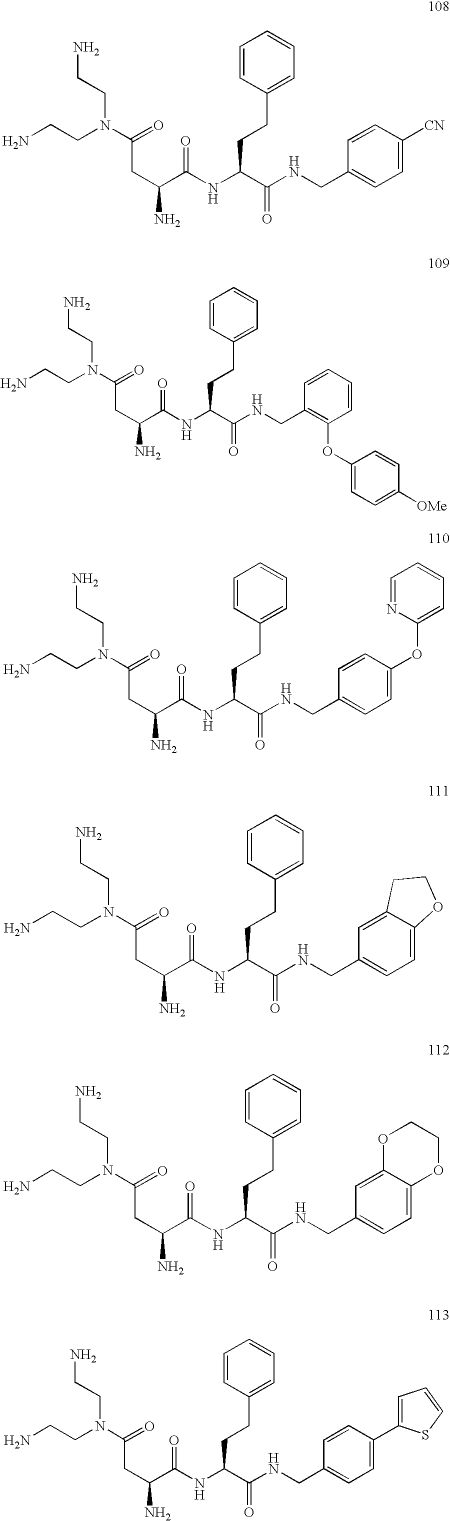

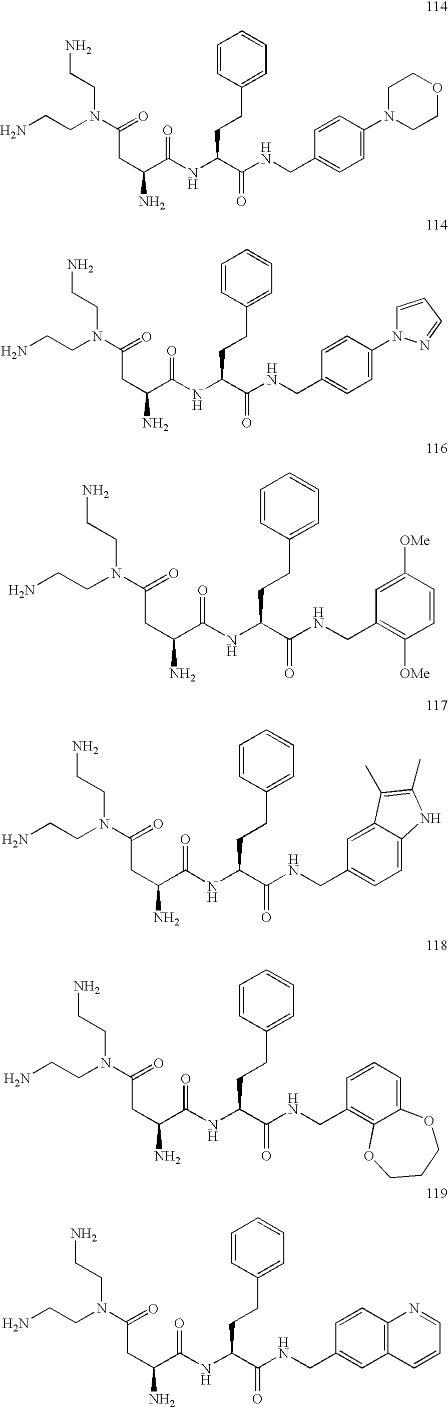

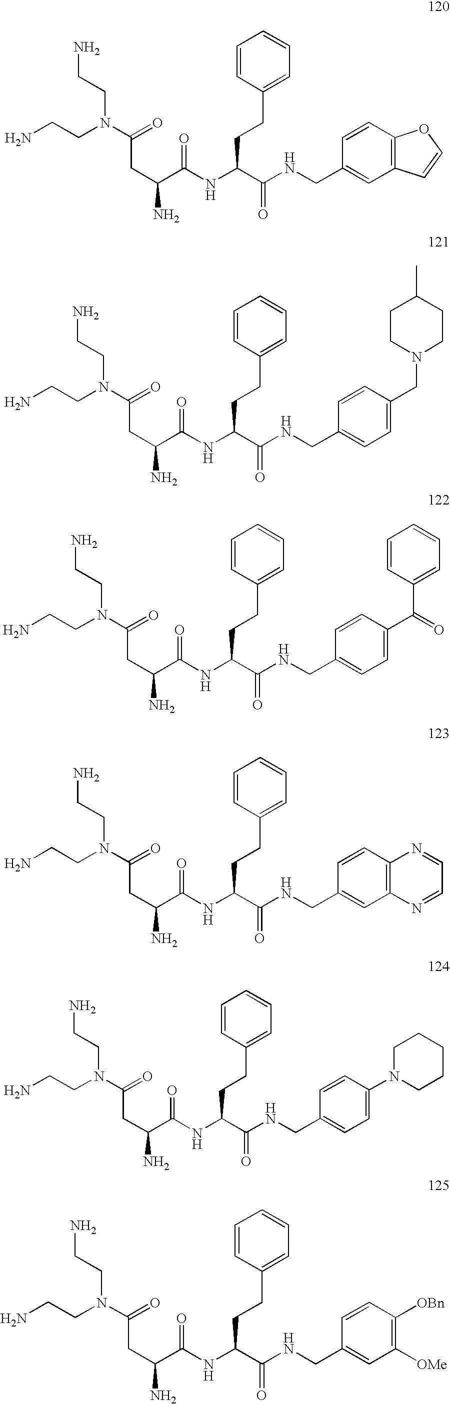

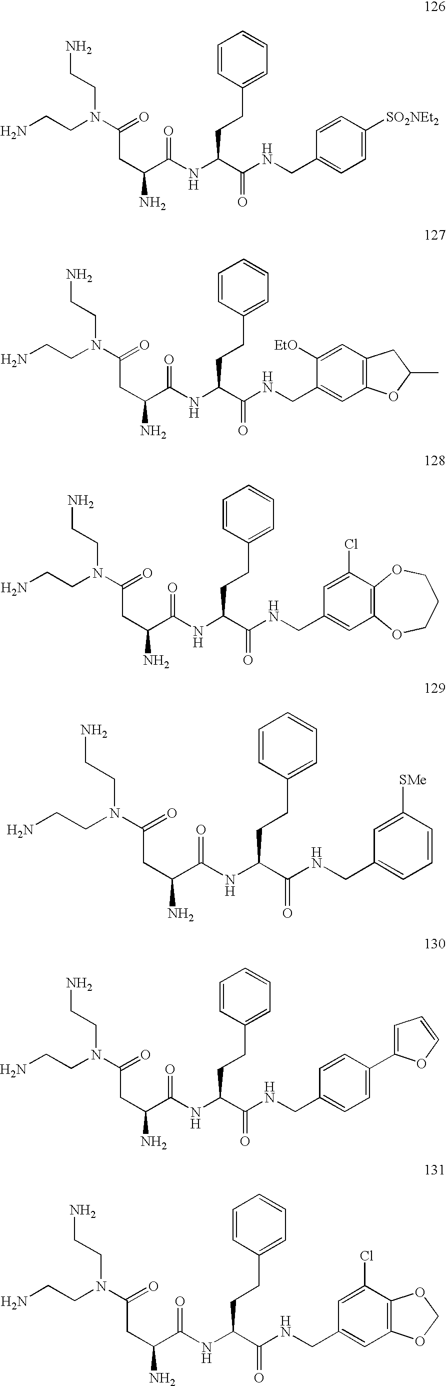

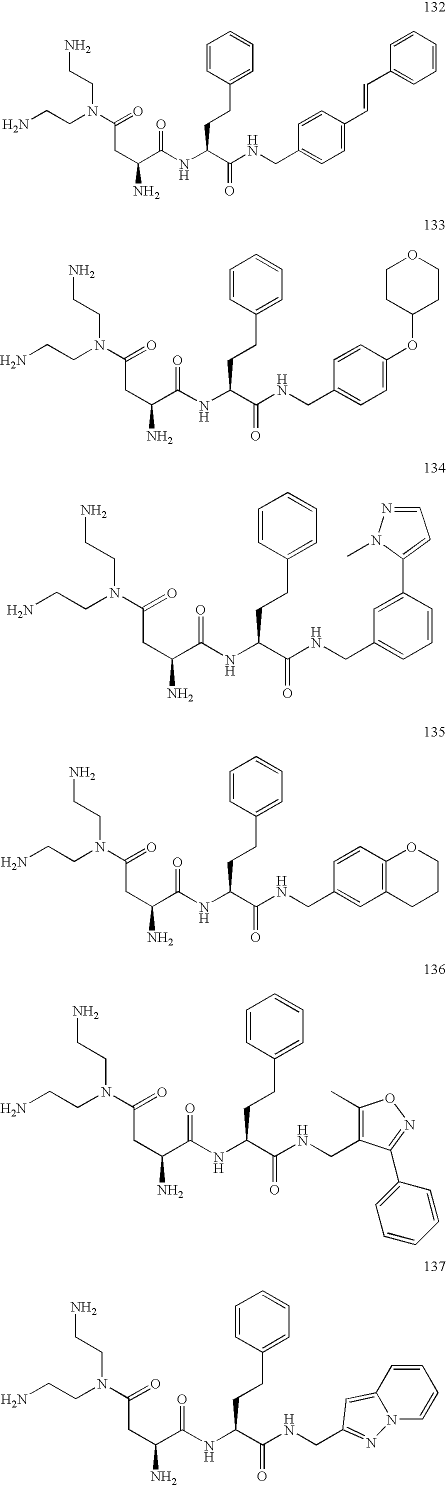

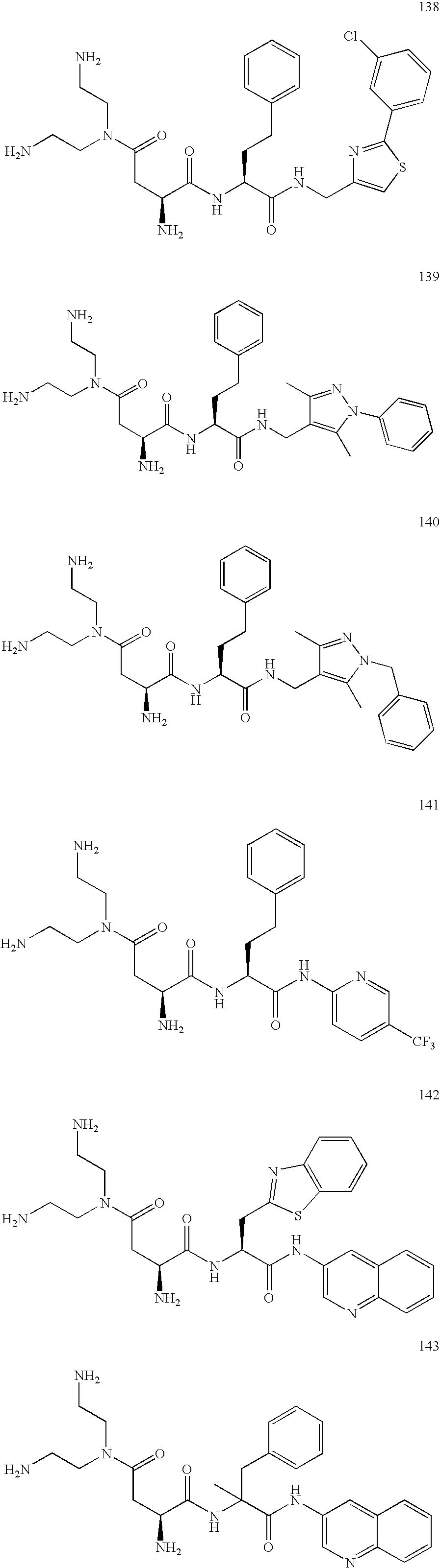

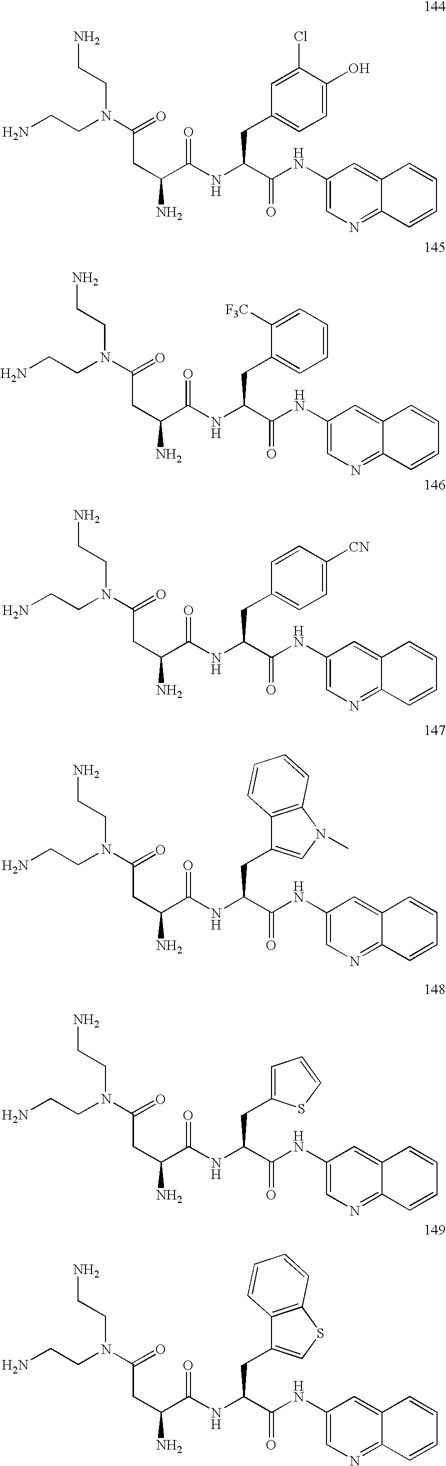

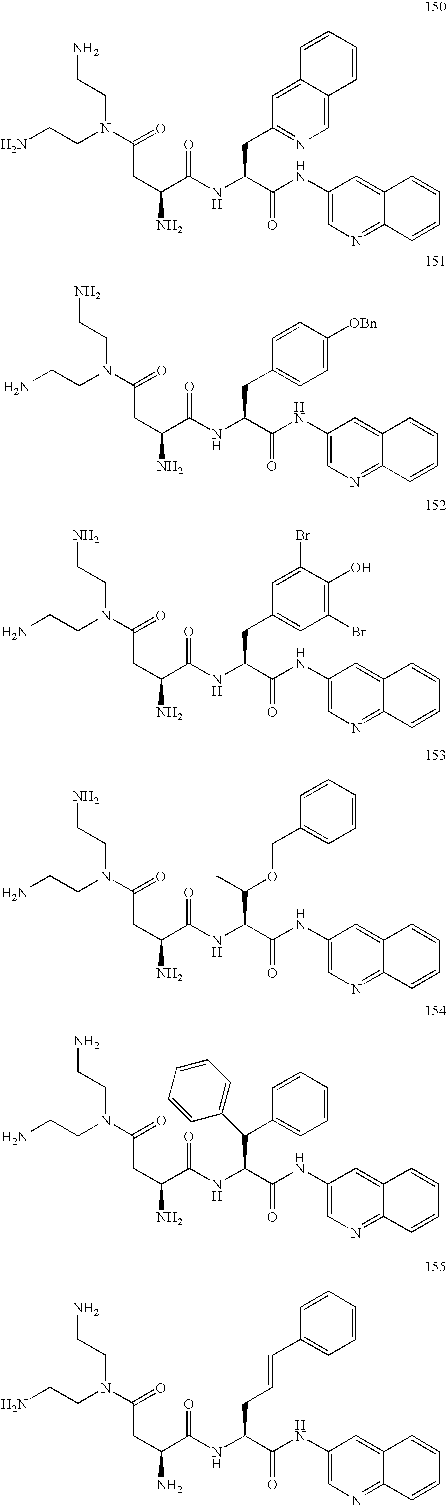

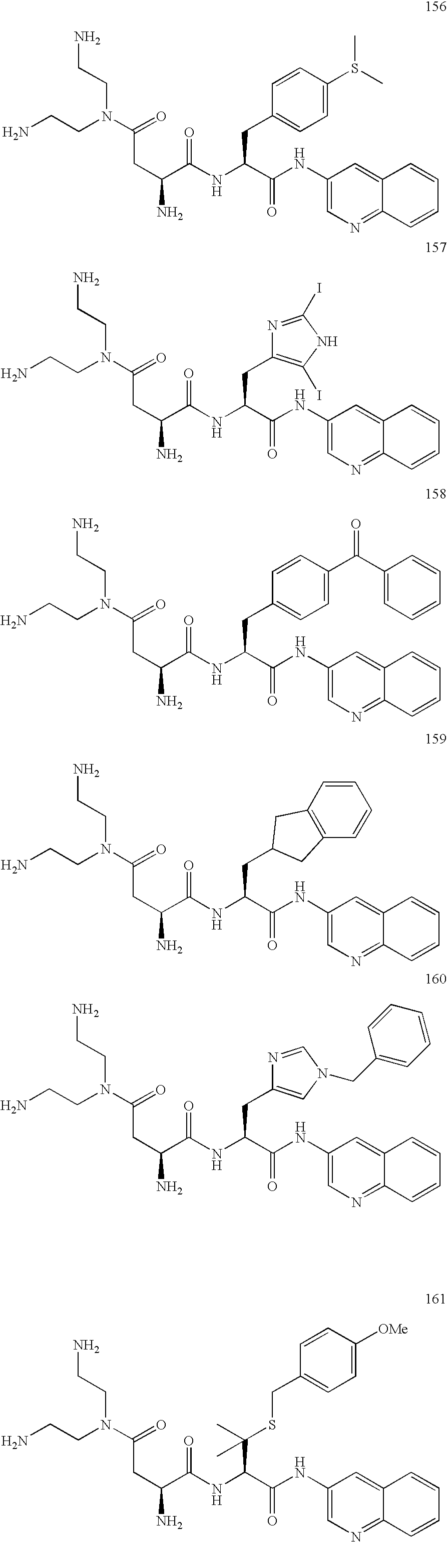

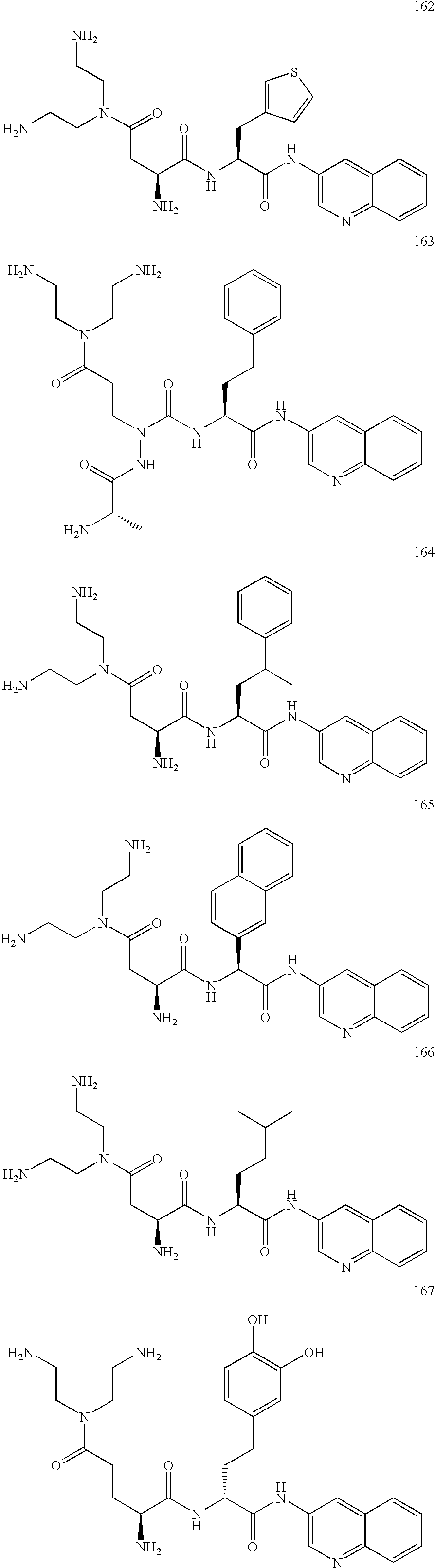

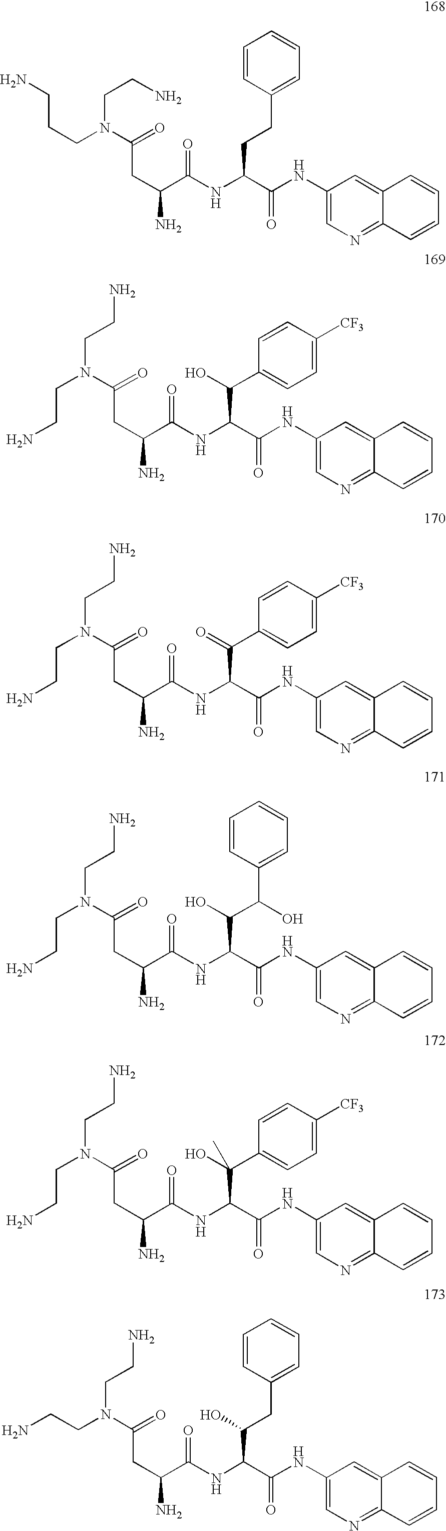

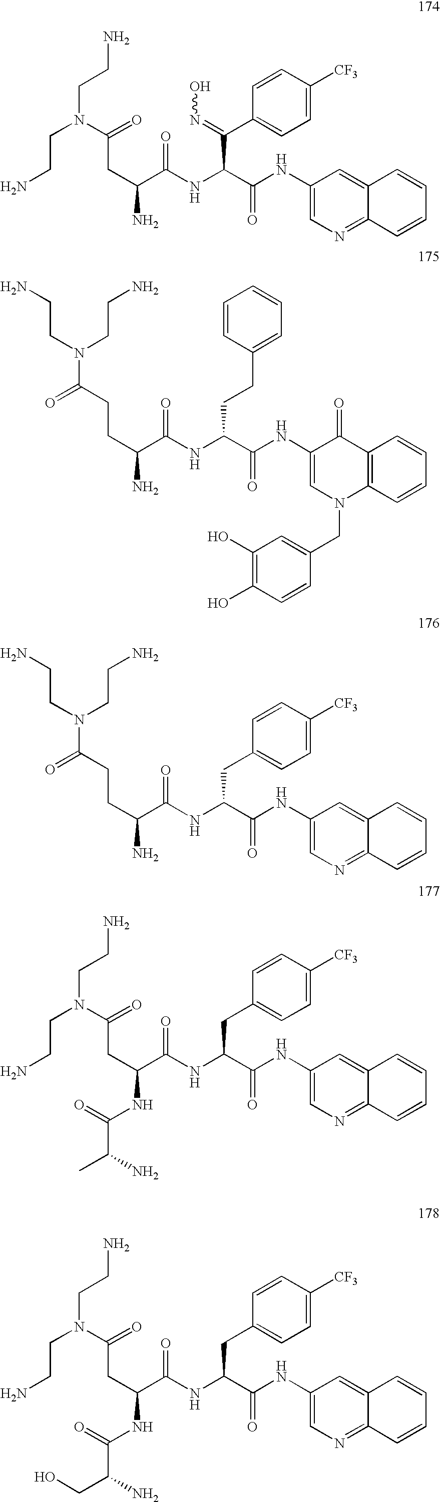

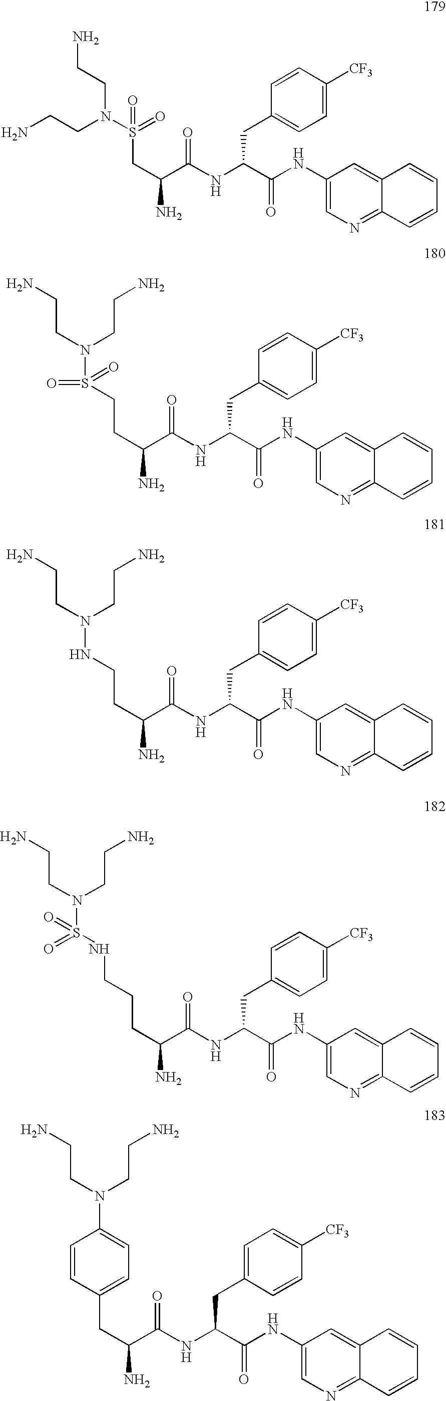

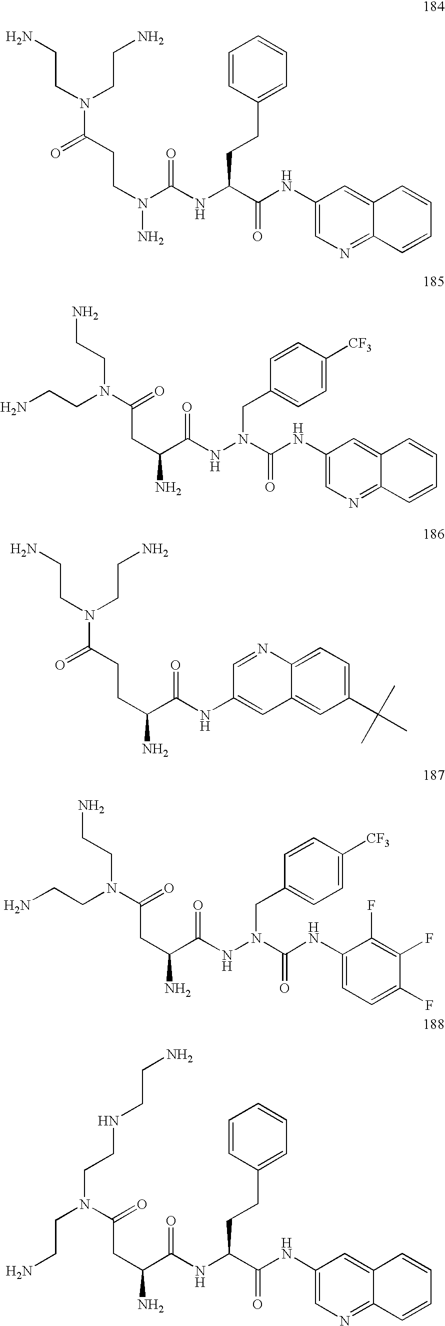

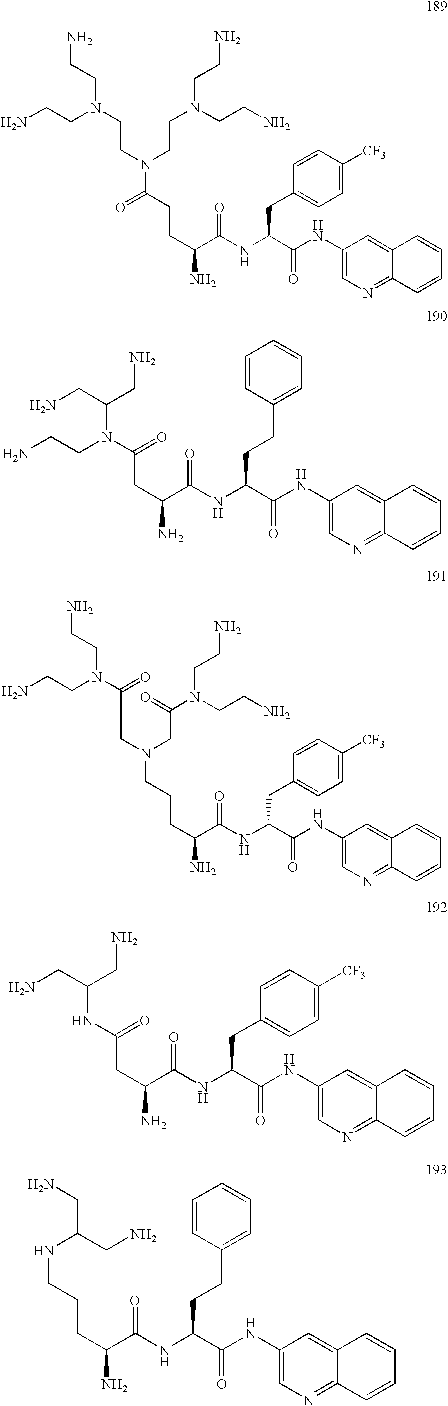

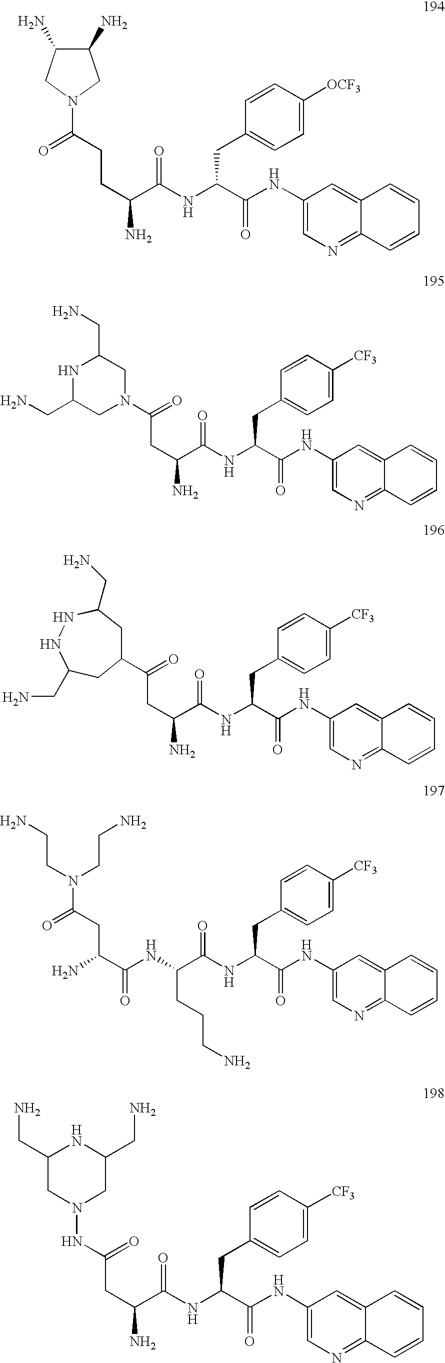

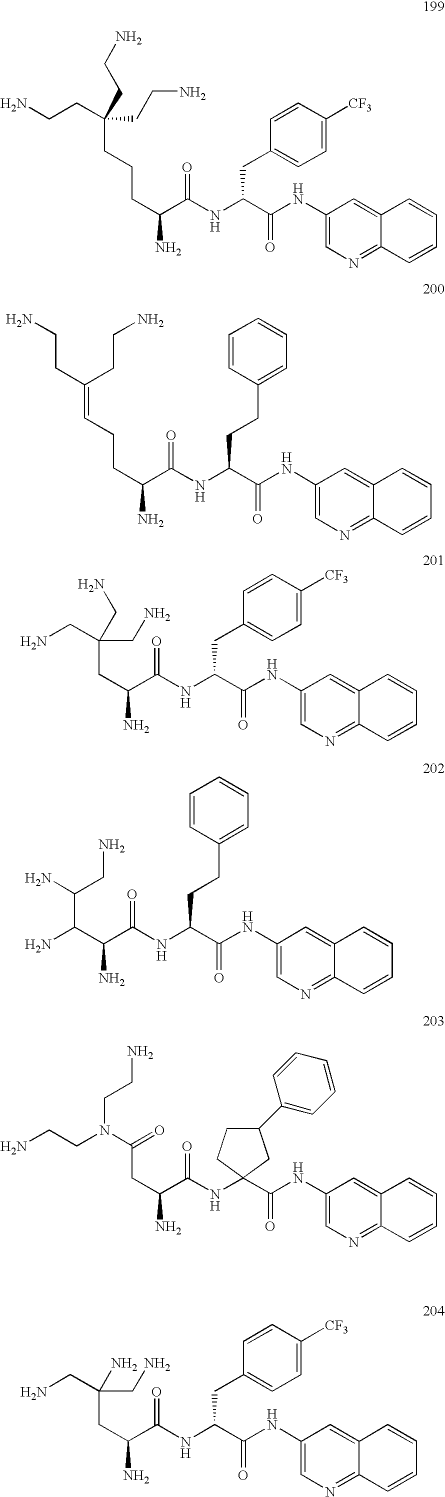

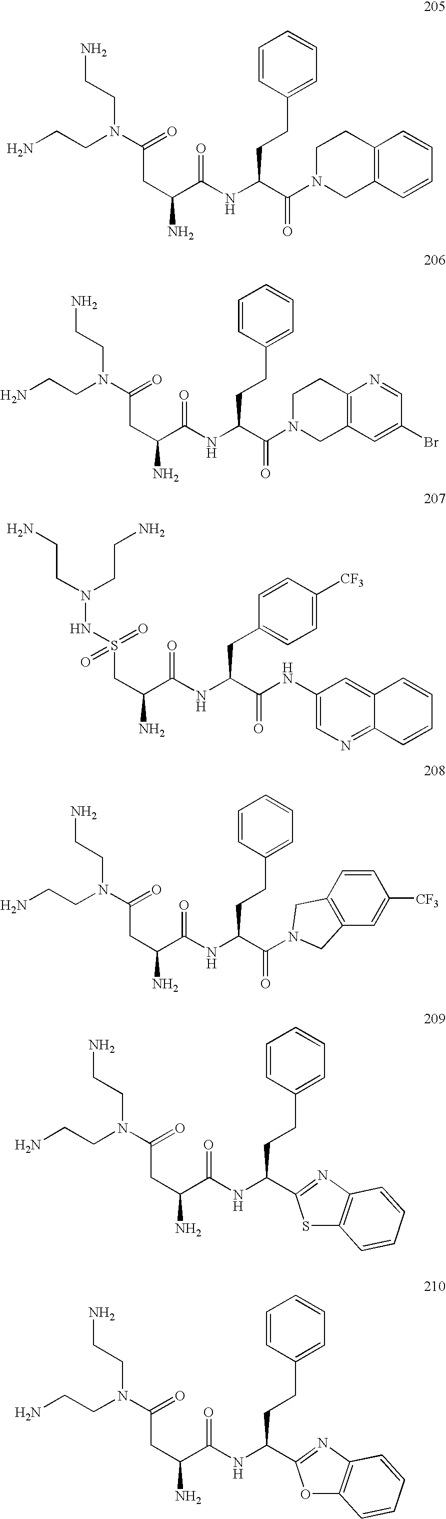

### Compound Preparation

The starting materials used in preparing the compounds of the invention are known, made by known methods, or are commercially available. It will be apparent to the skilled artisan that methods for preparing precursors and functionality related to the compounds claimed herein are generally described in the literature. The skilled artisan given the literature and this disclosure is well equipped to prepare any of the claimed compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March's Advanced Organic Chemistry: Reactions, Mechanisms, and Structure 6th Ed., John Wiley & Sons (2007), Carey and Sundberg, Advanced Organic Chemistry 5th Ed., Springer (2007) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 4th Ed., John Wiley & Sons (2006).

The following example schemes are provided for the guidance of the reader, and represent preferred methods for making the compounds exemplified herein. These methods are not limiting, and it will be apparent that other routes may be employed to prepare these compounds. Such methods specifically include solid phase based chemistries, including combinatorial chemistry. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application.

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

¹H nuclear magnetic resonance spectra (NMR) were measured in the indicated solvents on either a Bruker NMR spectrometer (Avance TM DRX500, 500 MHz for 1H) or Varian NMR spectrometer (Mercury 400BB, 400 MHz for 1H). Peak positions are expressed in parts per million (ppm) downfield from tetramethylsilane. The peak multiplicities are denoted as follows, s, singlet; d, doublet; t, triplet; m, multiplet.

The following abbreviations have the indicated meanings:
atm=atmosphere
Bn=benzyl
Boc₂O=di-tert-butyldicarbonate
brine=saturated aqueous sodium chloride
Cbz=carboxybenzyl
CbzOSu=N-(benzyl-oxycarbonyloxy)succinimide
CDI=1,1′-carbonyldiimidazole
CDMT=2-chloro-4,6-dimethoxy-1,3,5-triazine
DCM=dichloromethane
DIBAL=diisobutylaluminum hydride
DIPEA=diisopropylethylamine
DMAP=4-(dimethylamino)-pyridine
DMF=N,N-dimethylformamide
DMT-MM=4-(4,6-dimethoxy-1,3,5-triazin-2-yl)-4-methylmorpholinium chloride
EDC=1-ethyl-3-(3-dimethylaminopropyl) carbodiimide hydrochloride
ESIMS=electron spray mass spectrometry
EtOAc=ethyl acetate
EtOH=ethyl alcohol
HATU=2-(1H-7-azabenzotriazol-1-yl)—1, 1, 3, 3-tetramethyl uronium hexafluorophosphate methanaminium
HOBt=1-hydroxybenzotriazole
Lawesson's reagent=2,4-bis(4-methoxyphenyl)-1,3,2,4-dithiadiphosphetane-2,4-disulfide
MsCl=methanesulfonyl chloride
Na₂EDTA=disodium ethylene diamine tetraacetic acid
NMR=nuclear magnetic resonance
Pd/C=palladium on activated carbon
r.t.=room temperature
TBTU=O-(benzotriazol-1-yl)-N,N,N′,N′-tetramethyluronium tetrafluoroborate
TEA=triethylamine
TFA=trifluoroacetic acid
THF=tetrahydrofuran
Tr=triphenylmethyl
p-TsOH=para-toluenesulfonic acid
TLC=thin layer chromatography
TMS=trimethylsilyl
n-Bu=normal butyl

Synthesis of 6-[(1S)-2-[bis(2-azaniumylethyl)carbamoyl]-1-{[(1R)-1-carbamoyl-3-[4-(trifluoromethyl)phenyl]propyl]carbamoyl}ethan-1-aminium]quinolin-1-ium tetrachloride 45 is depicted below in scheme 1 and example 1

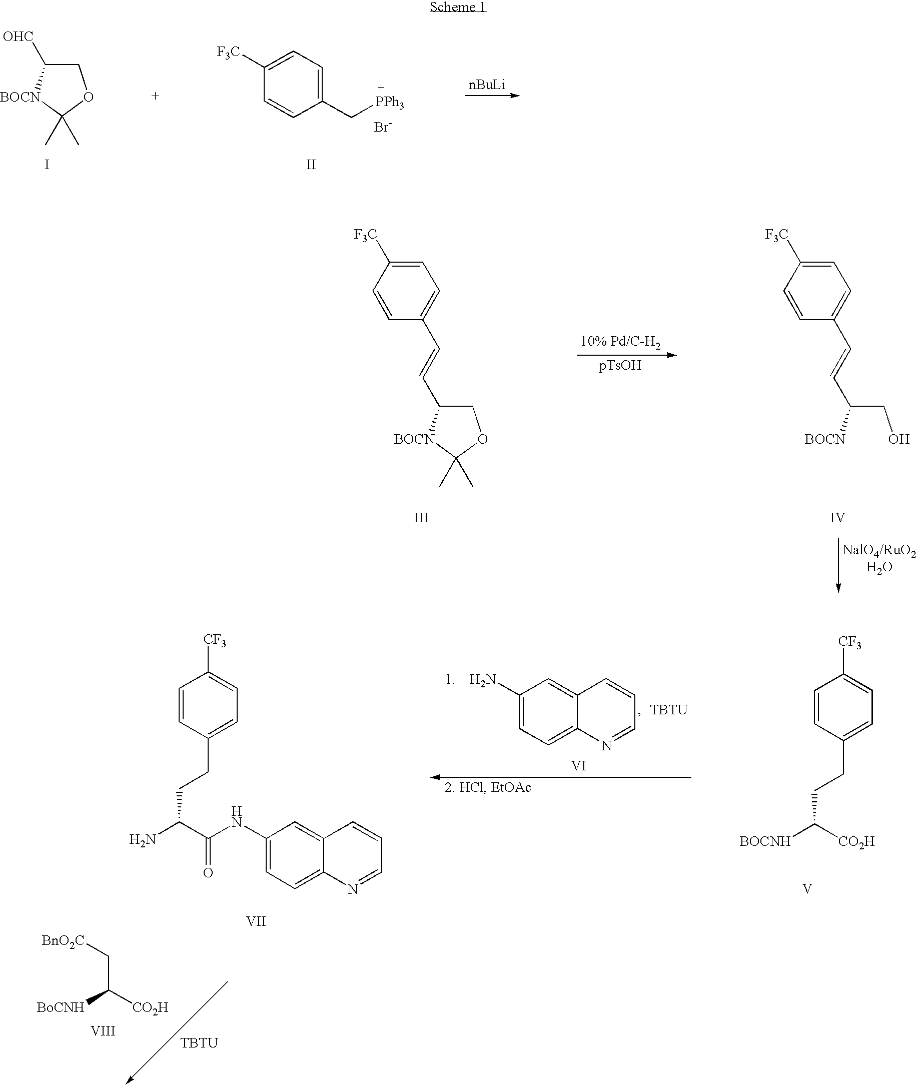

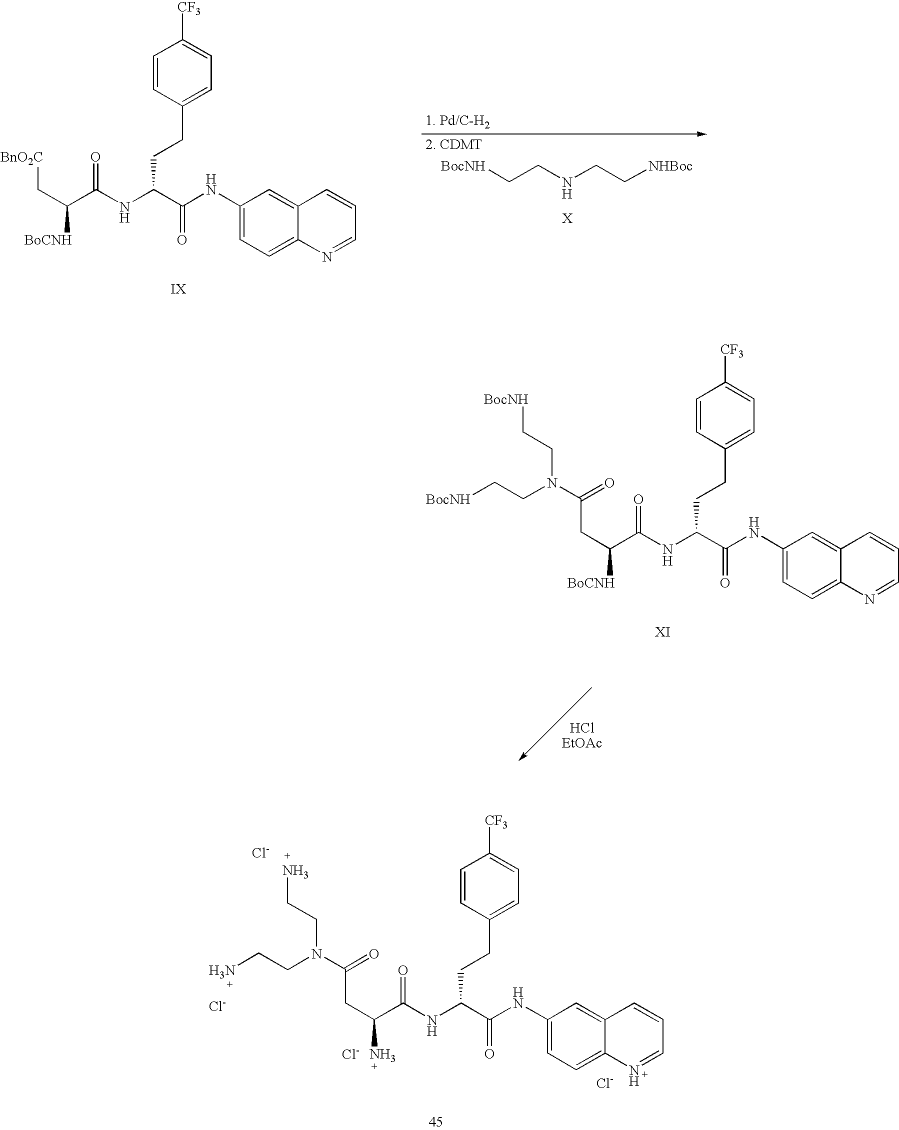

### Example 1

### Step 1

To a solution of triphenyl({[4-(trifluoromethyl)phenyl]methyl}) phosphonium bromide II (80.2 g; 0.16 mol) in THF (640 mL) under argon and cooled to −68° C. was added n-BuLi (100 mL; 0.56 mol; as 2.5 M solution in hexanes). After 10 minutes the reaction mixture was warmed to −40° C. until the precipitate disappeared. The mixture was cooled to −68° C. again and a solution of Garner's aldehyde I (36.7 g; 0.16 mol) (obtained from L-serine) in THF (50 mL) was added dropwise over 25 minutes. The reaction was warmed to r.t. and stirred overnight before quenching with methanol (250 mL) for an additional 30 minutes. The solvent was removed under reduced pressure and the residue was then purified on a silica gel column (20:1 hexane:EtOAc) to give (R,Z-E)-tert-butyl-2,2-dimethyl-4-(4-trifluoromethylstyryl)oxazolidine-3-carboxylate III as a light-yellow oil (47.3 g, 0.128 mol, 80% yield). ESIMS found for C₁₉H₂₄F₃NO₃ m/z 372.4 (M+H).

### Step 2

To a solution of the olefin III (47.2 g; 0.127 mol) in methanol (500 mL) was added 10% Pd/C (4 g) and para-toluenesulfonic acid monohydrate (0.24 g; 1.27 mmol). The suspension was stirred under hydrogen at normal pressure and r.t. overnight. The mixture was filtered through Celite and concentrated under reduced pressure to produce compound IV as a white solid (41.7 g, 125.1 mmol, 98% yield). ESIMS found for C₁₆H₂₂F₃NO₃ m/z 334.3 (M+H).

### Step 3

To a solution of tert-butyl (1R)-1-(hydroxymethyl)-3-[4-(trifluoromethyl)phenyl]propylcarbamate IV (41.3 g; 0.124 mol) in 60% aqueous acetone was added a solid sodium (meta)periodate (266 g; 1.24 mol) followed by ruthenium(II) oxide hydrate (1.65 g; 12.4 mmol). The greenish suspension was stirred for 3 h before adding propan-2-ol (500 mL) and stirring for an additional 30 min to consume excess oxidant. The resulting suspension was filtered through Celite, and the filtrate was concentrated under reduced vacuum to give a brown oil. To the brown foam was added 1 N HCl to pH=1 which was followed by extraction with EtOAc. The organic layer was washed with brine and dried with MgSO₄. The crude residue was then purified on a silica gel column (10:1 hexane:EtOAc) to obtain (2R)-2-[(tert-butoxycarbonyl)amino]-4-[4-(trifluoromethyl)phenyl]butanoic acid V (18 g; 51.8 mmol, 42% yield). ¹H NMR (CDCl₃) 1.46 (brs, 9H), 1.93-2.30 (m, 2H), 2.68-2.87 (m, 2H), 4.12-4.47 (m, 1H), 5.04-5.23 (m, 1H), 7.30 (d, J=8, 2H), 7.55 (d, J=8, 2H); ESIMS found for C₁₆H₂₀F₃NO₄ m/z 348.3 (M+H).

### Step 4

To a solution of (2R)-2-{[(tert-butoxy)carbonyl]amino}-4-[4-(trifluoromethyl)phenyl]butanoic acid V (0.97 g, 2.79 mmol) and 3-aminoquinoline VI (0.45 g, 3.10 mmol) in ethyl acetate (30 mL) was added DMT-MM (1.0 g, 3.63 mmol). After being stirred at r.t. overnight, the reaction was washed with water, 1N HCl, aq. sat. NaHCO₃, water and dried over Na₂SO₄. The solvent was removed under reduced pressure to afford tert-butyl N-[(1R)-1-[(quinolin-3-yl)carbamoyl]-3-[4-(trifluoromethyl)phenyl]propyl]carbamate (1.23 g, 2.59 mmol, 93% yield). ESIMS found for C₂₅H₂₆F₃N₃O₃ m/z 474 (M+H).

### Step 5

tert-butyl N-[(1R)-1-[(quinolin-3-yl)carbamoyl]-3-[4-(trifluoromethyl)phenyl]propyl]carbamate (1.23 g, 2.60 mmol) in trifluoroacetic acid (10 mL) and was stirred at r.t. for 1 h. The solvent was removed under reduced pressure before treating with DCM (2×20 mL) and evaporated. Crude VII was obtained as the trifluoroacetate before suspending in EtOAc (20 mL) and treating with TEA (0.72 mL, 5.2 mmol) while the mixture became homogeneous. This solution was used in the next step.

### Step 6

To the solution of (2S)-4-(benzyloxy)-2-{[(tert-butoxy)carbonyl]amino}-4-oxobutanoic acid VIII (430 mg, 1.33 mmol) in DCM (10 mL) was added DIPEA (0.65 mL, 3.75 mmol), (2R)-2-amino-N-(quinolin-6-yl)-4-[4-(trifluoromethyl)phenyl]butanamide VII (540 mg 1.21 mmol) and TBTU (428 mg, 1.33 mmol). The mixture was stirred at r.t. overnight. The reaction mixture was then washed with 1 M K₂CO₃, 1 M HCl, brine and dried over MgSO₄. The residue was then purified on a silica gel column (50:1 CHCl₃/methanol) to yield benzyl (3S)-3-{[(tert-butoxy)carbonyl]amino}-3-{[(1R)-1-[(quinolin-6-yl)carbamoyl]-3-[4-(trifluoromethyl)phenyl]propyl]carbamoyl}propanoate IX (680 mg, 1.00 mmol, 75% yield). ¹H NMR(CDCl₃) 1.46 (s, 9H), 1.88-2.20 (m, 2H), 2.40-2.64 (m, 1H), 2.66-2.88 (m, 1H), 2.92 (d, J=6 Hz, 1H), 3.27 (dd, J=5 Hz, J=17 Hz, 1H), 4.49-4.70 (m, 2H), 5.12 (d, J=5 Hz, 2H), 5.59 (d, J=8 Hz, 1H), 7.07 (d, J=8 Hz, 1H), 7.22-7.42 (m, 8H), 7.53 (s, 1H), 7.57 (s, 1H), 7.72 (dd, J=2 Hz, J=9 Hz, 1H), 8.00 (d, J=9 Hz, 1H), 8.08 (d, J=8 Hz, 1H), 8.41 (d, J=2 Hz, 1H), 8.82 (d, J=4 Hz, 1H), 8.89 (s, 1H); ¹⁹F NMR (DMSO-d₆)-61.73 (s, 3F); ESIMS found for C₃₆H₃₇F₃N₄O₆ m/z 679 (M+H).

### Step 7

To a solution of benzyl (3S)-3-{[(tert-butoxy)carbonyl]amino}-3-{[(1R)-1-[(quinolin-6-yl)carbamoyl]-3-[4-(trifluoromethyl)phenyl]propyl]carbamoyl}propanoate IX (570 mg, 0.84 mmol) in EtOH/water (15 mL/2 mL) under argon was added 10% Pd/C catalyst (catalytic amount). The mixture was stirred under an atmosphere of hydrogen at r.t. overnight. The mixture was then filtered through Celite and evaporated to dryness. The oily residue was suspended in ethyl ether and filtered to afford the free acid as a white crystalline solid (110 mg, 0.18 mmol, 32% yield). ESIMS found for C₂₉H₃₁F₃N₄O₆ m/z 589 (M+H).

### Step 8

To a solution of CDMT (37 mg, 0.20 mmol) in DCM (10 mL) and cooled to 0° C. was added N-methylmorpholine (0.023 mL, 0.20 mmol). The mixture was stirred for 10 min before adding (3S)-3-{[(tert-butoxy)carbonyl]amino}-3-{[(1R)-1-[(quinolin-6-yl)carbamoyl]-3-[4-(trifluoromethyl)phenyl]propyl]carbamoyl}propanoic acid (110 mg, 0.18 mmol). The solution was stirred for 60 min at 0° C. The tert-butyl N-t2-[(2-t[(tert-butoxy)carbonyl]amino}ethyl)amino]ethyl}carbamate X was then added and the mixture stirred at r.t. overnight. The solution was washed with 1 M aq. K₂CO₃, 1 M aq. HCl, brine and dried over anhydrous MgSO₄. The crude product was then purified on a silica gel column (100:1 CHCl₃/MeOH) and finally crystallized from ethyl ether/hexane to give tert-butyl N-[(1S)-2-[bis(2-{[(tert-butoxy)carbonyl]amino}ethyl)carbamoyl]-1-{[(1R)-1-[(quinolin-6-yl)carbamoyl]-3-[4-(trifluoromethyl)phenyl]propyl]carbamoyl}ethyl]carbamate XI (110 mg, 0.13 mmol, 72% yield). ¹H NMR (CDCl₃) 1.43 (s, 18H), 1.50 (s, 9H), 1.69 (brs, 2H), 2.03 (brs, 2H), 2.78 (brs, 2H), 3.23 (brs, 2H), 3.46 (brs, 4H), 4.60 (brs, 4H), 4.96-5.11 (m, 1H), 5.98-6.14 (m, 1H), 6.91-7.01 (m, 1H), 7.28-7.38 (m, 3H), 7.47-7.58 (m, 3H), 7.88-8.03 (m, 2H), 8.12 (d, J=7 Hz, 1H), 8.52 (s, 1H), 8.81 (brs, 1H), 9.31 (brs, 1H); ¹⁹F NMR (DMSO-d₆) −61.75 (s, 3F); ESIMS found for C₄₃H₅₈F₃N₇O₉ m/z 874 (M+H).

### Step 9

To a solution of tert-butyl N-[(1S)-2-[bis(2-{[(tert-butoxy)carbonyl]amino}ethyl)carbamoyl]-1-{[(1R)-1-[(quinolin-6-yl)carbamoyl]-3-[4-(trifluoromethyl)phenyl]propyl]carbamoyl}ethyl]carbamate XI (110 mg, 0.13 mmol) in EtOAc (5 mL) was added HCl (4.5 M solution in EtOAc, 5 mL). The reaction mixture was stirred for 15 min at r.t. before adding ethyl ether (20 mL). The precipitate was filtered and washed with ether to give 6-[(1S)-2-[bis(2-azaniumylethyl)carbamoyl]-1-{[(1R)-1-carbamoyl-3-[4-(trifluoromethyl)phenyl]propyl]carbamoyl}ethan-1-aminium]quinolin-1-ium tetrachloride 45 as a white crystalline solid (88 mg, 0.12 mmol, 92% yield). ¹H NMR (DMSO-d₆) 2.01-2.26 (m, 2H), 2.68-2.88 (m, 2H), 2.92-3.29 (m, 8H), 4.24-4.37 (m, 1H), 4.40-4.56 (m, 1H), 7.50 (d, J=8 Hz, 2H), 7.62 (d, J=8 Hz, 2H), 7.77-7.89 (m, 1H), 8.09 (brs, 3H), 8.17-8.24 (brs, 2H), 8.34 (brs, 6H), 8.61 (s, 1H), 8.82 (d, J=9 Hz, 1H), 9.03 (d, J=4 Hz, 1H), 9.16 (d, J=7 Hz, 1H), 10.90 (s, 1H); ¹⁹F NMR (DMSO-d₆)-60.06 (s, 3F); ESIMS found for C₂₈H₃₄F₃N₇O₃ m/z 574 (M+H).

The following compounds are prepared in accordance with the procedure described in the above example 1.

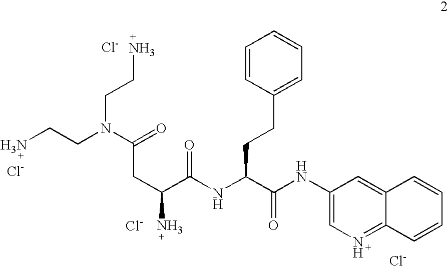

### 3-[(1S)-2-[bis(2-azaniumylethyl)carbamoyl]-1-{[(1S)-1-carbamoyl-3-phenylpropyl]carbamoyl}ethan-1-aminium]quinolin-1-ium tetrachloride 2

¹H NMR (DMSO-d₆) 2.02-2.22 (m, 2H), 2.65-2.87 (m, 2H), 2.96-3.05 (m, 2H), 3.06-3.27 (m, 4H), 3.52-3.59 (m, 4H), 4.38-4.45 (m, 1H), 4.47-4.53 (m, 1H), 7.16-7.31 (m, 5H), 7.63 (t, J=8 Hz, 1H), 7.71 (t, J=8 Hz, 1H), 7.97-8.02 (m, 2H), 8.04 (brs, 3H), 8.25 (brs, 3H), 8.36 (brs, 3H), 8.80 (s, 1H), 9.11 (d, J=3 Hz, 1H), 9.17 (d, J=7 Hz, 1H), 10.80 (s, 1H); ESIMS found for C₂₇H₃₅N₇O₃ m/z 506 (M+H).

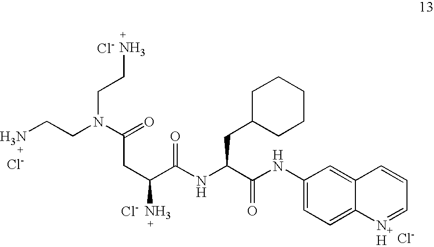

### 6-[(1S)-2-[bis(2-azaniumylethyl)carbamoyl]-1-{[(1S)-1-carbamoyl-2-cyclohexylethyl]carbamoyl}ethan-1-aminium]quinolin-1-ium tetrachloride 13

¹H NMR (DMSO-d₆) 0.91-0.99 (m, 2H), 1.14-1.24 (m, 4H), 1.43-1.50 (m, 1H), 1.60-1.80 (m, 6H), 2.99-3.02 (m, 2H), 3.08-3.15 (m, 2H), 3.22-3.28 (m, 2H), 3.53-3.70 (m, 3H), 4.32-4.35 (m, 1H), 4.52-4.57 (m, 1H), 7.89 (dd, J=9 Hz, J=5 Hz, 1H), 8.13 (brs, 3H), 8.20 (dd, J=9 Hz, J=2 Hz, 1H), 8.31 (s, 1H), 8.35 (brs, 6H), 8.36 (s, 1H), 8.70 (d, J=2 Hz, 1H), 8.95-8.99 (m, 2H), 9.07 (d, J=5 Hz, 1H), 10.82 (s, 1H); ESIMS found for C₂₆H₃₉N₇O₃ m/z 498 (M+H).

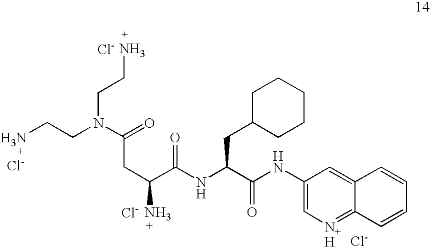

### 3-[(1S)-2-[bis(2-azaniumylethyl)carbamoyl]-1-{[(1S)-1-carbamoyl-2-cyclohexylethyl]carbamoyl}ethan-1-aminium]quinolin-1-ium tetrachloride 14

¹H NMR (DMSO-d₆) 0.92-0.97 (m, 2H), 1.15-1.24 (m, 4H), 1.46-1.52 (m, 1H), 1.60-1.78 (m, 6H), 2.98-3.02 (m, 2H), 3.06-3.16 (m, 2H), 3.21-3.29 (m, 2H), 3.55-3.72 (m, 4H), 4.29-4.35 (m, 1H), 4.51-4.56 (m, 1H), 7.68 (t, J=8 Hz, 1H), 7.78 (t, J=8 Hz, 1H), 8.00 (s, 1H), 8.09 (brs, 4H), 8.34 (brs, 6H), 8.94 (s, 1H), 9.00 (d, J=7 Hz, 1H), 9.22 (s, 1H), 10.90 (s, 1H); ESIMS found for C₂₆H₃₉N₇O₃ m/z 498 (M+H).

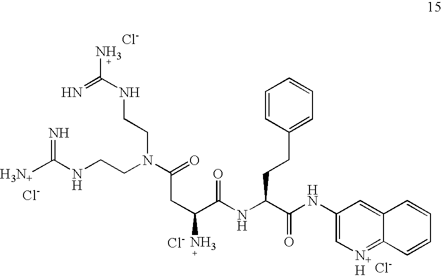

Prepared using procedures from Example 1, 5 and 8. 3-[(1S)-2-[bis(t2-[(azaniumylmethanimidoyl)amino]ethyl})carbamoyl]-1-{[(1S)-1-carbamoyl-3-phenylpropyl]carbamoyl}ethan-1-aminium]quinolin-1-ium tetrachloride 15

¹H NMR (DMSO-d₆) 1.99-2.21 (m, 2H), 2.62-2.72 (m, 1H), 2.74-2.87 (m, 1H), 3.11-3.20 (m, 2H), 3.26-3.33 (m, 2H), 3.33-3.69 (m, 6H), 4.29-4.40 (m, 1H), 4.43-4.51 (m1H), 6.81-7.54 (brs, 4H), 7.12-7.19 (m, 1H), 7.23-7.30 (m, 4H), 7.54-7.59 (m, 2H), 7.61-7.68 (m, 1H), 7.72-7.83 (m, 2H), 7.90-7.97 (m, 2H), 8.30 (s, 3H), 8.69 (s, 1H), 9.01 (s, 1H), 9.14 (d, J=7 Hz, 1H), 10.58 (s, 1H).

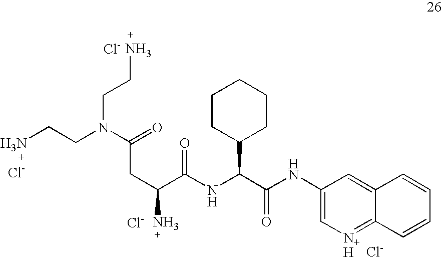

### 3-[(1S)-2-[bis(2-azaniumylethyl)carbamoyl]-1-{[(S)-carbamoyl (cyclohexyl)methyl]carbamoyl}ethan-1-aminium]quinolin-1-ium tetrachloride 26

¹H NMR (DMSO-d₆) 1.12-1.26 (m, 6H), 1.61-1.71 (m, 4H), 1.80 (d, J=8 Hz, 1H), 2.99-3.05 (m, 4H), 3.12-3.20 (m, 2H), 3.51-3.60 (m, 1H), 3.60-3.68 (m, 4H), 4.37-4.40 (m, 1H), 7.66 (t, J=8 Hz, 1H), 7.75 (t, J=8 Hz, 1H), 8.05 (s, 1H), 8.07 (s, 1H), 8.09 (brs, 3H), 8.34 (brs, 6H), 8.88 (d, J=7 Hz, 1H), 8.90 (s, 1H), 9.17 (d, J=2 Hz, 1H), 10.98 (s, 1H); ESIMS found for C₂₅H₃₇N₇O₃ m/z 484 (M+H).

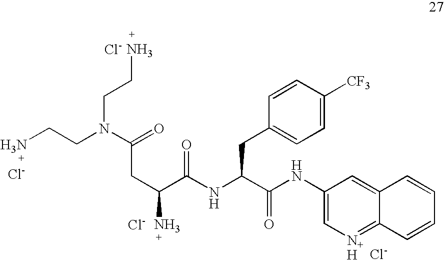

### 3-[(1S)-2-[bis(2-azaniumylethyl)carbamoyl]-1-{[(1S)-1-carbamoyl-2-[4-(trifluoromethyl)phenyl]ethyl]carbamoyl}ethan-1-aminium]quinolin-1-ium tetrachloride 27

¹H NMR (DMSO-d₆) 2.88-3.27 (m, 6H), 3.28-3.45 (m, 2H), 3.49-3.74 (m, 4H), 4.15-4.22 (m, 1H), 4.72-4.87 (m, 1H), 7.61-7.87 (m, 6H), 8.11 (brs, 3H), 8.15 (brs, 1H), 8.26 (brs, 3H), 8.35 (brs, 4H), 9.00 (s, 1H), 9.21 (d, J=7 Hz, 1H), 9.29 (d, J=2 Hz, 1H), 11.49 (s, 1H); ¹⁹F NMR (DMSO-d₆)-60.09 (s, 3F); ESIMS found for C₂₇H₃₂F₃N₇O₃ m/z 560 (M+H).

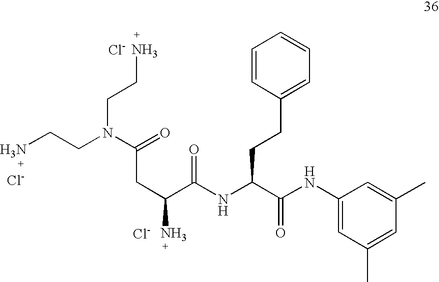

(1S)-2-[bis(2-azaniumylethyl)carbamoyl]-1-{[(1S)-1-[(3,5-dimethylphenyl)carbamoyl]-3-phenylpropyl]carbamoyl}ethan-1-aminium trichloride 36

¹H NMR (DMSO-d₆) 1.96-2.08 (m, 2H), 2.19-2.24 (m, 6H), 2.60-2.66 (m, 1H), 2.71-2.80 (m, 1H), 2.90-3.13 (m, 5H), 3.18-3.29 (m, 1H), 3.64-3.68 (m, 4H), 4.34-4.44 (m, 2H), 6.68 (brs, 1H), 7.14-7.19 (m, 1H), 7.21-7.31 (m, 7H), 8.15 (brs, 3H), 8.31-8.49 (m, 6H), 9.07-9.11 (m, 1H), 9.98 (brs, 1H); ESIMS found for C₂₆H₃₈N₆O₃ m/z 483 (M+H).

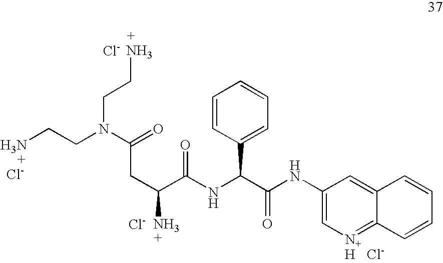

### 3-[(1S)-2-[bis(2-azaniumylethyl)carbamoyl]-1-{[carbamoyl(phenyl)methyl]carbamoyl}ethan-1-aminium]quinolin-1-ium tetrachloride 37

¹H NMR (DMSO-d₆) 2.92-3.19 (m, 6H), 3.47-3.59 (m, 4H), 4.43 (brs, 1H), 5.77 (brs, 1H), 7.52 (d, J=8 Hz, 1H), 7.39-7.45 (m, 2H), 7.60-7.68 (m, 3H), 7.73 (d, J=8 Hz, 1H), 7.97-8.04 (m, 2H), 8.08 (brs, 3H), 8.28 (brs, 3H), 8.33 (brs, 3H), 8.85 (s, 1H), 9.17 (brs, 1H0, 9.42 (d, J=8 Hz, 1H), 11.40 (s, 1H); ESIMS found for C₂₅H₃₁N₇O₃ m/z 478 (M+H).

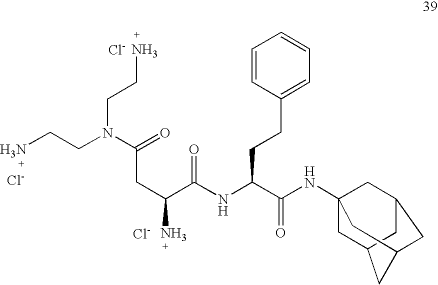

(1S)-1-{[(1S)-1-[(adamantan-1-yl)carbamoyl]-3-phenylpropyl]carbamoyl}-2-[bis(2-azaniumylethyl)carbamoyl]ethan-1-aminium trichloride 39

¹H NMR (DMSO-d₆) 1.15 (s, 1H), 1.48 (s, 1H), 1.51 (s, 1H), 1.69 (s, 2H), 1.72-1.84 (m, 9H), 1.87-1.96 (m, 4H), 2.04-2.07 (m, 1H), 3.00-3.12 (m, 4H), 3.17-3.24 (m, 2H), 3.58-3.63 (m, 4H), 4.32-4.35 (m, 1H), 4.50-4.53 (m, 1H), 7.18-7.30 (m, 5H), 7.92 (d, J=8 Hz, 1H), 8.03 (brs, 3H), 8.35 (brs, 6H), 8.85 (d, J=8 Hz, 1H); ESIMS found for C₂₈H₄₄N₆O₃ m/z 513 (M+H).

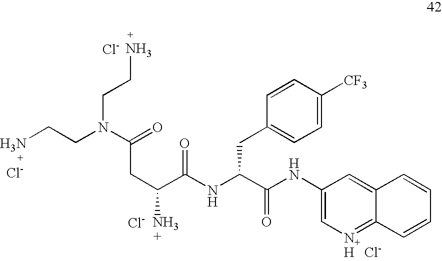

### 3-[(1R)-2-[bis(2-azaniumylethyl)carbamoyl]-1-{[(1R)-1-carbamoyl-2-[4-(trifluoromethyl)phenyl]ethyl]carbamoyl}ethan-1-aminium]quinolin-1-ium tetrachloride 42

¹H NMR (DMSO-d₆) 2.93-3.26 (m, 8H), 4.04-4.29 (m, 4H), 4.41-4.64 (m, 1H), 4.72-4.92 (m, 1H), 6.73-6.93 (m, 1H), 6.95-7.22 (m, 1H), 7.42-7.84 (m, 5H), 7.98-8.13 (m, 4H), 8.16-8.40 (m, 6H), 8.41-8.54 (m, 1H), 8.77-8.98 (m, 1H), 9.10-9.29 (m, 1H), 11.34 (brs, 1H); ¹⁹F NMR (DMSO-d₆)-60.09 (s, 3F); ESIMS found for C₂₇H₃₂F₃N₇O₃ m/z 560 (M+H).

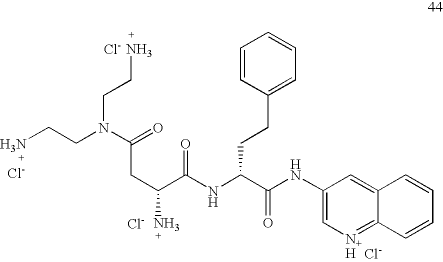

### 3-[(1R)-2-[bis(2-azaniumylethyl)carbamoyl]-1-{[(1R)-1-carbamoyl-3-phenylpropyl]carbamoyl}ethan-1-aminium]quinolin-1-ium tetrachloride 44

¹H NMR (DMSO-d₆) 1.95-2.19 (m, 2H), 2.59-2.82 (m, 2H), 2.95-3.02 (m, 2H), 3.03-3.15 (m, 4H), 3.48-3.66 (m, 4H), 4.31-4.40 (m, 1H), 4.45-4.55 (m, 1H), 7.09-7.31 (m, 5H), 7.65-7.83 (m, 2H), 8.04 (d, J=8 Hz, 2H), 8.84 (brs, 1H), 8.97-9.06 (m, 1H), 9.14 (brs, 1H), 10.93 (s, 1H); ESIMS found for C₂₇H₃₅N₇O₃ m/z 506 (M+H).

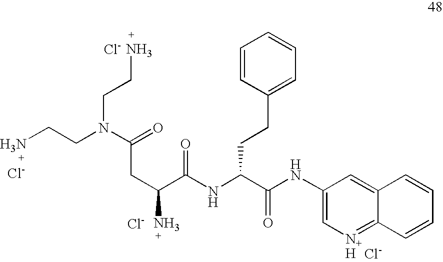

### 3-[(1S)-2-[bis(2-azaniumylethyl)carbamoyl]-1-{[(1R)-1-carbamoyl-3-phenylpropyl]carbamoyl}ethan-1-aminium]quinolin-1-ium tetrachloride 48

¹H NMR (DMSO-d₆) 2.02-2.19 (m, 2H), 2.61-2.78 (m, 2H), 2.96 (brs, 2H), 3.07 (brs, 2H), 3.14-3.30 (m, 2H), 3.58 (brs, 2H), 3.65 (brs, 2H), 4.34 (brs, 2H), 4.44 (brs, 2H), 7.10-7.15 (m, 1H), 7.22-7.26 (m, 1H), 7.74 (t, J=8 Hz, 1H), 7.85 (t, J=8 Hz, 1H), 8.12-8.21 (m, 5H), 8.41 (brs, 6H), 9.11 (bs, 1H), 9.20 (d, J=7 Hz, 1H), 9.40 (d, J=2 Hz, 1H), 11.30 (s, 1H); ESIMS found for C₂₇H₃₅N₇O₃ m/z 506 (M+H).

Synthesis of 3-[(1S)-3-[bis(2-azaniumylethyl)carbamothioyl]1-{[(1R)-1-carbamoyl-2-[4-(trifluoromethyl)phenyl]ethyl]carbamoyl}propan-1-aminium]quinolin-1-ium tetrachloride 12 is depicted below in scheme 2 and example 2

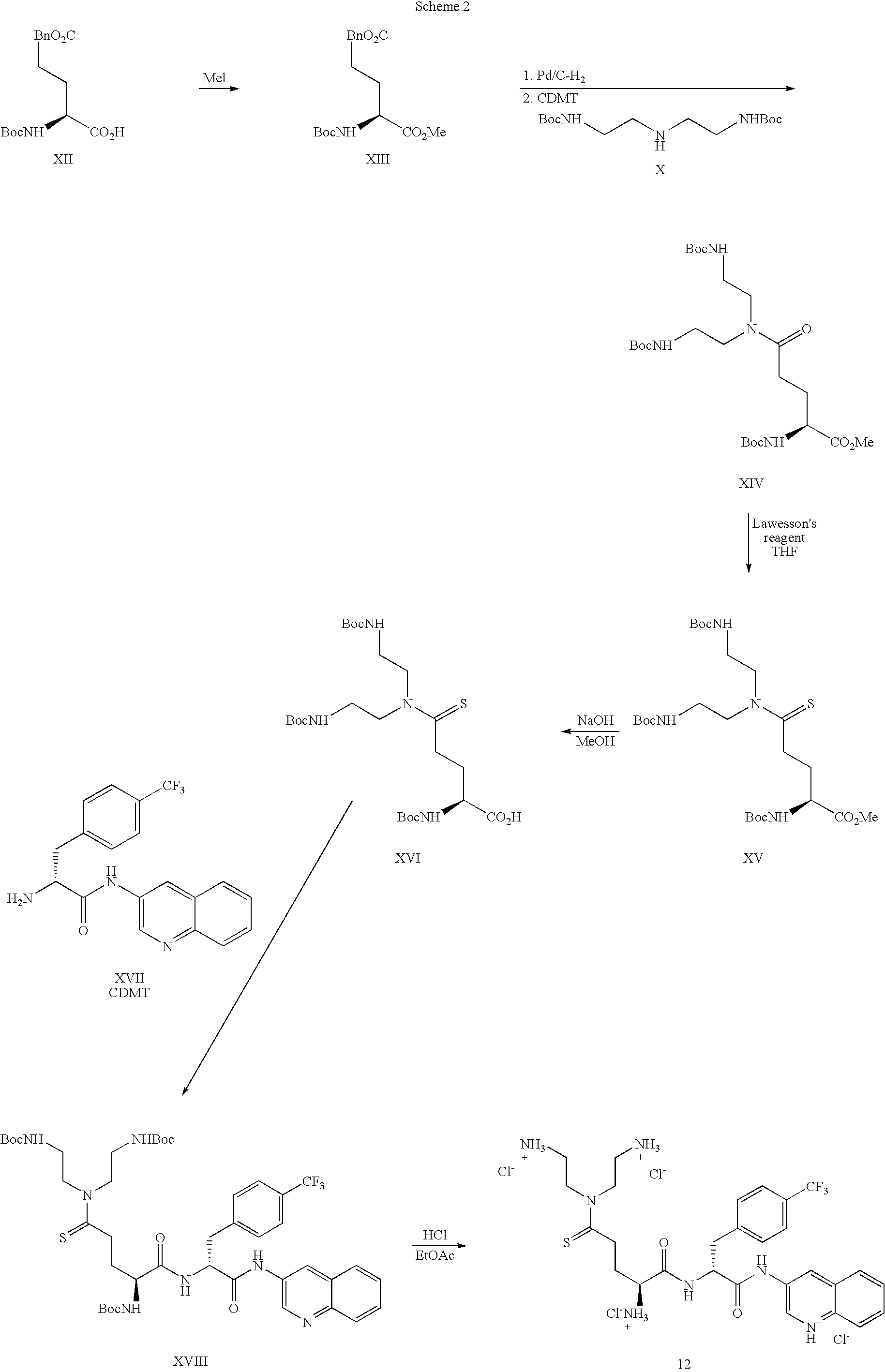

### Example 2

### Step 1

Methyl iodide (1.01 mL, 16.3 mmol) was added dropwise to a solution of (2S)-5-(benzyloxy)-2-{[(tert-butoxy)carbonyl]amino}-5-oxopentanoic acid XII (5.00 g, 14.82 mmol) and K₂CO₃ (2.25 g, 16.3 mmol) in DMF (25 mL) at r.t. The reaction mixture was stirred about 3 h at r.t. before adding additional methyl iodide (1.01 mL, 16.3 mmol). EtOAc was then added to the reaction and washed 3×10% Na₂S₂O₃ and dried over MgSO₄. The solvent was removed under reduced pressure and the crude product was purified on a silica gel column (100:1 and then 50:1 CHCl₃/MeOH) to give 5-benzyl 1-methyl (2S)-2-{[(tert-butoxy)carbonyl]amino}pentanedioate XIII (4.20 g, 12.23 mmol, 82% yield). ESIMS found for C₁₈H₂₅NO₆ m/z 352 (M+H).

### Step 2

To a solution of 5-benzyl 1-methyl (2S)-2-{[(tert-butoxy)carbonyl]amino}pentanedioate XIII (4.2 g, 12.23 mmol) in EtOH/water (40 mL/6 mL) under argon was added 10% Pd/C catalyst (catalytic amount). The mixture was stirred under an atmosphere of hydrogen for 6 h at r.t. The mixture was then filtered through Celite and evaporated to dryness to afford the free acid (3.0 g, 11.48 mmol, 32% yield). ESIMS found for C₃₂H₆₁N₇O₁₀ m/z 262 (M+H).

### Step 3

To a solution of CDMT (2.22 g, 12.62 mmol) in DCM (40 mL) and cooled to 0° C. was added N-methylmorpholine (1.38 mL, 12.63 mmol). The mixture was stirred for 10 min before adding (4S)-4-{[(tert-butoxy)carbonyl]amino}-5-methoxy-5-oxopentanoic acid (3.0 g, 11.48 mmol). The solution was stirred for 60 min at 0° C. The tert-butyl N-t2-[(2-{[(tert-butoxy)carbonyl]amino}ethyl)amino]ethyl}carbamate X was then added and the mixture stirred at r.t. overnight. The solution was washed with 1 M aq. K₂CO₃, 1 M aq. HCl, brine and dried over anhydrous MgSO₄. The crude product was crystallized from DCM/hexane to give methyl (2S)-4-[bis(2-{[(tert-butoxy)carbonyl]amino}ethyl)carbamoyl]-2-{[(tert-butoxy)carbonyl]amino}butanoate XIV (4.91 g, 8.98 mmol, 72% yield). ¹H NMR (DMSO-d₆) 1.35-1.47 (m, 27H), 1.80-1.91 (m, 1H), 2.19-2.32 (m, 1H), 2.33-2.42 (m, 1H), 2.46-2.57 (m, 1H), 3.11-3.38 (m, 6H), 3.40-3.53 (m, 1H), 3.54-3.62 (m, 1H), 3.73 (s, 3H), 4.26-4.38 (m, 1H), 4.99-5.09 (brs, 1H), 5.31-5.46 (m, 2H); ESIMS found for C₂₅H₄₆N₄O₉ m/z 547 (M+H).

### Step 4

To the solution of methyl (2S)-4-[bis(2-{[(tert-butoxy)carbonyl]amino}ethyl)carbamoyl]-2-{[(tert-butoxy)carbonyl]amino}butanoate XIV (610 mg, 1.12 mmol) in THF (10 mL) under argon was added Lawesson's reagent (680 mg, 1.68 mmol) and DMAP (13 mg, 0.11 mmol). The mixture was stirred at r.t. for 2 h and then refluxed over weekend. An additional two portions of Lawesson's reagent (900 mg, 2.24 mmol) and was added and the reaction was refluxed for another 4 h. The solvent was evaporated under reduced pressure and the crude product was purified on a silica gel column (1:6 EtOAc/hexane) to give methyl (2S)-4-[bis(2-{[(tert-butoxy)carbonyl]amino}ethyl)carbamothioyl]-2-{[(tert-butoxy) carbonyl]amino}butanoate XV (290 mg, 0.51 mmol, 45% yield). ESIMS found for C₂₅H₄₆N₄O₈S m/z 563 (M+H).

### Step 5

To the solution of the ester XV (290 mg, 0.51 mmol) in MeOH (10 mL) was added 4 M NaOH dropwise until pH=13. The mixture was stirred overnight at r.t. before evaporating the MeOH under reduced pressure. The residue was mixed with water and washed with ether. After acidifying to pH˜3 with 2 M HCl, the product was extracted with DCM, dried over MgSO₄ and concentrated under vacuum to give (2S)-4-[bis(2-{[(tert-butoxy)carbonyl]amino}ethyl)carbamothioyl]-2-{[(tert-butoxy)carbonyl]amino}butanoic acid XVI (250 mg, 0.45 mmol, 88% yield). ESIMS found for C₂₄H₄₄N₄O₈S m/z 549 (M+H).

### Step 6

To a solution of CDMT (86 mg, 0.49 mmol) in DCM (10 mL) and cooled to 0° C. was added N-methylmorpholine (0.2 mL, 1.86 mmol). The mixture was stirred for 10 min before adding (2S)-4-[bis(2-{[(tert-butoxy)carbonyl]amino}ethyl) carbamothioyl]-2-{[(tert-butoxy)carbonyl]amino}butanoic acid XVI (250 mg, 0.45 mmol). The solution was stirred for 60 min at 0° C. The (2R)-2-amino-N-(quinolin-3-yl)-3-[4-(trifluoromethyl)phenyl]propanamide XVII (210 mg, 0.49 mmol)was then added and the mixture stirred at r.t. overnight. The solution was washed with 1 M aq. K₂CO₃, 1 M aq. HCl, brine and dried over anhydrous MgSO₄. The crude product was then purified on a silica gel column (50:1 CHCl₃/MeOH) to give tert-butyl N-{2-[(2S)-2-{[(tert-butoxy)carbonyl]amino}-4-[N-(2-{[(tert-butoxy)carbonyl]amino}ethyl)methanethioamido]-N-[(1R)-1-[(quinolin-3-yl)carbamoyl]-2-[4-(trifluoromethyl)phenyl]ethyl]butanamide]ethyl}carbamate XVIII (210 mg, 0.24 mmol, 53% yield). ¹H NMR (CDCl₃) 1.34-1.41 (m, 9H), 1.42-1.56 (m, 18H), 2.02-2.14 (m, 2H), 2.51-2.74 (m, 1H), 2.82-3.00 (m, 1H), 3.21-3.41 (m, 3H), 3.46-3.53 (m, 1H), 3.54-3.72 (m, 2H), 3.73-4.00 (m, 2H), 4.21-4.43 (m, 2H), 4.88-5.00 (m, 1H), 5.49-5.71 (m, 1H), 7.44-7.54 (m, 2H), 7.56-7.70 (m, 3H), 7.81 (d, J=8 Hz, 1H), 8.05 (d, J=8 Hz, 1H), 8.74-8.9 (m, 2H), 9.01-9.09 (m, 1H); ¹⁹F NMR (DMSO-d₆)-61.75 (s, 3F); ESIMS found for C₄₃H₅₈F₃N₇O₈S m/z 890 (M+H).

### Step 7

To a solution of tert-butyl N-{2-[(2S)-2-{[(tert-butoxy)carbonyl]amino}-4-[N-(2-{[(tert-butoxy)carbonyl]amino}ethyl)methanethioamido]-N-[(1R)-1-[(quinolin-3-yl)carbamoyl]-2-[4-(trifluoromethyl)phenyl]ethyl]butanamide]ethyl}carbamate XVIII (210 mg, 0.24 mmol) in EtOAc (5 mL) was added HCl (4.5 M solution in EtOAc, 5 mL). The reaction mixture was stirred for 15 min at r.t. before adding ethyl ether (20 mL). The precipitate was filtered and washed with ether to give 3-[(1S)-3-[bis(2-azaniumylethyl)carbamothioyl]-1-{[(1R)-1-carbamoyl-2-[4-(trifluoromethyl)phenyl]ethyl]carbamoyl}propan-1-aminium]quinolin-1-ium methane tetrachloride 12 as a white crystalline solid (140 mg, 0.19 mmol, 79% yield). ¹H NMR (DMSO-d₆) 1.76-1.84 (m, 1H), 2.00-2.09 (m, 1H), 2.55-2.65 (m, 2H), 2.80-2.91 (m, 1H), 3.00-3.15 (m, 5H), 3.27-3.36 (m, 2H), 3.82-4.07 (m, 2H), 4.13-4.19 (brs, 1H), 4.89-4.94 (brs, 1H), 7.49-7.69 (m, 6H), 7.94 (t, J=8 Hz, 2H), 7.99-8.13 (brs, 3H), 8.64-8.74 (s, 1H), 8.95-9.03 (s, 1H), 9.19 (d, J=8 Hz, 1H), 10.89-10.99 (s, 1H); ¹⁹F NMR (DMSO-d₆)-60.12 (s, 3F).

The following compounds are prepared in accordance with the procedure described in the above example 2. Most examples would skip Step 4.

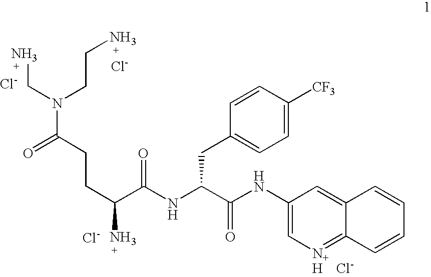

### 3-[(1S)-3-[bis(2-azaniumylethyl)carbamoyl]-1-{[(1R)-1-carbamoyl-2-[4-(trifluoromethyl)phenyl]ethyl]carbamoyl}propan-1-aminium]quinolin-1-ium tetrachloride 1

¹H NMR (DMSO-d₆) 1.64-1.95 (m, 2H), 2.78-3.18 (m, 6H), 3.26-3.43 (m, 2H), 3.47-3.69 (m, 4H), 3.77-4.10 (m, 1H), 4.75-5.08 (m, 1H), 7.60-7.63 (m, 4H), 7.64-7.82 (m, 2H), 7.92-8.16 (m, 5H), 8.35 (brs, 6H), 8.80-8.92 (m, 1H), 9.08-9.33 (m, 2H), 11.27 (brs, 1H); ¹⁹F NMR (DMSO-d₆)-60.06 (s, 3F); ESIMS found for C₂₈H₃₄F₃N₇O₃ m/z 574 (M+H).

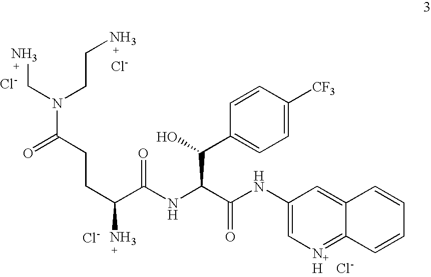

### 3-[(1S)-3-[bis(2-azaniumylethyl)carbamoyl]-1-{[(1S,2R)-1-carbamoyl-2-hydroxy-2-[4-(trifluoromethyl)phenyl]ethyl]carbamoyl]propan-1-aminium]quinolin-1-ium tetrachloride 3

¹H NMR (DMSO-d₆) 1.87-2.13 (m, 2H), 2.59-2.73 (m, 1H), 2.77-2.86 (m, 1H), 2.87-2.98 (m, 2H), 3.02-3.13 (m, 2H), 3.48-3.58 (m, 2H), 3.60-3.72 (m, 2H), 4.84 (dd, J=8 Hz, J=7 Hz, 1H), 5.46-5.58 (m, 1H), 6.03-6.43 (m, 1H), 7.59-7.80 (m, 4H), 7.88 (d, J=8 Hz, 2H), 7.94-8.03 (brs, 3H), 8.04-8.12 (m, 2H), 8.19-8.28 (brs, 3H), 8.27-8.41 (brs, 3H), 8.80-8.99 (m, 2H), 9.22 (s, 1H), 11.46 (s, 1H); ¹⁹F NMR (DMSO-d₆)-60.07 (s, 3F); ESIMS found for C₂₈H₃₄F₃N₇O₄ m/z 590 (M+H).

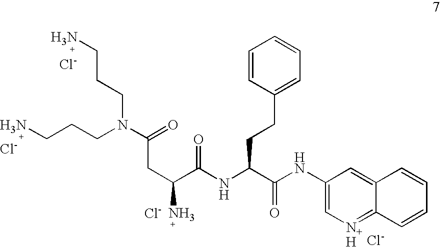

### 3-[(1S)-2-[bis(3-azaniumylpropyl)carbamoyl]-1-{[(1S)-1-carbamoyl-3-phenylpropyl]carbamoyl}ethan-1-aminium]quinolin-1-ium tetrachloride 7

¹H NMR (DMSO-d₆) 1.83-1.88 (m, 2H), 1.96-2.03 (m, 2H), 2.05-2.24 (m, 2H), 2.72-2.92 (m, 6H), 3.16-3.22 (m, 2H), 3.40-3.55 (m, 4H), 4.30-4.33 (m, 1H), 4.50-4.54 (m, 1H), 7.15-7.20 (m, 1H), 7.28-7.30 (m, 5H), 7.65-7.69 (m, 1H), 7.77-7.78 (m, 1H), 8.03-8.08 (m, 2H), 8.17 (brs 3H), 8.32 (brs 3H), 8.50 (brs, 3H), 8.96 (d, J=2 Hz, 1H), 9.26 (d, J=2 Hz, 1H), 11.02 (s, 1H); ESIMS found for C₂₉H₃₉N₇O₃ m/z 534 (M+H).

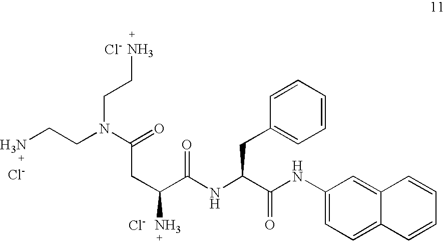

### (1S)-2-[bis(2-azaniumylethyl)carbamoyl]-1-{[(1S)-1-[(naphthalen-2-yl)carbamoyl]-2-phenylethyl]carbamoyl}ethan-1-aminium trichloride 11

¹H NMR (DMSO-d₆) 3.78-3.21 (m, 8H), 3.46-3.81 (m, 4H), 4.16 (brs, 1H), 4.72 (brs, 1H), 7.05-7.53 (m, 7H), 7.67 (brs, 1H), 7.80 (brs, 3H), 8.00-8.54 (m, 10H), 9.07 (brs, 1H), 10.60 (brs, 1H); ESIMS found for C₂₇H₃₄N₆O₃ m/z 491 (M+H).

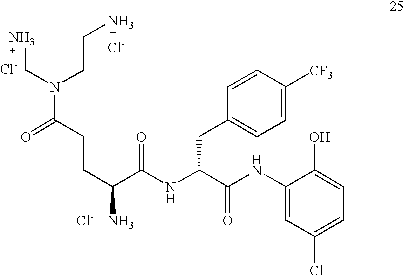

### (1S)-3-[bis(2-azaniumylethyl)carbamoyl]-1-{[(1R)-1-[(5-chloro-2-hydroxyphenyl)carbamoyl]-2-[4-(trifluoromethyl)phenyl]ethyl]carbamoyl}propan-1-aminium trichloride 25

¹H NMR (DMSO-d₆) 1.62-1.72 (m, 1H), 1.75-1.82 (m, 1H), 2.37-2.49 (m, 2H), 2.92-3.06 (m, 6H), 3.46-3.61 (m, 4H), 3.89 (brs, 1H), 5.05-5.12 (m, 1H), 6.93-7.01 (m, 2H), 7.59-7.65 (m, 3H), 7.97 (d, J=2 Hz, 1H), 8.01 (brs, 3H), 8.26 (brs, 3H), 8.32 (brs, 3H), 9.04 (d, J=9 Hz, 1H), 9.75 (s, 1H), 10.38 (s, 1H), 11.98 (brs, 1H); ESIMS found for C₂₅H₃₂N₆O₄ClF₃ m/z 573 (M+H).

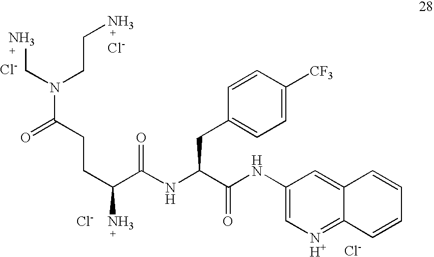

### 3-[(1S)-3-[bis(2-azaniumylethyl)carbamoyl]-1-{[(1S)-1-carbamoyl-2-[4-(trifluoromethyl)phenyl]ethyl]carbamoyl}propan-1-aminium]quinolin-1-ium tetrachloride 28

¹H NMR (DMSO-d₆) 1.64-1.95 (m, 2H), 2.78-3.18 (m, 6H), 3.26-3.43 (m, 2H), 3.47-3.69 (m, 4H), 3.77-4.10 (m, 1H), 4.75-5.08 (m, 1H), 7.60-7.63 (m, 4H), 7.64-7.82 (m, 2H), 7.92-8.16 (m, 5H), 8.35 (brs, 6H), 8.80-8.92 (m, 1H), 9.08-9.33 (m, 2H), 11.27 (brs, 1H); ¹⁹F NMR (DMSO-d₆)-60.06 (s, 3F); ESIMS found for C₂₈H₃₄F₃N₇O₃ m/z 574 (M+H).

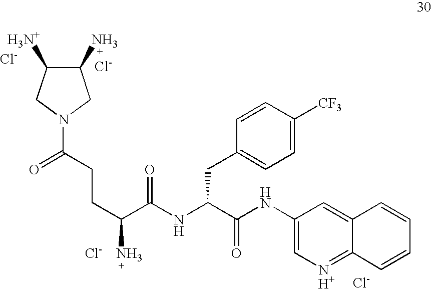

### 3-[(1S)-1-{[(1R)-1-carbamoyl-2-[4-(trifluoromethyl)phenyl]ethyl]carbamoyl}-4-[(3R,4S)-3,4-diazaniumylpyrrolidin-1-yl]-4-oxobutan-1-aminium]quinolin-1-ium tetrachloride 30

¹H NMR (DMSO-d₆) 1.56-1.85 (m, 4H), 2.04-2.28 (m, 2H), 2.89-3.14 (m, 2H), 3.30-3.40 (m, 2H), 3.97-4.10 (m, 3H), 4.81-5.05 (m, 1H), 7.68-7.91 (m, 4H), 8.06-8.20 (m, 4H), 8.30 (brs, 3H), 8.92 (brs, 3H), 8.99-9.12 (m, 4H), 9.24 (d, J=8 Hz, 1H), 9.31-9.38 (m, 1H), 11.51 (brs, 1H); ¹⁹F NMR (DMSO-d₆)-60.06 (s, 3F); ESIMS found for C₂₈H₃₂F₃N₇O₃ m/z 572 (M+H).

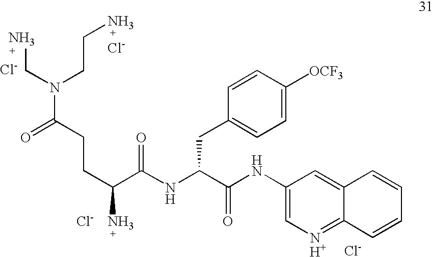

### 3-[(1S)-3-[bis(2-azaniumylethyl)carbamoyl]-1-{[(1R)-1-carbamoyl-2-[4-(trifluoromethoxy)phenyl]ethyl]carbamoyl}propan-1-aminium]quinolin-1-ium tetrachloride 31

¹H NMR (DMSO-d₆) 1.66-1.93 (m, 2H), 2.80-3.14 (m, 6H), 3.21-3.37 (m, 2H), 3.50-3.78 (m, 4H), 3.88-4.01 (m, 1H), 4.81-4.97 (m, 1H), 7.26 (d, J=8 Hz, 2H), 7.53 (d, J=8 Hz, 2H), 7.63-7.84 (m, 2H), 8.03 (s, 1H), 8.06 (brs, 3H), 8.10 (s, 1H), 8.35 (brs, 6H), 8.89 (d, J=2 Hz, 1H), 9.19 (s, 1H), 9.24 (d, J=2 Hz, 1H), 11.35 (s, 1H); ¹⁹F NMR (DMSO-d₆) −56.13 (s, 3F); ESIMS found for C₂₈H₃₄F₃N₇O₄ m/z 590 (M+H).

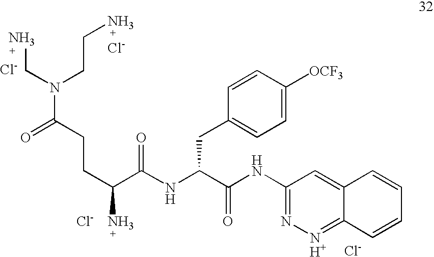

### 3-[(1S)-3-[bis(2-azaniumylethyl)carbamoyl]1-{[(1R)-1-carbamoyl-2-[4-(trifluoromethoxy)phenyl]ethyl]carbamoyl}propan-1-aminium]cinnolin-1-ium tetrachloride

¹H NMR (CD₃OD) 1.65-1.79 (m, 1H), 1.90-2.10 (m, 1H), 2.64-2.74 (m, 2H), 3.15-3.26 (m, 4H), 3.60-3.75 (m, 4H), 4.08-4.16 (m, 1H), 4.20-4.36 (m, 1H), 4.95-5.00 (m, 1H), 5.04-5.13 (m, 1H), 7.22 (d, J=8 Hz, 2H), 7.50 (d, J=8 Hz, 2H), 7.83-7.88 (m, 2H), 7.96-8.02 (m, 1H), 8.35-8.40 (m, 1H), 8.86 (s, 1H); ¹⁹F NMR (DMSO-d₆)-58.82 (s, 3F); ESIMS found for C₂₇H₃₃F₃N₈O₄ m/z 591 (M+H).

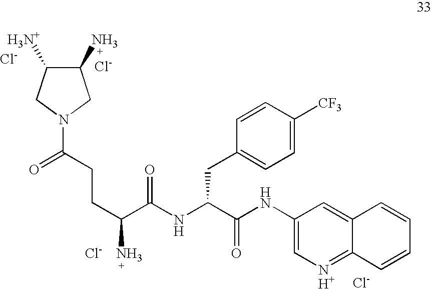

### 3-[(1S)-1-{[(1R)-1-carbamoyl-2-[4-(trifluoromethyl)phenyl]ethyl]carbamoyl}-4-[(3S,4S)-3,4-diazaniumylpyrrolidin-1-yl]-4-oxobutan-1-aminium]quinolin-1-ium tetrachloride 33

¹H NMR (DMSO-d₆) 1.56-1.85 (m, 4H), 2.04-2.28 (m, 2H), 2.89-3.14 (m, 2H), 3.30-3.40 (m, 2H), 3.97-4.10 (m, 3H), 4.81-5.05 (m, 1H), 7.68-7.91 (m, 4H), 8.06-8.20 (m, 4H), 8.30 (brs, 3H), 8.92 (brs, 3H), 8.99-9.12 (m, 4H), 9.24 (d, J=8 Hz, 1H), 9.31-9.38 (m, 1H), 11.51 (brs, 1H); ¹⁹F NMR (DMSO-d₆)-60.06 (s, 3F); ESIMS found for C₂₈H₃₂F₃N₇O₃ m/z 572 (M+H).

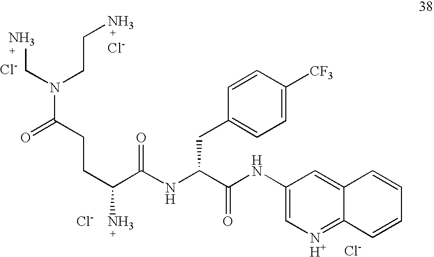

### 3-[(1R)-3-[bis(2-azaniumylethyl)carbamoyl]-1-{[(1R)-1-carbamoyl-2-[4-(trifluoromethyl)phenyl]ethyl]carbamoyl}propan-1-aminium]quinolin-1-ium tetrachloride 38

¹H NMR (DMSO-d₆) 1.88-2.04 (m, 2H), 2.86-3.19 (m, 6H), 3.21-3.41 (m, 2H), 3.47-3.60 (m, 2H), 3.63-3.74 (m, 2H), 3.83-3.95 (m, 1H), 4.75-5.02 (m, 1H), 7.60-7.71 (m, 3H), 7.72-7.85 (m, 3H), 7.98-8.16 (m, 5H), 8.36 (brs, 6H), 8.93 (brs, 1H), 9.19-9.30 (m, 1H), 9.53 (d, J=7 Hz, 1H), 11.66 (brs, 1H); ESIMS found for C₂₈H₃₄F₃N₇O₃ m/z 574 (M+H).

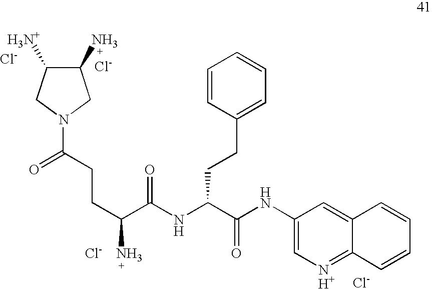

### 3-[(1S)-1-{[(1R)-1-carbamoyl-3-phenylpropyl]carbamoyl}-4-[(3S,4S)-3,4-diazaniumylpyrrolidin-1-yl]-4-oxobutan-1-aminium]quinolin-1-ium tetrachloride 41

¹H NMR (DMSO-d₆) 2.13-2.20 (m, 1H), 2.40-2.46 (m, 2H), 2.57-2.85 (m, 2H), 3.46-3.64 (m, 2H), 3.79-3.88 (m, 3H), 3.93-4.13 (m, 5H), 4.48-4.57 (m, 1H), 7.12-7.20 (m, 1H), 7.21-7.35 (m, 4H), 7.65-7.73 (m, 1H), 7.75-7.84 (m, 1H), 8.02-8.15 (m, 1H), 8.44 (brs, 3H), 8.89 (brs, 3H), 8.94 (brs, 1H), 9.01 (brs, 3H), 9.19-9.37 (m, 2H), 11.11 (brs, 1H); ESIMS found for C₂₈H₃₅N₇O₃ m/z 518 (M+H).

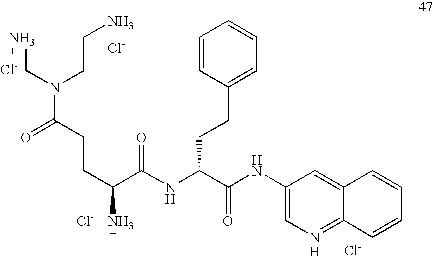

### 3-[(1S)-3-[bis(2-azaniumylethyl)carbamoyl]-1-{[(1R)-1-carbamoyl-3-phenylpropyl]carbamoyl}propan-1-aminium]quinolin-1-ium tetrachloride 47

¹H NMR (DMSO-d₆) 1.98-2.18 (m, 4H), 2.55-2.67 (m, 2H), 2.69-2.80 (m, 2H), 2.87-2.97 (m, 2H), 2.97-3.07 (m, 2H), 3.56-3.66 (m, 4H), 4.00-4.05 (m, 1H), 4.45-4.55 (m, 1H), 7.12-7.28 (m, 5H), 7.62 (t, J=7 Hz, 1H), 7.72 (t, J=7 Hz, 1H), 7.95 (brs, 3H), 7.98 (d, J=9 Hz, 1H), 8.01 (d, J=8 Hz, 1H), 8.28 (brs, 3H), 8.40 (brs, 3H), 8.81 (s, 1H), 9.14 (d, J=2 Hz, 1H), 9.21 (d, J=7 Hz, 1H), 10.94 (s, 1H); ESIMS found for C₂₈H₃₇N₇O₃ m/z 520 (M+H).

Synthesis of 3-[(1S)-3-[bis(2-azaniumylethyl)carbamoyl]-1-{[(1E)-1-carbamoyl-2-[4-(trifluoromethyl)phenyl]eth-1-en-1-yl]carbamoyl}propan-1-aminium]quinolin-1-ium tetrachloride 4 is depicted below in scheme 3 and example 3.

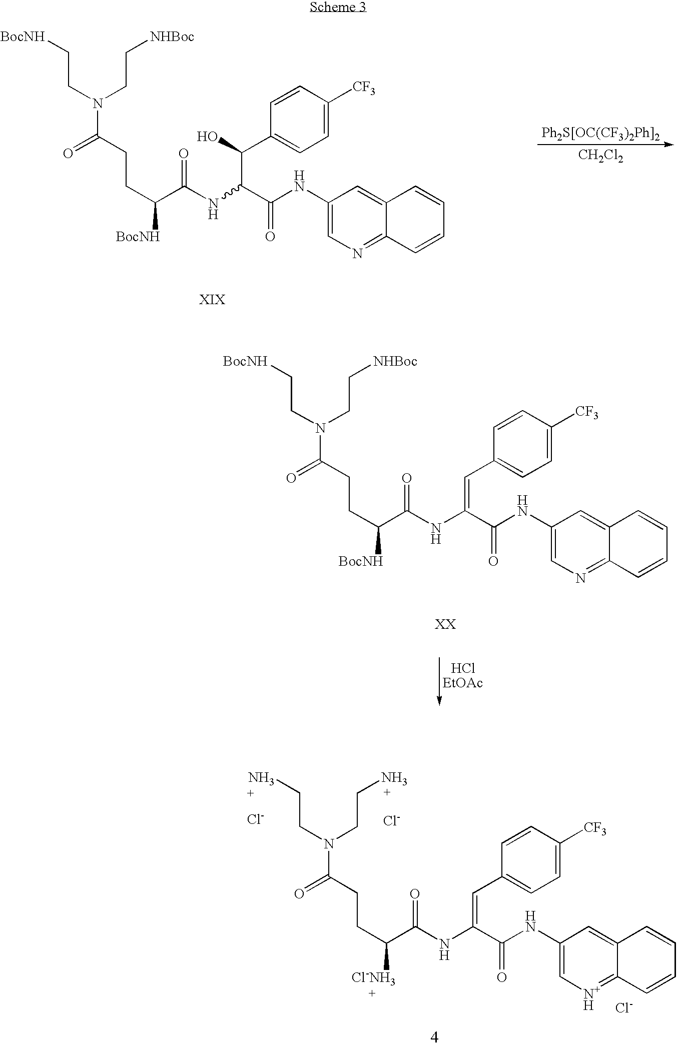

### Example 3

Preparation of tert-butyl N-{2-[(2S)-2-{[(tert-butoxy)carbonyl]amino}-N-(2-{[(tert-butoxy)carbonyl]amino}ethyl)-N′-[(2S)-2-hydroxy-1-[(quinolin-3-yl)carbamoyl]-2-[4-(trifluoromethyl)phenyl]ethyl]pentanediamido]ethyl}carbamate XIX was performed following procedures listed in example 2.

### Step 1

To a solution of compound XIX (0.26 g, 0.29 mmol) in dry DCM (5 mL) was added Martin's sulfurane (0.29 g, 0.43 mmol). The reaction mixture was stirred overnight at r.t. before the solvent was removed under reduced pressure. The residue was purified on a silica gel column (100:1 DCM:MeOH) to produce tert-butyl N-t2-[(2S)-2-{[(tert-butoxy)carbonyl]amino}-N-(2-{[(tert-butoxy)carbonyl]amino}ethyl)-N′-[(1E)-1-[(quinolin-3-yl)carbamoyl]-2-[4-(trifluoromethyl)phenyl]eth-1-en-1-yl]pentanediamido]ethyl}carbamate XX (0.13 g, 0.15 mmol, 52% yield). ESIMS found for C₄₃H₅₆F₃N₇O₉ m/z 872 (M+H).

### Step 2

Procedure can be found in examples 1-2. The final compound 4 was isolated as the hydrochloride salt. ¹H NMR (DMSO-d₆) 2.71-2.83 (m, 1H), 2.87-3.16 (m, 5H), 3.45-3.68 (m, 6H), 4.11-4.25 (m, 1H), 7.28 (s, 1H), 7.56-7.67 (m, 1H), 7.70-7.84 (m, 3H), 7.88-8.12 (m, 7H), 8.29 (brs, 3H), 8.57 (brs, 3H), 8.87 (brs, 1H), 9.25 (brs, 1H), 10.64 (brs, 1H), 10.96 (brs, 1H); ¹⁹F NMR (DMSO-d₆)-60.48 (s, 3F); ESIMS found for C₂₈H₃₂F₃N₇O₃ m/z 572 (M+H).

Synthesis of 3-[(1S)-3-[bis(2-azaniumylethyl)carbamoyl]-1-{[(1S)-1-carbamoyl-2-[4-(trifluoromethyl)phenyl]ethyl](methyl)carbamoyl}propan-1-aminium]quinolin-1-ium tetrachloride 5 is depicted below in scheme 4 and example 4.

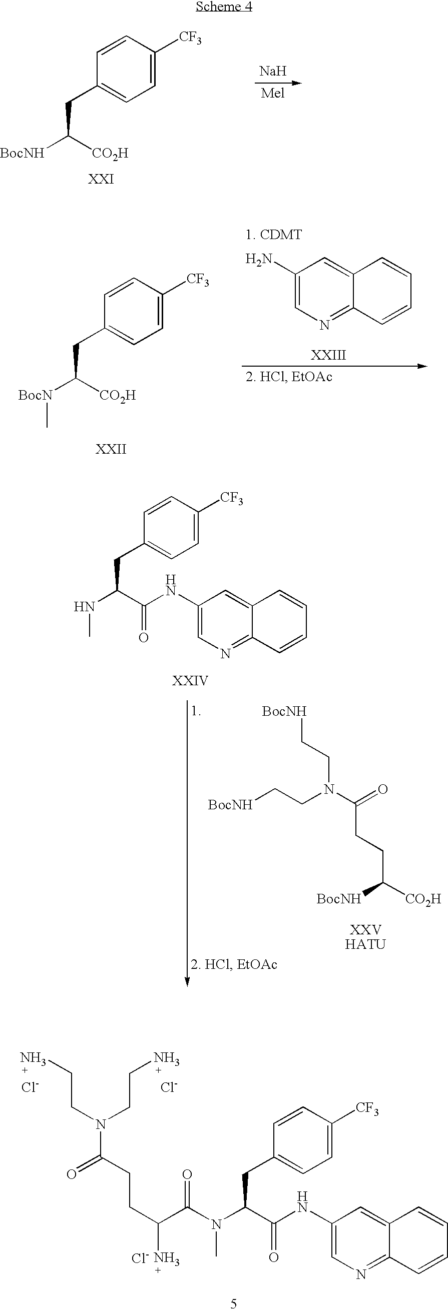

### Example 4

### Step 1

To a solution of (2R)-2-[(tert-butoxycarbonyl)amino]-3-[4-(trifluoromethyl)phenyl]propanoic acid XXI (1 g, 3 mmol) in dry THF (10 mL) was added sodium hydride (60% suspension in mineral oil) (0.72 g, 18 mmol; 6 eq. of pure NaH) in portions. Methyl iodide (1.12 mL, 18 mmol) was then added and the mixture was stirring at r.t. for 3 days. The mixture was then treated with water before removing the THF under reduced pressure. The aqueous phase was acidified and extracted 2× EtOAc. The combined EtOAc was washed with sodium thiosulfate, dried and evaporated under reduced pressure. The residue was crystallized to produce (2R)-2-[(tert-butoxycarbonyl)(methyl)amino]-3-[4-(trifluoromethyl)phenyl]-propanoic acid XXII (0.73 g, 2 mmol, 70% yield).

### Step 2-5

Procedures can be found in examples 1-2. The final compound 5 was isolated as the hydrochloride salt. ¹H NMR (DMSO-d₆) 2.64-287 (m, 2H), 2.89-3.00 (m, 2H), 3.00-3.05 (m, 2H), 3.14 (s, 3H), 3.20-3.46 (m, 2H), 3.47-3.74 (m, 6H), 4.40 (s, 1H), 5.32 (s, 1H), 7.53-7.80 (m, 6H), 7.95-8.15 (m, 5H), 8.25-8.45 (m, 6H), 8.89 (s, 1H), 9.18 (s, 1H), 11.02 (s, 1H); ¹⁹F NMR (DMSO-d₆)-60.10 (s, 3F); ESIMS found for C₃₀H₃₇F₃N₆O₃ m/z 588 (M+).

Synthesis of 3-[(1S)-4-[bis({2-[(azaniumylmethanimidoyl)amino]ethyl}) amino]-1-{[(1R)-1-carbamoyl-3-phenylpropyl]carbamoyl}butan-1-aminium]quinolin-1-ium tetrachloride 6 is depicted below in scheme 5 and example 5.

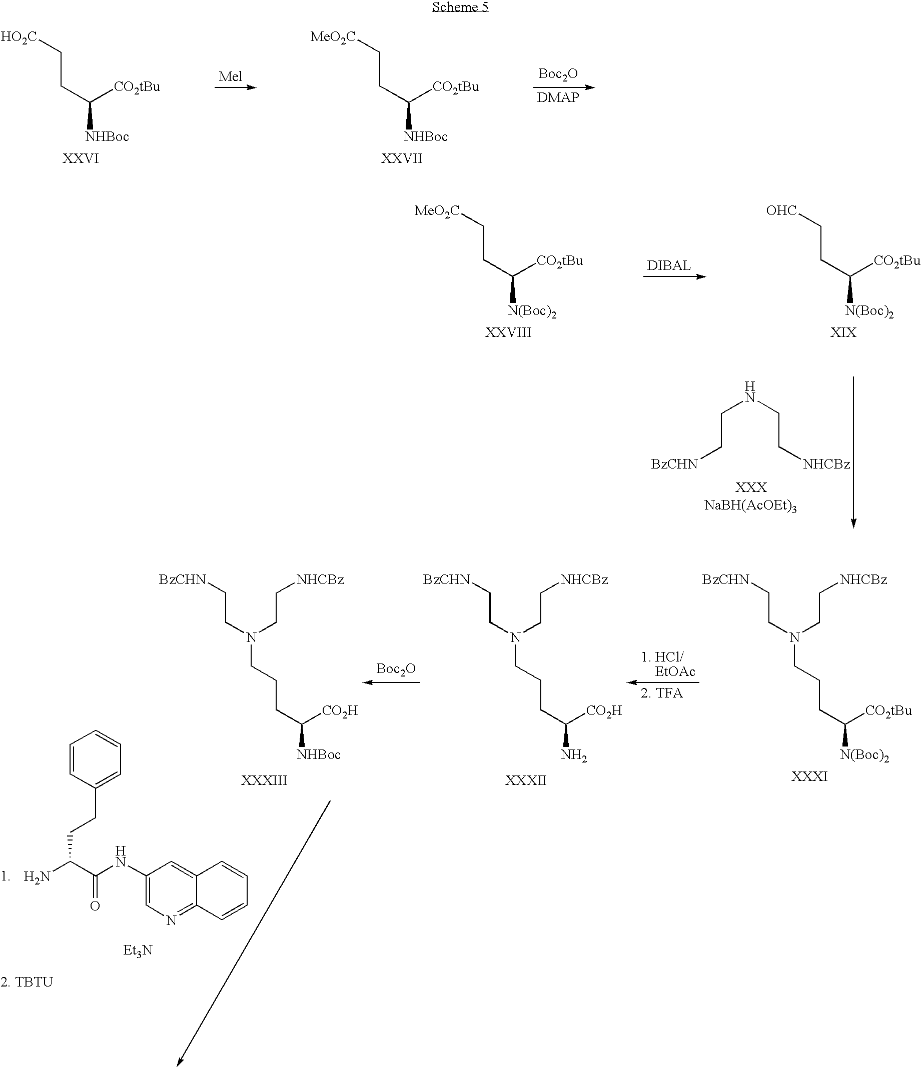

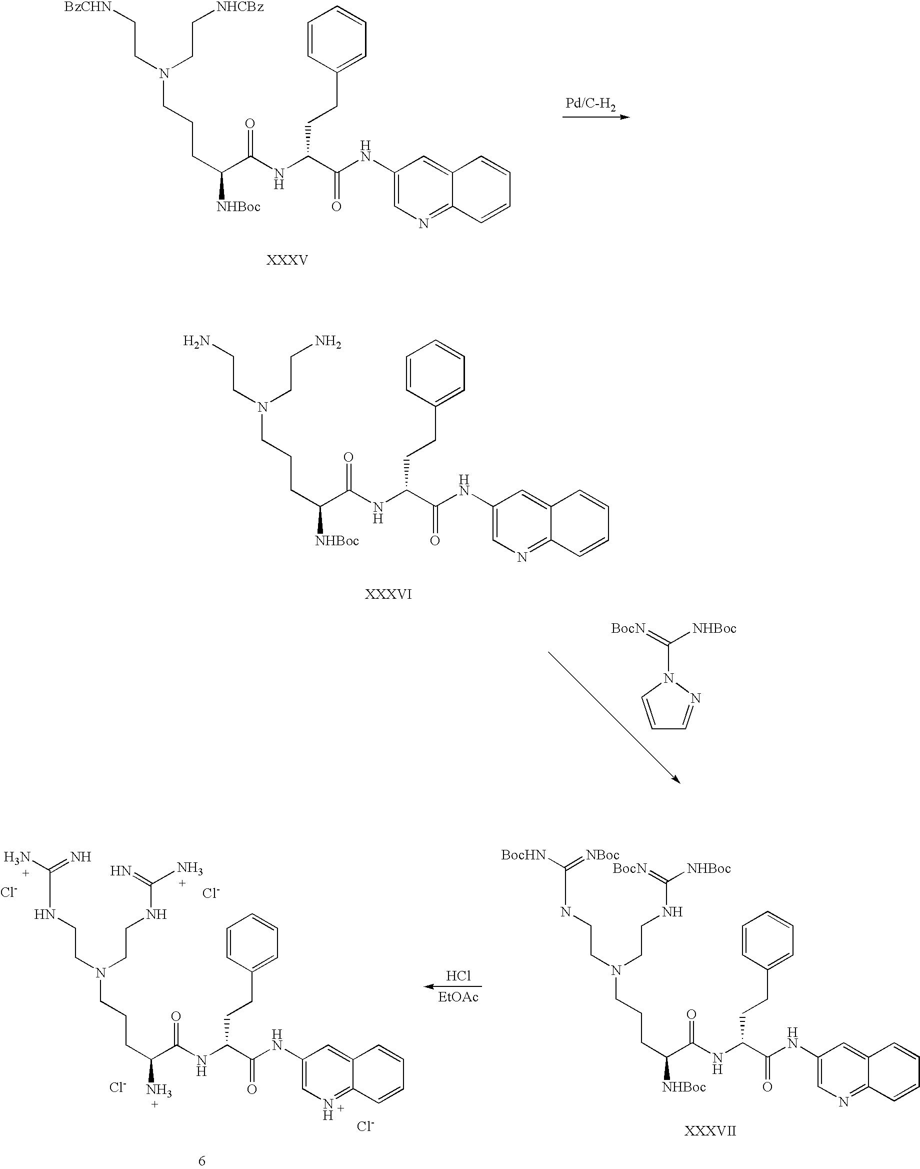

### Example 5

### Step 1

To a solution of Boc-glutamic acid tert-butyl ester XXVI (50 g, 164.8 mmol) and K₂CO₃ (34.2 g, 247.2 mmol) in DMF (250 mL) was added MeI (10.8 ml, 173.1 mmol) dropwise. The reaction mixture was stirred at r.t. for 2 h before adding ethyl acetate. The organic extract was washed 10% Na₂S₂O₃ (3×) and dried over MgSO₄. The solvent was removed under reduced pressure and the crude product was crystallized from hexane to give the product XXVII as a white solid (50.7 g, 159.8 mmol, 95% yield). ¹H NMR (CDCl₃) 1.44 (s, 9H), 1.46 (s, 9H), 1.86-1.96 (m, 1H), 2.08-2.20 (m, 1H), 2.32-2.46 (m, 2H), 3.46 (m, 2H), 3.68 (s, 3H), 4.17-4.21 (m, 1H); ESIMS found for C₁₅H₂₇NO₆ m/z 318 (M+H).

### Step 2

To a solution of 1-tert-butyl 5-methyl (2S)-2-{[(tert-butoxy)carbonyl]amino}pentanedioate XXVII (50.7 g, 159.8 mmol), TEA (26.6 mL, 191.7 mmol) and DMAP (19.5 g, 159.8 mmol) in MeCN (480 mL) was added di-tert-butyl dicarbonate (69.7 g, 319.5 mmol). The reaction mixture was stirred at r.t. overnight before adding additional TEA (11.1 mL, 79.0 mmol), DMAP (9.8 g, 79.9 mmol) and Boc₂O (34.8 g, 159.8 mmol) and stirring for another 2 days. The solvent was removed under reduced pressure and residue was purified on a silica gel column (1:100-1:50-1:30 EtOAc:hexane) to give pure product XXVIII as colorless oil. (50.0 g, 119.8 mmol, 75% yield). ¹H NMR(CDCl₃) 1.44 (s, 9H), 1.49 (s, 18H), 2.15 (ddd, J=3 Hz, J=8 Hz, J=19 Hz, 1H), 2.33-2.46 (m, 3H), 3.66 (s, 3H), 4.75 (m, 1H); ESIMS found for C₂₀H₃₅NO₈ m/z 857 (2M+23).

### Step 3

To a solution of 1-tert-butyl 5-methyl (2S)-2-{bis[(tert-butoxy)carbonyl]amino}pentanedioate XXVIII (50.0 g, 119.8 mmol) in dry ethyl ether (120 mL) at −78° C. under Ar was added a solution of DIBAL (65.0 mL, 65.0 mmol). The reaction mixture was stirred 1.5-2.5 hours at −78° C. The mixture was treated with MeOH (240 mL) and allowed to warm to r.t. The suspension was filtered through Celite and washed with methanol. The solvent was removed under reduced pressure and the residue was purified on a silica gel column (1:20 EtOAc:hexane) to give pure product XXIX as a colorless oil. (37.1 g, 95.8 mmol, 80% yield). ¹H NMR (CDCl₃) 1.44 (s, 9H), 1.47 (s, 18H), 2.07-2.15 (m, 1H), 2.37-2.56 (m, 3H), 4.70 (dd, J=5 Hz, J=10 Hz, 1H), 9.73 (s, 1H); ESIMS found for C₁₉H₃₃NO₇ m/z 410 (M+23).

### Step 4

To a solution of benzyl N-{2-[(2-{[(benzyloxy)carbonyl]amino}ethyl)amino]ethyl}carbamate XXX (13.34 g, 35.92 mmol) in dry DCM (100 mL) was added acetic acid (9.34 mL, 163.25 mmol). The mixture was cooled with water/ice bath before adding tert-butyl (2S)-2-{bis[(tert-butoxy)carbonyl]amino}-5-oxopentanoate XXIX. The reaction mixture was stirred for 1 h at 0° C. and then sodium triacetoxyborohydride (10.37 g, 48.98 mmol) was added in portions. The reaction mixture was stirred at r.t. overnight. The reaction was washed with water, 1 M HCl, brine and dried over MgSO₄. The solvent was removed under reduced pressure and product was purified on a silica gel column (ethyl acetate:hexane (1:15→1:10→1:10→1:1 EtOAc:hexane→100% EtOAc) to give the protected amino acid XXXI as yellow oil (15.12 g, 20.35 mmol, 57% yield). ¹H NMR (CDCl₃) 1.44 (s, 9H), 1.50 (s, 18H), 1.70-1.98 (m, 4H), 2.00-2.16 (m, 2H), 3.15 (brs, 4H), 3.56 (brs, 4H), 4.55-4.67 (m, 1H), 5.08 (s, 4H), 6.36 (brs, 2H), 7.32 (brs, 10H); ESIMS found for C₃₉H₅₈N₄O₁₀ m/z 743 (M+H).

### Step 5

To a solution of tert-butyl (2S)-5-[bis(2-{[(benzyloxy)carbonyl]amino}ethyl)amino]-2-{bis[(tert-butoxy)carbonyl]amino}pentanoate XXXI (3.00 g, 4.04 mmol) in ethyl acetate (20 mL) was added HCl (3.5 M solution in EtOAc, 20 mL). The reaction mixture was stirred for 30 min at r.t. before adding ethyl ether (about 50 mL). The precipitate was filtered and washed with ether to give (2S)-5-[bis(2-{[(benzyloxy)carbonyl]amino}ethyl)amino]-2-{bis[(tert-butoxy)carbonyl]amino}pentanoic acid as a white crystalline solid (1.82 g, 3.14 mmol, 78% yield).

### Step 6

A solution of (2S)-5-[bis(2-{[(benzyloxy)carbonyl]amino}ethyl)amino]-2-{bis[(tert-butoxy)carbonyl]amino}pentanoic acid (1.82 g, 3.14 mmol) in TFA (20 mL) was stirred overnight. The TFA was removed under reduced pressure to give (2S)-2-amino-5-[bis(2-{[(benzyloxy)carbonyl]amino}ethyl)amino]pentanoic acid XXXII as a light brown foam (1.70 g, 2.83 mmol, 90% yield). ESIMS found for C₂₅H₃₄N₄O₆ m/z 487 (M+H).

### Step 7

To a solution of (2S)-2-amino-5-[bis(2-{[(benzyloxy)carbonyl]amino}ethyl)amino]pentanoic acid XXXII (1.70 g, 2.83 mmol) in water (20 mL) was added K₂CO₃ followed by a solution of Boc₂O (0.80 g, 3.68 mmol) in acetone (15 mL). The reaction mixture was stirred for 1 h with additional portions of K₂CO₃ being added to maintain the pH of 10. The mixture was stirred overnight and then the acetone was evaporated under reduced pressure and alkalized to pH=12. The aqueous residue was washed with diethyl ether (2×) and acidified with 6 N HCl to pH=2. The aqueous phase was washed with DCM (4×) and the combined DCM extracts were washed with brine and dried over MgSO₄. The solvent was removed under reduced pressure and product was purified on a silica gel column (100:1→50:1→30:1→20:1 EtOAc:MeOH) to give (2S)-5-[bis(2-{[(benzyloxy)carbonyl]amino}ethyl)amino]-2-{[(tert-butoxy)carbonyl]amino}pentanoic acid XXXIII (1.35 g, 3.30 mmol, 81% yield). ¹H NMR (CDCl₃) 1.41 (s, 9H), 1.83 (brs, 4H), 3.25 (brs, 6H), 3.55 (brs, 4H), 4.23 (brs, 1H), 5.07 (s, 4H), 5.72 (brs, 1H), 6.10 (brs, 1H), 6.68 (brs, 1H), 7.32 (brs, 10H); ESIMS found for C₃₀H₄₂N₄O₈ m/z 587 (M+H).

### Step 8-9

Procedures can be found in examples 1-2.

### Step 10

A solution of pyrazolecarboxamidine (1.15 g, 3.70 mmol) and tert-butyl N-[(1S)-4-[bis(2-aminoethyl)amino]-1-{[(1R)-3-phenyl-1-[(quinolin-3-yl)carbamoyl]propyl]carbamoyl}butyl]carbamate XXXVI (0.75 g, 1.20 mmol) in THF/MeOH (10 mL/10 mL) was stirred at r.t. overnight. The solvent was removed under vacuum and the residue was dissolved in DCM washed with 1 M HCl, brine and dried over MgSO₄. The crude product was purified on a silica gel column (1:1→3:1→5:1 EtOAc:hexane ˜100% EtOAc ˜100:1 EtOAc/MeOH) to give tert-butyl N-[(1Z)-{[(tert-butoxy)carbonyl]amino}({2-[(2-{[(1Z)-{[(tert-butoxy)carbonyl]amino}({[(tert-butoxy)carbonyl]imino})methyl]amino}ethyl)[(4S)-4-{[(tert-butoxy)carbonyl]amino}-4-{[(1R)-3-phenyl-1-[(quinolin-3-yl)carbamoyl]propyl]carbamoyl}butyl]amino]ethyl}amino)methylidene]carbamate XXXVII (120 mg, 0.11 mmol, 15% yield). ESIMS found for C₅₆H₈₄N₁₀O₁₂ m/z 1090 (M+H).

### Step 11

Procedure can be found in examples 1-2. The final compound 6 was isolated as the hydrochloride salt. ¹H NMR (DMSO-d₆) 1.58-1.81 (m, 4H), 1.82-1.90 (m, 1H), 1.91-1.99 (m, 1H), 2.41-2.50 (m, 1H), 2.52-2.63 (m, 1H), 3.09 (brs, 6H), 3.24-3.31 (m, 1H), 3.52 (brs, 3H), 3.86 (brs, 1H), 4.26-4.33 (m, 1H), 6.92 (brs, 1H), 7.04 (brs, 4H), 7.38 (brs, 3H), 7.49-7.55 (m, 1H), 7.59-7.65 (m, 1H), 7.81-7.91 (m, 2H), 7.93-7.98 (m, 1H), 8.34 (brs, 3H), 8.42 (brs, 3H), 8.73 (s, 1H), 8.85 (s, 1H), 9.16 (s, 1H), 10.99 (s, 1H), 11.09 (s, 1H), 11.26 (brs, 1H); ESIMS found for C₃₀H₄₃N₁₁O₂ m/z 590 (M+).

The following compound was prepared in accordance with the procedure described in the above example 5.

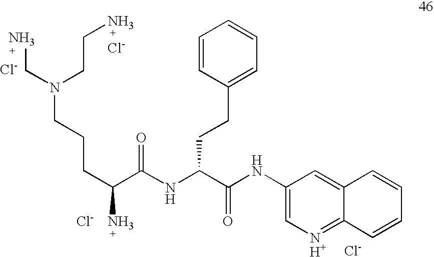

### 3-[(1S)-4-[bis(2-azaniumylethyl)amino]-1-{[(1R)-1-carbamoyl-3-phenylpropyl]carbamoyl}butan-1-aminium]quinolin-1-ium tetrachloride 46

¹H NMR (DMSO-d₆) 1.78-2.00 (m, 4H), 2.01-2.20 (m, 2H), 2.61-2.82 (m, 4H), 3.21-3.40 (m, 4H), 3.53-3.69 (m, 4H), 4.01 (brs, 1H), 4.51-4.59 (m, 1H), 7.16-7.21 (m, 1H), 7.22-7.32 (m, 4H), 7.62 (t, J=8 Hz, 1H), 7.71 (t, J=8 Hz, 1H), 7.97 (d, J=8 Hz, 1H), 8.00 (d, J=8 Hz, 1H), 8.41 (brs, 9H), 8.77 (s, 1H), 9.11 (brs, 1H), 9.24 (d, J=8 Hz, 1H), 10.89 (s, 1H); ESIMS found for C₂₈H₃₉N₇O₂ m/z 506 (M+H).

Synthesis of 3-[(1S)-4-[bis({2-[(azaniumylmethanimidoyl)amino]ethyl}) amino]-1-{[(1R)-1-carbamoyl-3-phenylpropyl]carbamoyl}butan-1-aminium]quinolin-1-ium tetrachloride 8 is depicted below in scheme 6 and example 6.

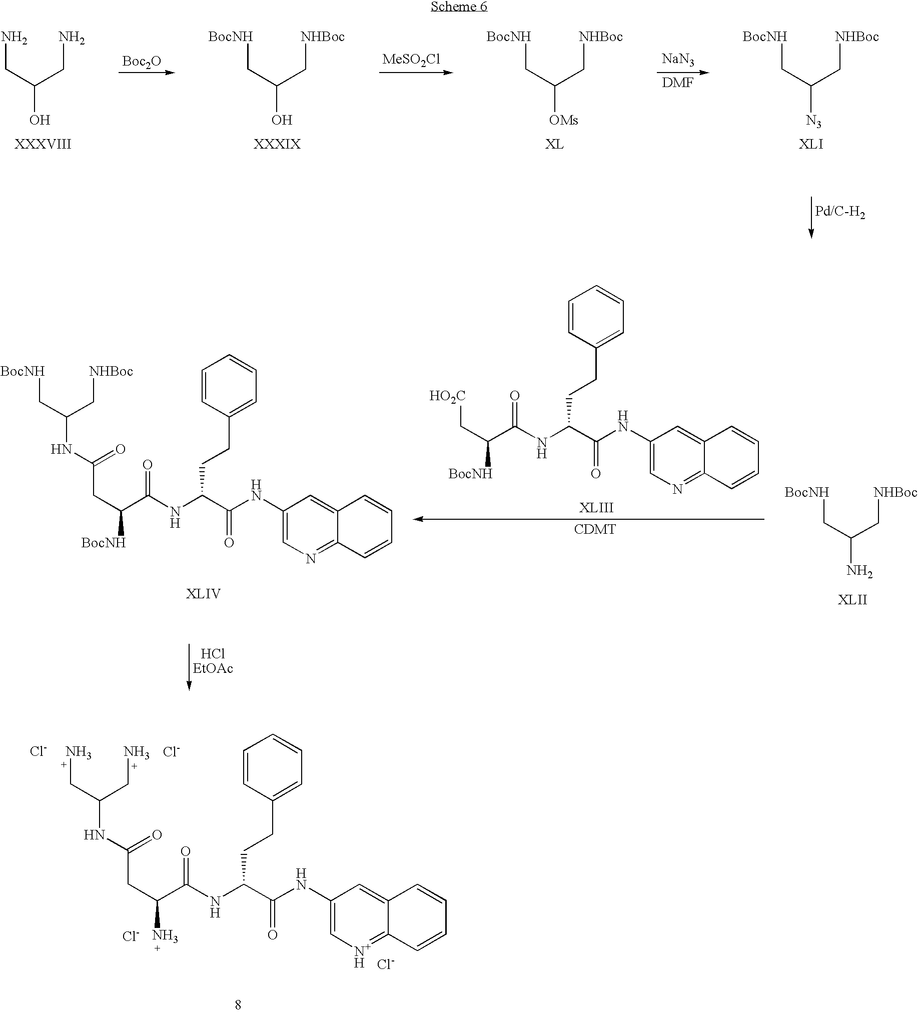

### Example 6

### Step 1

To a solution of 1,3-diamine-2-hydroxypropane (10 g, 110 mmol) in 5% NaHCO₃ (pH˜9) was added a solution of Boc₂O (97 g, 440 mmol) in acetone (200 mL). The reaction mixture was stirred overnight. The acetone was evaporated under vacuum and aqueous residue was washed 5× EtOAc. The organic layer was washed with brine and dried over MgSO₄. The solvent was removed under reduced pressure to give crude product. The product was purified on a silica gel column (1:200→1:150→1:120→100:1→80:1→50:1 MeOH:DCM) to give the pure tert-butyl N-(3-{[(tert-butoxy)carbonyl]amino}-2-hydroxypropyl)carbamate XXXIX as white solid (20.10 g, 69.3 mmol, 62% yield). ESIMS found for C₁₃H₂₆N₂O₅ m/z 291 (M+H).

### Step 2

To a solution of tert-butyl N-(3-{[(tert-butoxy)carbonyl]amino}-2-hydroxypropyl)carbamate XXXIX (1.83 g, 6.3 mmol) in DCM was added TEA (1.38 mL, 10 mmol) was added. The mixture was cooled to 10° C. before adding mesyl chloride (0.77 mL, 10 mmol) dropwise. The reaction mixture was stirred for 30 min and then the solvent was removed under reduced pressure. The residue was dissolved in DCM, washed 1 M HCl (3×), 5% NaHCO₃ and dried over MgSO₄. The solvent was again removed under vacuum to give tert-butyl N-(3-{[(tert-butoxy)carbonyl]amino}-2-(methanesulfonyloxy)propyl)carbamate XL (2.31 g, 6.3 mmol, 99% yield). ESIMS found for C₁₄H₂₈N₂O₇S m/z 369 (M+H).

### Step 3

To a solution of tert-butyl N-(3-{[(tert-butoxy)carbonyl]amino}-2-(methanesulfonyloxy)propyl)carbamate XL (2.31 g, 6.6 mmol) in DMF was added NaN₃. The mixture was heated overnight at 60° C., diluted with DCM and washed with 10% Na₂S₂O₃ (5×), 5% NaHCO₃, brine and dried over MgSO₄. The solvent was evaporated under vacuum to give crude product (1.75 g). The product was purified on a silica gel column (1:10 EtOAc:hexane) to give the pure tert-butyl N-(2-azido-3-{[(tert-butoxy)carbonyl]amino}propyl)carbamate XLI as white crystals (1.33 g, 4.2 mmol, 67% yield). ¹H NMR(CDCl₃) 1.47 (s, 18H), 3.07-3.26 (m, 2H), 3.27-3.53 (m, 2H), 3.59-3.75 (m, 1H), 5.06 (brs, 2H); ESIMS found for C₁₃H₂₅N₅O₄ m/z 316 (M+H).

### Step 4

To a solution of the azide XLI (1.33 g, 4.22 mmol) in a mixture of ethanol/water (9:1) was added a catalytic amount of Pd/C. The mixture was stirred under hydrogen overnight. The mixture was filtered through a pad of Celite and the filtrate was concentrated to dryness under vacuum to give tert-butyl N-(2-amino-3-t[(tert-butoxy) carbonyl]amino}propyl)carbamate XLII (0.85 g, 2.94 mmol, 70% yield). ¹H NMR (CDCl₃) 1.46 (s, 18H), 2.88-3.00 (m, 1H), 3.00-3.27 (m, 4H), 5.12 (brs, 2H); ESIMS found for C₁₃H₂₇N₃O₄ m/z 290 (M+H).

### Steps 5-6

Procedures can be found in examples 1-2. The final compound 8 was isolated as the hydrochloride salt. ¹H NMR (DMSO-d₆) 1.98-2.07 (m, 1H), 2.10-2.17 (m, 1H), 2.65-2.71 (m, 1H), 2.75-2.85 (m, 2H), 2.91-2.96 (m, 2H), 3.04-3.12 (m, 2H), 4.20-4.31 (m, 2H), 4.40-4.52 (m, 2H), 7.13-7.19 (m, 1H), 7.25-7.26 (m, 4H), 7.61 (dd, J=8 Hz, J=8 Hz, 1H), 7.69 (dd, J=8 Hz, J=7 Hz, 1H), 7.97-8.09 (m, 2H), 8.25 (brs, 6H), 8.20-8.32 (m, 3H), 8.63 (d, J=8 Hz, 1H), 8.78 (s, 1H), 9.06-9.10 (m, 1H), 9.23 (d, J=7 Hz, 1H), 10.86 (s, 1H); ESIMS found for C₂₆H₃₃N₇O₃ m/z 492 (M+H).

Synthesis of 3-[(1S)-3-[bis({[bis(2-azaniumylethyl)carbamoyl]methyl}) carbamoyl]-1-{[(1S)-1-carbamoyl-3-phenylpropyl]carbamoyl}propan-1-aminium]quinolin-1-ium hexachloride 9 is depicted below in scheme 7 and example 7.

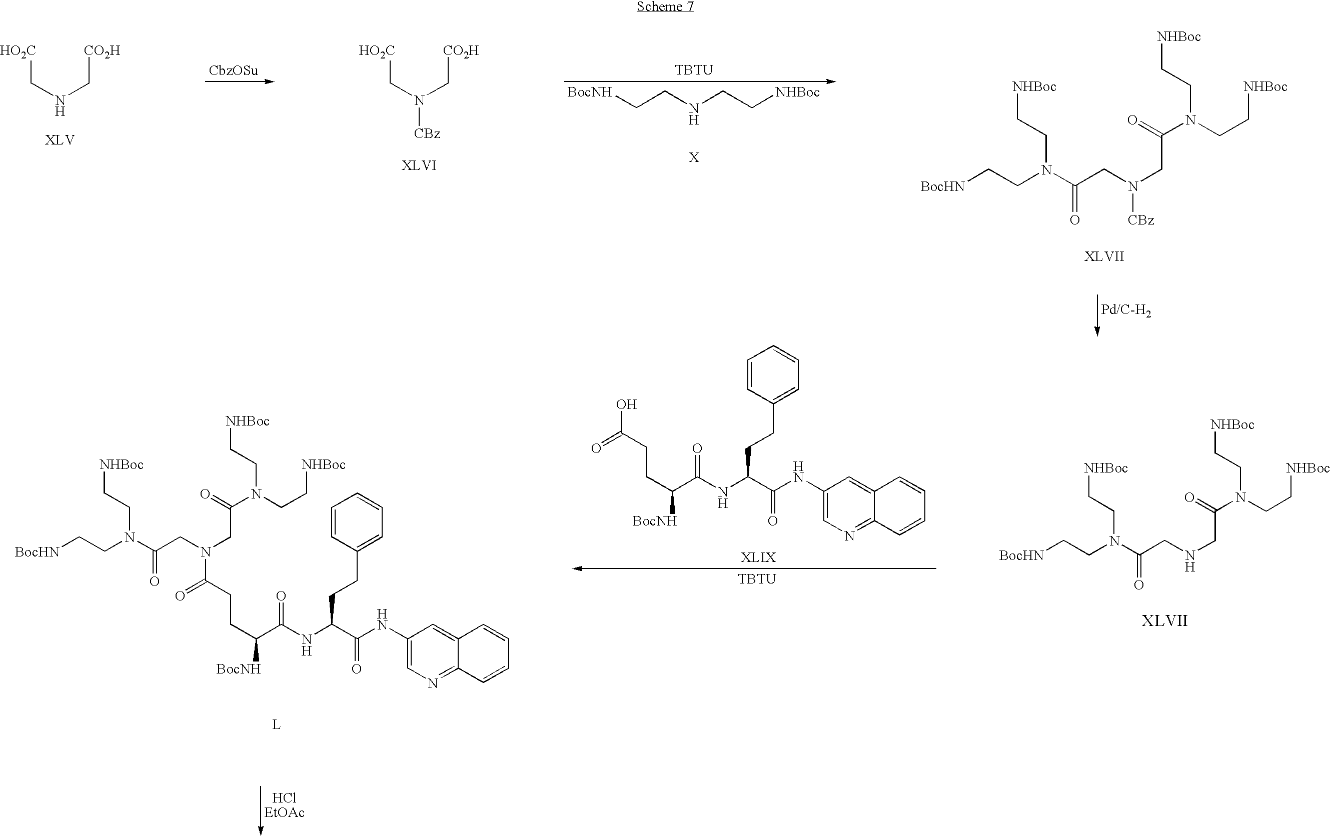

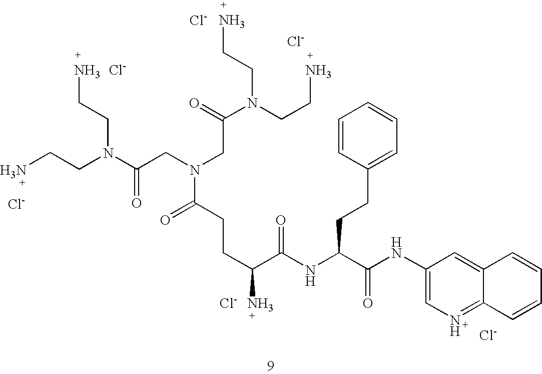

### Example 7

### Step 1

To the solution of iminodiacetic acid XLV (10 g, 75.13 mmol) and K₂CO₃ (41.53 g, 300.52 mmol) in water (225 mL) was added a solution of CBzOsu (20.6 g, 82.64 mmol) in acetone (150 mL). The mixture was stirred at r.t. overnight. The acetone was evaporated under reduced pressure and the remaining water was washed with ethyl ether (2×). The aqueous layer was acidified to pH=2 with 2 M aq. HCl and then saturated with NaCl, washed with EtOAc (3×). The combined EtOAc was dried over MgSO₄ and evaporated under reduced pressure to give 2-{[(benzyloxy)carbonyl](carboxymethyl)amino}acetic acid XLVI (15 g, 56.17 mmol, 75% yield). ESIMS found for C₁₂H₁₃NO₆ m/z 290 (M+Na).

### Step 2

To the solution of 2-{[(benzyloxy)carbonyl] (carboxymethyl)amino}acetic acid XLVI (2 g, 7.5 mmol) in DCM (25 mL) was added DIPEA (3.26 mL, 18.75 mmol), tert-butyl N-{2-[(2-{[(tert-butoxy)carbonyl]amino}ethyl)amino]ethyl}carbamate X (5.69 g 18.75 mmol) and TBTU (6.02 g, 18.75 mmol). The mixture was stirred at r.t. overnight. The reaction mixture was then washed with 1 M K₂CO₃, 1 M HCl, brine and dried over MgSO₄. The residue was purified on a silica gel column (1:20 EtOAc:hexane) to give tert-butyl N-[2-(2-{[(benzyloxy)carbonyl]({[bis(2-{[(tert-butoxy)carbonyl]amino}ethyl)carbamoyl]methyl})amino}-N-(2-{[(tert-butoxy)carbonyl]amino}ethyl)acetamido)ethyl]carbamate XLVII (5.09 g, 6.07 mmol, 81% yield). ¹H NMR (CDCl₃) 1.36-1.49 (m, 36H), 3.08-3.59 (m, 20H), 4.02-4.35 (m, 4H), 5.16 (s, 2H), 7.30-7.39 (m, 5H); ESIMS found for C₄₀H₆₇N₇O₁₂ m/z 838 (M+H).

### Step 3

To the solution of compound XLVII (5.09 g, 6.07 mmol) in EtOH/water (50 mL/8 mL) under an argon atmosphere was added 10% Pd/C (catalytic amount). The reaction was flushed with hydrogen and stirred overnight in r.t. The catalyst was removed by filtration through Celite and the solvents removed under reduced pressure to give tert-butyl N-{2-[2-({[bis(2-{[(tert-butoxy)carbonyl]amino}ethyl)carbamoyl]methyl}amino)-N-(2-{[(tert-butoxy)carbonyl]amino}ethyl)acetamido]ethyl}carbamate XLVIII (4.02 g, 5.71 mmol, 94% yield). ESIMS found for C₃₂H₆₁N₇O₁₀ m/z 704 (M+H).

### Step 4

To the solution of (4S)-4-{[(tert-butoxy)carbonyl]amino}-4-t[(1S)-3-phenyl-1-[(quinolin-3-yl)carbamoyl]propyl]carbamoyl}butanoic acid XLIX (290 mg; 0.54 mmol) in DCM (10 mL) and DIPEA (0.113 mL; 0.65 mmol) was added the amine XLVIII (460 mg; 0.65 mmol) and TBTU (210 mg; 0.65 mmol). The reaction mixture was stirred overnight before it was diluted with DCM (40 mL), washed once with water, 1 M aqueous HCl (2×), 5% NaHCO₃ (2×), water and dried over anhydrous MgSO₄. The solvent was evaporated under reduced pressure and crude product crystallized from EtOAc/hexane to give pure tert-butyl N-(2-{2-[(2S)—N-{[bis(2-{[(tert-butoxy)carbonyl]amino}ethyl) carbamoyl]methyl}-2-{[(tert-butoxy)carbonyl]amino}-N′-[(1S)-3-phenyl-1-[(quinolin-3-yl)carbamoyl]propyl]pentanediamido]-N-(2-{[(tert-butoxy)carbonyl]amino}ethyl)acetamido}ethyl)carbamate L as white solid (150 mg; 0.123 mmol; 22.8% yield). ¹H NMR (CDCl₃) 1.33-1.46 (m, 1H), 1.97-2.06 (m, 1H), 2.07-2.16 (m, 1H), 2.21-2.32 (m, 1H), 2.36-2.49 (brs, 2H), 2.60-2.70 (m, 1H), 2.72-2.85 (m, 2H), 3.13-3.52 (m, 16H), 4.01-4.20 (brs, 2H), 4.25-4.47 (m, 3H), 4.53-4.66 (m, 1H), 5.21-5.35 (m, 1H), 5.38-5.51 (brs, 1H), 5.69-5.81 (m, 1H), 5.82-5.92 (brs, 1H), 6.04-6.11 (brs, 1H), 7.18-7.24 (m, 2H), 7.25-7.31 (m, 6H), 7.50 (dd, J=7 Hz, 1H), 7.59 (dd, J=7 Hz, 1H), 7.78 (d, J=8 Hz, 1H), 8.03 (d, J=9 Hz, 1H), 8.73-8.84 (m, 1H); ESIMS found for C₆₁H₉₃N₁₁O₁₅ m/z 1220 (M+H).

### Steps 5

Procedure can be found in examples 1-2. The final compound 9 was isolated as the hydrochloride salt. ¹H NMR (DMSO-d₆) 1.84-1.97 (m, 1H), 1.98-2.06 (m, 2H), 2.07-2.19 (m, 1H), 2.61-2.72 (m, 1H), 2.76-2.84 (m, 2H), 2.86-2.96 (m, 2H), 2.97-3.05 (m, 4H), 3.07-3.19 (m, 2H), 3.44-3.56 (m, 6H), 3.57-3.68 (m, 3H), 4.24-4.44 (brs, 3H), 4.50-4.63 (brs, 3H), 7.11-7.17 (m, 1H), 7.21-7.30 (m, 4H), 7.65 (dd, J=7 Hz, J=7 Hz, 1H), 7.74 (dd, J=7 Hz, J=7 Hz, 1H), 8.01-8.10 (m, 5H), 8.11-8.17 (brs, 3H), 8.18-8.25 (brs, 3H), 8.27-8.40 (brs, 3H), 8.41-8.55 (brs, 3H), 8.82-8.90 (brs, 1H), 9.12-9.18 (brs, 1H), 9.24 (d, J=7 Hz, 1H), 11.21-11.31 (brs, 1H); ESIMS found for C₃₆H₅₃N₁₁O₅ m/z 720 (M+H).

Synthesis of 3-[(1S)-2-[N′,N′-bis(2-azaniumylethyl)hydrazinecarbonyl]-1-{[(1S)-1-carbamoyl-2-[4-(trifluoromethyl)phenyl]ethyl]carbamoyl}ethan-1-aminium]quinolin-1-ium tetrachloride 10 is depicted below in scheme 8 and example 8.

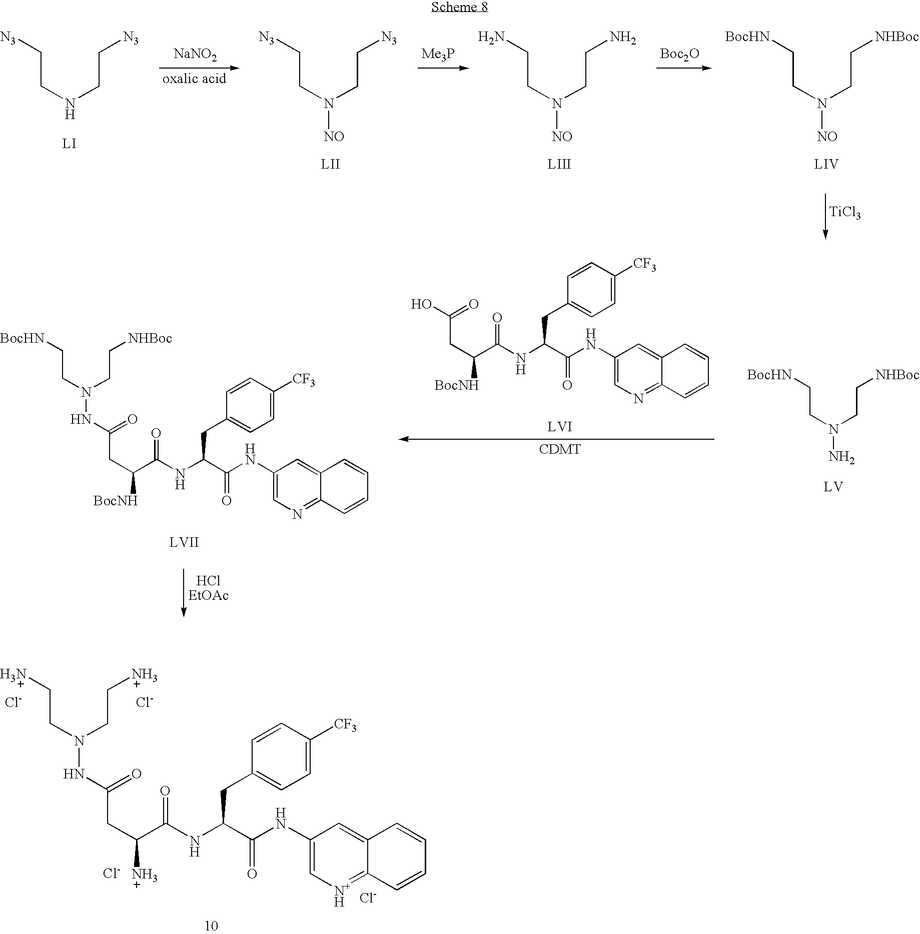

### Example 8

### Step 1

A suspension of sodium nitrate (13.4 g; 0.194 mol), oxalic acid (24.4 g; 0.194 mol) and N,N-bis(2-azidoethyl)amine LI (15 g; 0.097 mol) in DCM (300 mL) was stirred vigorously at r.t. for 2.5 h. Silica gel (20 g) and hexane (200 mL) was added to the reaction mixture and the resulting suspension was filtered. The solids were washed (1:1 hexane/DCM, 200 mL). The solvent was evaporated under reduced pressure to afford N,N-bis(2-azidoethyl)-N-nitrosoamine LII (15 g; 0.081 mol; 84% yield).

### Step 2

To a solution of N,N-bis(2-azidoethyl)-N-nitrosoamine LII (15 g; 0.081 mol) in dry THF was slowly added trimethylphosphine (1 M solution in THF; excess) while cooling the mixture reaction in cold water bath. The mixture was stirred overnight. The solution was evaporated under reduced pressure and crude N,N-bis(2-aminoethyl)-N-nitrosoamine LIII (12 g; quantitative yield) was used directly for step 3. ESIMS found for C₄H₁₂N₄O m/z 132.9 (M+H).

### Step 3

To a solution of the crude N,N-bis(2-aminoethyl)-N-nitrosoamine LIII (0.081 mol) in acetone (160 mL) was added 1 M aq. NaHCO₃ until the pH was 9-10. Di-tert-butyl dicarbonate (53 g; 0.243 mol) was the added in portions and stirred for 3 h. The acetone was evaporated and aqueous solution was extracted with EtOAc (3×). The combined organic phase was dried over MgSO₄ and then evaporated. The crude product was purified on a silica gel column (10:1→2:1 hexane/EtOAc) to give tert-butyl N-t2-[(2-t[(tert-butoxy)carbonyl]amino}ethyl)(nitroso)amino]ethyl}carbamate LIV (23.66 g; 0.071 mol; 88% yield). ¹H NMR (CDCl₃) 1.39 (s, 9H), 1.41 (s, 9H), 3.24 (dt, J=6 Hz, J=5 Hz, 2H), 3.52 (dt, J=6 Hz, J=5 Hz, 2H), 3.71 (t, J=6 Hz, 2H), 4.20 (t, J=6 Hz, 2H), 4.93 (brs, 1H), 5.07 (brs, 1H); ESIMS found for C₁₄H₂₈N₄O₅ m/z 333.2 (M+H).

### Step 4

To a solution of tert-butyl N-{2-[(2-{[(tert-butoxy)carbonyl]amino}ethyl)(nitroso)amino]ethyl}carbamate LIV (2 g; 6.02 mmol) in methanol (15 mL) was added a solution of titanium (III) chloride (3.7 g; 24.07 mmol) in water (20 mL). The mixture was stirred for 1.5 h and then cooled in a ice/water bath before adding KOH (12 g) in portions for 40 min. Stirring was continued for an additional 1 h at r.t. The reaction was filtered, solvent evaporated and purified on a silica gel column (1:1 chloroform/methanol) to obtain crude tert-butyl N-{2-[1-(2-{[(tert-butoxy)carbonyl]amino}ethyl)hydrazin-1-yl]ethyl}carbamate LV (0.85 g) used directly for step 5. ESIMS found for C₁₅H₃₂N₃O₄ m/z 319.4 (M+H).

### Step 5

Procedure can be found in examples 1-2.

### Step 6

Procedure can be found in examples 1-2. The final compound 10 was isolated as the hydrochloride salt. ¹H NMR (DMSO-d₆) 2.70 (dd, J=8 Hz, J=16 Hz, 1H), 2.85-2.94 (m, 5H), 2.99-3.04 (m, 4H), 3.08 (dd, J=11 Hz, J=14 Hz, 1H), 3.25-3.28 (m, 1H), 4.15-4.19 (m, 1H), 4.79-4.84 (m, 1H), 7.64 (d, J=8 Hz, 2H), 7.62-7.66 (m, 1H), 7.70 (d, J=8 Hz, 2H), 7.77 (dd, J=8 Hz, J=8 Hz, 1H), 7.97 (brs, 6H), 8.04 (d, J=9 Hz, 1H), 8.06 (d, J=9 Hz, 1H), 8.29 (brs, 6H), 8.87 (s, 1H), 9.19-9.21 (m, 2H), 9.68 (s, 1H), 11.51 (s, 1H); ESIMS found for C₂₇H₃₃F₃N₈O₃ m/z 575.5 (M+H).

Synthesis of 3-[(1S)-4-[2,1-bis(2-azaniumylethyl)carbamimidamido]-1-{[(1S)-1-carbamoyl-3-phenylpropyl]carbamoyl}butan-1-aminium]quinolin-1-ium tetrachloride 16 is depicted below in scheme 9 and example 9.

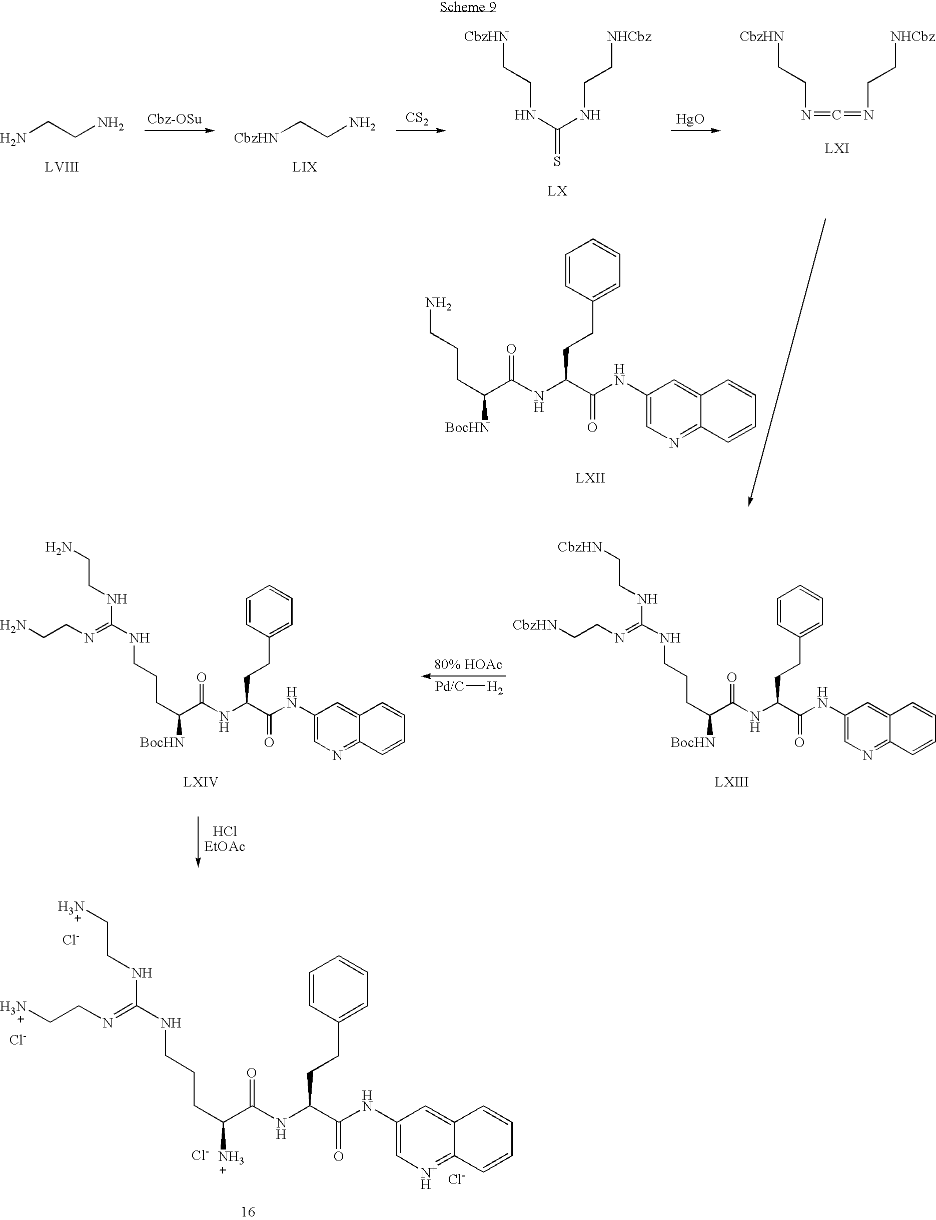

### Example 9

### Step 1

To a solution of 2-aminoethylamine LVIII (150 mL; 2.25 mol) in chloroform (1.5 L), cooled to 0° C. was added a solution of carbobenzoxy N-hydroxysuccinimide (112.14 g; 0.45 mol) in water (0.5 L) with vigorously stirring for one hour. The solid was filtered and the solution was washed with brine (3×), once with water and dried over anhydrous MgSO₄. The solvent was evaporated under reduced pressure and the residue was purified on a silica gel column (100:1→30:1 DCM:MeOH) to yield benzyl 2-aminoethylcarbamate LIX as a colorless viscous oil (350 g; 1.80 mol; 80% yield). ¹H NMR (CDCl₃) 2.79-2.83 (m, 2H), 3.21-3.28 (m, 2H), 5.10 (s, 2H), 5.19 (brs, 1H), 7.29-7.34 (m, 1H), 7.35-7.38 (m, 4H); ESIMS found for C₁₀H₁₄N₂O₂ m/z 195.2 (M+H).

### Step 2

To a suspension of benzyl 2-aminoethylcarbamate LIX (3 g; 15.45 mmol) in anhydrous ethanol (20 mL) was added carbon disulfide (0.47 mL; 7.73 mmol). The mixture was heated under gently reflux for 22 h. After cooling to r.t., the product precipitated and was filtered, washed with anhydrous ethanol (3×) and air dried. Benzyl N-(2-t[(2-{[(benzyloxy)carbonyl]amino}ethyl)carbamothioyl]amino}ethyl)carbamate LX was obtained as white solid (2.97 g; 6.90 mmol; 89.3% yield). ¹H NMR (DMSO-d₆) 3.10-3.14 (m, 4H), 3.37-3.42 (m, 4H), 5.00 (s, 4H), 7.30-7.37 (m, 12H), 7.54 (brs, 2H); ESIMS found for C₂₁H₂₆N₄O₄S m/z 431.4 (M+H).

### Step 3

To a suspension of benzyl N-(2-{[(2-{[(benzyloxy)carbonyl]amino}ethyl) carbamothioyl]amino}ethyl)carbamate LX (500 mg; 1.16 mmol) in DCM (20 mL) was added yellow mercuric (II) oxide (580 mg; 2.67 mmol). The mixture was stirred for 72 h before filtering the solid. The solution was evaporated under reduced pressure and the crude benzyl N-[2-({[(2-{[(benzyloxy)carbonyl]amino}ethyl)imino]methylidene}amino)ethyl]carbamate LXI was used for step 4 without further purification. ESIMS found for C₂₁H₂₄N₄O₄ m/z 397.4 (M+H).

### Step 4

To a solution of crude carbodiimide LXI in dry THF (30 mL) was added and tert-butyl N-[(1S)-4-amino-1-{[(1S)-3-phenyl-1-[(quinolin-3-yl)carbamoyl]propyl]carbamoyl}butyl]carbamate LXII (790 mg; 1.5 mmol). The mixture was refluxed for 20 h. After cooling, the solvent was evaporated under vacuum to afford a brown foam. The residue was dissolved in DCM and washed with 1 M HCl (2×), water and dried over anhydrous MgSO₄. The solvent was removed under vacuum and the residue was purified on a silica gel column (100% CHCl₃→50:3 CHCl₃/MeOH) to give benzyl N-{2-[(Z)-{[(2-t[(benzyloxy) carbonyl]amino}ethyl)amino] ({[(4S)-4-{[(tert-butoxy)carbonyl]amino}-4-{[(1S)-3-phenyl-1-[(quinolin-3-yl)carbamoyl]propyl]carbamoyl}butyl]amino})methylidene}amino]ethyl}carbamate LXIII as an amorphous white solid (424 mg; 0.463 mmol; 40% yield for 2 steps). ESIMS found for C₅₀H₆₁N₉O₈ m/z 916.7 (M+H).

### Step 5

To a solution of benzyl N-{2-[(Z)-{[(2-{[(benzyloxy)carbonyl]amino}ethyl)amino] ({[(4S)-4-{[(tert-butoxy)carbonyl]amino}-4-{[(1S)-3-phenyl-1-[(quinolin-3-yl) carbamoyl]propyl]carbamoyl}butyl]amino})methylidene}amino]ethyl}carbamate LXIII (420 mg; 0.46 mmol) in 80% of acetic acid (25 mL) was added 10% Pd/C (catalytic amount). The mixture was stirred under hydrogen for three days before filtering through Celite and concentrated under reduced pressure. The residue was co-evaporated with toluene (3×) and dried under vacuum to give tert-butyl N-[(1S)-4-[2,1-bis(2-aminoethyl)carbamimidamido]-1-{[(1S)-3-phenyl-1-[(quinolin-3-yl)carbamoyl]propyl]carbamoyl}butyl]carbamate LXIV as a off-white foam (260 mg; 0.40 mmol; 87% yield). ESIMS found for C₃₄H₄₉N₉O₄ m/z 648.8 (M+H).

### Step 6

Procedure can be found in examples 1-2. The final compound 16 was isolated as the hydrochloride salt. ¹H NMR (DMSO-d₆) 1.73-1.80 (m, 2H), 1.84-1.93 (m, 2H), 2.04-2.11 (m, 1H), 2.13-2.19 (m, 1H), 2.69-2.75 (m, 1H), 2.82-2.88 (m, 1H), 3.01-3.10 (m, 4H), 3.38-3.43 (m, 2H), 3.60-3.66 (m, 4H), 4.09-4.13 (m, 1H) 4.57-4.62 (m, 1H), 7.15-7.19 (m, 1H), 7.22-7.33 (m, 4H), 7.72 (dd, J=8 Hz, J=8 Hz, 1H), 7.82 (dd, J=8 Hz, J=8 Hz, 1H), 8.07 (brs, 2H), 8.14-8.16 (m, 3H), 8.33 (brs, 6H), 8.44 (brs, 3H), 8.99 (s, 1H), 9.28 (s, 1H), 9.33 (d, J=7 Hz, 1H), 11.44 (s, 1H); ESIMS found for C₂₉H₄₁N₉O₂ m/z 548.7 (M+H).

Synthesis of 3-[(1R)-2-[bis(2-azaniumylethyl)sulfamoyl]-1-{[(1S)-1-carbamoyl-2-[4-(trifluoromethyl)phenyl]ethyl]carbamoyl}ethan-1-aminium]quinolin-1-ium tetrachloride 17 is depicted below in scheme 10 and example 10.

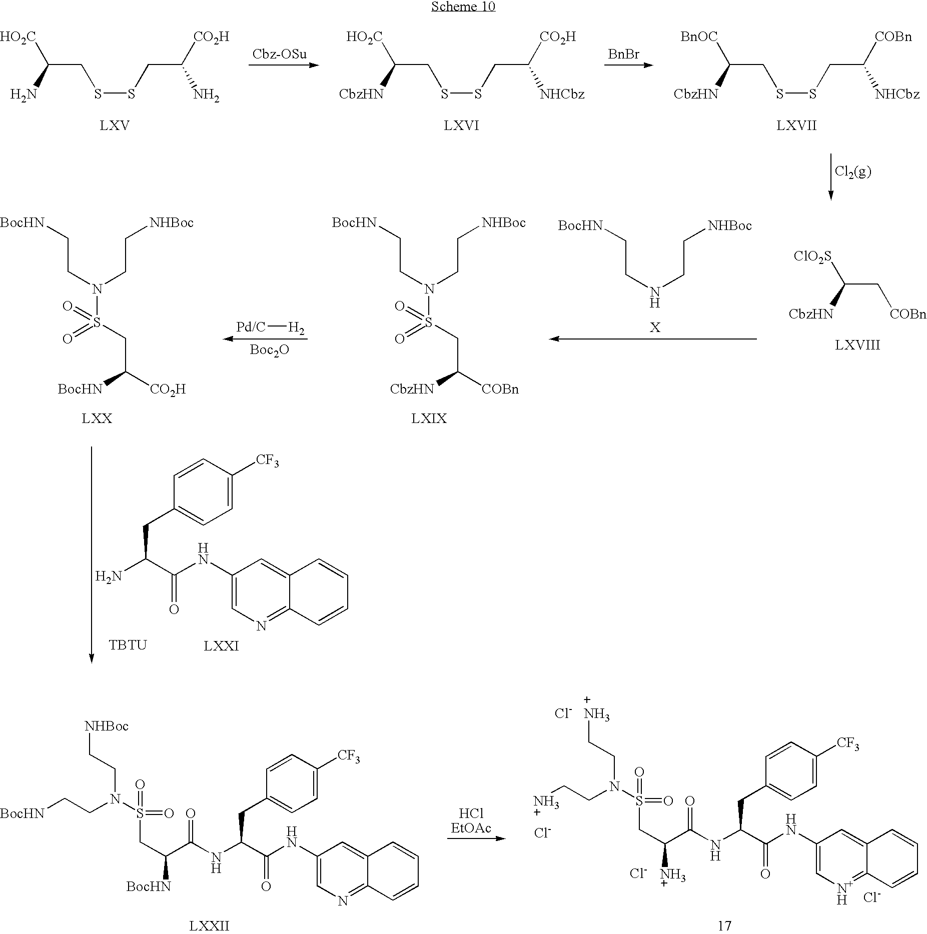

### Example 10

### Step 1

To a solution of L-Cystine (3 g; 12.5 mmol) in acetone (25 mL) was added M NaOH (20 mL), water (16 mL) and carbobenzoxy N-hydroxysuccinimide (7.5 g; 30 mmol). The mixture was stirred overnight at r.t. before the acetone was removed. The remaining aqueous phase was adjusted to pH=11 with 1 M NaOH, washed with diethyl ether and acidified to pH˜5. The white precipitate was filtered, washed with water and dried. The crude product was purified on a silica gel column (100% CHCl₃→100:7 CHCl₃/MeOH) to give (2S)-2-{[(benzyloxy)carbonyl]amino}-3-{[(2S)-2-{[(benzyloxy)carbonyl]amino}-2-carboxyethyl]disulfanyl}propanoic acid LXVI (3.52 g; 6.92 mmol; 55% yield). ¹H NMR (DMSO-d₆) 2.91 (dd, J=13 Hz, J=10 Hz, 2H), 3.14 (dd, J=13 Hz, J=4 Hz, 2H), 4.27 (ddd, J=13 Hz, J=10 Hz, J=4 Hz, 2H), 5.00-5.06 (m, 4H), 7.29-7.32 (m, 2H), 7.34-7.37 (m, 8H), 7.74 (d, J=8 Hz, 2H), 13.03 (brs, 2H); ESIMS found for C₂₂H₂₄N₂O₈S₂ m/z 509.2 (M+H).

### Step 2

To a solution of (2S)-2-{[(benzyloxy)carbonyl]amino}-3-t[(2S)-2-{[(benzyloxy)carbonyl]amino}-2-carboxyethyl]disulfanyl}propanoic acid LXVI (5.6 g; 11 mmol) and anhydrous potassium carbonate (6.08 g; 44.0 mmol) in DMF (50 mL) cooled to 0° C. was added benzyl bromide (7.8 mL; 66.0 mmol). The mixture was stirred overnight at r.t. Water (150 mL) was added, and solution was extracted once with DCM (40 mL). The solvent was evaporated under reduced pressure and the residue was dissolved in diethyl ether (60 mL). The ether was washed with 10% aq Na₂S₂O₃ until all the DMF was removed and once with water. The organic phase was dried over anhydrous MgSO₄, evaporated and the residue purified on a silica gel column (100% CHCl₃→200:3 CHCl₃:MeOH). Benzyl (2S)-3-{[(2S)-3-(benzyloxy)-2-{[(benzyloxy)carbonyl]amino}-3-oxopropyl]disulfanyl}-2-{[(benzyloxy)carbonyl]amino}propanoate LXVII was obtained as a light-green viscous oil (5.9 g; 8.56 mmol; 77.8% yield). ¹H NMR(CDCl₃) 3.04-3.18 (m, 4H), 4.62-4.76 (m, 2H), 5.12 (s, 2H), 5.17 (s, 2H), 5.62-5.76 (m, 2H), 7.28-7.40 (m, 20H); ESIMS found for C₃₆H₃₆N₂O₈S₂ m/z 689.5 (M+H).

### Step 3

To a solution of Benzyl (2S)-3-{[(2S)-3-(benzyloxy)-2-{[(benzyloxy) carbonyl]amino}-3-oxopropyl]disulfanyl}-2-{[(benzyloxy)carbonyl]amino}propanoate LXVII (5.85 g; 8.50 mmol) in carbon tetrachloride (60 mL) and anhydrous ethanol (15 mL) was bubbled gaseous chlorine for 40 minutes while cooling in an ice/water bath. The excess chlorine was removed by bubbling argon through the mixture. The solvent was removed under reduced pressure to give crude benzyl (2S)-2-{[(benzyloxy)carbonyl]amino}-3-(chlorosulfonyl)propanoate LXVIII as a white solid (5.95 g; 12.7 mmol; 75% yield). ESIMS found for C₁₈H₁₈ClNO₆S m/z 412.3/414.3 (³⁵Cl/³⁷Cl) (M+H).

### Step 4

To a solution of crude compound LXVIII (5.93 g; 12.66 mmol) in DCM (90 mL) cooled in an ice/water bath was added tert-butyl N-{2-[(2-t[(tert-butoxy)carbonyl]amino}ethyl)amino]ethyl}carbamate X (4.8 g; 15.84 mmol). After 10 min, TEA (3 mL; 21.6 mmol) was added and after another 30 min the reaction was warmed to r.t. and stirred overnight. DCM (100 mL) was then added and washed with 1 M HCl (2×150 mL), 5% aq NaHCO₃ (100 mL) and dried over anhydrous MgSO₄. The solvent was removed under reduced pressure and the residue was purified on a silica gel column (100% hexane→3:4 hexane/EtOAc). The product was further crystallized from hexane to give benzyl (2S)-2-{[(benzyloxy)carbonyl]amino}-3-[bis(2-{[(tert-butoxy)carbonyl]amino}ethyl) sulfamoyl]propanoate LXIX (1.82 g; 2.68 mmol; 21% yield). ESIMS found for C₃₂H₄₆N₄O₁₀S m/z 679.5 (M+H).

### Step 5

To a solution of benzyl (2S)-2-{[(benzyloxy)carbonyl]amino}-3-[bis(2-{[(tert-butoxy)carbonyl]amino}ethyl) sulfamoyl]propanoate LXIX (1.80 g; 2.65 mmol) in EtOAc (45 mL) was added TEA (0.4 mL; 2.9 mmol), di-tert-butyl dicarbonate (633 mg; 2.9 mmol) and 10% Pd/C (200 mg). The mixture was stirred under an hydrogen atmosphere overnight at r.t. before filtering through Celite and concentrating under reduced pressure. The product was purified on a silica gel column (100% CHCl₃→100:3 CHCl₃:MeOH) to obtain (2S)-3-[bis(2-{[(tert-butoxy)carbonyl]amino}ethyl)sulfamoyl]-2-{[(tert-butoxy)carbonyl]amino}propanoic acid LXX as colorless viscously oil (1.24 g; 2.23 mmol; 84.4% yield). ESIMS found for C₂₂H₄₂N₄O₁₀S m/z 555.6 (M+H).

### Step 6

Procedure can be found in examples 1-2.

### Step 7

Procedure can be found in examples 1-2. The final compound 17 was isolated as the hydrochloride salt. ¹H NMR (DMSO-d₆) 3.02-3.07 (m, 4H), 3.18 (dd, J=8 Hz, J=14 Hz, 1H), 3.27-3.34 (m, 1H), 3.61-3.64 (m, 4H), 3.68 (dd, J=8 Hz, J=14 Hz, 1H), 3.86 (dd, J=4 Hz, J=14 Hz, 1H), 4.37-4.43 (m, 1H), [4.81 (dd, J=8 Hz, J=14 Hz, 1^{st} rotamer), 4.87 (dd, J=8 Hz, J=14 Hz, 2^{nd} rotamer), 1H], [7.58 (d, J=8 Hz, 2^{nd} rotamer), 7.63 (d, J=8 Hz, 1^{st} rotamer), 2H], 7.64-7.66 (m, 3H), 7.72 (dd, J=7 Hz, J=8 Hz, 1H), [7.99 (d, J=8 Hz, 2^{nd} rotamer), 8.03 (d, J=8 Hz, 1^{st} rotamer), 1H], 8.02 (d, J=7 Hz, 1H), [8.22 (brs, 1^{st} rotamer), 8.25 (brs, 2 rotamer), 6H], [8.61 (brs, 1^{st} rotamer), 8.64 (brs, 2 rotamer), 3H], [8.77 (s, 2^{nd} rotamer), 8.80 (s, 1^{st} rotamer), 1H], 9.12 (s, 1H), 9.55 (d, J=7 Hz, 1H), [11.12 (s, 2^{nd} rotamer), 11.22 (s, 1^{st} rotamer), 1H]; ¹⁹F NMR (DMSO-d₆)-60.06 (1^{st} rotamer), −60.13 (2^{nd} rotamer) (s, 3F); ESIMS found for C₂₆H₃₂F₃N₇O₄S m/z 596.6 (M+H).

Synthesis of 3-[(1S)-2-[bis(2-aminioethyl)carbamoyl]-1-{[(1S)-1-carbamoyl-2-(piperidin-1-ium-4-yl)ethyl]carbamoyl}ethanaminium]quinolin-1-ium pentachloride 18 is depicted below in scheme 11 and example 11.

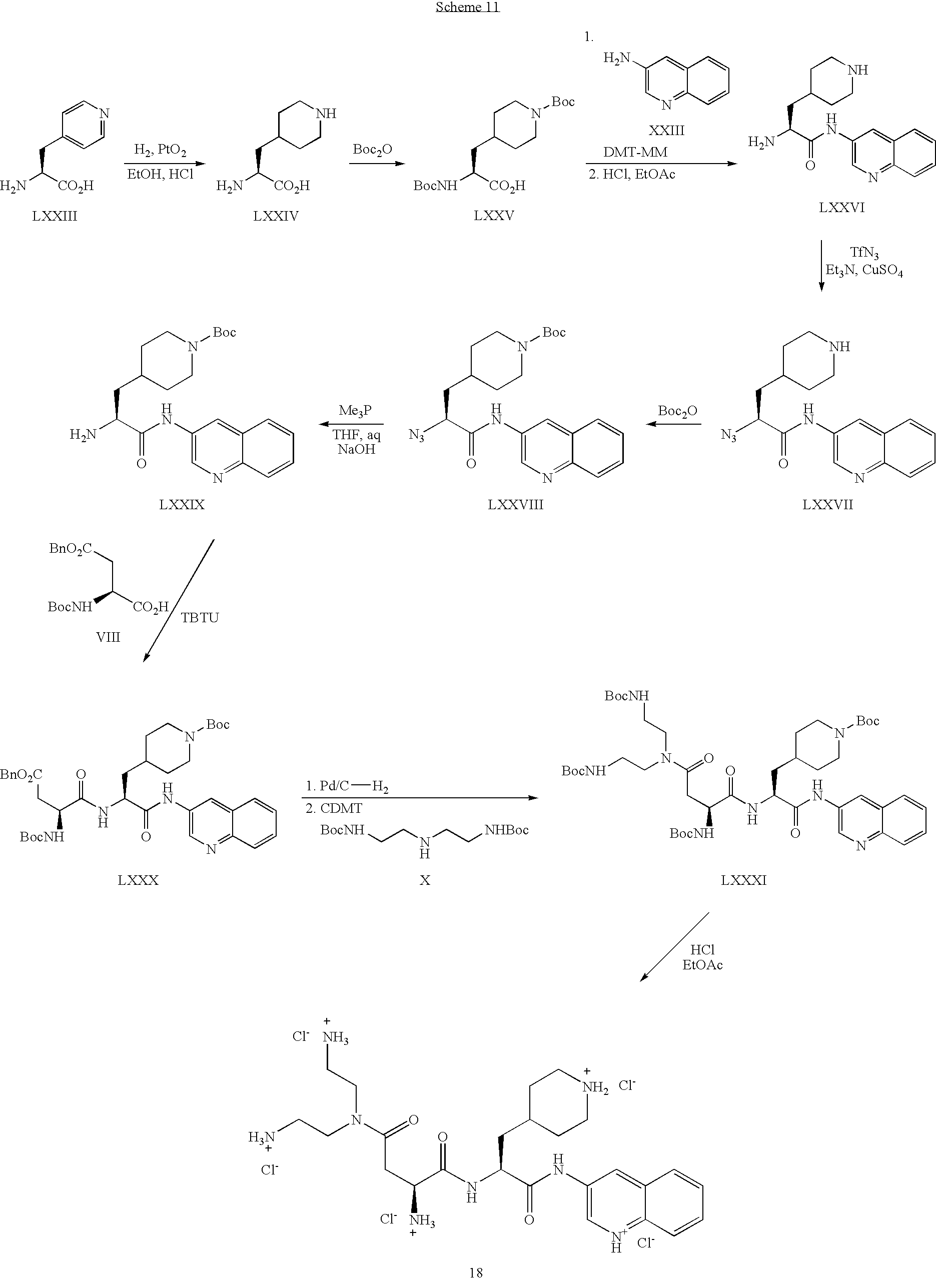

### Example 11

### Step 1

To a solution of (2S)-2-amino-3-(pyridin-4-yl)propanoic acid LXXIII (660 mg, 4.0 mmol) in ethanol (120 mL) was added 1 N HCl (10 mL) and PtO₂ (150 mg). The mixture was vigorously shaken 70 psi H₂ in a Parr apparatus for 48 h. The mixture was filtered through Celite and the filtrate was concentrated to dryness giving crude (2S)-2-amino-3-(piperidin-4-yl)propanoic acid LXXIV as the hydrochloride salt (988 mg). ESIMS found for C₈H₁₆NO₂ m/z 172.0 (M+).

### Step 2

To a suspension of (2S)-2-amino-3-(piperidin-4-yl)propanoic acid LXXIV (988 mg) in DCM (30 mL) was added TEA (3.3 mL, 24 mmol) and Boc₂O (2.0 g, 8.8 mmol). The mixture was stirred at r.t. overnight. It was evaporated to dryness under reduced pressure before adding water (100 mL) and extracting with diethyl ether. Water layer was separated and acidified with 1 N HCl until pH=3 and extracted with ethyl acetate. The organic layer was dried over Na₂SO₄ and concentrated to dryness to obtain crude (2S)-2-{[(tert-butoxy)carbonyl]amino}-3-{1-[(tert-butoxy)carbonyl]piperidin-4-yl}propanoic acid LXXV (1.25 g). ESIMS found for C₁₈H₃₂N₂O₆ m/z 373 (M+H).

### Step 3

To a solution of (2S)-2-{[(tert-butoxy)carbonyl]amino}-3-t1-[(tert-butoxy)carbonyl]piperidin-4-yl}propanoic acid LXXV (450 mg 1.21 mmol) and 3-aminoquinoline (187 mg, 1.30 mmol) in DCM (20 mL) was added DMT-MM (387 mg, 1.4 mmol). The mixture was stirred at r.t. overnight. The reaction was washed with water, 1 N HCl, satd. aq. NaHCO₃, water and dried over Na₂SO₄. The product was purified on a silica gel column (1:1 EtOAc:hexane) to give tert-butyl 4-[(2S)-2-{[(tert-butoxy)carbonyl]amino}-2-[(quinolin-3-yl)carbamoyl]ethyl]piperidine-1-carboxylate (425 mg, 0.85 mmol, 70% yield). ¹H NMR (DMSO-d₆) 1.02-1.10 (m, 2H), 1.40 (s, 18H), 1.54-1.73 (m, 9H), 4.22-4.25 (m, 1H), 7.20 (d, J=8 Hz, 1H), 7.55-7.66 (m, 2H), 7.94 (t, J=8 Hz, 2H), 8.69 (d, J=2 Hz, 1H), 8.93 (d, J=2 Hz, 1H), 10.44 (s, 1H). ESIMS found for C₂₇H₃₈N₄O₅ m/z 499 (M+H).

### Step 4

To a solution of tert-butyl 4-[(2S)-2-{[(tert-butoxy)carbonyl]amino}-2-[(quinolin-3-yl)carbamoyl]ethyl]piperidine-1-carboxylate (425 mg, 0.85 mmol) was added HCl/EtOAc (5 M solution, 6 mL) at r.t. overnight. The precipitate was filtered, washed with ethyl acetate, diethyl ether and dried to give crude (2S)-2-amino-3-(piperidin-4-yl)-N-(quinolin-3-yl)propanamide LXXVI as the hydrochloride salt (351 mg). ESIMS found for C₁₇H₂₂N₄O m/z 299 (M+H).

### Step 5

To a solution of (2S)-2-amino-3-(piperidin-4-yl)-N-(quinolin-3-yl) propanamide LXXVI in a mixture of methanol (15 mL) and water (5 mL) was added Et₃N (0.60 mL, 4.24 mmol) and CuSO₄ (20 mg). The mixture was treated at 0° C. with freshly prepared solution of triflic azide (5.9 mmol) in DCM (10 mL). The mixture was stirred at r.t. for 48 h. The solvent was evaporated under reduced pressure and dissolved in EtOAc, washed with satd. aq. NaHCO₃, water and dried over Na₂SO₄. The solvent was removed under vacuum to give crude (2S)-2-azido-3-(piperidin-4-yl)-N-(quinolin-3-yl) propanamide LXXVII (320 mg). ESIMS found for C₁₇H₂₀N₆O m/z 325 (M+H).

### Step 6

To a solution of (2S)-2-azido-3-(piperidin-4-yl)-N-(quinolin-3-yl) propanamide LXXVII in DCM (10 mL) was added TEA (0.60 mL, 4.25 mmol) followed by Boc₂O (202 mg, 0.93 mmol). The mixture was stirred at r.t. overnight before the solvent was removed under reduced pressure. The residue was purified on a silica gel column (1:1→2:1 EtOAc:hexane) to give tert-butyl 4-[(2S)-2-azido-2-[(quinolin-3-yl)carbamoyl]ethyl]piperidine-1-carboxylate LXXVIII (262 mg, 0.62 mmol, 73% yield for 3 steps). ¹H NMR (DMSO-d₆) 1.11-1.22 (m, 2H), 1.45 (s, 9H), 1.60-1.79 (m, 9H), 4.30-4.39 (m, 1H), 7.53-7.67 (m, 2H), 7.97 (t, J=8 Hz, 2H), 8.70 (d, J=2 Hz, 1H), 8.95 (d, J=2 Hz, 1H), 10.38 (s, 1H). ESIMS found for C₂₂H₂₈N₆O₃ m/z 425 (M+H).

### Step 7

To a solution of tert-butyl 4-[(2S)-2-azido-2-[(quinolin-3-yl)carbamoyl]ethyl]piperidine-1-carboxylate LXXVIII (262 mg, 0.62 mmol) in THF (20 mL) and 0.1 M NaOH (2.0 mL) was added Me₃P (1 M in THF, 0.65 mL). The reaction was stirred at r.t. overnight. The solvent was removed under reduced pressure to give crude tert-butyl 4-[(2S)-2-amino-2-[(quinolin-3-yl)carbamoyl]ethyl]piperidine-1-carboxylate LXXIX (275 mg), which was directly used in step 8. ESIMS found for C₂₂H₃₀N₄O₃ m/z 399 (M+H).

### Step 8-10

Procedures can be found in examples 1-2.

### Step 11

Procedure can be found in examples 1-2. The final compound 18 was isolated as the hydrochloride salt. ¹H NMR (DMSO-d₆) 1.38-1.46 (m, 2H), 1.78-1.88 (m, 2H), 2.82-2.92 (m, 2H), 3.01-3.20 (m, 6H), 3.21-3.29 (m, 4H), 3.58-3.66 (m, 4H), 4.34-4.40 (m, 1H), 4.60-4.66 (m, 1H), 7.65-7.69 (m, 1H), 7.74-7.78 (m, 1H), 8.05-8.09 (m, 5H), 8.10 (brs, 3H), 8.47 (brs, 3H), 8.78-8.82 (m, 1H), 8.90 (d, J=2 Hz, 1H), 8.98-9.01 (m, 1H), 9.10 (d, J=7 Hz, 1H), 9.22 (d, J=2 Hz, 1H), 11.05 (s, 1H). ESIMS found for C₂₅H₃₈N₈O₃ m/z 499 (M+H).

Synthesis of 3-[(2S)-2-[(1S)-3-[(2-aminioethyl)[2-(trimethylaminio)ethyl]carbamoyl]-1-formamidopropan-1-aminium]-3-[4-(trifluoromethyl)phenyl]propanamido]quinolin-1-ium tetrachloride 19 is depicted below in scheme 12 and example 12.

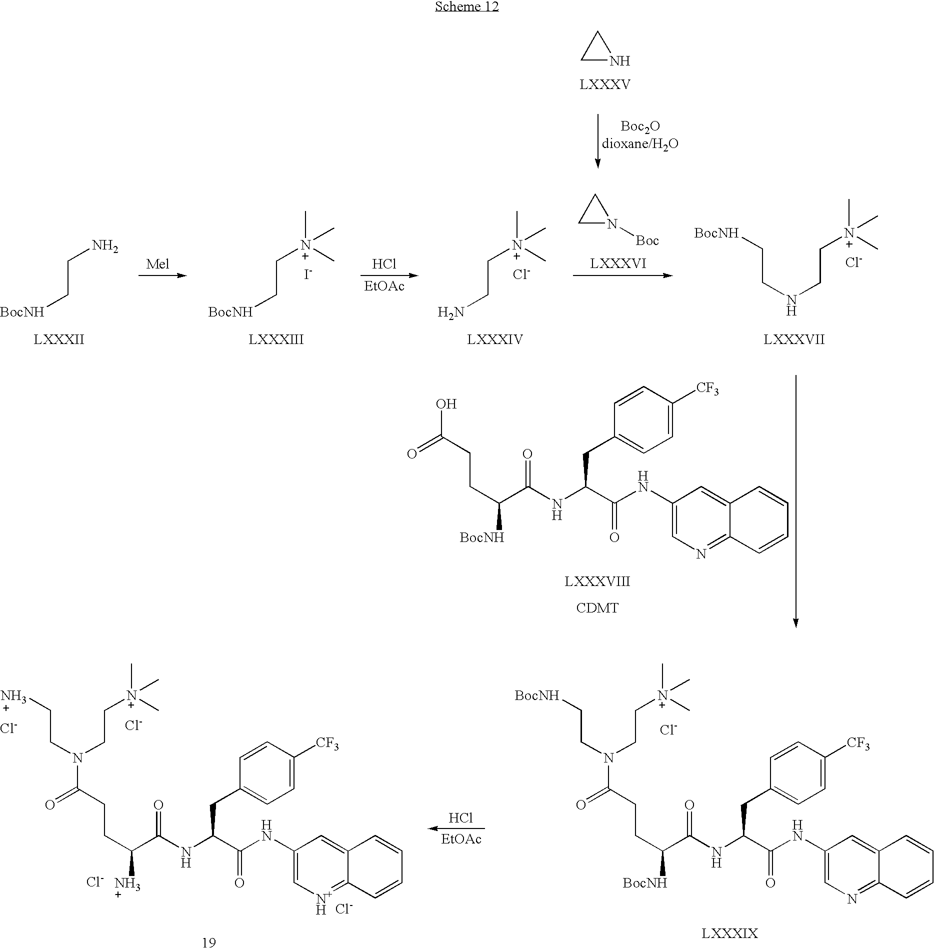

### Example 12

### Step 1

To a solution of tert-butyl N-(2-aminoethyl)carbamate LXXXII (2.6 g; 16.35 mmol) in MeI (5 mL) was added anhydrous potassium carbonate (4.7 g; 34.0 mmol). The mixture was stirred for 24 h at r.t. The MeI was removed under reduced pressure and the residue was crystallized from ethanol and then triturated with ethyl acetate to give tert-butyl N-[2-(trimethylaminio)ethyl]carbamate iodide LXXXIII (3.2 g; 13.40 mmol 82% yield). ¹H NMR (CDCl₃) 1.42 (s, 9H), 3.49 (s, 9H), 3.68-3.72 (m, 2H) 3.82 (t, J=5 Hz, 2H), 5.85-5.92 (m, 1H); ESIMS found for C₁₀H₂₃N₂O₂ m/z 203.3 (M+).

### Step 2

To a solution of tert-butyl N-[2-(trimethylaminio)ethyl]carbamate iodide LXXXIII (3.2 g; 13.4 mmol) in ethyl acetate cooled in ice/water bath was added HCl (3.5 M solution in EtOAc). The reaction mixture was stirred by 30 min at r.t. The white precipitate was filtered and washed with ether to give (2-aminioethyl)trimethylazanium dichloride LXXXIV (1.9 g; quantitative). ESIMS found for C₅H₁₅N₂ m/z 102.9 (M+).

### Step 3

To a solution of aziridine (100 g; 2.3 mol) in dioxane (2 L) and water (1 L) the aziridine (100 g; 2.30 mol), cooled to 0° C. in ice water bath, was added di-tert-butyl dicarbonate (530 g; 2.41 mol) in portion over 2 h. The mixture was stirred at r.t. overnight. tert-Butyl aziridine-1-carboxylate LXXXVI was isolated as a mixture with dioxane by distillation.

### Step 4

To the solution tert-butyl aziridine-1-carboxylate LXXXVI (excess) in dioxane was added (2-aminioethyl)trimethylazanium dichloride LXXXIV (0.25 g; 1.8 mmol). The mixture was refluxed for 3 days. The solvent was removed under reduced pressure and the residue was purified on a silica gel column (100:1 CHCl₃:MeOH) to give crude tert-butyl N-(2-{[2-(trimethylaminio)ethyl]amino}ethyl)carbamate chloride LXXXVII was used directly for step 5.

### Step 5

Procedure can be found in examples 1-2.

### Step 6

Procedure can be found in examples 1-2. The final compound 19 was isolated as the hydrochloride salt. ESIMS found for C₃₁H₄₁F₃N₇O₃ m/z 616 (M+).

Synthesis of 3-[(1S)-3-[bis(2-aminioethyl)carbamoyl]-1-{[1-carbamoyl-2-(piperidin-1-ium-1-yl)ethyl]carbamoyl}propan-1-aminium]quinolin-1-ium pentachloride 20 is depicted below in scheme 13 and example 13.

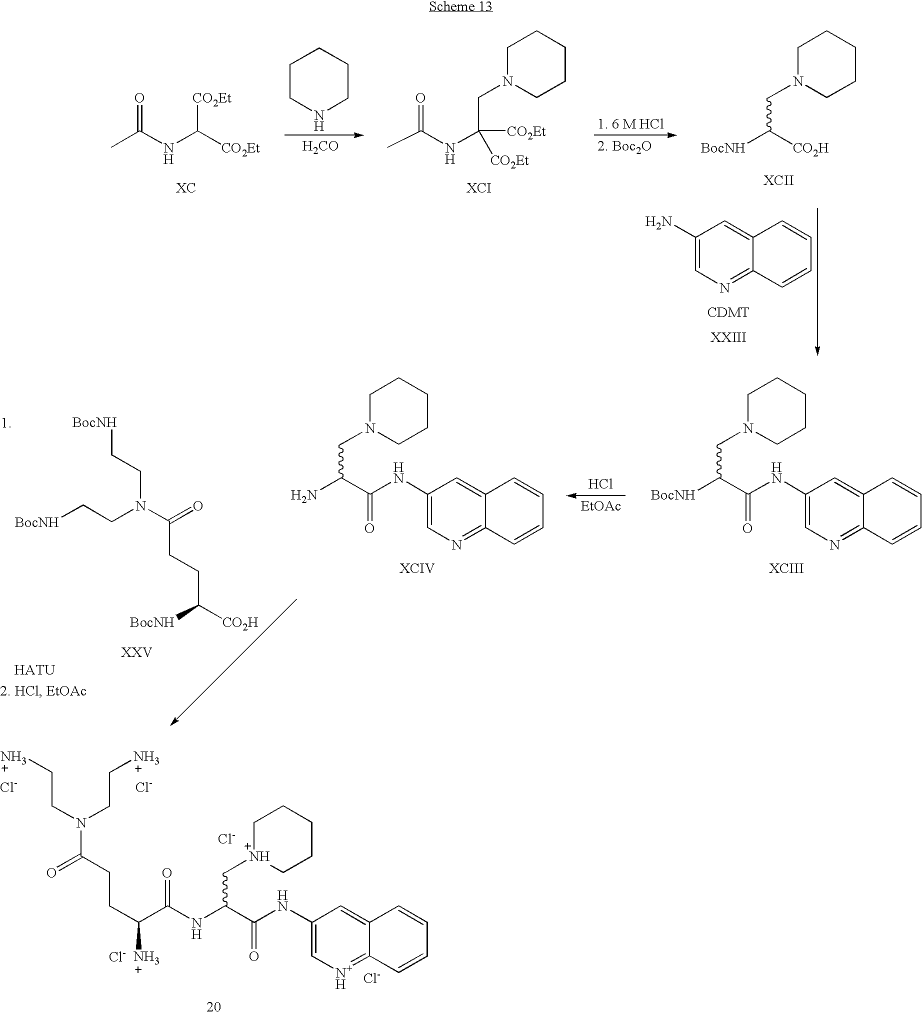

### Example 13

### Step 1

To a solution of diethyl acetamidomalonate XC (1.75 g, 8.05 mmol) in THF (20 mL) was added piperidine (0.62 mL, 6.7 mmol) and 36% aqueous solution formaldehyde (0.23 mL, 8.25 mmol). The reaction mixture was stirred at 60° C. for 5 min. The mixture was cooled to −5° C. and kept at this temperature overnight. The precipitate was filtered to produce 1,3-diethyl 2-acetamido-2-(piperidin-1-ylmethyl)propanedioate XCI as a white crystallized solid (1.01 g, 3.21 mmol, 40% yield). ¹H NMR (CDCl₃) 1.15-1.30 (m, 6H), 1.31-1.56 (m, 6H), 2.03 (s, 3H), 2.30-2.51 (m, 4H), 3.25 (s, 2H), 4.13-4.32 (m, 4H), 6.99 (brs, 1H); ESIMS found for C₁₅H₂₆N₂O₅ m/z 315 (M+H).

### Step 2

A solution of 1,3-diethyl 2-acetamido-2-(piperidin-1-ylmethyl) propanedioate XCI (1.01 g, 3.21 mmol) in 6 M HCl (20 mL) was refluxed overnight. The reaction mixture was alkalized with 4 M NaOH to pH=11 before adding a solution of Boc₂O (1.40 g, 6.42 mmol) in acetone (25 mL). The reaction mixture was stirred overnight at r.t. The acetone was evaporated under reduced pressure and the remaining water was washed with ethyl ether (2×) and acidified to pH=8 with 2 M aqueous HCl. The water was evaporated under reduced pressure and solid residue was purified on a silica gel column (30:1 CHCl₃:MeOH) to give 2-{[(tert-butoxy)carbonyl]amino}-3-(piperidin-1-yl)propanoic acid XCII (0.44 g, 1.61 mmol, 50% yield). ¹H NMR (CDCl₃) 1.41 (s, 9H), 1.77-2.01 (m, 4H), 2.87-3.05 (m, 2H), 3.33-3.42 (m, 2H), 3.44 (brs, 4H), 4.07-4.20 (m, 1H), 5.85 (brs, 1H); ESIMS found for C₁₃H₂₄N₂O₄ m/z 273 (M+H).

### Step 3

To a solution of 2-{[(tert-butoxy)carbonyl]amino}-3-(piperidin-1-yl)propanoic acid XCII (440 mg, 1.61 mmol) in DCM (15 mL) was added DIPEA (0.33 mL, 1.93 mmol), 3-aminoquinoline XXIII (255 mg 1.77 mmol) and TBTU (568 mg, 1.77 mmol). The mixture was stirred at r.t. overnight. The mixture was washed with 1 M K₂CO₃, 1 M HCl, brine and dried over MgSO₄. Product was purified on a silica gel column (200:1→100:1 CHCl₃:MeOH) and then crystallized from ether to give tert-butyl N-[2-(piperidin-1-yl)-1-[(quinolin-3-yl)carbamoyl]ethyl]carbamate XCIII (450 mg, 1.13 mmol, 70% yield). ¹H NMR(CDCl₃) 1.48 (s, 9H), 1.55-1.67 (m, 4H), 1.68-1.83 (m, 4H), 2.47-2.65 (m, 2H), 2.82-2.98 (m, 2H), 4.39 (brs, 1H), 5.62 (brs, 1H), 7.54 (dd, J=7 Hz, J=7 Hz, 1H), 7.58-7.69 (m, 1H), 7.81 (d, 8 Hz, 1H), 8.03 (d, J=8 Hz, 1H), 8.74 (brs, 2H), 11.78 (brs, 1H); ESIMS found for C₂₂H₃₀N₄O₃ m/z 399 (M+H).

### Step 4

To a solution of tert-butyl N-[2-(piperidin-1-yl)-1-[(quinolin-3-yl)carbamoyl]ethyl]carbamate XCIII (450 mg, 1.13 mmol) in ethyl acetate (10 mL) was added HCl (4.5 M solution in EtOAc, 10 mL). The reaction mixture was stirred for 20 min at r.t. before adding ethyl ether (20 mL). The precipitate was filtered and washed with ether to give 2-amino-3-(piperidin-1-yl)-N-(quinolin-3-yl)propanamide XCIV as a white crystalline solid (400 mg, 1.07 mmol, 94.7% yield). ESIMS found for C₁₇H₂₂N₄O m/z 299 (M+H).

### Step 5

Procedure can be found in examples 1-2.

### Step 6

Procedure can be found in examples 1-2. The final compound 20 was isolated as the hydrochloride salt. ¹H NMR (DMSO-d₆) 1.36-1.47 (m, 1H), 1.66-1.74 (m, 1H), 1.76-1.90 (m, 4H), 1.93-2.00 (m, 1H), 2.03-2.15 (m, 2H), 2.61-2.82 (m, 2H), 2.89-2.99 (m, 2H), 3.02-3.11 (m, 2H), 3.44-3.57 (m, 4H), 3.58-3.82 (m, 4H), 3.94-4.04 (m, 1H), 5.00-5.07 (m, 1H), 5.21-5.30 (m, 1H), 7.61-7.79 (m, 2H), 7.94-8.11 (m, 3H), 8.25-8.42 (m, 3H), 8.50-8.68 (m, 3H), 8.82 (brs, 1H), [9.23 (s, 1^{st} diastereoisomer); 9.19 (s, 2^{nd} diastereoisomer), 1H], 9.68-9.53 (m, 1H), [10.21 (brs, 1^{st} diastereoisomer); 10.07 (brs, 2^{nd} diastereoisomer), 1H], [11.73 (s, 1^{st} diastereoisomer); 11.33 (s, 2^{nd} diastereoisomer), 1H]; ESIMS found for C₂₆H₄₀N₈O₃ m/z 513 (M+H).

Synthesis of 3-{[bis(2-aminioethyl)carbamoyl]({[(1S)-1-carbamoyl-2-[4-(trifluoromethyl)phenyl]ethyl]carbamoyl})methanaminium}quinolin-1-ium tetrachloride 21 is depicted below in scheme 14 and example 14.

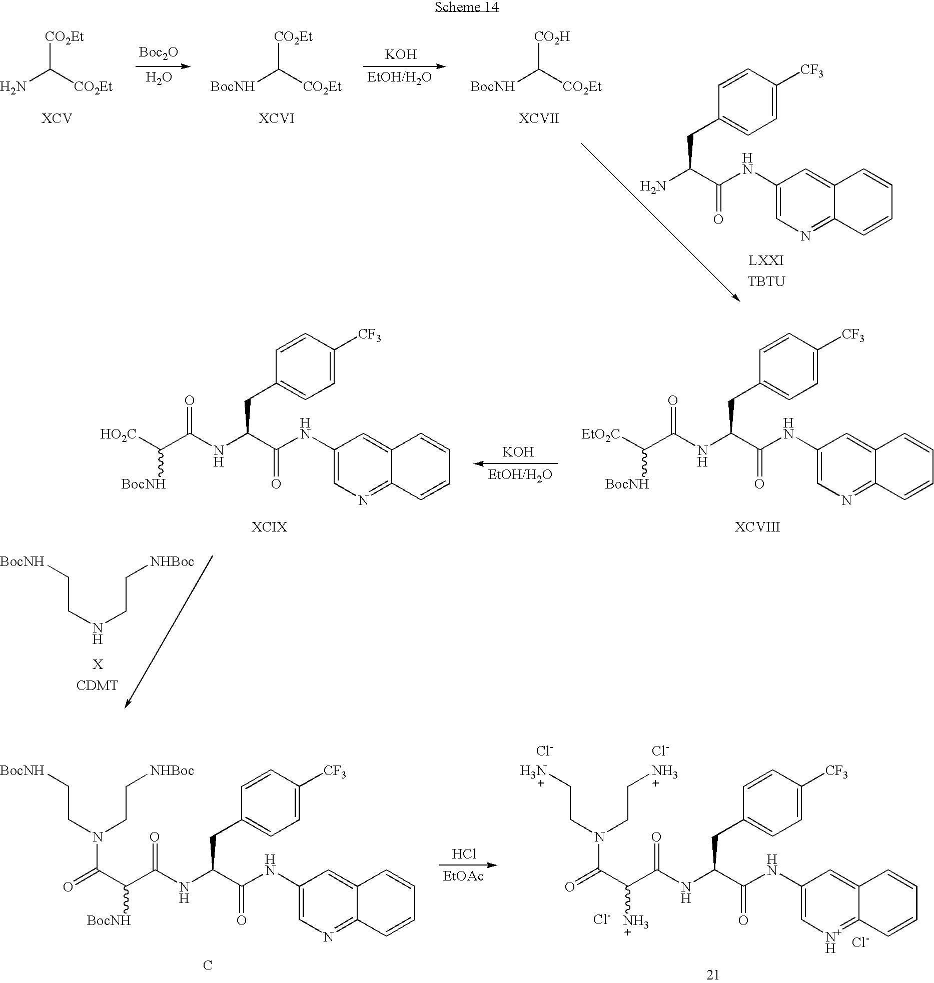

### Example 14

### Step 1

To a solution of diethyl aminomalonate hydrochloride XCV (2.0 g, 9.45 mmol) in water (45 mL) was added 1 M NaOH to pH˜8. Boc₂O (3.72 g, 17.0 mmol) in acetone (15 mL) was then added. The reaction mixture was stirred for 2 days before the acetone was evaporated under reduced pressure. The residue was washed by diethyl ether, and the organic layer was evaporated under vacuum to give the crude 1,3-diethyl 2-{[(tert-butoxy)carbonyl]amino}propanedioate XCVI as a colorless oil (2.22 g, 8 mmol, 85% yield). The crude product was used directly in step 2. ESIMS found for C₁₂H₂₁NO₆ m/z 276 (M+H).

### Step 2

To a solution of 1,3-diethyl 2-{[(tert-butoxy)carbonyl]amino}propanedioate XCVI (2.22 g, 8 mmol) in a mixture of ethanol/water (45 mL/5 mL) was added KOH (0.45 g, 8 mmol) in water (3 mL) dropwise. The reaction mixture was stirred for 1.5 hours. The ethanol was evaporated and the residue was acidified to pH=2 by 2 M HCl and washed by DCM. The organic layer was washed with brine and dried over MgSO₄. The solvent was evaporated to give 2-{[(tert-butoxy)carbonyl]amino}-3-ethoxy-3-oxopropanoic acid XCVII as crystals (1.68 g, 6.8 mmol, 85% yield). ¹H NMR (CDCl₃) 1.31 (t, J=7 Hz, 3H), 1.41-1.45 (m, 9H), 4.23-4.31 9m, 2H), 4.76 (d, J=4 Hz, 1H), 7.77 (d, J=4 Hz, 1H), 10.84 (brs, 1H); ESIMS found for C₁₀H₁₇NO₆ m/z 248 (M+H).

### Step 3

To a solution of 2-{[(tert-butoxy)carbonyl]amino}-3-ethoxy-3-oxopropanoic acid XCVII (0.5 g; 2.02 mmol) and DIPEA (1.20 mL; 7.07 mM) in DCM (30 mL) was added (2S)-2-amino-N-(quinolin-3-yl)-3-[4-(trifluoromethyl)phenyl]propanamide LXXI (0.88 g; 2.02 mmol) and TBTU (0.68 g; 2.12 mmol). The reaction mixture was stirred overnight, diluted with DCM (30 mL), washed with 1 M aq NaOH (2×), 1 M aqueous HCl (2×), brine and dried over anhydrous MgSO₄. The solvent was evaporated and the crude product was crystallized from DCM/hexane to give ethyl 2-{[(tert-butoxy)carbonyl]amino}-2-{[(1S)-1-[(quinolin-3-yl)carbamoyl]-2-[4-(trifluoromethyl)phenyl]ethyl]carbamoyl}acetate XCVIII as yellow crystals (0.90 g, 1.53 mmol, 76% yield). ESIMS found for C₂₉H₃₁F₃N₄O₆ m/z 589 (M+H).

### Step 4

To a solution of ethyl 2-{[(tert-butoxy)carbonyl]amino}-2-t[(1S)-1-[(quinolin-3-yl)carbamoyl]-2-[4-(trifluoromethyl)phenyl]ethyl]carbamoyl}acetate XCVIII (0.90 g, 1.53 mmol) in a mixture of ethanol/water (45 mL/5 mL) was added KOH (0.103 g, 1.84 mmol) in water (10 mL) dropwise. The reaction mixture was stirred for 1.5 h. The ethanol was evaporated under reduced pressure and the residue was acidified to pH=2 by 2 M HCl. The aqueous solution was extrated with DCM. The DCM extract was then washed with brine and dried over MgSO₄. The solvent was evaporated to give 2-t[(tert-butoxy)carbonyl]amino}-2-{[(1S)-1-[(quinolin-3-yl)carbamoyl]-2-[4-(trifluoromethyl)phenyl]ethyl]carbamoyl}acetic acid XCIX (0.45 g, 0.80 mmol, 52% yield). The crude product was used directly for step 5.

### Step 5

Procedure can be found in examples 1-2.

### Step 6

Procedure can be found in examples 1-2. The final compound 21 was isolated as the hydrochloride salt. ¹H NMR (DMSO-d₆) 2.82-2.98 (m, 2H), 2.98-3.12 (m, 2H), 3.13-3.21 (m, 2H), 3.56-3.91 (m, 4H), 4.74-4.89 (m, 1H), 5.09-5.23 (m, 1H), 7.52-7.61 (m, 1H), 7.61-7.72 (m, 4H), 7.91-8.11 (m, 5H), 8.25-8.31 (brs, 3H), 8.66 (d, J=8 Hz, 1H), 8.71-8.77 (brs, 1H), 8.77-8.83 (brs, 1H), 8.96-9.04 (m, 1H), 9.40 (d, J=8 Hz, 1H), 9.67 (d, J=8 Hz, 1H), 10.88 (s, 1H), 10.93 (s, 1H); ESIMS found for C₂₆H₃₀F₃N₇O₃ m/z 546 (M+H).

Synthesis of 3-[(1S)-3-(aminiomethyl)-1-{[(1R)-1-carbamoyl-3-phenylpropyl]carbamoyl}butane-1,4-bis(aminium)]quinolin-1-ium tetrachloride 23 is depicted below in scheme 15 and example 15.

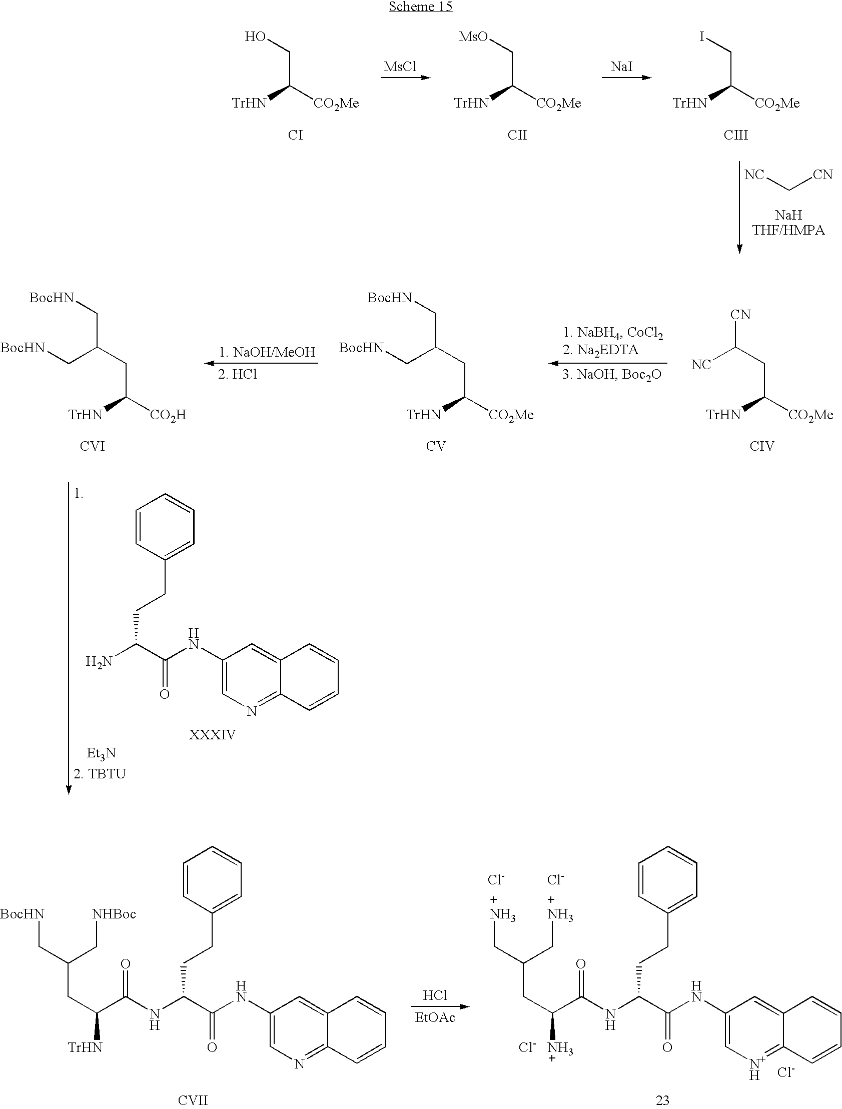

### Example 15

### Step 1

To a solution of methyl (2S)-3-hydroxy-2-(tritylamino)propanoate CI (24.35 g; 67.37 mmol) and TEA (11.2 mL; 80.8 mmol) in DCM (330 mL) cooled to 0° C. was added methanesulfonyl chloride (6.3 mL; 80.8 mmol) dropwise. The mixture reaction was stirred at r.t. overnight before being diluted with DCM (120 mL), washed with water, 5% aq NaHCO₃, 0.5 M aq KHSO₄, water, and dried over anhydrous MgSO₄. The solvent was removed under reduced pressure to give methyl (2S)-3-[(methylsulfonyl)oxy]-2-(tritylamino)propanoate CII as a off-white foam (22.2 g; 50.51 mmol; 75% yield). ¹H NMR (CDCl₃) 2.89 (d, J=10 Hz, 1H), 3.00 (s, 3H), 3.28 (s, 3H), 3.64-3.68 (m, 1H), 4.25 (dd, J=6 Hz, J=10 Hz, 1H), 4.43 (dd, J=4 Hz, J=10 Hz, 1H), 7.20 (ddd, J=8 Hz, J=8 Hz, J=1 Hz, 3H), 7.28 (dd, J=8 Hz, J=8 Hz, 6H), 7.49 (ddd, J=8 Hz, J=8 Hz, J=1 Hz, 6H); ESIMS found for C₂₄H₂₅NO₅S m/z 440.1 (M+H).

### Step 2

To a solution of methyl (2S)-3-[(methylsulfonyl)oxy]-2-(tritylamino) propanoate CII (22 g; 0.05 mol) in acetone (700 mL) was added sodium iodide (150 g; 1.0 mol) and stirred at r.t. for one week under argon. The acetone was evaporated and the residue was dissolved in diethyl ether (1.5 L). The solids were filtered and the solvent reduced to 1 L before washing with 10% aq Na₂S₂O₃ (3×) and water (200 mL). The solvent was removed under reduced pressure and the residue purified on a silica gel column (10:1→2:1 hexane/EtOAc) and then crystallized from hexane. The methyl (2R)-3-iodo-2-(tritylamino)propanoate CIII was obtained as yellow foam (21 g; 0.045 mol; 89% yield). ¹H NMR (CDCl₃) [2.25-2.28 (m, 1^{st} rotamer), 2.89 (d, J=10 Hz, 2^{nd} rotamer), 1H], [2.54 (dd, J=13 Hz, J=6 Hz), 3.21 (dd, J=10 Hz, J=8 Hz), 1^{st} rotamer, 1H], [2.69 (dd, J=12 Hz, J=8 Hz), 3.35 (dd, J=10 Hz, J=3 Hz), 2^{nd} rotamer, 1H], [3.31 (s, 1^{st} rotamer), 3.77 (s, 2^{nd} rotamer), 3H], [3.48 (ddd, J=10 Hz, J=7 Hz, J=3 Hz, 2^{nd} rotamer), 4.39 (dd, J=8 Hz, J=6 Hz, 1^{st} rotamer), 1H], 7.18-7.22 (m, 3H), 7.29 (dd, J=8 Hz, J=7 Hz, 6H), [7.45 (d, J=8 Hz, 1^{st} rotamer), 7.50 (d, J=8 Hz, 2^{nd} rotamer), 6H].

### Step 3

To a solution of malononitrile (0.71 g; 10.6 mmol) in mixture of THF (30 mL) and HMPA (20 mL) was added sodium hydride 60% (0.43 g; 10.6 mmol) and stirred for 30 min. To this mixture was added a solution of methyl (2R)-3-iodo-2-(tritylamino)propanoate CIII (5.0 g; 10.6 mmol) in THF (52 mL) and stirred overnight at r.t. The reaction was quenched with saturated aqueous ammonium chloride and extracted with diethyl ether (5×). The combined organic layers were washed with saturated aqueous ammonium chloride and dried over anhydrous MgSO₄. The solvent was removed and the residue was purified on a silica gel column (3:1 hexane/EtOAc) and then crystallized from hexane/EtOAc to give the methyl (2S)-4,4-dicyano-2-(tritylamino)butanoate CIV (2.62 g; 6.4 mmol; 60% yield). ¹H NMR (CDCl₃) 3.02-3.06 (m, 1H), 2.70-2.75 (m, 1H), 2.81-2.86 (m, 1H), 2.95 (dd, J=12 Hz, J=6 Hz, 1H), 3.76 (s, 3H), 4.51 (d, J=6 Hz, 1H), 7.23 (t, J=8 Hz, 3H), 7.32 (dd, J=8 Hz, J=8 Hz, 6H), 7.43 (d, J=8 Hz, 6H); ESIMS found for C₂₆H₂₃N₃O₂ m/z 432.3 (M+Na).

### Step 4

To a solution of methyl (2S)-4,4-dicyano-2-(tritylamino)butanoate CIV (1.0 g; 2.44 mmol) in methanol (15 mL) and THF (5 mL) was added cobalt (II) chloride hexahydrate (2.9 g; 12.2 mmol) and cooled to −10° C. After 5 min, sodium borohydride (0.92 g; 24.4 mmol) was added and after 20 min the cooling bath was removed and the reaction was stirred for one h at r.t. To the reaction mixture was added 4 M NaOH until pH˜10. Disodium EDTA (4.6 g; 12.2 mmol) and di-tert-butyl dicarbonate (1.3 g; 5.86 mmol) were added and the mixture was stirred overnight at r.t. The solids were filtered and washed with methanol. The solvent was evaporated and the remaining aqueous solution was acidified with 2 M HCl to pH˜6 and extracted with DCM. The combined organic layers were dried over anhydrous MgSO₄ and removed under reduced pressure. The residue was purified on a silica gel column (4:1→1:1 hexane/EtOAc) to afford methyl (2S)-5-{[(tert-butoxy) carbonyl]amino}-4-({[(tert-butoxy)carbonyl]amino}methyl)-2-[(triphenylmethyl)amino]pentanoate CV (0.19 g; 0.31 mmol; 13.7% yield). ESIMS found for C₃₆H₄₇N₃O₆ m/z 618.6 (M+H).

### Step 5

To a solution of methyl (2S)-5-{[(tert-butoxy) carbonyl]amino}-4-({[(tert-butoxy)carbonyl]amino}methyl)-2-[(triphenylmethyl)amino]pentanoate CV (180 mg; 0.29 mmol) in methanol (15 mL) was added 4 M aq NaOH (3 mL). The mixture was stirred at r.t. for 1 h. Water (20 mL) was then added and the methanol was removed under reduce pressure. The aqueous solution was acidified with 2 M HCl to pH˜5-6 and extracted with DCM. The combined organic layers were dried over anhydrous MgSO₄ and evaporated to obtain the crude (2S)-5-{[(tert-butoxy)carbonyl]amino}-4-({[(tert-butoxy)carbonyl]amino}methyl)-2-[(triphenylmethyl)amino]pentanoic acid CVI (145 mg). This material was used directly for step 6. ESIMS found for C₃₅H₄₅N₃O₆ m/z 604.4 (M+H).

### Step 6

Procedure can be found in examples 1-2.

### Step 7

Procedure can be found in examples 1-2. The final compound 23 was isolated as the hydrochloride salt. ¹H NMR (CD₃OD) 0.83-0.94 (m, 1H), 2.12-2.33 (m, 2H), 2.76-2.94 (m, 2H), 3.10-3.41 (m, 6H), 4.54-4.61 (m, 1H) 4.87-4.91 (m, 1H), 7.14-7.22 (m, 1H), 7.26-7.29 (m, 4H), 7.60 (ddd, J=8 Hz, J=7 Hz, J=1 Hz, 1H), 7.70 (ddd, J=8 Hz, J=7 Hz, J=1 Hz, 1H), 7.88 (dd, J=8 Hz, J=1 Hz, 1H), 7.99 (dd, J=8 Hz, J=1 Hz, 1H), 8.64 (d, J=2 Hz, 1H), 8.94 (d, J=2 Hz, 1H); ESIMS found for C₂₅H₃₂N₆O₂ m/z 449.5 (M+H).

The following compound was prepared in accordance with the procedure described in the above example 15.

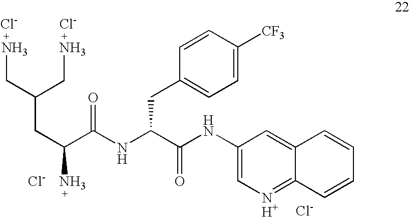

### 3-[(1S)-3-(azaniumylmethyl)-1-{[(1R)-1-carbamoyl-2-[4-(trifluoromethyl)phenyl]ethyl]carbamoyl}butane-1,4-bis(aminium)]quinolin-1-ium tetrachloride 22

¹H NMR (CD₃OD) 0.83-0.93 (m, 1H), 2.44-2.52 (m, 1H), 2.59-2.68 (m, 1H), 2.92-3.44 (m, 6H), 4.79-4.99 (m, 2H), 7.56-7.63 (m, 4H), 7.71 (dd, J=8 Hz, J=8 Hz, 1H), 7.83 (dd, J=8 Hz, J=7 Hz, 1H), 7.98 (d, J=7 Hz), 8.06 (d, J=8 Hz), [8.72 (brs, 1^{st} rotamer), 8.76 (brs, 2^{nd} rotamer), 1H], 9.06 (brs, 1H); ¹⁹F NMR (CD₃OD)-63.40 (1^{st} rotamer), −63.33 (2^{nd} rotamer) (s, 3F); ESIMS found for C₂₅H₂₉F₃N₆O₂ m/z 503.5 (M+H).

Synthesis of 3-azaniumyl-1-[(4S)-4-azaniumyl-4-{[(1R)-1-[(quinolin-1-ium-3-yl)carbamoyl]-2-[4-(trifluoromethyl)phenyl]ethyl]carbamoyl}butyl]pyrrolidin-1-ium tetrachloride 24 is depicted below in scheme 16 and example 16.

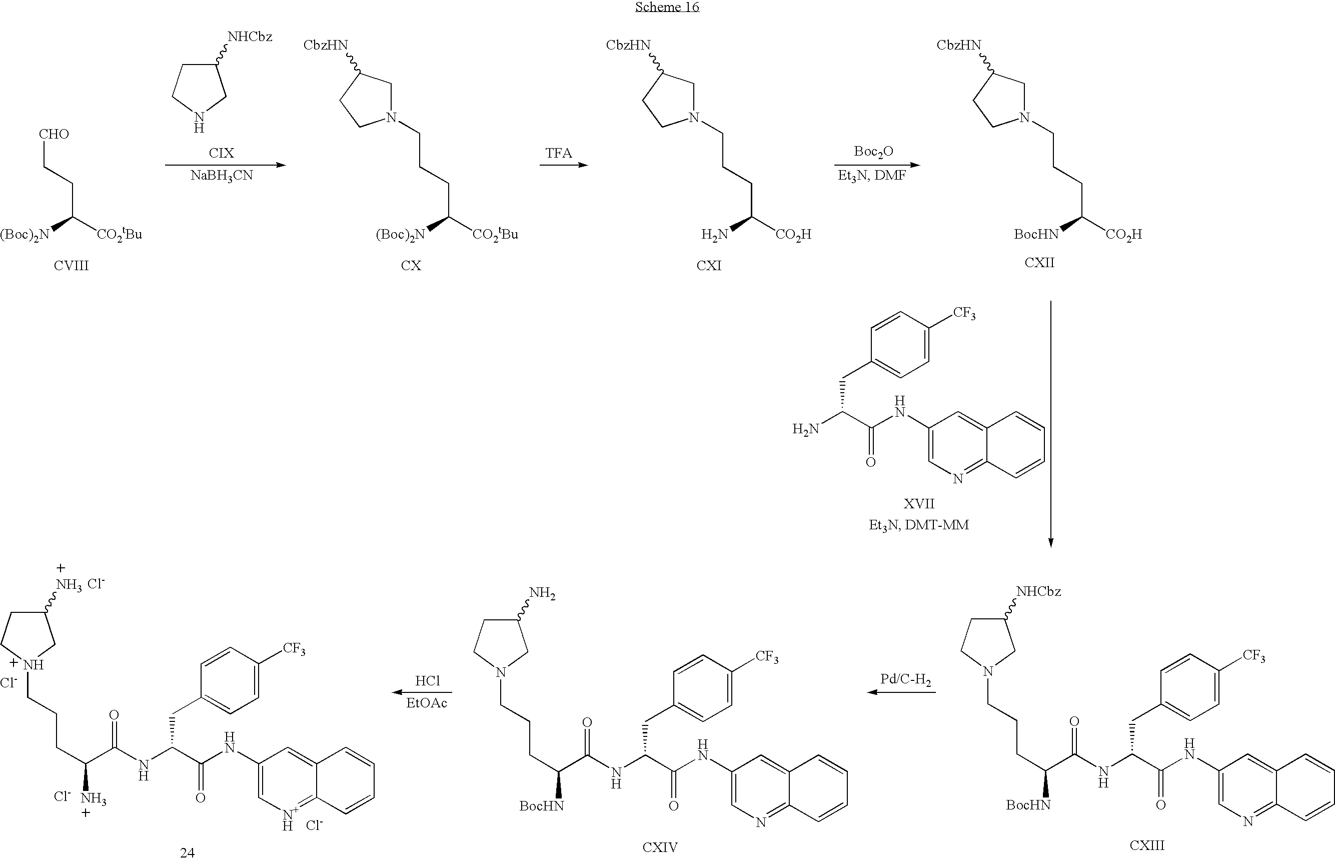

### Example 16

### Step 1

To a solution of benzyl N-(pyrrolidin-3-yl)carbamate CIX (622 mg, 2.4 mmol) in DCM (15 mL), cooled down to 0° C. was added acetic acid (0.4 mL, 11 mmol) followed by tert-butyl (2S)-2-{bis[(tert-butoxy)carbonyl]amino}-5-oxopentanoate CVIII (860 mg, 2.2 mmol). The reaction mixture was stirred at 0° C. for 1 h and then sodium cyanoborohydride (208 mg, 3.30 mmol) was added. The mixture was left with stirring overnight at r.t. DCM (30 mL) was added and the mixture was washed with water and brine. The organic layer was dried over Na₂SO₄ and evaporated to dryness to obtain crude tert-butyl (2S)-5-(3-{[(benzyloxy)carbonyl]amino}pyrrolidin-1-yl)-2-{bis[(tert-butoxy)carbonyl]amino}pentanoate CX (1.22 g, 2.06 mmol, 93.7% yield). ESIMS found for C₃₁H₄₉N₃O₈ m/z 592 (M+H).

### Step 2-3

A solution of tert-butyl (2S)-5-(3-{[(benzyloxy)carbonyl]amino}pyrrolidin-1-yl)-2-{bis[(tert-butoxy)carbonyl]amino}pentanoate CX (1.22 g, 2.06 mmol) in trifluoroacetic acid (10 mL) was stirred overnight at r.t. The reaction mixture was concentrated to dryness to give (2S)-2-amino-5-(3-{[(benzyloxy)carbonyl]amino}pyrrolidin-1-yl)pentanoic acid CXI, which was dissolved in DMF (15 mL). TEA (1.4 mL, 10 mmol) was added at r.t. followed by Boc₂O (460 mg, 2.2 mmol) and the mixture was stirred overnight. The solvent was evaporated under reduced pressure and the residue was treated with water (50 mL) and 1N HCl to adjust the pH to 3. The mixture was extracted with ethyl acetate and the organic layer was dried over Na₂SO₄ and concentrated to dryness to give crude (2S)-5-(3-{[(benzyloxy)carbonyl]amino}pyrrolidin-1-yl)-2-{[(tert-butoxy)carbonyl]amino}pentanoic acid CXII (1.0 g). ESIMS found for C₂₂H₃₃N₃O₆ m/z 436 (M+H).

### Step 4

To a solution of (2S)-5-(3-{[(benzyloxy)carbonyl]amino}pyrrolidin-1-yl)-2-{[(tert-butoxy)carbonyl]amino}pentanoic acid CXII (1.0 g) in DCM (10 mL) was added DMT-MM (636 mg, 2.3 mmol). In a separate flask, (2R)-2-amino-N-(quinolin-3-yl)-3-[4-(trifluoromethyl)phenyl]propanamide XVII as the trifluoroacetate salt (700 mg, 1.48 mmol) was suspended in DCM (10 mL) and treated with TEA (0.41 mL, 3.0 mmol) while the mixture became homogeneous. The two solutions were combined and were allowed to react at r.t. overnight. The reaction mixture was washed with water, satd. NaHCO₃ and water and dried over Na₂SO₄. The solvent was removed under reduced pressure and residue was purified on a silica gel column (1:1 hexane:EtOAc→100% EtOAc→5:1 EtOAc:MeOH) to give tert-butyl N-[(1S)-4-(3-{[(benzyloxy)carbonyl]amino}pyrrolidin-1-yl)-1-{[(1R)-1-[(quinolin-3-yl)carbamoyl]-2-[4-(trifluoromethyl)phenyl]ethyl]carbamoyl}butyl]carbamate CXIII (390 mg, 0.50 mmol, 20% yield). ¹H NMR (DMSO-d₆) 1.09-1.21 (m, 2H), 1.33 (s, 9H), 1.54-1.60 (m, 2H), 2.01-2.12 (m, 2H), 2.20 (brs, 2H), 2.68-2.74 (m, 2H), 2.96-3.05 (m, 1H), 3.85-3.89 (m, 2H), 3.92-3.98 (m, 1H), 4.10-4.16 (m, 2H), 4.82-4.88 (m, 1H), 5.00 (s, 2H), 7.08 (d, J=7 Hz, 1H), 7.20-7.68 (m, 10H), 7.95-8.02 (m, 4H), 8.52 (d, J=7 Hz, 1H), 8.71 (d, J=2 Hz, 1H), 8.98 (d, J=2 Hz, 1H), 10.40 (s, 1H); ESIMS found for C₄₁H₄₇N₆O₆F₃ m/z 777 (M+H).

### Step 5

To a solution of tert-butyl N-[(1S)-4-(3-{[(benzyloxy)carbonyl]amino}pyrrolidin-1-yl)-1-{[(1R)-1-[(quinolin-3-yl)carbamoyl]-2-[4-(trifluoromethyl)phenyl]ethyl]carbamoyl}butyl]carbamate CXIII (170 mg, 0.22 mmol) in methanol (20 mL) was added catalytic amount of 10% Pd/C catalyst. The mixture was hydrogenated at normal pressure for 48 h. The catalyst was filtered through Celite and the filtrate was concentrated to give tert-butyl N-[(1S)-4-(3-aminopyrrolidin-1-yl)-1-{[(1R)-1-[(quinolin-3-yl)carbamoyl]-2-[4-(trifluoromethyl)phenyl]ethyl]carbamoyl}butyl]carbamate CXIV (120 mg, 0.19 mmol, 86.4% yield). ESIMS found for C₃₃H₄₁N₆O₄F₃ m/z 643 (M+H).

### Step 6

Procedure can be found in examples 1-2. The final compound 24 was isolated as the hydrochloride salt. ¹H NMR (CD₃OD) 1.48-1.73 (m, 4H), 2.05-2.11 (m, 1H), 2.11-2.22 (m, 1H), 3.00-3.11 (m, 2H), 3.13-3.21 (m, 2H), 3.32-3.40 (m, 1H), 3.85-3.97 (m, 4H), 4.03-4.17 (m, 1H), 4.95-4.88 (m, 1H), 7.60-7.70 (m, 6H), 7.98-8.05 (m, 2H), 8.29 (brs, 3H), 8.57 (brs, 1.5H 1^{st} diastereoisomer), 8.69 (brs, 1.5H 2^{nd} diastereoisomer), 8.74 (d, J=2 Hz, 1H), 9.08 (d, J=2 Hz, 1H), 9.22 (d, J=7 Hz, 1H), 11.12 (s, 1H); ESIMS found for C₂₈H₃₃N₆O₂F₃ m/z 543 (M+H).

Synthesis of 3-[(1S)-4-{[bis(2-azaniumylethyl)carbamoyl]amino}-1-{[(1R)-1-carbamoyl-2-[4-(trifluoromethyl)phenyl]ethyl]carbamoyl}butan-1-aminium]quinolin-1-ium tetrachloride 29 is depicted below in scheme 17 and example 17.

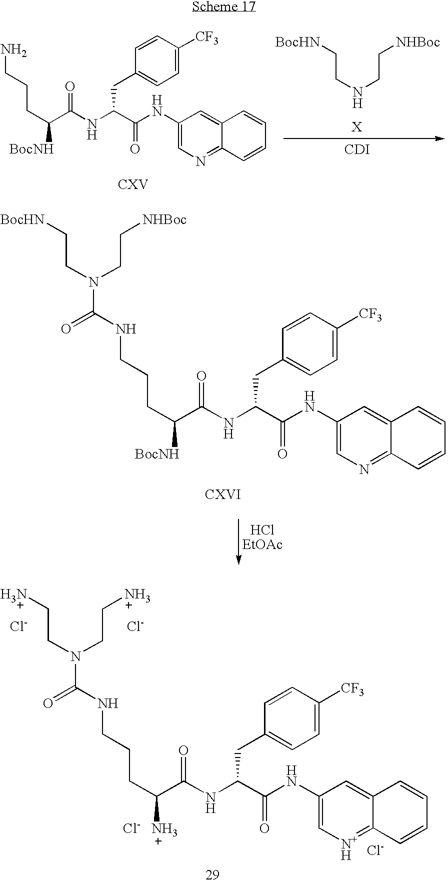

### Example 17

### Step 1

To a solution of carbonyldiimidazole (204 mg, 1.25 mmol) in DCM (20 mL) was added tert-butyl N-[(1S)-4-amino-1-{[(1R)-1-[(quinolin-3-yl)carbamoyl]-2-[4-(trifluoromethyl)phenyl]ethyl]carbamoyl}butyl]carbamate CXV (687 mg, 1.20 mmol) and stirred at r.t. for 1 h. To this mixture was added tert-butyl N-{2-[(2-{[(tert-butoxy)carbonyl]amino}ethyl)amino]ethyl}carbamate X (436 mg, 1.44 mmol) and the reaction was stirred overnight at r.t. The mixture was diluted DCM and washed with 1 M HCl, brine, dried over MgSO₄ and purified on a silica gel column (100:1→70:1→50:1 CHCl₃/MeOH) to give product tert-butyl N-[2-({[(4S)-4-{[(tert-butoxy)carbonyl]amino}-4-{[(1R)-1-[(quinolin-3-yl)carbamoyl]-2-[4-(trifluoromethyl)phenyl]ethyl]carbamoyl}butyl]carbamoyl}(2-{[(tert-butoxy)carbonyl]amino}ethyl)amino)ethyl]carbamate CXVI (500 mg, 0.55 mmol, 44% yield). ESIMS found for C₄₄H₆₁F₃N₈O₉ m/z 904 (M+H).

### Step 2

Procedure can be found in examples 1-2. The final compound 29 was isolated as the hydrochloride salt. ¹H NMR (CD₃OD) 0.90-1.27 (m, 2H), 1.38-1.68 (m, 2H), 2.72-3.04 (m, 6H), 3.34-3.55 (m, 6H), 4.61-4.75 (m, 1H), 4.88-5.03 (m, 1H), 6.81-6.95 (m, 1H), 7.01-7.19 (m, 1H), 7.42-7.55 (m, 1H), 7.57-7.68 (m, 4H), 7.70-7.82 (m, 2H), 8.21 (brs, 7H), 8.24 (brs, 3H), 8.92 (d, J=2 Hz, 1H), 9.19 (d, J=8 Hz, 1H), 9.27 (d, J=2 Hz, 1H), 11.39 (s, 1H); ¹⁹F NMR (DMSO-d₆)-60.08 (s, 3F); ESIMS found for C₂₉H₃₇F₃N₈O₃ m/z 604 (M+H).

The following compound was prepared in accordance with the procedure described in the above example 17.

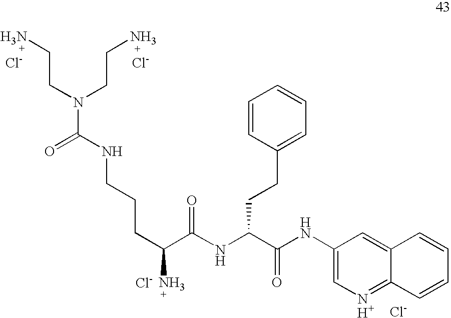

### 3-[(1S)-4-{[bis(2-azaniumylethyl)carbamoyl]amino}-1-{[(1R)-1-carbamoyl-3-phenylpropyl]carbamoyl}butan-1-aminium]quinolin-1-ium tetrachloride 44

¹H NMR (DMSO-d₆) 1.40-1.66 (m, 2H), 1.75-1.90 (m, 2H), 1.94-2.18 (m, 2H), 2.57-2.77 (m, 2H), 2.81-2.96 (m, 2H), 3.08 (brs, 2H), 3.38-3.51 (m, 4H), 3.95-4.01 (m, 1H), 4.43-4.55 (m, 2H), 7.04-7.10 (m, 1H), 7.11-7.18 (m, 1H), 7.20-7.28 (m, 4H), 7.71 (dd, J=7 Hz, 1H), 7.81 (dd, J=7 Hz, 1H), 8.08-8.24 (m, 8H), 8.40 (brs, 3H), 9.02 (brs, 1H), 9.25 (d, J=7 Hz, 1H), 9.34 (brs, 1H), 11.21 (s, 1H); ESIMS found for C₂₉H₄₀N₈O₃ m/z 549 (M+H).

Synthesis of 2-[bis(2-azaniumylethyl)carbamoyl]-1-t[(1S)-1-[methyl(2-{[4-(trifluoromethyl)phenyl]formamido}ethyl)carbamoyl]-3-phenylpropyl]carbamoyl}ethan-1-aminium trichloride 34 is depicted below in scheme 18 and example 18.

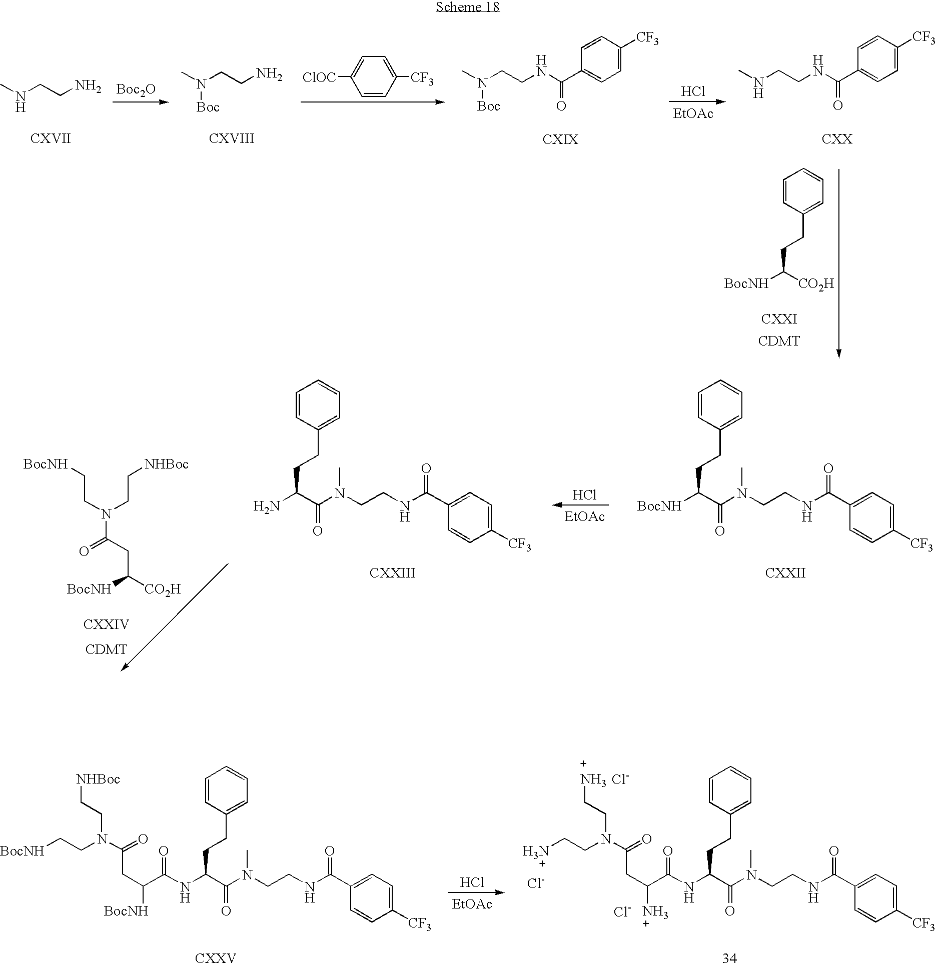

### Example 18

### Step 1

To a solution of N-methylethylenediamine (11.8 mL, 134.9 mmol) in acetonitrile (300 mL), cooled to −30° C. was added TEA (7.46 mL, 53.9 mmol) and then a solution of Boc₂O (9.81 g, 45 mmol) in acetonitrile was added dropwise. The mixture was stirred for 2 h at r.t. and then filtered through Celite. The residue was purified on a silica gel column (1:50→1:20→1:10 EtOAc:hexane) to give tert-butyl N-(2-aminoethyl)-N-methylcarbamate CXVIII as a yellow oil (5.2 g, 29.9 mmol, yield 66%). ESIMS found for C₈H₁₈N₂O₂ m/z 175 (M+H).

### Step 2

To a solution of compound tert-butyl N-(2-aminoethyl)-N-methylcarbamate CXVIII (1.00 g, 5.74 mmol) in DCM (30 mL) was added TEA (0.87 mL, 6.32 mmol) and cooled to 0° C. To this mixture was added a solution of 4-(trifluoromethyl)benzoyl chloride (1.32 g, 6.32 mmol) in DCM (10 mL) dropwise. The reaction mixture was stirred overnight. Ethyl ether was added and the product precipitated (1.06 g, 2.88 mmol, 50% yield). The crude tert-butyl N-methyl-N-(2-{[4-(trifluoromethyl)phenyl]formamido}ethyl)carbamate CXIX was used in step 3 without any purification. ESIMS found for C₁₆H₂₁F₃N₂O₃ m/z 369 (M+Na).

### Step 3

A solution of tert-butyl N-methyl-N-(2-{[4-(trifluoromethyl)phenyl]formamido}ethyl)carbamate CXIX (0.53 g, 1.44 mmol) in 3 M HCl in EtOAc (20 mL) was stirred for about 2 h. The solvent was evaporated under vacuum to give N-[2-(methylamino)ethyl]-4-(trifluoromethyl)benzamide CXX as white crystals (0.32 g, 1.30 mmol, 90% yield). ¹H NMR (DMSO-d₆) 2.57 (s, 3H), 3.03-3.06 (m, 2H), 3.57-3.61 (m, 2H), 7.87 (d, J=8 Hz, 2H), 8.12 (d, J=8 Hz, 2H), 8.89 (brs, 2H), 9.06-9.09 (m, 1H); ESIMS found for C₁₁H₁₃F₃N₂O m/z 247 (M+H).

### Step 4

A solution of CDMT (0.28 g, 1.6 mmol) in DCM (30 mL) was cooled to 0° C. before adding N-methylmorpholine (0.44 mL, 4 mmol). After 15 min, (2S)-2-t[(tert-butoxy)carbonyl]amino}-4-phenylbutanoic acid CXXI (0.42 g, 1.5 mmol) was added and the solution was stirred for an additional 40 min. After that time, N-[2-(methylamino)ethyl]-4-(trifluoromethyl)benzamide CXX (0.32 g, 1.3 mmol) was added and the mixture stirred at r.t. overnight. The mixture was washed with 1 M HCl (5×), 1 M K₂CO₃ (5×), brine and dried over MgSO₄. The solvent was evaporated under vacuum and the solid residue was crystallized from EtOAc/hexane to give tert-butyl N-[(1S)-1-[methyl(2-{[4-(trifluoromethyl)phenyl]formamido}ethyl)carbamoyl]-3-phenylpropyl]carbamate CXXII as white solid (0.65 g, 1.23 mmol, 95% yield). ¹H NMR (DMSO-d₆) 1.34 (s, 9H), 1.66-1.80 (m, 2H), 2.54-2.66 (m, 2H), 2.93 (s, 3H), 3.44-3.52 (m, 2H), 3.74-3.83 (m, 2H), 4.25-4.32 (m, 1H), 6.99-7.14 (m, 7H), 7.74 (d, J=8 Hz, 2H), 7.91 (d, J=8 Hz, 2H), 8.63-8.71 (m, 1H); ESIMS found for C₂₆H₃₂F₃N₃O₄ m/z 530 (M+Na).

### Step 5

A solution of tert-butyl N-[(1S)-1-[methyl(2-{[4-(trifluoromethyl)phenyl]formamido}ethyl)carbamoyl]-3-phenylpropyl]carbamate CXXII (0.65 g, 1.23 mmol) in 1 M HCl in diethyl ether was stirred overnight. The solvent was evaporated under vacuum to give (2S)-2-amino-N-methyl-4-phenyl-N-(2-{[4-(trifluoromethyl)phenyl]formamido}ethyl)butanamide CXXIII as white crystals (0.46 g, 1.14 mmol, 93% yield). ¹H NMR (DMSO-d₆) 1.87-2.02 (m, 2H), 2.55-2.74 (m, 2H), 3.00 (s, 3H), 3.44-3.59 (m, 2H), 3.88-3.94 (m, 2H), 4.27 (brs, 1H), 7.18-7.28 (m, 6H), 7.71 (d, J=8 Hz, 2H), 7.95 (d, J=8 Hz, 2H), 8.29 (brs, 3H), 8.86-8.88 (m, 1H); ESIMS found for C₂₁H₂₄F₃N₃O₂ m/z 408 (M+H).

### Step 6

A solution of CDMT (0.22 g, 1.26 mmol) in DCM (30 mL) was cooled to 0° C. before adding N-methylmorpholine (0.3 mL, 2.62 mmol). After 15 min, (2S)-3-[bis(2-{[(tert-butoxy)carbonyl]amino}ethyl)carbamoyl]-2-{[(tert-butoxy)carbonyl]amino}propanoic acid CXXIV (0.62 g, 1.20 mmol) was added and the solution was stirred for additional 40 min. After that time, (2S)-2-amino-N-methyl-4-phenyl-N-(2-{[4-(trifluoromethyl)phenyl]formamido}ethyl)butanamide CXXIII (0.56 g, 1.26 mmol) was added and the mixture stirred at r.t. overnight. The mixture was washed with 1 M HCl (5×), 1 M K₂CO₃ (5×), brine and dried over MgSO₄. The solvent was evaporated under vacuum and the residue was purified on a silica gel column (50:1 DCM/methanol) to give tert-butyl N-[(1S)-2-[bis(2-{[(tert-butoxy)carbonyl]amino}ethyl)carbamoyl]-1-{[(1S)-1-[methyl(2-{[4-(trifluoromethyl)phenyl]formamido}ethyl)carbamoyl]-3-phenylpropyl]carbamoyl}ethyl]carbamate as yellow solid (0.78 g, 0.86 mmol, 68% yield). ESIMS found for C₄₄H₆₄F₃N₇O₁₀ m/z 908 (M+H).

### Step 7

Procedure can be found in examples 1-2. The final compound 34 was isolated as the hydrochloride salt. ¹H NMR (DMSO-d₆) 2.97 (s, 3H), 2.97-3.18 (m, 6H), 3.18-3.52 (m, 8H), 3.52-3.74 (m, 4H), 4.21-4.38 (m, 1H), 4.53-4.71 (m, 1H), 7.04-7.27 (m, 5H), 7.73 (d, J=8 Hz, 1H), 7.83 (d, J=8 Hz, 1H), 7.97 (d, J=8 Hz, 1H), 8.07 (d, J=8 Hz, 1H), 8.07-8.50 (m, 9H), 8.82 (d, J=8 Hz, 1H), 8.82-8.90 (m, 1H); ESIMS found for C₂₉H₄₀F₃N₇O₄ m/z 608 (M+H).

The following compound was prepared in accordance with the procedure described in the above example 18.

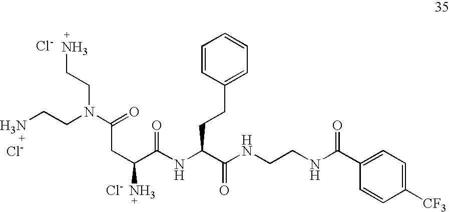

### (1S)-2-[bis(2-azaniumylethyl)carbamoyl]-1-{[(1S)-3-phenyl-1-[(2-{[4-(trifluoromethyl)phenyl]formamido}ethyl)carbamoyl]propyl]carbamoyl}ethan-1-aminium

¹H NMR (DMSO-d₆) 1.74-2.05 (m, 2H), 2.53-2.69 (m, 2H), 2.91-3.19 (m, 8H), 3.55-3.70 (m, 6H), 4.18 (brs, 1H), 4.32 (brs, 1H), 7.10-7.28 (m, 5H), 7.77 (d, J=8 Hz, 2H), 8.06 (d, J=8 Hz, 2H), 8.16 (brs, 3H), 8.35 (brs, 7H), 8.88 (brs, 2H); ¹⁹F NMR (DMSO-d₆)-60.67 (s, 3F); ESIMS found for C₂₈H₃₈F₃N₇O₄ m/z 594 (M+H).

Synthesis of 3-[(4S)-4-{[(1R)-1-carbamoyl-3-phenylpropyl]carbamoyl}butane-1,2,4-tris(aminium)]quinolin-1-ium tetrachloride 40 is depicted below in scheme 19 and example 19.

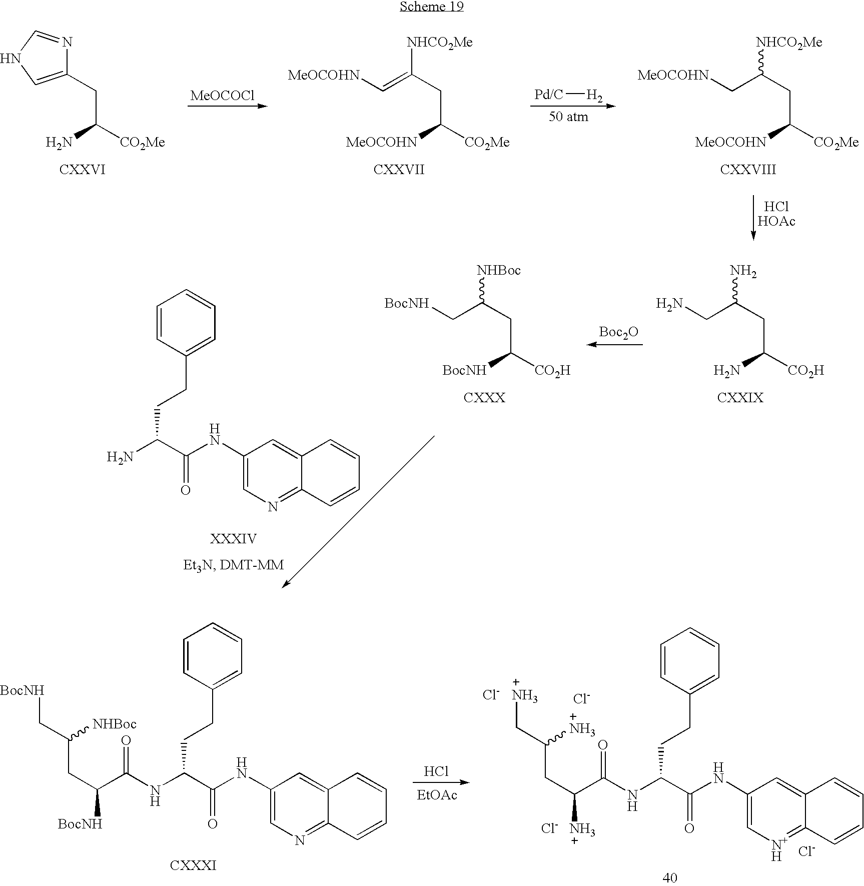

### Example 19

### Step 1

A solution of histidine methyl ester CXXVI (500 mg, 2.96 mmol) in EtOAc (20 mL) and water (5 mL) was cooled to 0° C. before adding a solution of methyl chloroformate (2.3 mL, 29.6 mmol) in EtOAc (20 mL) and a solution of NaHCO₃ (2.5 g, 29.6 mmol) in water (25 mL). After the addition was complete, the mixture was stirred at 0° C. for 2 h and then at r.t. overnight. The organic layer was separated, washed with water, dried over Na₂SO₄ and concentrated. The residue was purified on a silica gel column (1:2 hexane:EtOAc) to produce methyl (2S,4Z)-2,4,5-tris[(methoxycarbonyl)amino]pent-4-enoate CXXVII (430 mg, 1.29 mmol, 44% yield). ¹H NMR (DMSO-d₆) 2.35-2.41 (m, 1H), 2.76-2.80 (m, 1H), 3.57-3.62 (m, 12H), 4.07-4.12 (m, 1H), 6.02 (d, J=10 Hz, 1H), 7.45 (d, J=8 Hz, 1H), 8.15 (brs, 1H), 8.64 (brs, 1H). ESIMS found for C₁₂H₁₉N₃O₈ m/z 333 (M+).

### Step 2

To a solution of methyl (2S,4Z)-2,4,5-tris[(methoxycarbonyl)amino]pent-4-enoate CXXVII (624 mg, 1.87 mmol) in ethanol (15 mL) was added 10% Pd/C catalyst (60 mg). The mixture was placed in an autoclave and exposed to 50 atm of H₂ with stirring at r.t. for 36 h. Reaction mixture was filtered through Celite and concentrated to dryness to give crude methyl (2S)-2,4,5-tris[(methoxycarbonyl)amino]pentanoate CXXVIII (540 mg). ESIMS found for C₁₂H₂₁N₃O₈ m/z 358 (M+Na).

### Step 3-4

A solution of methyl (2S)-2,4,5-tris[(methoxycarbonyl)amino]pentanoate CXXVIII in acetic acid (3 mL) and conc. HCl (7 mL) and refluxed for 60 h. The reaction mixture was concentrated to dryness giving crude (2S)-2,4,5-triaminopentanoic acid CXXIX as the hydrochloride salt (350 mg). CXXIX was dissolved in DMF (8 mL) before adding TEA (1.3 mL, 9.3 mmol) followed by Boc₂O (1.36 g, 6.54 mmol). The reaction mixture was stirred at r.t. for 48 h. The solvent was evaporated under reduced pressure and the residue was dissolved in water, washed with diethyl ether and acidified with 1 N HCl until pH=2.5. The aqueous phase was further extracted with ethyl acetate. The combined EtOAc was dried over Na₂SO₄ and concentrated. The residue was purified on a silica gel column (20:1 EtOAc:MeOH) to give (2S)-2,4,5-tris({[(tert-butoxy)carbonyl]amino}) pentanoic acid CXXX (200 mg, 0.44 mmol, 23% yield). ¹H NMR (DMSO-d₆) 1.38 (s, 27H), 1.84-1.92 (m, 1H), 2.07-2.12 (m, 1H), 2.86-2.98 (m, 2H), 3.99-4.08 (m, 1H), 6.83-6.89 (m, 0.3H 1^{st} diastereoisomer), 6.95-6.98 (m, 0.7 Hz 2^{nd} diastereoisomer), 7.00-7.05 (m, 1H), 7.17 (s, 0.3H), 7.82 (s, 0.7H). ESIMS found for C₂₀H₃₇N₃O₈ m/z 470 (M+Na).

### Step 5

Procedure can be found in previous examples.

### Step 6

Procedure can be found in previous examples. The final compound 40 was isolated as the hydrochloride salt. ¹H NMR (DMSO-d₆) 2.02-2.38 (m, 4H), 2.64-2.80 (m, 2H), 3.24-3.40 (m, 2H), 4.33-4.37 (m, 1H), 4.55-4.58 (m, 1H), 7.17-7.22 (m, 1H), 7.20-7.29 (m, 5H), 7.62-7.67 (m, 1H), 7.71-7.76 (m, 1H), 7.98-8.05 (m, 2H), 8.63 (brs, 6H), 8.79 (brs, 3H), 8.81 (s, 1H), 9.13 (d, J=2 Hz, 1H), 9.47 (d, J=7 Hz, 1H), 10.93 (s, 1H); ESIMS found for C₂₄H₃₀N₆O₂ m/z 435 (M+H).

Scheme 20 describes an example for the preparation of a parallel synthesis library of polyamine EPIs. Thus the carboxylic acid CXXXII was coupled using standard methods with a variety of CAP amines CXXXIII to give the polyamine EPI CXXXIV.

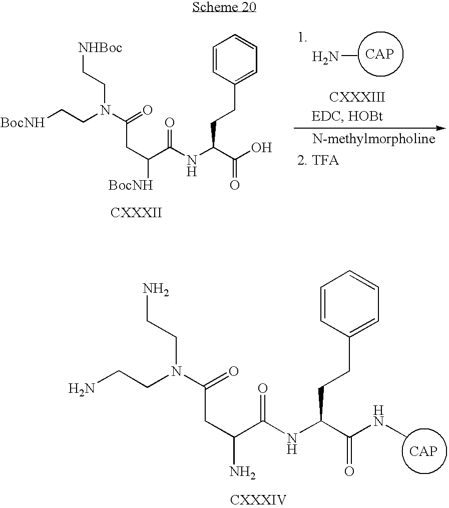

TABLE 1
The following compounds are prepared in accordance with the
procedure described as in the above scheme 20

| Compound # | CAP amine CXXXIII | ESIMS found |
|---|---|---|
| 49 | 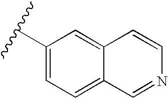 | 506.2 (M + H) |
| 52 | 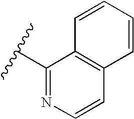 | 506.2 (M + H) |
| 56 | 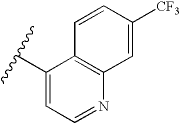 | 574.2 (M + H) |
| 57 | 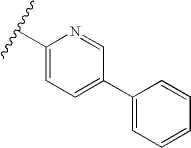 | 532.3 (M + H) |
| 60 | 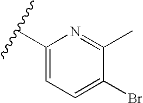 | 548.2 (M + H) |
| 61 | 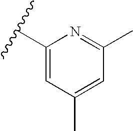 | 506.2 (M + Na) |
| 62 | 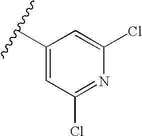 | 524.2 (M + H) |
| 68 | 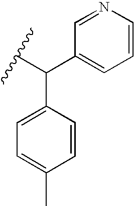 | |
| 69 | 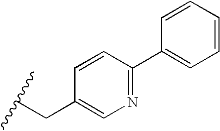 | 546.3 (M + H) |
| 70 | 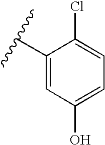 | 505.2 (M + H) |
| 71 | 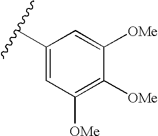 | 545.5 (M + H) |
| 72 | 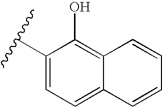 | 521.5 (M + H) |
| 74 | 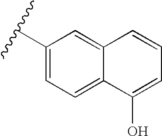 | 521.3 (M + H) |
| 75 | 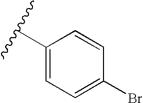 | 533.2 (M + H) |
| 77 | 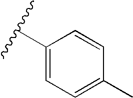 | 469.3 (M + H) |
| 79 | 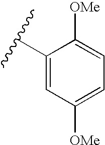 | 515.3 (M + H) |
| 80 | 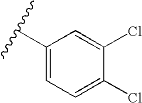 | 523.2 (M + H) |
| 82 | 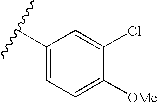 | 519.4 (M + H) |
| 83 | 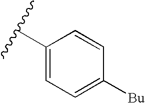 | 511.5 (M + H) |
| 84 | 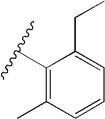 | 497.5 (M + H) |
| 85 | 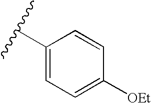 | 499.5 (M + H) |
| 88 | 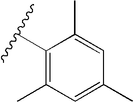 | 497.5 (M + H) |
| 89 | 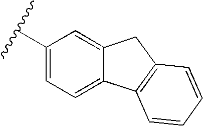 | 543.3 (M + H) |
| 91 | 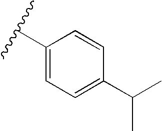 | 497.3 (M + H) |
| 93 | 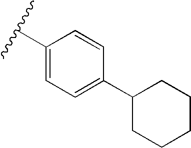 | 537.3 (M + H) |
| 96 | 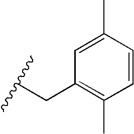 | 497.5 (M + H) |
| 97 | 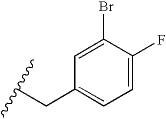 | 565.3 (M + H) |
| 101 | 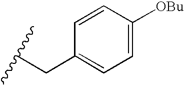 | 541.3 (M + H) |
| 102 | 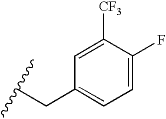 | 555.3 (M + H) |
| 103 | 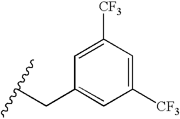 | |
| 106 | 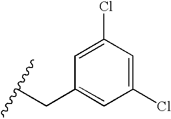 | 537.4 (M + H) |
| 107 | 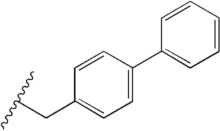 | 545.4 (M + H) |
| 108 | 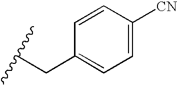 | 494.2 (M + H) |
| 109 | 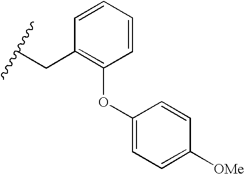 | 591.5 (M + H) |
| 110 | 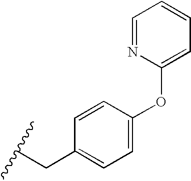 | 562.5 (M + H) |
| 111 | 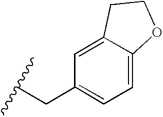 | 511.5 (M + H) |
| 112 | 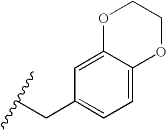 | 527.3 (M + H) |
| 113 | 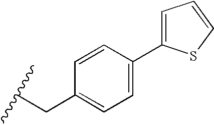 | 551.2 (M + H) |
| 114 | 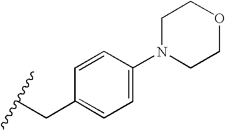 | 554.2 (M + H) |
| 115 | 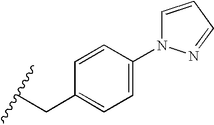 | 535.3 (M + H) |
| 116 | 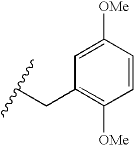 | 529.5 (M + H) |
| 117 | 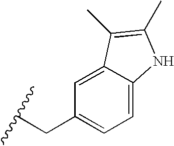 | 536.6 (M + H) |
| 118 | 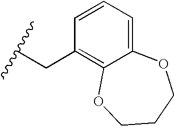 | 541.3 (M + H) |
| 119 | 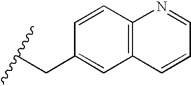 | 260.7 (M + H)/2 |
| 122 | 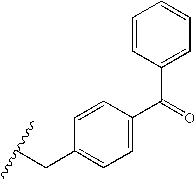 | 573.5 (M + H) |
| 123 | 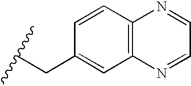 | 521.4 (M + H) |
| 124 | 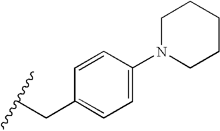 | 552.3 (M + H) |
| 125 | 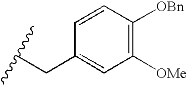 | 605.3 (M + H) |
| 126 | 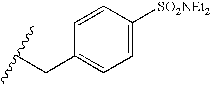 | 604.3 (M + H) |
| 127 | 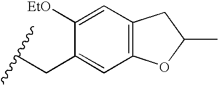 | 569.3 (M + H) |
| 129 | 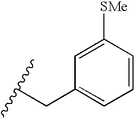 | 515.3 (M + H) |
| 130 | 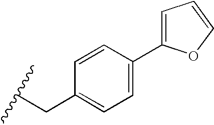 | 535.3 (M + H) |
| 131 | 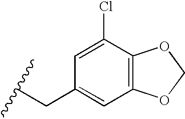 | 547.4 (M + H) |
| 132 | 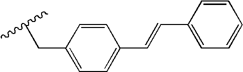 | 571.3 (M + H) |
| 133 | 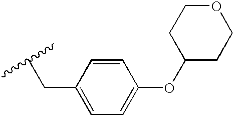 | 569.3 (M + H) |
| 134 | 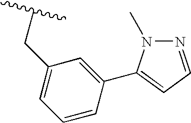 | 549.3 (M + H) |
| 135 | 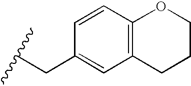 | 525.5 (M + H) |
| 136 | 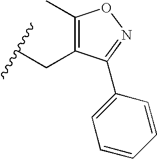 | 550.3 (M + H) |
| 137 | 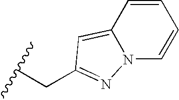 | 509.4 (M + H) |
| 138 | 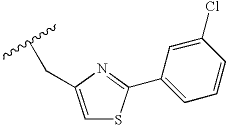 | 586.2 (M + H) |
| 140 | 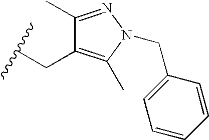 | 577.3 (M + H) |
| 141 | 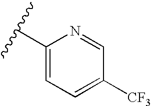 | 524.2 (M + H) |

### Methods of Treatment

Some embodiments include a method of inhibiting a bacterial efflux pump comprising administering to a subject infected with bacteria, a compound according to any of the structures described above. Other embodiments include a method of treating or preventing a bacterial infection comprising administering to a subject infected with bacteria or subject to infection with bacteria, a compound according to any of the structures described above in combination with another anti-bacterial agent.

### Microbial Species

The microbial species to be inhibited through the use of efflux pump inhibitors, such as the above-described EPIs, can be from other bacterial groups or species, such as one of the following: Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

A particularly appropriate example of a microbe appropriate for the use of an efflux pump inhibitor of the preferred embodiments is a pathogenic bacterial species, Pseudomonas aeruginosa, which is intrinsically resistant to many of the commonly used antibacterial agents. Exposing this bacterium to an efflux pump inhibitor can significantly slow the export of an antibacterial agent from the interior of the cell or the export of siderophores. Therefore, if another antibacterial agent is administered in conjunction with the efflux pump inhibitor of preferred embodiments, the antibacterial agent, which would otherwise be maintained at a very low intracellular concentration by the export process, can accumulate to a concentration, which will inhibit the growth of the bacterial cells. This growth inhibition can be due to either bacteriostatic or bactericidal activity, depending on the specific antibacterial agent used. While P. aeruginosa is an example of an appropriate bacterium, other bacterial and microbial species may contain similar broad substrate pumps, which actively export a variety of antimicrobial agents, and thus can also be appropriate targets.

### Antimicrobial Agents

In particular embodiments various antibacterial agents can be used in combination with the efflux pump inhibitors described herein. These include quinolones, tetracyclines, glycopeptides, aminoglycosides, β-lactams, rifamycins, macrolides/ketolides, oxazolidinones, coumermycins, and chloramphenicol. In particular embodiments, an antibiotic of the above classes can be, for example, one of the following.

Beta-Lactam Antibiotics

Beta-lactam antibiotics include, but are not limited to, imipenem, meropenem, biapenem, cefaclor, cefadroxil, cefamandole, cefatrizine, cefazedone, cefazolin, cefixime, cefmenoxime, cefodizime, cefonicid, cefoperazone, ceforanide, cefotaxime, cefotiam, cefpimizole, cefpiramide, cefpodoxime, cefsulodin, ceftazidime, cefteram, ceftezole, ceftibuten, ceftizoxime, ceftriaxone, cefuroxime, cefuzonam, cephaacetrile, cephalexin, cephaloglycin, cephaloridine, cephalothin, cephapirin, cephradine, cefmetazole, cefoxitin, cefotetan, azthreonam, carumonam, flomoxef, moxalactam, amidinocillin, amoxicillin, ampicillin, azlocillin, carbenicillin, benzylpenicillin, carfecillin, cloxacillin, dicloxacillin, methicillin, mezlocillin, nafcillin, oxacillin, penicillin G. piperacillin, sulbenicillin, temocillin, ticarcillin, cefditoren, SC004, KY-020, cefdinir, ceftibuten, FK-312, S-1090, CP-0467, BK-218, FK-037, DQ-2556, FK-518, cefozopran, ME1228, KP-736, CP-6232, Ro 09-1227, OPC-20000, and LY206763.

Macrolides

Macrolides include, but are not limited to, azithromycin, clarithromycin, erythromycin, oleandomycin, rokitamycin, rosaramicin, roxithromycin, and troleandomycin.

Ketolides

Ketolides include, but are not limited to, telithromycin and cethrimycin.

Quinolones

Quinolones include, but are not limited to, amifloxacin, cinoxacin, ciprofloxacin, enoxacin, fleroxacin, flumequine, lomefloxacin, nalidixic acid, norfloxacin, ofloxacin, levofloxacin, oxolinic acid, pefloxacin, rosoxacin, temafloxacin, tosufloxacin, sparfloxacin, clinafloxacin, moxifloxacin; gemifloxacin; garenofloxacin; PD131628, PD138312, PD140248, Q-35, AM-1155, NM394, T-3761, rufloxacin, OPC-17116, DU-6859a (see, e.g. Sato, K. et al., 1992, Antimicrob Agents Chemother. 37:1491-98), and DV-7751a (see, e.g., Tanaka, M. et al., 1992, Antimicrob. Agents Chemother. 37:2212-18).

Tetracyclines, Glycylcyclines and Oxazolidinones

Tetracyclines, glycylcyclines, and oxazolidinones include, but are not limited to, chlortetracycline, demeclocycline, doxycycline, lymecycline, methacycline, minocycline, oxytetracycline, tetracycline, tigecycline, linezolide, and eperozolid.

Aminoglycosides

Aminoglycosides include, but are not limited to amikacin, arbekacin, butirosin, dibekacin, fortimicins, gentamicin, kanamycin, meomycin, netilmicin, ribostamycin, sisomicin, spectinomycin, streptomycin, and tobramycin.

Lincosamides

Lincosamides include, but are not limited to, clindamycin and lincomycin.

Efflux pumps export substrate molecules from the cytoplasm in an energy-dependent manner, and the exported substrate molecules can include antibacterial agents. Such efflux pump inhibitors are useful, for example, for treating microbial infections by reducing the export of a co-administered antimicrobial agent or by preventing the export of a compound synthesized by microbes (e.g. bacteria) to allow or improve their growth. While the endogenous substrates of efflux pumps are not yet identified, there are some indications that efflux pumps may be important for bacterial virulence. Thus, also disclosed herein are compositions that include such efflux pump inhibitors and methods for treating microbial infections using those compositions.

In some embodiments, a method is provided for treating a microbial infection in an animal, specifically including in a mammal, by treating an animal suffering from such an infection with an antimicrobial agent and an efflux pump inhibitor, which increase the susceptibility of the microbe for that antimicrobial agent. Such efflux pump inhibitors can be selected from any of the compounds generically or specifically described herein. In this way a microbe involved in the infection can be treated using the antimicrobial agent in smaller quantities, or can be treated with an antimicrobial agent, which is not therapeutically effective when used in the absence of the efflux pump inhibitor. Thus, this method of treatment is especially appropriate for the treatment of infections involving microbial strains that are difficult to treat using an antimicrobial agent alone due to a need for high dosage levels (which can cause undesirable side effects), or due to lack of any clinically effective antimicrobial agents. However, it is also appropriate for treating infections involving microbes that are susceptible to particular antimicrobial agents as a way to reduce the dosage of those particular agents. This can reduce the risk of side effects. It is also appropriate for treating infections involving microbes that are susceptible to particular antimicrobial agents as a way of reducing the frequency of selection of resistant microbes. In particular embodiments the microbe is a bacterium, which may, for example, be from any of the groups or species indicated above.

In some embodiments, a method is provided for prophylactic treatment of a mammal. In this method, an antimicrobial agent and an efflux pump inhibitor is administered to a mammal at risk of a microbial infection, e.g. a bacterial infection. The efflux pump inhibitor can be selected from any of the compounds generically or specifically described herein.

In some embodiments, a method is provided for enhancing the antimicrobial activity of an antimicrobial agent against a microbe, in which such a microbe is contacted with an efflux pump inhibitor, and an antibacterial agent. The efflux pump inhibitor can be selected from any of the compounds generically or specifically described herein. Thus, this method makes an antimicrobial agent more effective against a cell, which expresses an efflux pump when the cell is treated with the combination of an antimicrobial agent and an efflux pump inhibitor. In particular embodiments the microbe is a bacterium or a fungus, such as any of those indicated above; the antibacterial agent can be selected from a number of structural classes of antibiotics including, e.g. beta-lactams, glycopeptides, aminoglycosides, quinolones, oxazolidinones, tetracyclines, rifamycins, coumermycins, macrolides, and chloramphenicol. In particular embodiments an antibiotic of the above classes can be as stated above.

In other embodiments, a method is provided for suppressing growth of a microbe, e.g. a bacterium, expressing an efflux pump, e.g. a non-tetracycline-specific efflux pump. As illustrated by the case where the microbe is a bacterium, the method involves contacting that bacterium with an efflux pump inhibitor, in the presence of a concentration of antibacterial agent below the MIC of the bacterium. The efflux pump inhibitor can be selected from any of the compounds generically or specifically described herein. This method is useful, for example, to prevent or cure contamination of a cell culture by a bacterium possessing an efflux pump. However, it applies to any situation where such growth suppression is desirable.

In some embodiments, any of the compounds generically or specifically described herein may be administered as an efflux pump inhibitor either alone or, more preferably, in conjunction with another therapeutic agent. In some embodiments, any of the compounds generically or specifically described herein may be administered as an efflux pump inhibitor in conjunction with any of the antibacterial agents specifically or generically described herein, as well as with any other antibacterial agent useful against the species of bacterium to be treated, when such bacteria do not utilize an efflux pump resistance mechanism. In some embodiments, the antibacterial agents are administered at their usual recommended dosages. In other embodiments, the antibacterial agents are administered at reduced dosages, as determined by a physician. For all conventional antibacterials on the market, and many in clinical development, dosage ranges and preferred routes of administration are well established, and those dosages and routes can be used in conjunction with the efflux pump inhibitors of the preferred embodiments. Reduced dosages of the antibacterials are contemplated due to the increased efficacy of the antibacterial when combined with an efflux pump inhibitor.

Potential efflux pump inhibitor compounds can be tested for their ability to inhibit multi-drug resistance efflux pumps of various microbes using the methods described herein as well as those known in the art. For example, treatment of P. aeruginosa with a test compound allows obtaining one or more of the following biological effects:

1) P. aeruginosa strains will become susceptible to antibiotics that could not be used for treatment of pseudomonad infections, or become more susceptible to antibiotics, which do inhibit pseudomonal growth.

2) P. aeruginosa strains will become more susceptible to antibiotics currently used for treatment of pseudomonad infections.

3) Inhibition of the pump will result in a decreased frequency of resistance development to antibiotic, which is a substrate of the pump.

Obtaining even one of these effects provides a potential therapeutic treatment for infections by this bacterium. Also, similar pumps are found in other microorganisms. Some or all of the above effects can also be obtained with those microbes, and they are therefore also appropriate targets for detecting or using efflux pump inhibitors.

### Administration

The efflux pump inhibitors are administered at a therapeutically effective dosage, e.g. a dosage sufficient to provide treatment for the disease states previously described. While human dosage levels have yet to be optimized for the compounds of the preferred embodiments, generally, a daily dose for most of the inhibitors described herein is from about 0.05 mg/kg or less to about 100 mg/kg or more of body weight, preferably from about 0.10 mg/kg to 10.0 mg/kg of body weight, and most preferably from about 0.15 mg/kg to 1.0 mg/kg of body weight. Thus, for administration to a 70 kg person, the dosage range would be about 3.5 mg per day or less to about 7000 mg per day or more, preferably from about 7.0 mg per day to 700.0 mg per day, and most preferably from about 10.0 mg per day to 100.0 mg per day. The amount of active compound administered will, of course, be dependent on the subject and disease state being treated, the severity of the affliction, the manner and schedule of administration and the judgment of the prescribing physician; for example, a likely dose range for oral administration can be from about 70 mg per day to 700 mg per day, whereas for intravenous administration a likely dose range can be from about 700 mg per day to 7000 mg per day, the active agents being selected for longer or shorter plasma half-lives, respectively. Screening techniques described herein for the compounds of preferred embodiments can be used with other efflux pump inhibitors described herein to establish the efficacy of those inhibitors in comparison to reference compounds, and the dosage of the inhibitor can thus be adjusted to achieve an equipotent dose to the dosages of reference compound.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administration are customary in treating the indication.

Pharmaceutically acceptable compositions include solid, semi-solid, liquid and aerosol dosage forms, such as, e.g. tablets, capsules, powders, liquids, suspensions, suppositories, aerosols or the like. The compounds can also be administered in sustained or controlled release dosage forms, including depot injections, osmotic pumps, pills, transdermal (including electrotransport) patches, and the like, for prolonged and/or timed, pulsed administration at a predetermined rate. Preferably, the compositions are provided in unit dosage forms suitable for single administration of a precise dose.

The compounds can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g. sodium acetate, sodium citrate, cyclodextrine derivatives, sorbitan monolaurate, triethanolamine acetate, triethanolamine oleate, and the like). Generally, depending on the intended mode of administration, the pharmaceutical formulation will contain about 0.005% to 95%, preferably about 0.5% to 50% by weight of a compound of the preferred embodiments. Actual methods of preparing such dosage forms are known, or will be apparent, to those skilled in this art; for example, see Remington's Pharmaceutical Sciences, Mack Publishing Company, Easton, Pa.

In addition, the compounds can be co-administered with, and the pharmaceutical compositions can include, other medicinal agents, pharmaceutical agents, adjuvants, and the like. Suitable additional active agents include, for example, antimicrobial agents as described above. When used, other active agents may be administered before, concurrently, or after administration of an efflux pump inhibitor of the preferred embodiments. In some embodiments, an efflux pump inhibitor is co-administered with one or more other antimicrobial agents. By “co-administer” it is meant that the efflux pump inhibitors are administered to a patient such that the present compounds as well as the co-administered compound may be found in the patient's bloodstream at the same time, regardless of when the compounds are actually administered, including simultaneously. In one advantageous embodiment, the pharmacokinetics of the efflux pump inhibitors and the co-administered antimicrobial agent are substantially the same.

Thus, in the preferred embodiments, an efflux pump inhibitor compound as set forth herein can be administered through a first route of administration, and the antimicrobial agent can be administered through a second route. Thus, for example, an efflux pump inhibitor can be administered via a pulmonary route, e.g. through a nebulizer, atomizer, mister, aerosol, dry powder inhaler, or other suitable device or technique, and the antimicrobial can be administered via the same or a different route, e.g. orally, parenterally, intramuscularly, intraperitoneally, intratracheally, intravenously, subcutaneously, transdermally, or as a rectal or vaginal suppository. The blood levels of drugs are affected by the route of administration. Thus, in one preferred embodiment, when the efflux pump inhibitor is administered by a first route, and the antibiotic or antimicrobial through a second route, the dosages or dosage forms are adjusted, as appropriate, to match the pharmcokinetic profiles of each drug. This may also be done when both drugs are administered by the same route. In either event, conventional techniques, including controlled release formulations, timing of administration, use of pumps and depots, and/or use of biodegradable or bioerodible carriers can be used to match the pharmacokinetic of the two active moieties.

In one preferred embodiment, the compositions will take the form of a unit dosage form such as a pill or tablet and thus the composition may contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule. Unit dosage forms in which the two active ingredients (inhibitor and antimicrobial) are physically separated are also contemplated; e.g. capsules with granules of each drug; two-layer tablets; two-compartment gel caps, etc.

Liquid pharmaceutically administrable compositions can, for example, be prepared by dissolving, dispersing, etc. an active compound as defined above and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of active compound contained in such parenteral compositions is highly dependent on the specific nature thereof, as well as the activity of the compound and the needs of the subject. However, percentages of active ingredient of 0.01% to 10% in solution are employable, and will be higher if the composition is a solid, which will be subsequently diluted to the above percentages. In some embodiments, the composition will comprise 0.2-2% of the active agent in solution.

Efflux pump inhibitors (EPIs) as described herein, including any of the compounds generically or specifically described herein, can also be administered to the respiratory tract as an aerosol. For the purposes of delivery to the respiratory tract, any of the inhaler designs known in the art may be used. In some embodiments, a metered dose inhaler (MDI) is used. A typical MDI for use with the EPIs described herein comprises the EPI compound suspended or dissolved in a pressurized liquid propellant, with or without other excipients. When the MDI inhaler is activated, a metered amount of the propellant is released and rapidly evaporates due to the sudden reduction in pressure. The process causes an aerosol cloud of drug particles to be released that can be inhaled by the patient.

Solid compositions can be provided in various different types of dosage forms, depending on the physicochemical properties of the drug, the desired dissolution rate, cost considerations, and other criteria. In one of the embodiments, the solid composition is a single unit. This implies that one unit dose of the drug is comprised in a single, physically shaped solid form or article. In other words, the solid composition is coherent, which is in contrast to a multiple unit dosage form, in which the units are incoherent.

Examples of single units which may be used as dosage forms for the solid composition include tablets, such as compressed tablets, film-like units, foil-like units, wafers, lyophilized matrix units, and the like. In a preferred embodiment, the solid composition is a highly porous lyophilized form. Such lyophilizates, sometimes also called wafers or lyophilized tablets, are particularly useful for their rapid disintegration, which also enables the rapid dissolution of the active compound.

On the other hand, for some applications the solid composition may also be formed as a multiple unit dosage form as defined above. Examples of multiple units are powders, granules, microparticles, pellets, beads, lyophilized powders, and the like. In one embodiment, the solid composition is a lyophilized powder. Such a dispersed lyophilized system comprises a multitude of powder particles, and due to the lyophilization process used in the formation of the powder, each particle has an irregular, porous microstructure through which the powder is capable of absorbing water very rapidly, resulting in quick dissolution.

Another type of multiparticulate system which is also capable of achieving rapid drug dissolution is that of powders, granules, or pellets from water-soluble excipients which are coated with the drug, so that the drug is located at the outer surface of the individual particles. In this type of system, the water-soluble low molecular weight excipient is useful for preparing the cores of such coated particles, which can be subsequently coated with a coating composition comprising the drug and, preferably, one or more additional excipients, such as a binder, a pore former, a saccharide, a sugar alcohol, a film-forming polymer, a plasticizer, or other excipients used in pharmaceutical coating compositions.

For purposes of co-administration of an EPI as described herein and another anti-bacterial compound, the EPI can be administered by the same route as the other anti-bacterial compound, either simultaneously or sequentially. In some embodiments, the EPI and other anti-bacterial compound or compounds are both administered intravenously (i.v.), either mixed in a fixed drug formulation or present in separate formulations. In other embodiments, the EPI and other anti-bacterial compound or compounds are both administered orally, either in the same fixed formulation or in separate formulations. In still other embodiments, the EPI and other anti-bacterial compound or compounds are both administered intramuscularly (i.m.), again either mixed in a fixed drug formulation or present in separate formulations.

In some embodiments, the EPI and other anti-bacterial compound to be co-administered are administered by separate routes. For example, the EPI may be administered by inhalation while the other anti-bacterial compound is administered i.v., i.m., or orally. Any other possible combination of separate route administration is also contemplated.

The preferred embodiments also include any of the novel compounds disclosed herein per se, as well as any of the efflux pump inhibitors disclosed herein in unit dosage forms combined with or for co-administration with an antimicrobial, as well as methods of treating an animate or inanimate subject or object with those efflux pump inhibitors, preferably in combination with an antimicrobial. Metered dose inhalers or other delivery devices containing both an efflux pump inhibitor as described herein as well as an antimicrobial are also preferred embodiments

### Examples

EPI activity was recorded as concentration of an EPI compound that is necessary to increase susceptibility to levofloxacin of the strain of P. aeruginosa, PAM1723, overexpressing the MexAB-OprM efflux pump eight-fold. The levofloxacin potentiating activity of the test compounds was assessed by the checkerboard assay (Antimicrobial Combinations, Antibiotics in Laboratory Medicine, Ed. Victor Lorian, M.D., Fourth edition, 1996, pp 333-338, which is incorporated herein by reference in its entirety) using a broth microdilution method performed as recommended by the NCCLS (National Committee for Clinical Laboratory Standards (NCCLS), 1997, Methods for Dilution of Antimicrobial Susceptibility Tests for Bacteria That Grow Aerobically, Fourth Edition; Approved Standard. NCCLS Document M7-A4, Vol 17 No. 2, which is incorporated herein by reference in its entirety). In this assay, multiple dilutions of two drugs, namely an EPI and levofloxacin, were tested, alone and in combination, at concentrations equal to, above and below their respective minimal inhibitory concentrations (MICs). All EPI compounds were readily soluble in water and stock solutions were prepared at a final concentration of 10 mg/ml. Stock solutions were further diluted, according to the needs of the particular assay, in Mueller Hinton Broth (MHB). Stock solution was stored at −80° C.

The checkerboard assay was performed in microtiter plates. Levofloxacin was diluted in the x-axis, each column containing a single concentration of levofloxacin. EPIs were diluted in the y-axis, each row containing a single concentration of an EPI. The result of these manipulations was that each well of the microtiter plate contained a unique combination of concentrations of the two agents. The assay was performed in MHB with a final bacterial inoculum of 5 times 10⁵ CFU/ml (from an early-log phase culture). Microtiter plates were incubated during 20 h at 35° C. and were read using a microtiterplate reader (Molecular Devices) at 650 nm as well as visual observation using a microtiter plate-reading mirror. The MIC (here referred to as MPC; see infra) was defined as the lowest concentration of antibiotics, within the combination, at which the visible growth of the organism was completely inhibited.

### Example 1

### Potentiation of Levofloxacine (MPC₈) by Polybasic Efflux Pump Inhibitors

TABLE 2

| Compound | MPC₈ (μg/mL) | MPC₃₂ (μg/mL) |
|---|---|---|
| 1 | 0.3 | 0.6 |
| 2 | 1.25 | 5 |
| 3 | 1.25 | >10 |
| 4 | 1.25 | 2.5 |
| 5 | 0.6 | >10 |
| 6 | 2.5 | >10 |
| 8 | 1.25 | 5 |
| 10 | 0.6 | 2.5 |
| 12 | 1.25 | 2.5 |
| 11 | 2.5 | >10 |
| 13 | 1.25 | 5 |
| 14 | 2.5 | >10 |
| 15 | 5 | >10 |
| 17 | 2.5 | 5 |
| 19 | 10 | >10 |
| 20 | 10 | >10 |
| 21 | 2.5 | 5 |
| 22 | 0.3 | 0.3 |
| 23 | 1.25 | >10 |
| 24 | 0.6 | >10 |
| 25 | 0.3 | 1.25 |
| 27 | 0.6 | >10 |
| 28 | 1.25 | >10 |
| 30 | 2.5 | 5 |
| 31 | 0.3 | 0.6 |
| 32 | 1.25 | 5 |
| 33 | 2.5 | 2.5 |
| 38 | 0.3 | 0.6 |
| 39 | 10 | >40 |
| 40 | 20 | >20 |
| 41 | 5 | 10 |
| 42 | 0.3 | 0.6 |
| 43 | 0.6 | >40 |
| 44 | 0.6 | 1.25 |
| 45 | 0.6 | 0.6 |
| 46 | 0.6 | >40 |
| 47 | 0.6 | 2.5 |
| 48 | 1.25 | 2.5 |
| 56 | 1.25 | >10 |
| 57 | 1.25 | >10 |
| 83 | 0.63 | >10 |
| 85 | 5 | >10 |
| 107 | 2.5 | >10 |
| 109 | 2.5 | >10 |
| 122 | 2.5 | 5 |
| 138 | 2.5 | >10 |

In the experiment depicted in Table 2, potentiating activities of selected inhibitors are reported as Minimum Potentiating Concentration MPC₈ values (or MPC₃₂) which correspond to the lowest concentration of the inhibitor required to achieve antibacterial activity in combination with the concentration of levofloxacin equal to ⅛ (or 1/32) of the levofloxacin concentration required to achieve the same antibacterial effect alone (MIC of levofloxacin).

### Example 2

### Pharmacokinetics of Polybasic Efflux Pump Inhibitors in Rats after IV Infusion

TABLE 3

| | Dose | Clearance^{a} | C ₘₐₓ |
|---|---|---|---|
| Compound | (mg/kg) | (L/h/kg) | (μg/mL) |
| 2 | 10 | 1.40 | 26.0 |
| 3 | 20 | 1.2 | 27.8 |
| 8 | 20 | 9.20 | 19.5 |
| 28 | 10 | 14.15 | 3.62 |
| 30 | 10 | 3.36 | 7.61 |
| 31 | 10 | 1.80 | 21.9 |
| 33 | 10 | 6.32 | 3.98 |
| 41 | 10 | 2.72 | 8.1 |
| 44 | 2 | 1.48 | 2.68 |
| 46 | 2 | 0.81 | 4.0 |
| 47 | 5 | 1.70 | 8.1 |
| 48 | 5 | 1.56 | 7.2 |

^{a}free drug clearance

In the experiment depicted in Table 3, rat serum pharmacokinetics of selected inhibitor compounds was evaluated after 1.5-hour IV infusion of 1.5 ml solution of corresponding efflux pump inhibitor in 0.9% saline. Depending on the concentration used the total infused dose was 2, 5, 10 or 20 mg/kg. A two-compartment model was used to fit the data and calculate PK parameters. Compounds 2, 3, 46 and 48 showed particularly attractive pharmacokinetic profiles.

Although the invention has been described with reference to embodiments and examples, it should be understood that numerous and various modifications can be made without departing from the spirit of the invention. Accordingly, the invention is limited only by the following claims.

What is claimed is:

1. A compound having the structure of formula I, II or III: 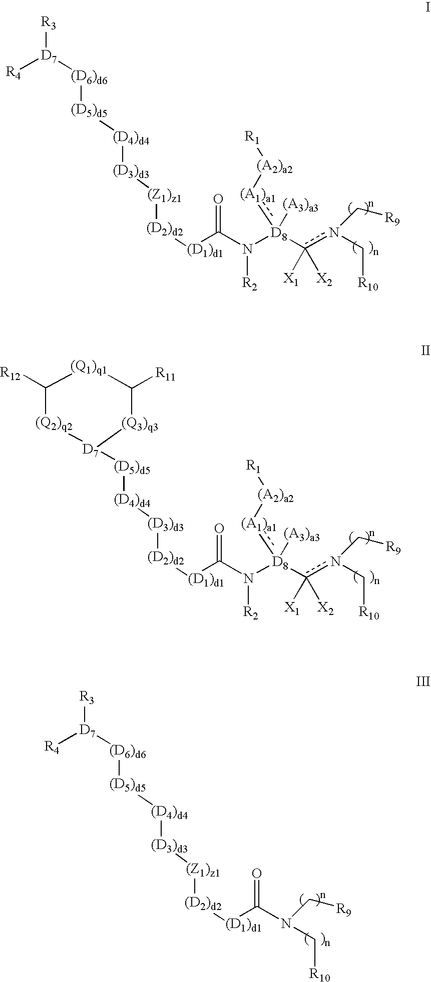
or a pharmaceutically acceptable salt or pro-drug thereof wherein;
each bond represented by a dashed and solid line represents a bond selected from the group consisting of a single bond and a double bond;
each R₁ is independently selected from C₁-C₆ alkyl, C₃-C₇ carbocyclyl, heterocyclyl, aryl and heteroaryl, each optionally substituted with up to 3 substituents independently selected from the group consisting of a halide, alkyl, carbocyclyl, —(CH₂)ₙaryl, —OR₂, —OR₁₀, —S(R₂)₂, —SO₂NHR₁₀, —(CH₂)ₙSH, —CF₃, —OCF₃, —N(R₂)₂, —NO₂, —CN, —CO₂alkyl, —CO₂aryl and —C(O)aryl;
each R₂ is independently selected from H and C₁-C₆ alkyl;
R₃ is selected from —(CH₂)ₙCHR₅R₆, —(CH₂)ₙNR₅R₆, and —(CH₂)ₘC(═O)NR₅R₆;
each R₄ is independently selected from —NHR₂, —(CH₂)ₙCHR₅R₆, —(CH₂)ₙNR₅R₆, —(CH₂)ₘC(═O)NR₅R₆, and —C(═NR₅)NR₅R₅;
each R₅ is independently selected from H and —(CH₂)ₘNH₂;
each R₆ is independently selected from —(CH₂)ₙNHR₇, —(CH₂)ₙNHC(═NH)NH₂, —(CH₂)ₙNHC(R₂)═NH, —(CH₂)ₙC(═NH)NH₂, and —(CH₂)ₙN⁺(CH₃)₃;
each R₇ is independently selected from H, alkyl, —C(═O)CH(R₁₃)(NH₂), —C(═O)A₂CH₂NH₂, Alanine, Arginine, Asparagine, Aspartic acid, Glutamic acid, Glutamine, Cysteine, Glycine, Histidine, Isoleucine, Leucine, Lysine, Methionine, Phenylalanine, Proline, Serine, Threonine, Tryptophan, Tyrosine, and Valine;
R₈ is selected from H, alkyl, aryl, SH and OH;
R₉ is selected from H, C₁-C₆ alkyl, C₃-C₁₀ carbocyclyl, heterocyclyl, aryl, heteroaryl, and —NHC(O)-aryl, each optionally substituted with up to 3 substituents independently selected from the group consisting of a halide, alkyl, carbocyclyl, —(CH₂)ₙR₁, —(CH═CH)ₙR₁, —OR₂, —OR₁, ═O, —S(R₂)₂, —SR₁, —SO₂NR₁R₂, —(CH₂)ₙSH, —CF₃, —OCF₃, —N(R₂)₂, —NO₂, —CN, —(C═X)R₁, —(C═X)R₂, —CO₂alkyl, —CO₂aryl, heteroaryl optionally substituted with C₁-C₆ alkyl, and aryl optionally substituted with C₁-C₆ alkyl;
R₁₀ is selected from C₁-C₆ alkyl, C₃-C₁₀ carbocyclyl, heterocyclyl, aryl, heteroaryl, and —NHC(O)-aryl, each optionally substituted with up to 3 substituents independently selected from the group consisting of a halide, alkyl, carbocyclyl, —(CH₂)ₙR₁, —OR₂, —OR₁, ═O, —S(R₂)₂, —SR₁, —SO₂NR₁R₂, —(CH₂)ₙSH, —CF₃, —OCF₃, —N(R₂)₂, —NO₂, —CN, —(C═X)R₁, —(C═X)R₂, —CO₂alkyl, and —CO₂aryl;
R₉ and R₁₀ are optionally linked to form a ring;
R₁₁ is selected from H, —(CH₂)ₙNHR₂ and —(CH₂)ₙCHR₅R₆;
R₁₂ is selected from —(CH₂)ₙNHR₂ and —(CH₂)ₙCHR₅R₆;
R₁₃ is selected from —(CH₂)ₙCHR₅(CH₂)ₙNH₂, —(CH₂)ₘNR₅(CH₂)ₙNH₂ and —(CH₂)ₘC(═O)NR₅(CH₂)ₙNH₂;
A₁ is —[C(R₂R₈)]ₘ or ═CR₂[C(R₂R₈)]ₘ—, wherein if A₁ is ═CR₂[C(R₂R₈)]ₘ—, then a3 is 0;
A₂ is —(CH₂)ₘ, —C(═X)—, —O(CH₂)ₙ—, —S(CH₂)ₙ—, —CH═CH—, —C(═N—OR₂)—, or —NR₂—;
A₃ is H, C₁-C₆ alkyl, a lone electron pair when D₈ is N, or A₃ is —CH₂-bonded to A₁, A₂ or R₁ to form a ring;
a1, a2 and a3 are independently equal to 0 or 1;
D₁ is selected from —CH₂—, —N(NHR₇)—, —CH(NHR₇)—, —CH[(CH₂)ₘNHR₇]—, —CH(R₂)—, and —CH(CH₂SH)—;
D₂, D₃, D₄, D₅ and D₆ are independently selected from the group consisting of —(CH₂)ₘ—, —CH(R₂)—, —CH(NHR₇)—, —N(R₅)—, —O—, —S—, —C(═X)—, —S(═O)— and —SO₂—, wherein any two atoms of D₂, D₃, D₄, D₅ and D₆ are optionally linked to form a three, four, five or six membered saturated ring;
D₇ is selected from N, ═C< where the carbon forms a double bond with an adjacent carbon in one of D₁-D₆, CH and CR₄;
D₈ is selected from C and N;
d1, d2, d3, d4, d5 and d6 are independently equal to 0 or 1;
Q₁ is selected from —CH₂—, —N(R₂)N(R₂)—, and —N(R₂)—;
Q2 and Q3 are independently selected from the group consisting of —CH₂— and —N(R₂)—;
with the proviso that no more than one of Q1, Q2, and Q3 comprises a nitrogen;
q1, q2, and q3 are independently equal to 0 or 1;
X₁ and X₂ are each hydrogen or taken together are ═O or ═S,
or X₁ is hydrogen and X₂ is —O— or —S— bonded to R₁₀ to form a 5- or 6-membered heterocyclyl,
or X₁ is absent and X₂ is —O— or —S— bonded to R₁₀ to form a 5- or 6-membered heterocyclyl or heteroaryl, wherein when X₁ is absent, the bond to nitrogen represented by a dashed and solid line is a double bond;
each X is independently O or S;
Z₁ is an aryl, heteroaryl, carbocyclyl, or heterocyclyl;
z1 is 0 or 1;
if z1 is 0 then at least two from the group consisting of d1, d2, d3, d4, d5 and d6 are equal to 1, if z1 is 1 then at least one from the group consisting of d1, d2, d3, d4, d5 and d6 is equal to 1;
each n is independently an integer of 0 to 4; and
each m is independently an integer of 1 to 3.

2. The compound of claim 1, wherein the compound has the structure of Formula I.

3. The compound of claim 1, wherein the compound has the structure of Formula II.

4. The compound of claim 1, wherein the compound has the structure of Formula III.

5. The compound of claim 1 wherein R₁ is selected from C₁-C₆ alkyl and C₃-C₇ carbocyclyl.

6. The compound of claim 1 wherein R₁ is selected from C₃-C₄ alkyl and C₅-C₆ carbocyclyl.

7. The compound of claim 1 wherein R₁ is selected from aryl and heteroaryl, each optionally substituted with up to 3 substituents independently selected from the group consisting of halide, alkyl, carbocyclyl, —(CH₂)ₙaryl, —OR₂, —OR₁₀, —S(R₂)₂, —SO₂NHR₁₀, —(CH₂)ₙSH, —CF₃, —OCF₃, —N(R₂)₂, —NO₂, —CN, and —CO₂alkyl, and —CO₂aryl.

8. The compound of claim 1 wherein R₁ is aryl optionally substituted with up to 3 substituents independently selected from the group consisting of halide, alkyl, —OR₂, CF₃, and CN.

9. The compound of claim 1 wherein R₂ is selected from H and C₁-C₃ alkyl.

10. The compound of claim 1 wherein R₂ is selected from H and Me.

11. The compound of claim 1 wherein R₂ is H.

12. The compound of claim 1 wherein R₃ is —(CH₂)ₙCHR₅R₆ wherein n is 0 to 2, R₅ is H, and R₆ is —(CH₂)ₘNHR₇ wherein m is 1 or 2 and R₇ is H.

13. The compound of claim 1 wherein R₃ is —(CH₂)ₙCHR₅R₆ wherein n is 0 to 2, R₅ is H, and R₆ is —(CH₂)ₘNHR₇ wherein m is 1 or 2 and R₇ is Alanine, Arginine, Asparagine, Aspartic acid, Glutamic acid, Glutamine, Cysteine, Glycine, Histidine, Isoleucine, Leucine, Lysine, Methionine, Phenylalanine, Proline, Serine, Threonine, Tryptophan, Tyrosine, and Valine.

14. The compound of claim 1 wherein R₃ is —(CH₂)ₙCHR₅R₆ wherein n is 0 to 2, R₅ is H, and R₆ is —(CH₂)ₘNHR₇ wherein m is 1 or 2 and R₇ is —C(O)CH(R₁₃)(NH₂).

15. The compound of claim 1 wherein R₃ is —(CH₂)ₙCHR₅R₆ wherein n is 0 to 2, R₅ is H, and R₆ is —(CH₂)ₘNHR₇, wherein m is 1 or 2 and R₇ is —C(O)A₂CH₂NH₂.

16. The compound of claim 1 wherein R₃ is —(CH₂)ₙCHR₅R₆ wherein n is 0 to 2, R₅ is H, and R₆ is —(CH₂)ₘNHC(═NH)NH₂ wherein m is 1 or 2.

17. The compound of claim 1 wherein R₃ is —(CH₂)ₙCHR₅R₆ wherein n is 0 to 2, R₅ is H, and R₆ is (CH₂)ₘNHC(R₂)═NH wherein m is 1 or 2 and R₂ is selected from H, Me and Et.

18. The compound of claim 1 wherein R₃ is —(CH₂)ₙCHR₅R₆ wherein n is 0 to 2, R₅ is H, and R₆ is —(CH₂)ₘC(═NH)NH₂ wherein m is 1 or 2.

19. The compound of claim 1 wherein R₃ is —(CH₂)ₙCHR₅R₆ wherein n is 0 to 2, R₅ is —(CH₂)ₘNH₂, R₆ is —(CH₂)ₘNHR₇, R₇ is H, and m is 1 or 2.

20. The compound of claim 1 wherein R₃ is —(CH₂)ₙNR₅R₆ wherein n is 0 to 2, R₅ is H, and R₆ is —(CH₂)ₘNHR₇ wherein R₇ is H and m is 1 or 2.

21. The compound of claim 1 wherein R₃ is —(CH₂)ₙNR₅R₆ wherein n is 0 to 2, R₅ is H, and R₆ is —(CH₂)ₘNHR₇ wherein m is 1 or 2 and R₇ is selected from Alanine, Arginine, Asparagine, Aspartic acid, Glutamic acid, Glutamine, Cysteine, Glycine, Histidine, Isoleucine, Leucine, Lysine, Methionine, Phenylalanine, Proline, Serine, Threonine, Tryptophan, Tyrosine, and Valine.

22. The compound of claim 1 wherein R₃ is —(CH₂)ₙNR₅R₆ wherein n is 0 to 2, R₅ is H, and R₆ is —(CH₂)ₘNHR₇ wherein m is 1 or 2 and R₇ is —C(O)CH(R₁₃)(NH₂).

23. The compound of claim 1 wherein R₃ is —(CH₂)ₙNR₅R₆ wherein n is 0 to 2, R₅ is H, and R₆ is —(CH₂)ₘNHR₇ wherein m is 1 or 2 and R₇ is —C(O)A₂CH₂NH₂.

24. The compound of claim 1 wherein R₃ is —(CH₂)ₙNR₅R₆ wherein n is 0 to 2, R₅ is H, and R₆ is —(CH₂)ₘNHC(═NH)NH₂ wherein m is 1 or 2.

25. The compound of claim 1 wherein R₃ is —(CH₂)ₙNR₅R₆ wherein n is 0 to 2, R₅ is H, and R₆ is (CH₂)ₘNHC(R₂)═NH wherein m is 1 or 2 and R₂ is selected from H, Me and Et.

26. The compound of claim 1 wherein R₃ is —(CH₂)ₙNR₅R₆ wherein n is 0 to 2, R₅ is H, and R₆ is —(CH₂)ₘC(═NH)NH₂ wherein m is 1 or 2.

27. The compound of claim 1 wherein R₃ is —(CH₂)ₘC(═O)NR₅R₆ wherein R₅ is —(CH₂)ₘNH₂, R₆ is —(CH₂)ₘNHR₇, R₇ is H, and m is 1 or 2.

28. The compound of claim 1 wherein R₃ is —(CH₂)ₘC(═O)NR₅R₆ wherein R₅ is H, R₆ is —(CH₂)ₘNHR₇, R₇ is H, and m is 1 or 2.

29. The compound of claim 1 wherein R₃ is —(CH₂)ₘC(═O)NR₅R₆ wherein R₅ is H, R₆ is —(CH₂)ₘNHR₇, R₇ is selected from Alanine, Arginine, Asparagine, Aspartic acid, Glutamic acid, Glutamine, Cysteine, Glycine, Histidine, Isoleucine, Leucine, Lysine, Methionine, Phenylalanine, Proline, Serine, Threonine, Tryptophan, Tyrosine, and Valine, and m is 1 or 2.

30. The compound of claim 1 wherein R₃ is —(CH₂)ₘC(═O)NR₅R₆ wherein R₅ is H, R₆ is —(CH₂)ₘNHR₇, R₇ is —C(O)CH(R₁₃)(NH₂), and m is 1 or 2.

31. The compound of claim 1 wherein R₃ is —(CH₂)ₘC(═O)NR₅R₆ wherein R₅ is H, R₆ is —(CH₂)ₘNHR₇, R₇ is —C(O)A₂CH₂NH₂, and m is 1 or 2.

32. The compound of claim 1 wherein R₃ is —(CH₂)ₘC(═O)NR₅R₆ wherein R₅ is H, R₆ is —(CH₂)ₘNHC(═NH)NH₂, and m is 1 or 2.

33. The compound of claim 1 wherein R₃ is —(CH₂)ₘC(═O)NR₅R₆ wherein R₅ is H, R₆ is (CH₂)ₘNHC(R₂)═NH, R₂ is selected from H, Me and Et, and m is 1 or 2.

34. The compound of claim 1 wherein R₃ is —(CH₂)ₘC(═O)NR₅R₆ wherein R₅ is H, R₆ is —(CH₂)ₘC(═NH)NH₂, and m is 1 or 2.

35. The compound of claim 1 wherein R₃ is —(CH₂)ₘC(═O)NR₅R₆ wherein R₅ is —(CH₂)ₘNH₂, R₆ is —(CH₂)ₘNHR₇, R₇ is H, and m is 1 or 2.

36. The compound of claim 1 wherein R₄ is —(CH₂)ₙCHR₅R₆ wherein R₅ is H, R₆ is —(CH₂)ₘNHR₇, R₇ is H, n is 0 to 2, and m is 1 or 2.

37. The compound of claim 1 wherein R₄ is —(CH₂)ₙCHR₅R₆ wherein n is 0 to 2, R₅ is H, R₆ is —(CH₂)ₘNHR₇, m is 1 or 2, and R₇ is selected from Alanine, Arginine, Asparagine, Aspartic acid, Glutamic acid, Glutamine, Cysteine, Glycine, Histidine, Isoleucine, Leucine, Lysine, Methionine, Phenylalanine, Proline, Serine, Threonine, Tryptophan, Tyrosine, and Valine.

38. The compound of claim 1 wherein R₄ is —(CH₂)ₙCHR₅R₆ wherein n is 0 to 2, R₅ is H, R₆ is —(CH₂)ₘNHR₇, R₇ is —C(O)CH(R₁₃)(NH₂), and m is 1 or 2.

39. The compound of claim 1 wherein R₄ is —(CH₂)ₙCHR₅R₆ wherein n is 0 to 2, R₅ is H, R₆ is —(CH₂)ₘNHR₇, R₇ is —C(O)A₂CH₂NH₂, and m is 1 or 2.

40. The compound of claim 1 wherein R₄ is —(CH₂)ₙCHR₅R₆ wherein n is 0 to 2, R₅ is H, R₆ is —(CH₂)ₘNHC(═NH)NH₂, and m is 1 or 2.

41. The compound of claim 1 wherein R₄ is —(CH₂)ₙCHR₅R₆ wherein n is 0 to 2, R₅ is H, R₆ is (CH₂)ₘNHC(R₂)═NH, R₂ is selected from H, Me and Et, and m is 1 or 2.

42. The compound of claim 1 wherein R₄ is —(CH₂)ₙCHR₅R₆ wherein n is 0 to 2, R₅ is H, R₆ is —(CH₂)ₘC(═NH)NH₂, and m is 1 or 2.

43. The compound of claim 1 wherein R₄ is —(CH₂)ₙCHR₅R₆ wherein n is 0 to 2, R₅ is —(CH₂)ₘNH₂, R₆ is —(CH₂)ₘNHR₇, R₇ is H, and m is 1 or 2.

44. The compound of claim 1 wherein R₄ is —(CH₂)ₙNR₅R₆ wherein n is 0 to 2, R₅ is H, R₆ is —(CH₂)ₘNHR₇, R₇ is H and m is 1 or 2.

45. The compound of claim 1 wherein R₄ is —(CH₂)ₙNR₅R₆ wherein n is 0 to 2, R₅ is H, R₆ is —(CH₂)ₘNHR₇, R₇ is selected from Alanine, Arginine, Asparagine, Aspartic acid, Glutamic acid, Glutamine, Cysteine, Glycine, Histidine, Isoleucine, Leucine, Lysine, Methionine, Phenylalanine, Proline, Serine, Threonine, Tryptophan, Tyrosine, and Valine and m is 1 or 2.

46. The compound of claim 1 wherein R₄ is —(CH₂)ₙNR₅R₆ wherein n is 0 to 2, R₅ is H, R₆ is —(CH₂)ₘNHR₇, R₇ is —C(O)CH(R₁₃)(NH₂) and m is 1 or 2.

47. The compound of claim 1 wherein R₄ is —(CH₂)ₙNR₅R₆ wherein n is 0 to 2, R₅ is H, R₆ is —(CH₂)ₘNHR₇, R₇ is —C(O)A₂CH₂NH₂, and m=1 or 2.

48. The compound of claim 1 wherein R₄ is —(CH₂)ₙNR₅R₆ wherein n is 0 to 2, R₅ is H, R₆ is —(CH₂)ₘNHC(═NH)NH₂, and m is 1 or 2.

49. The compound of claim 1 wherein R₄ is —(CH₂)ₙNR₅R₆ wherein n is 0 to 2, R₅ is H, R₆ is (CH₂)ₘNHC(R₂)═NH, R₂ is selected from H, Me and Et, and m is 1 or 2.

50. The compound of claim 1 wherein R₄ is —(CH₂)ₙNR₅R₆ wherein n is 0 to 2, R₅ is H, R₆ is —(CH₂)ₘC(═NH)NH₂, and m is 1 or 2.

51. The compound of claim 1 wherein R₄ is —(CH₂)ₘC(═O)NR₅R₆ wherein R₅ is —(CH₂)ₘNH₂, R₆ is —(CH₂)ₘNHR₇, R₇ is H, and m is 1 or 2.

52. The compound of claim 1 wherein R₄ is —(CH₂)ₘC(═O)NR₅R₆ wherein R₅ is H, R₆ is —(CH₂)ₘNHR₇, R₇ is H, and m is 1 or 2.

53. The compound of claim 1 wherein R₄ is —(CH₂)ₘC(═O)NR₅R₆ wherein R₅ is H, R₆ is —(CH₂)ₘNHR₇, R₇ is selected from Alanine, Arginine, Asparagine, Aspartic acid, Glutamic acid, Glutamine, Cysteine, Glycine, Histidine, Isoleucine, Leucine, Lysine, Methionine, Phenylalanine, Proline, Serine, Threonine, Tryptophan, Tyrosine, and Valine, and m is 1 or 2.

54. The compound of claim 1 wherein R₄ is —(CH₂)ₘC(═O)NR₅R₆ wherein R₅ is H, R₆ is —(CH₂)ₘNHR₇, R₇ is —C(O)CH(R₁₃)(NH₂), and m is 1 or 2.

55. The compound of claim 1 wherein R₄ is —(CH₂)ₘC(═O)NR₅R₆ wherein R₅ is H, R₆ is —(CH₂)ₘNHR₇, R₇ is —C(O)A₂CH₂NH₂, and m is 1 or 2.

56. The compound of claim 1 wherein R₄ is —(CH₂)ₘC(═O)NR₅R₆ wherein R₅ is H, R₆ is —(CH₂)ₘNHC(═NH)NH₂, and m is 1 or 2.

57. The compound of claim 1 wherein R₄ is —(CH₂)ₘC(═O)NR₅R₆ wherein R₅ is H, R₆ is (CH₂)ₘNHC(R₂)═NH, R₂ is selected from H, Me and Et, and m is 1 or 2.

58. The compound of claim 1 wherein R₄ is —(CH₂)ₘC(═O)NR₅R₆ wherein R₅ is H, R₆ is —(CH₂)ₘC(═NH)NH₂, and m is 1 or 2.

59. The compound of claim 1 wherein R₄ is —(CH₂)ₘC(═O)NR₅R₆ wherein R₅ is —(CH₂)ₘNH₂, R₆ is —(CH₂)ₘNHR₇, R₇ is H, and m is 1 or 2.

60. The compound of claim 1 wherein R₄ is —C(═NR₅)NR₅R₅ wherein R₅ is H.

61. The compound of claim 1 wherein R₄ is —C(═NR₅)NR₅R₅ wherein R₅ is —(CH₂)ₘNH₂.

62. The compound of claim 1 wherein R₄ is —NHR₂ wherein R₂ is H.

63. The compound of claim 1 wherein R₄ is —NHR₂ wherein R₂ is C₁-C₆ alkyl.

64. The compound of claim 1 wherein R₉ is selected from H, C₁-C₆ alkyl and C₃-C₇ carbocyclyl.

65. The compound of claim 1 wherein R₉ is H.

66. The compound of claim 1 wherein R₁₀ is selected from C₁-C₆ alkyl and C₃-C₇ carbocyclyl.

67. The compound of claim 1 wherein R₁₀ is selected from heterocyclyl, aryl and heteroaryl, each optionally substituted with up to 3 substituents independently selected from the group consisting of a halide, alkyl, carbocyclyl, —(CH₂)ₙR₁]-OR₂, —OR₁, —S(R₂)₂, —SO₂NHR₁—(CH₂)ₙSH, —CF₃, —OCF₃, —N(R₂)₂, —NO₂, —CN, —(C═X)R₁, —(C═X)R₂, —CO₂alkyl, and —CO₂aryl.

68. The compound of claim 1 wherein R₁₀ is aryl, optionally substituted with up to 3 substituents independently selected from the group consisting of a halide, alkyl, carbocyclyl, —(CH₂)ₙR₁, —OR₂, —OR₁, —S(R₂)₂, —SO₂NHR₁, —CF₃, —OCF₃, and —CN.

69. The compound of claim 1 wherein R₁₀ is heteroaryl, optionally substituted with up to 3 substituents independently selected from the group consisting of a halide, alkyl, carbocyclyl, —(CH₂)ₙR₁, —OR₂, —OR₁, —S(R₂)₂, —SO₂NHR₁, —CF₃, —OCF₃, and —CN.

70. The compound of claim 1 wherein R₉ and R₁₀ are linked to form a ring selected from the group consisting of: 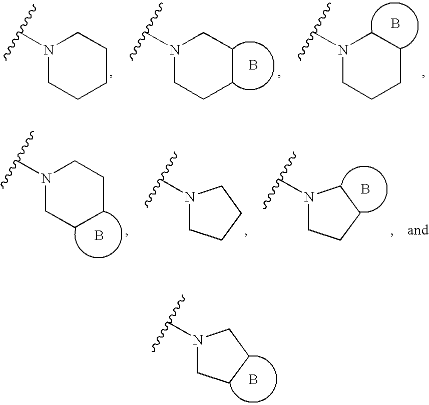
each of which are optionally substituted with up to 3 substituents independently selected from the group consisting of a halide, alkyl, carbocyclyl, —(CH₂)ₙR₁, —(CH═CH)ₙR₁, —OR₂, —OR₁, —S(R₂)₂, —SO₂NHR₁, —(CH₂)ₙSH, —CF₃, —OCF₃, —N(R₂)₂, —NO₂, —CN, —(C═X)R₁, —(C═X)R₂, —CO₂alkyl, —CO₂aryl, heteroaryl optionally substituted with C₁-C₆ alkyl, and aryl optionally substituted with C₁-C₆ alkyl, wherein ring B is C₃-C₇ carbocyclyl, heterocyclyl, aryl or heteroaryl.

71. The compound of claim 1 wherein R₁₁ is H.

72. The compound of claim 1 wherein R₁₁ is —(CH₂)ₙCHR₅R₆ wherein n is 0 to 2, R₅ is H, R₆ is —(CH₂)ₘNHR₇, R₇ is H, and m is 1 or 2.

73. The compound of claim 1 wherein R₁₁ is —(CH₂)ₙCHR₅R₆ wherein n is 0 to 2, R₅ is H, R₆ is —(CH₂)ₘNHR₇, R₇ is selected from Alanine, Arginine, Asparagine, Aspartic acid, Glutamic acid, Glutamine, Cysteine, Glycine, Histidine, Isoleucine, Leucine, Lysine, Methionine, Phenylalanine, Proline, Serine, Threonine, Tryptophan, Tyrosine, and Valine, and m is 1 or 2.

74. The compound of claim 1 wherein R₁₁ is —(CH₂)ₙCHR₅R₆ wherein n is 0 to 2, R₅ is H, R₆ is —(CH₂)ₘNHR₇, R₇ is —C(O)CH(R₁₃)(NH₂), and m is 1 or 2.

75. The compound of claim 1 wherein R₁₁ is —(CH₂)ₙCHR₅R₆ wherein n is 0 to 2, R₅ is H, R₆ is —(CH₂)ₘNHR₇, R₇ is —C(O)A₂CH₂NH₂, and m is 1 or 2.

76. The compound of claim 1 wherein R₁₁ is —(CH₂)ₙCHR₅R₆ wherein n is 0 to 2 R₅ is H, R₆ is —(CH₂)ₘNHC(═NH)NH₂, and m is 1 or 2.

77. The compound of claim 1 wherein R₁₁ is —(CH₂)ₙCHR₅R₆ wherein n is 0 to 2, R₅ is H, R₆ is (CH₂)ₘNHC(R₂)═NH, R₂ is selected from H, Me and Et, and m is 1 or 2.

78. The compound of claim 1 wherein R₁₁ is —(CH₂)ₙCHR₅R₆ wherein n is 0 to 2, R₅ is H, R₆ is —(CH₂)ₘC(═NH)NH₂, and m is 1 or 2.

79. The compound of claim 1 wherein R₁₁ is —(CH₂)ₙCHR₅R₆ wherein n is 0 to 2 R₅ is —(CH₂)ₘNH₂, R₆ is —(CH₂)ₘNHR₇, R₇ is H, and m is 1 or 2.

80. The compound of claim 1 wherein R₁₁ is —(CH₂)ₙNHR₂ wherein R₂ is H and n is 0 to 2.

81. The compound of claim 1 wherein R₁₁ is —(CH₂)ₙNHR₂ wherein R₂ is C₁-C₃ alkyl and n is 0 to 2.

82. The compound of claim 1 wherein R₁₂ is —(CH₂)ₙCHR₅R₆ wherein n is 0 to 2, R₅ is H, R₆ is —(CH₂)ₘNHR₇, R₇ is H, and m is 1 or 2.

83. The compound of claim 1 wherein R₁₂ is —(CH₂)ₙCHR₅R₆ wherein n is 0 to 2, R₅ is H, R₆ is —(CH₂)ₘNHR₇, R₇ is selected from Alanine, Arginine, Asparagine, Aspartic acid, Glutamic acid, Glutamine, Cysteine, Glycine, Histidine, Isoleucine, Leucine, Lysine, Methionine, Phenylalanine, Proline, Serine, Threonine, Tryptophan, Tyrosine, and Valine, and m is 1 or 2.

84. The compound of claim 1 wherein R₁₂ is —(CH₂)ₙCHR₅R₆ wherein n is 0 to 2, R₅ is H, R₆ is —(CH₂)ₘNHR₇, R₇ is —C(O)CH(R₁₃)(NH₂), and m is 1 or 2.

85. The compound of claim 1 wherein R₁₂ is —(CH₂)ₙCHR₅R₆ wherein n is 0 to 2, R₅ is H, R₆ is —(CH₂)ₘNHR₇, R₇ is —C(O)A₂CH₂NH₂, and m is 1 or 2.

86. The compound of claim 1 wherein R₁₂ is —(CH₂)ₙCHR₅R₆ wherein n is 0 to 2, R₅ is H, R₆ is —(CH₂)ₘNHC(═NH)NH₂, and m is 1 or 2.

87. The compound of claim 1 wherein R₁₂ is —(CH₂)ₙCHR₅R₆ wherein n is 0 to 2, R₅ is H, R₆ is (CH₂)ₘNHC(R₂)═NH, R₂ is selected from H, Me and Et, and m is 1 or 2.

88. The compound of claim 1 wherein R₁₂ is —(CH₂)ₙCHR₅R₆ wherein n is 0 to 2, R₅ is H, R₆ is —(CH₂)ₘC(═NH)NH₂, and m is 1 or 2.

89. The compound of claim 1 wherein R₁₂ is —(CH₂)ₙCHR₅R₆ wherein n is 0 to 2, R₅ is —(CH₂)ₘNH₂, R₆ is —(CH₂)ₘNHR₇, R₇ is H and m is 1 or 2.

90. The compound of claim 1 wherein R₁₂ is —(CH₂)ₙNHR₂ wherein R₂ is H and n is 0 to 2.

91. The compound of claim 1 wherein R₁₂ is —(CH₂)ₙNHR₂ wherein R₂ is C₁-C₃ alkyl and n is 0 to 2.

92. The compound of claim 1 wherein R₁₃ is —(CH₂)ₙCHR₅(CH₂)ₙNH₂ wherein R₅ is H and n is 0 to 2.

93. The compound of claim 1 wherein R₁₃ is —(CH₂)ₙCHR₅(CH₂)ₙNH₂ wherein R₅ is —(CH₂)ₘNH₂, m is 1 or 2, and n is 0 to 2.

94. The compound of claim 1 wherein R₁₃ is —(CH₂)ₘNR₅(CH₂)ₙNH₂ wherein R₅ is H, m is 1 or 2, and n is 0 to 2.

95. The compound of claim 1 wherein R₁₃ is —(CH₂)ₘNR₅(CH₂)ₙNH₂ wherein R₅ is —(CH₂)ₘNH₂, m is 1 or 2, and n is 0 to 2.

96. The compound of claim 1 wherein R₁₃ is —(CH₂)ₘC(═O)NR₅(CH₂)ₙNH₂ wherein R₅ is H, m is 1 or 2, and n is 0 to 2.

97. The compound of claim 1 wherein R₁₃ is —(CH₂)ₘC(═O)NR₅(CH₂)ₙNH₂ wherein R₅ is (CH₂)ₘNH₂, m is 1 or 2 and n is 0 to 2.

98. The compound of claim 1 wherein A₁ is —(CH₂)ₘ— wherein m is 1 or 2; A₃ is H; a1 is 1; and a2 is 0.

99. The compound of claim 1 wherein A₂ is —C(═X); A₃ is H; a1 is 0; and a2 is 1.

100. The compound of claim 1 wherein A₂ is —C(═N—OR₂)— wherein R₂ is H or Me; A₃ is H; a1 is 0; and a2 is 1.

101. The compound of claim 1 wherein A₁ is —(CH₂)ₘ— wherein m is 1 or 2; A₂ is selected from —O(CH₂)ₙ— or —S(CH₂)ₙ— wherein n is 0; A₃ is H; and a1 and a2 are equal to 1.

102. The compound of claim 1 wherein A₁ is —[C(R₂R₈)]ₘ— wherein m is 1 or 2,
each R₂ is H, and each R₈ is independently selected from H, SH and OH with the proviso that at least one R₈ is SH or OH; A₃ is H; a1 is 1; and a2 is 0.

103. The compound of claim 1 wherein A₃ is Me or Et.

104. The compound of claim 1 wherein A₁ is —(CH₂)ₘ— wherein m is 1 or 2; a1 is 1; a2 is 0; A₃ is —CH₂— bonded to R₁ to form a ring; and R₁ is aryl optionally substituted with up to 2 substituents independently selected from the group consisting of halide, alkyl, OMe, CF₃, OCF₃, and CN.

105. The compound of claim 1 wherein A₁ is —(CH₂)ₘ— wherein m is 1 to 3; a1 is 1; a2 is 0; A₃ is —CH₂— bonded to A₁ to form a 4, 5, or 6 membered ring.

106. The compound of claim 1 wherein D₁ is —CH(NHR₇)— wherein R₇ is H; D₂ is —(CH₂)ₘ— wherein m is 1 to 3; D₇ is N; d1 and d2 are equal to 1; and z1, d3, d4, d5 and d6 are equal to 0.

107. The compound of claim 1 wherein D₁ is —CH(NHR₇)— wherein R₇ is selected from Alanine, Arginine, Asparagine, Aspartic acid, Glutamic acid, Glutamine, Cysteine, Glycine, Histidine, Isoleucine, Leucine, Lysine, Methionine, Phenylalanine, Proline, Serine, Threonine, Tryptophan, Tyrosine and Valine; D₂ is —(CH₂)ₘ wherein m is 1 to 3; D₇ is N;
d1 and d2 are equal to 1; and z1, d3, d4, d5 and d6 are equal to 0.

108. The compound of claim 1 wherein D₁ is —CH(NHR₇)— wherein R₇ is —C(═O)CH(R₁₃)(NH₂); D₂ is —(CH₂)ₘ— wherein m is 1 to 3; D₇ is N; d1 and d2 are equal to 1; and z1, d3, d4, d5 and d6 are equal to 0.

109. The compound of claim 1 wherein D₁ is —CH(NHR₇)— wherein R₇ is —C(═O)A₂CH₂NH₂; D₂ is —(CH₂)ₘ wherein m is 1 to 3; D₇ is N; A₂ is selected from —(CH₂)ₘ—, —C(═X)—, —O(CH₂)ₙ—, —S(CH₂)ₙ—, —CH═CH— and —C(═N—OR₂)— wherein m is 1 to 3 and n is 0 to 3; d1 and d2 are equal to 1; and z1, d3, d4, d5 and d6 are equal to 0.

110. The compound of claim 1 wherein D₁ is —CH(NHR₇)— wherein R₇ is H; D₂ is —(CH₂)ₘ wherein m is 1 to 3; D₇ is CH; d1 and d2 are equal to 1; and z1, d3, d4, d5 and d6 are equal to 0.

111. The compound of claim 1 wherein D₁ is —CH(NHR₇)— wherein R₇ is H; D₂ is —CH(R₂)— wherein R₂ is selected from Me and Et; D₃ is —(CH₂)ₘ— wherein m is 1 to 3; D₇ is N;
d1, d2 and d3 are equal to 1; and z1, d4, d5 and d6 are equal to 0.

112. The compound of claim 1 wherein D₁ is —CH(NHR₇)— wherein R₇ is H; D₂ is —(CH₂)ₘ— wherein m is 1 to 3; D₃ is —C(═X)—; D₇ is N; d1, d2 and d3 are equal to 1; and z1, d4, d5 and d6 are equal to 0.

113. The compound of claim 1 wherein D₁ is —CH(NHR₇)— wherein R₇ is selected from Alanine, Arginine, Asparagine, Aspartic acid, Glutamic acid, Glutamine, Cysteine, Glycine, Histidine, Isoleucine, Leucine, Lysine, Methionine, Phenylalanine, Proline, Serine, Threonine, Tryptophan, Tyrosine and Valine; D₂ is —(CH₂)ₘ wherein m is 1 to 3; D₃ is —C(═X)—; D₇ is N; d1, d2 and d3 are equal to 1; and z1, d4, d5 and d6 are equal to 0.

114. The compound of claim 1 wherein D₁ is —CH(NHR₇)— wherein R₇ is —C(═O)CH(R₁₃)(NH₂); D₂ is —(CH₂)ₘ— wherein m is 1 to 3; D₃ is —C(═X)—; D₇ is N; d1, d2 and d3 are equal to 1; and z1, d4, d5 and d6 are equal to 0.

115. The compound of claim 1 wherein D₁ is —CH(NHR₇)— wherein R₇ is —C(═O)A₂CH₂NH₂; D₂ is —(CH₂)ₘ wherein m is 1 to 3; D₃ is —C(═X)—; D₇ is N; A₂ is selected from —(CH₂)ₘ—, —C(═X)—, —O(CH₂)ₙ—, —S(CH₂)ₙ—, —CH═CH— and —C(═N—OR₂)— wherein m is 1 to 3 and n is 0 to 3; d1, d2 and d3 are equal to 1; and z1, d4, d5 and d6 are equal to 0.

116. The compound of claim 1 wherein D₁ is —CH(NHR₇)— wherein R₇ is H; D₂ is —C(═X)—; D₇ is N; d1 and d2 are equal to 1; and z1, d3, d4, d5 and d6 are equal to 0.

117. The compound of claim 1 wherein D₁ is —CH(NHR₇)— wherein R₇ is H; D₂ is —(CH₂)ₘ— wherein m is 1 to 3; D₃ is —C(═X)—; D₄ is —N(R₅)— wherein R₅ is H or —(CH₂)ₘNH₂ and m is 1 to 3; D₇ is N or CH; d1, d2, d3 and d4 are equal to 1; and z1, d5 and d6 are equal to 0.

118. The compound of claim 1 wherein D₁ is —CH(NHR₇)— wherein R₇ is H; D₂ is —(CH₂)ₘ— wherein m is 1 to 3; D₃ is —N(R₅)— wherein R₅ is H or —(CH₂)ₘNH₂ and m is 1 to 3;
D₇ is ═C< where the carbon forms a double bond with an adjacent carbon in D₃; d1, d2 and d3 are equal to 1; and z1, d4, d5 and d6 are equal to 0.

119. The compound of claim 1 wherein D₁ is —CH(NHR₇)— wherein R₇ is H; D₂ is —(CH₂)ₘ wherein m is 1 to 3; D₇ is CH or CR₄; d1 and d2 are equal to 1; and z1, d3, d4, d5 and d6 are equal to 0.

120. The compound of claim 1 wherein D₁ is —CH(NHR₇)— wherein R₇ is H; D₂ is —(CH₂)ₘ— wherein m is 1 to 3; D₃ is —N(R₅)— wherein R₅ is H or —(CH₂)ₘNH₂ and m is 1 to 3; D₄ is —C(═X)—; D₇ is N or CH; d1, d2, d3 and d4 are equal to 1; and z1, d5 and d6 are equal to 0.

121. The compound of claim 1 wherein D₁ is —CH(NHR₇)— wherein R₇ is selected from Alanine, Arginine, Asparagine, Aspartic acid, Glutamic acid, Glutamine, Cysteine, Glycine, Histidine, Isoleucine, Leucine, Lysine, Methionine, Phenylalanine, Proline, Serine, Threonine, Tryptophan, Tyrosine and Valine; D₂ is —(CH₂)ₘ wherein m is 1 to 3; D₃ is —N(R₅)— wherein R₅ is H or —(CH₂)ₘNH₂ and m=1 to 3; D₄ is —C(═X)—; D₇ is N or CH; d1, d2, d3 and d4 are equal to 1; and z1, d5 and d6 are equal to 0.

122. The compound of claim 1 wherein D₁ is —CH(NHR₇)— wherein R₇ is —C(═O)CH(R₁₃)(NH₂); D₂ is —(CH₂)ₘ wherein m is 1 to 3; D₃ is —N(R₅)— wherein R₅ is H or —(CH₂)ₘNH₂ and m=1 to 3; D₄ is —C(═X)—; D₇ is N or CH; d1, d2, d3 and d4 are equal to 1;
and z1, d5 and d6 are equal to 0.

123. The compound of claim 1 wherein D₁ is —CH(NHR₇)— wherein R₇ is —C(═O)A₂CH₂NH₂; D₂ is —(CH₂)ₘ— wherein m is 1 to 3; D₃ is —N(R₅)—R₅ is H or —(CH₂)ₘNH₂ and m is 1 to 3; D₄ is —C(═X)—; D₇ is N or CH; A₂ is selected from —(CH₂)ₘ—, —C(═X)—, —O(CH₂)ₙ₋, —S(CH₂)ₙ—, —CH═CH— and —C(═N—OR₂)— wherein m is 1 to 3 and n is 0 to 3; d1, d2, d3 and d4 are equal to 1; and z1, d5 and d6 are equal to 0.

124. The compound of claim 1 wherein D₁ is —CH(NHR₇)— wherein R₇ is H; D₂ is —(CH₂)ₘ— wherein m is 1 to 3; D₃ is —S(═O)— or —SO₂—; D₇ is N; d1, d2 and d3 are equal to 1;
and z1, d4, d5 and d6 are equal to 0.

125. The compound of claim 1 wherein D₁ is —CH(NHR₇)— wherein R₇ is H; D₂ is —(CH₂)ₘ— wherein m is 1 to 3; D₃ is —S(═O)— or —SO₂—; D₄ is —N(R₅)— wherein R₅ is H or —(CH₂)ₘNH₂ and m is 1 to 3; D₇ is N; d1, d2, d3 and d4 are equal to 1; and z1, d5 and d6 are equal to 0.

126. The compound of claim 1 wherein D₁ is —CH(NHR₇)— wherein R₇ is H; D₂ is —(CH₂)ₘ— wherein m is 1 to 3; D₃ is —N(R₅)— wherein R₅ is H or —(CH₂)ₘNH₂ and m is 1 to 3; D₄ is —S(═O)— or —SO₂—; D₇ is N; d1, d2, d3 and d4 are equal to 1; and z1, d5 and d6 are equal to 0.

127. The compound of claim 1 wherein D₁ is —CH[(CH₂)ₘNHR₇]— wherein R₇ is H and m is 1 to 3; D₂ is —N(R₅)— wherein R₅ is H; D₃ is —C(═X)—; D₄ is —CH(NHR₇)— wherein R₇ is H; D₅ is —C(═X)—; D₇ is N; X is O; d1, d2, d3, d4 and d5 are equal to 1; and z1 and d6 are equal to 0.

128. The compound of claim 1 wherein D₁ is —CH(NHR₇)— wherein R₇ is H; D₂ is —(CH₂)ₘ— wherein m is 1 to 3; D₇ is ═C< where the carbon forms a double bond with an adjacent carbon in D₂; d1 and d2 are equal to 1; and z1, d3, d4, d5 and d6 are equal to 0.

129. The compound of claim 1 wherein D₁ is —CH(NHR₇)— wherein R₇ is H; D₂ is —(CH₂)ₘ— wherein m is 1 to 3; Z₁ is an aryl; z1, d1 and d2 are equal to 1; and d3, d4, d5 and d6 are equal to 0.

130. The compound of claim 1 wherein D₁ is —CH(NHR₇)— wherein R₇ is H; Z₁ is an aryl; m is 1 to 3; z1 and dl are equal to 1; and d2, d3, d4, d5 and d6 are equal to 0.

131. The compound of claim 1 wherein D₁ is —CH(NHR₇)— wherein R₇ is H; D₃ is —(CH₂)ₘ— wherein m is 1 to 3; Z₁ is an aryl; z1, d1 and d3 are equal to 1; and d2, d4, d5 and d6 are equal to 0.

132. The compound of claim 1 wherein D₁ is —CH(NHR₇)— wherein R₇ is H; D₂ is —(CH₂)ₘ wherein m is 1 to 3; Z₁ is a carbocyclyl; z1, d1 and d2 are equal to 1; and d3, d4, d5 and d6 are equal to 0.

133. The compound of claim 1 wherein D₁ is —CH(NHR₇)— wherein R₇ is H; Z1 is a carbocyclyl; m is 1 to 3; z1 and d1 are equal to 1; and d2, d3, d4, d5 and d6 are equal to 0.

134. The compound of claim 1 wherein D₁ is —CH(NHR₇)— wherein R₇ is H; D₃ is —(CH₂)ₘ— wherein m is 1 to 3; Z1 is a carbocyclyl; z1, d1 and d3 are equal to 1; and d2, d4, d5 and d6 are equal to 0.

135. The compound of claim 1 wherein D₈ is N.

136. The compound of claim 1 wherein D₈ is C.

137. The compound of claim 1 wherein A₁ is —(CH₂)ₘ— wherein m is 1 to 3; D₈ is C; and A₁ is ═CR₂[C(R₂R₈)]ₘ—.

138. The compound of claim 1 wherein Q₁ is —N(R₂)— wherein R₂ is H or C₁-C₆ alkyl; Q2 and Q3 are —CH₂—; D₇ is N; and q1, q2, and q3 are equal to 1.

139. The compound of claim 1 wherein Q₁ is —N(R₂)— wherein R₂ is H or C₁-C₆ alkyl; Q2 and Q3 are —CH₂—; D₇ is CH; and q1, q2, and q3 are equal to 1.

140. The compound of claim 1 wherein Q₁ is —N(R₂)N(R₂)— wherein R₂ is H or C₁-C₆ alkyl; Q2 and Q3 is —CH₂—; D₇ is CH; and q1, q2, and q3 are equal to 1.

141. The compound of claim 1 wherein Q2 and Q3 are —CH₂—; D₇ is N; q1 is 0; and q2 and q3 are equal to 1.

142. The compound of claim 1 wherein X₁ and X₂ are taken together to form ═O or ═S.

143. The compound of claim 1 wherein X₁ is absent, X₂ is —O— or —S— bonded to R₁₀ to form a 5- or 6-membered heterocyclyl or heteroaryl, and the bond to nitrogen represented by a dashed and solid line is a double bond.

144. The compound of claim 1 having a structure selected from the group consisting of:

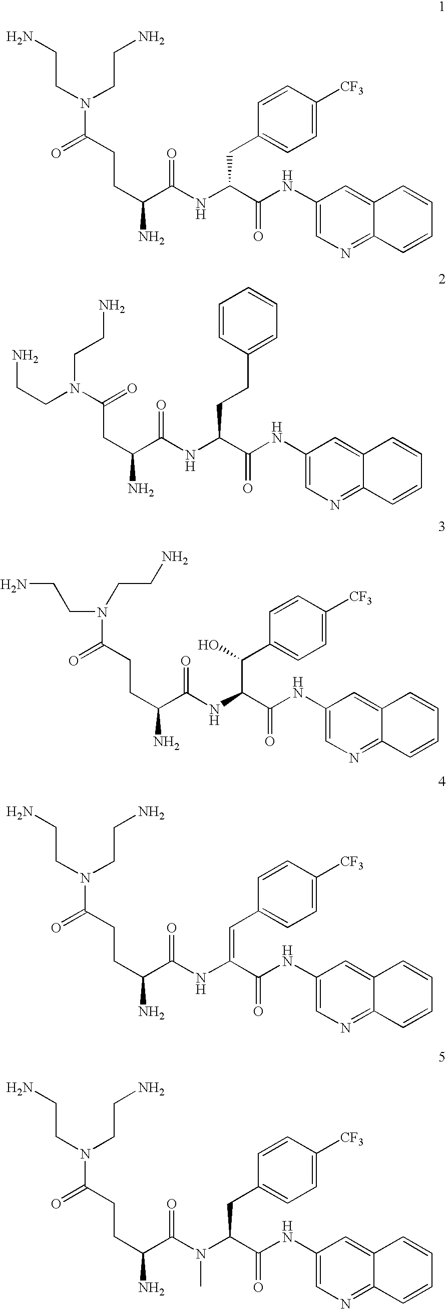

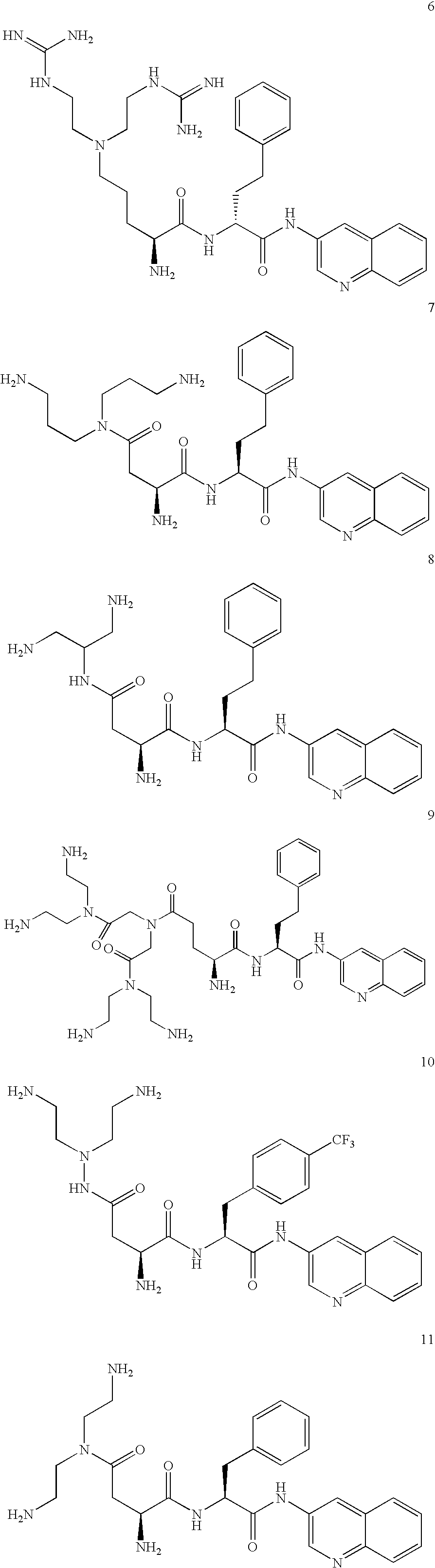

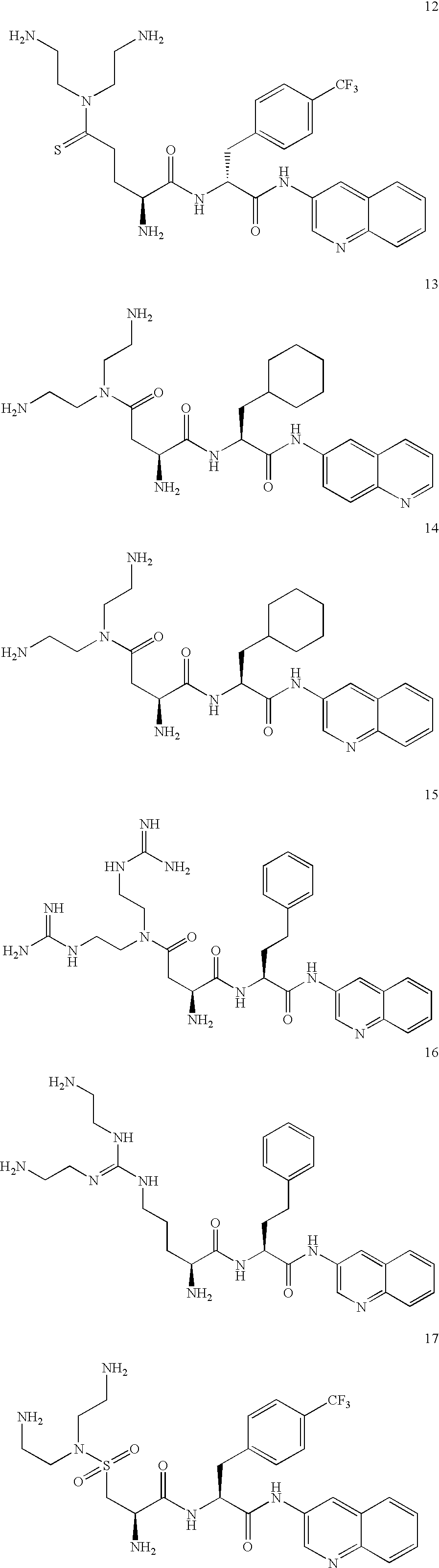

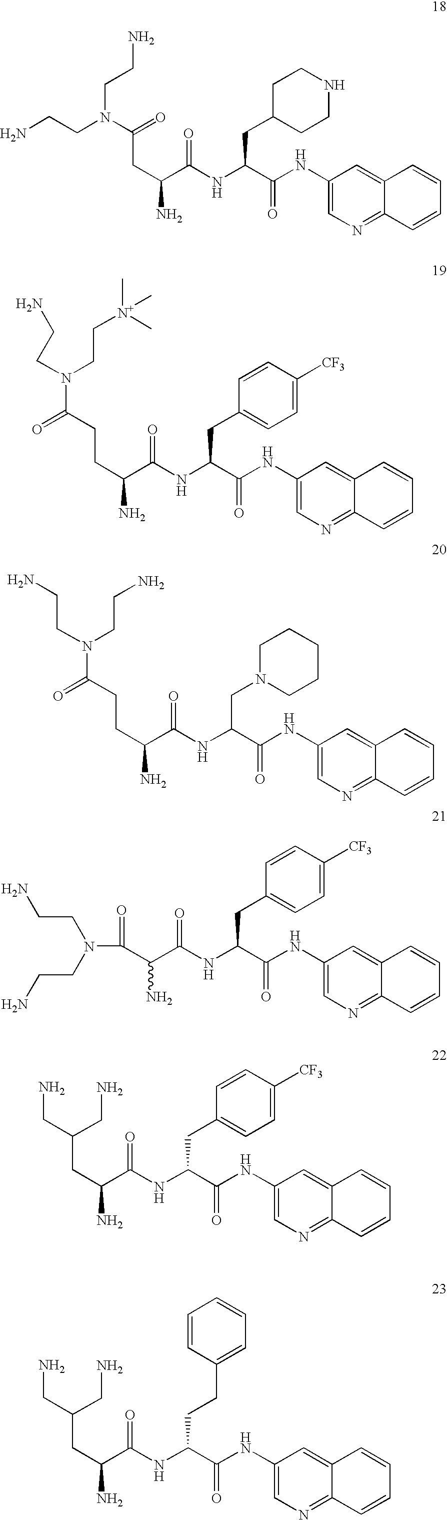

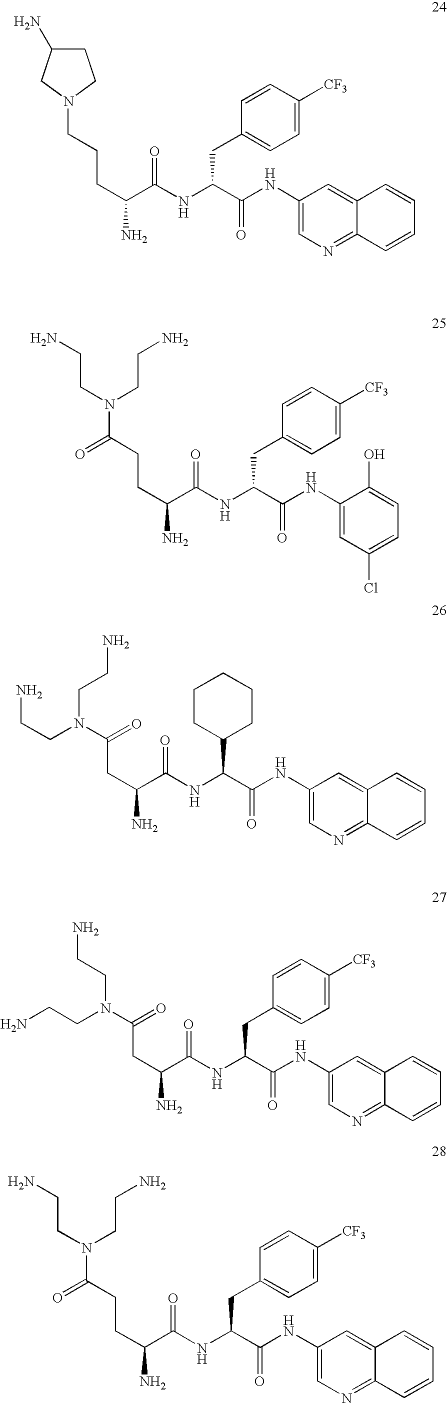

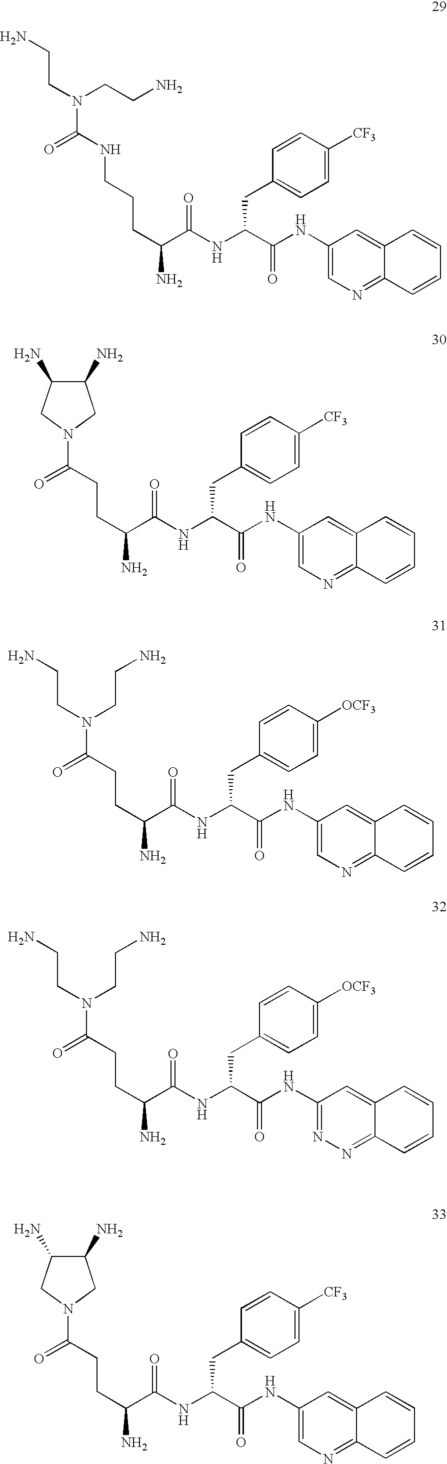

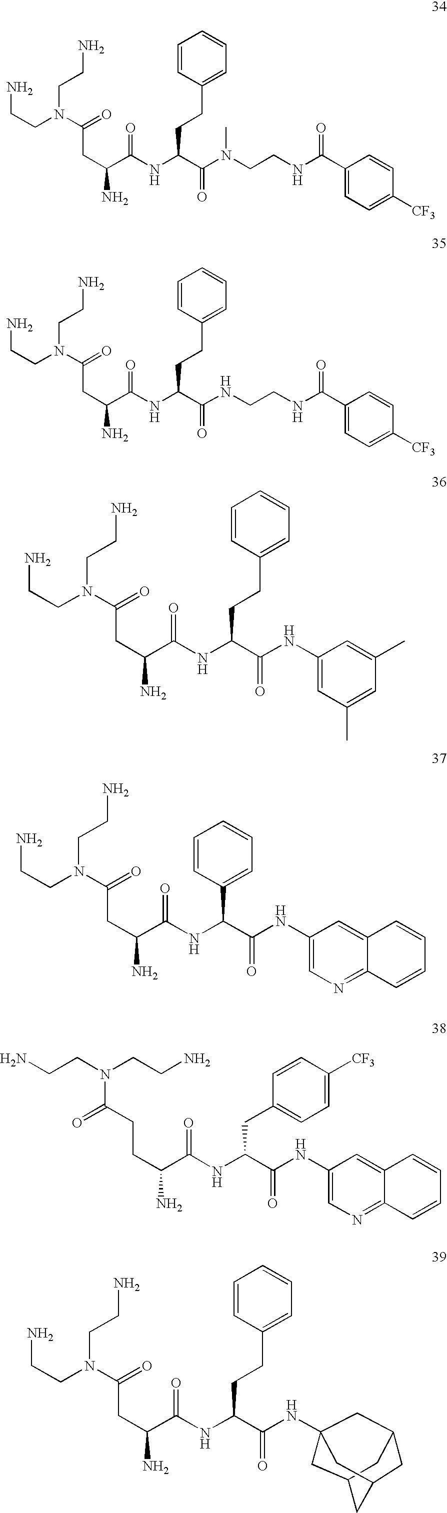

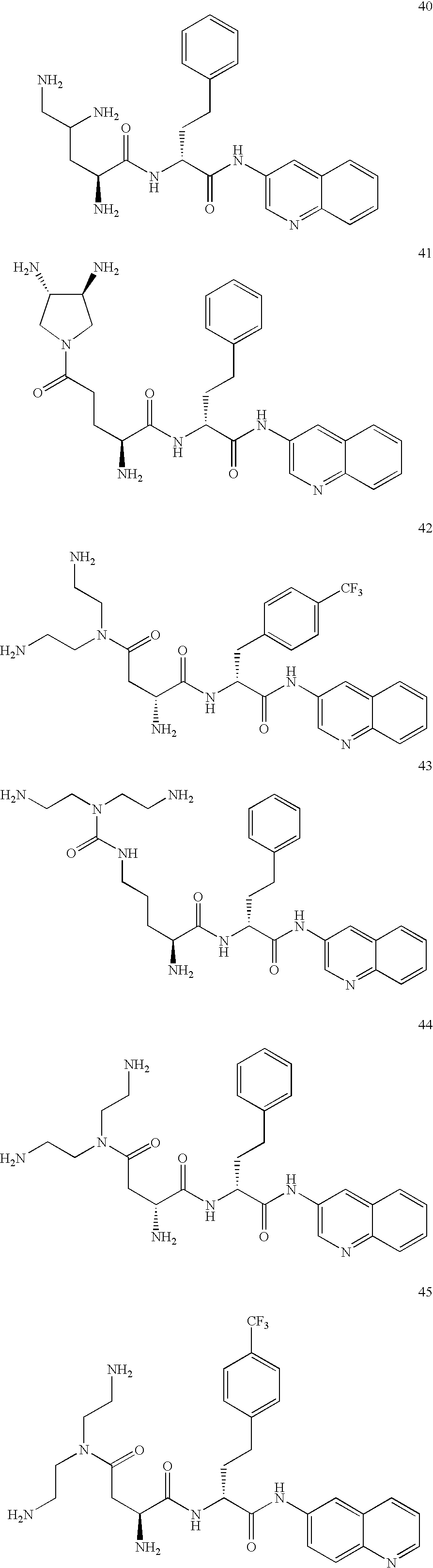

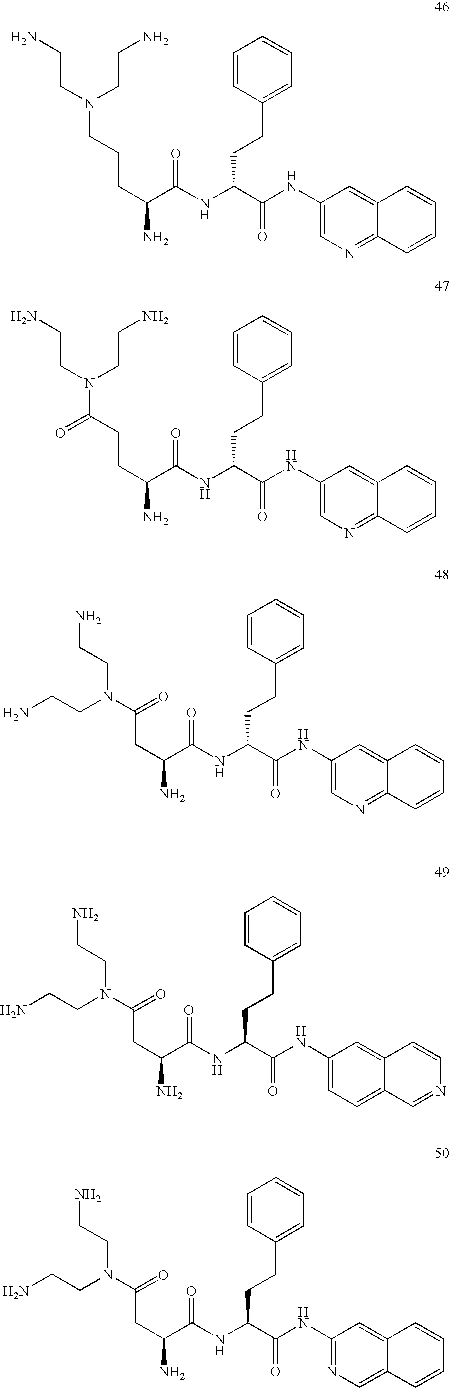

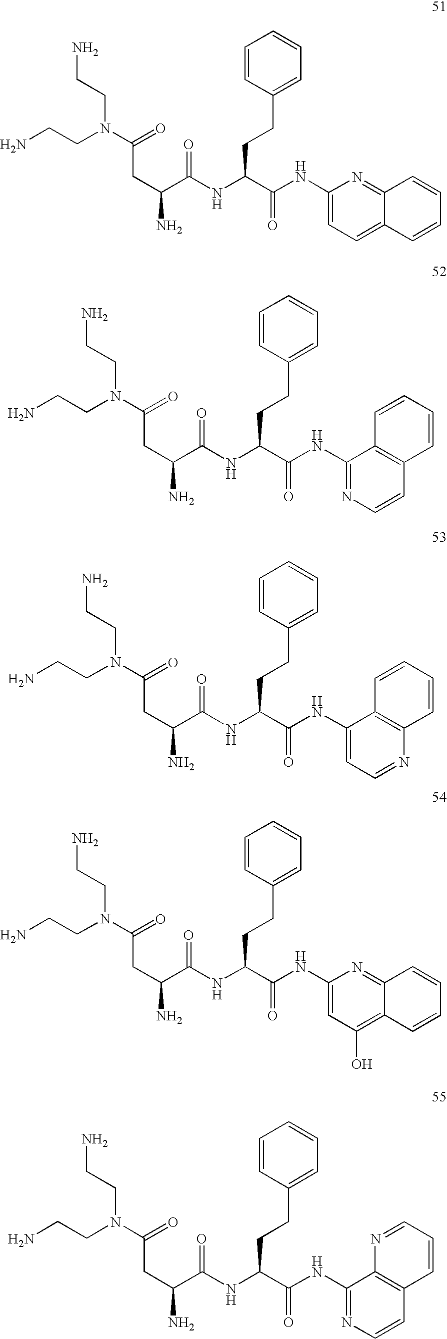

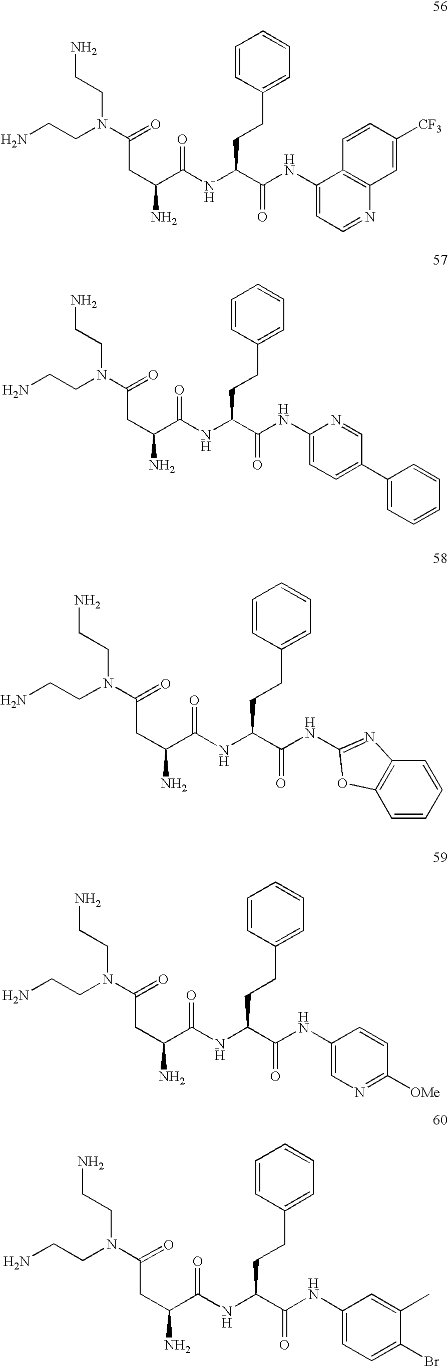

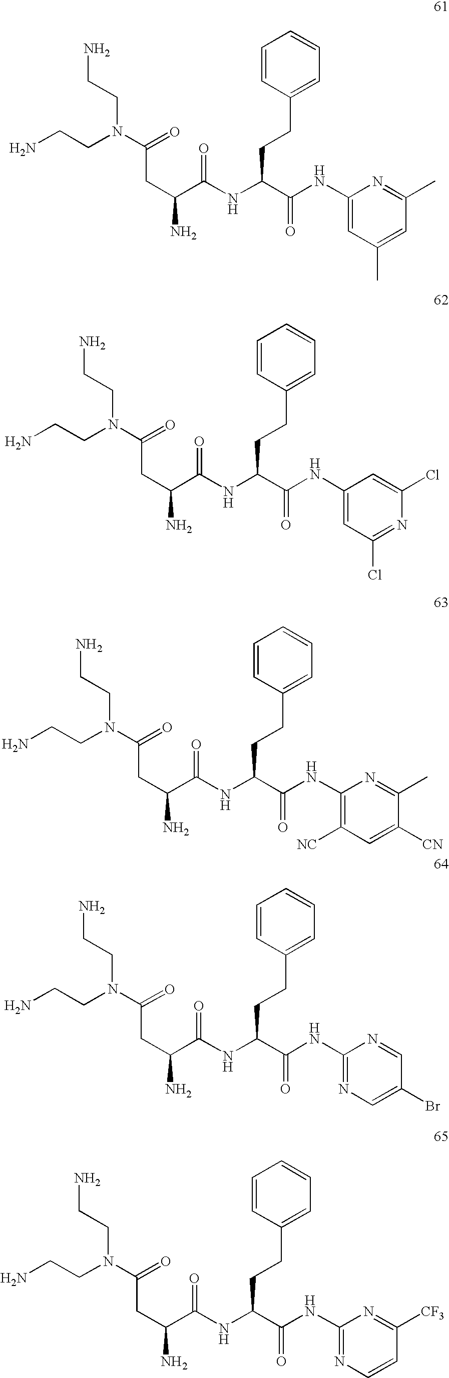

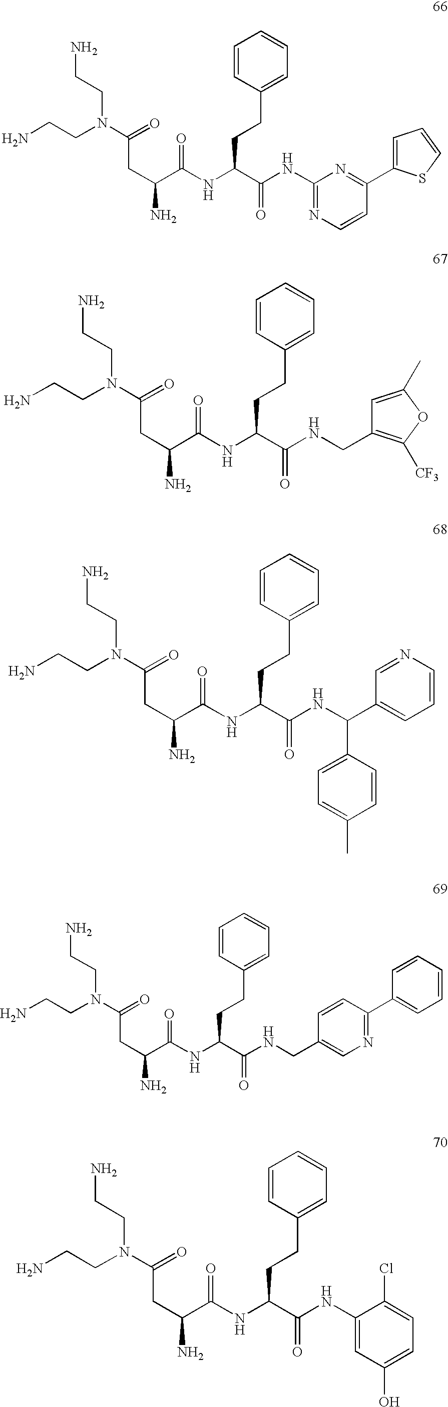

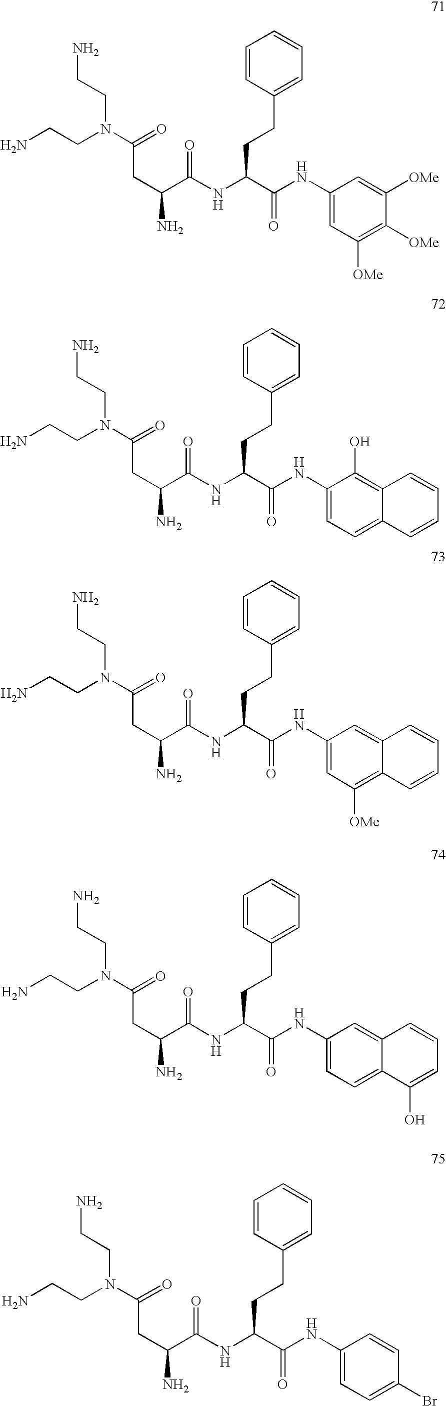

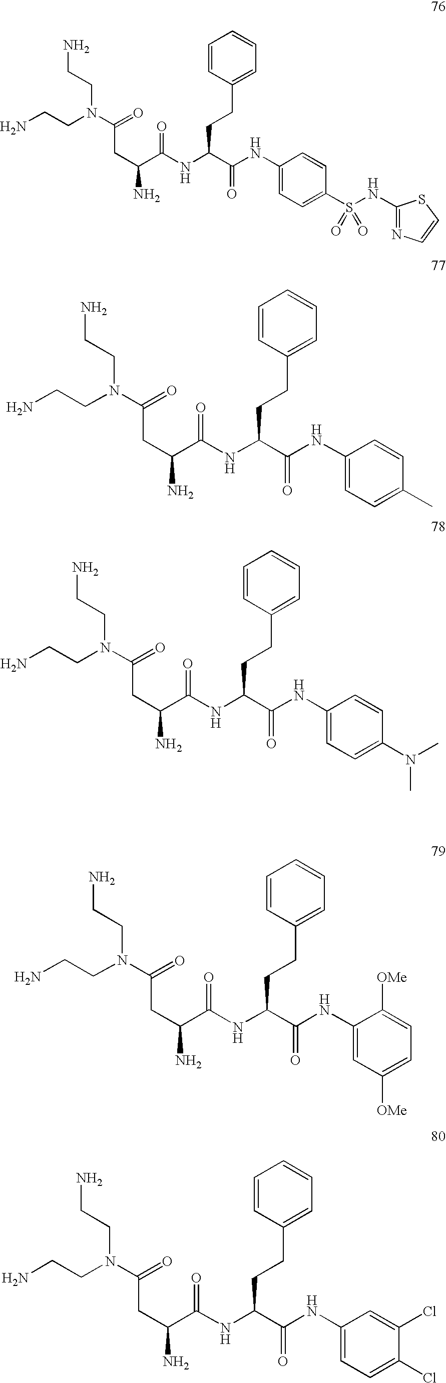

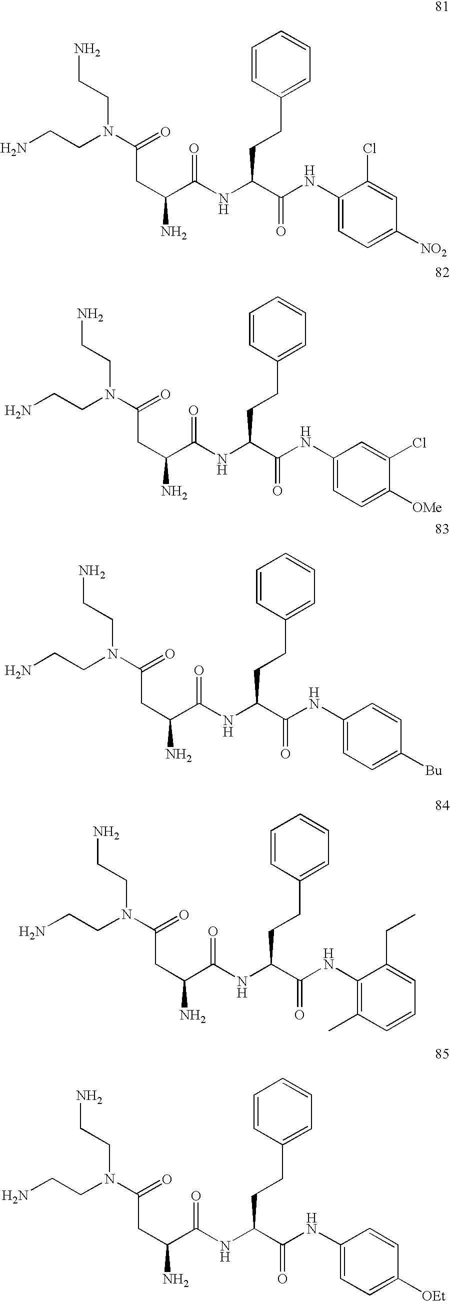

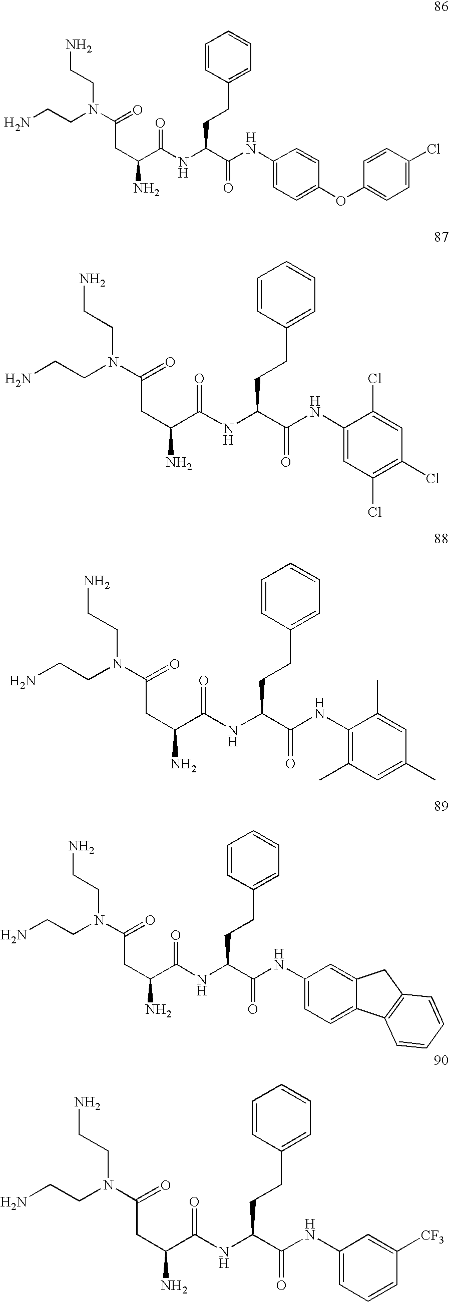

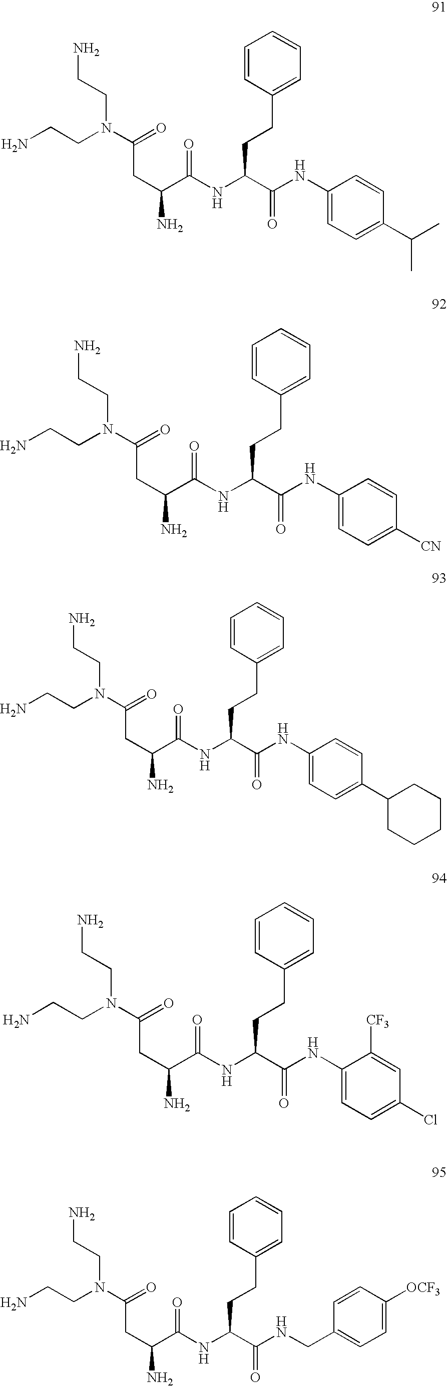

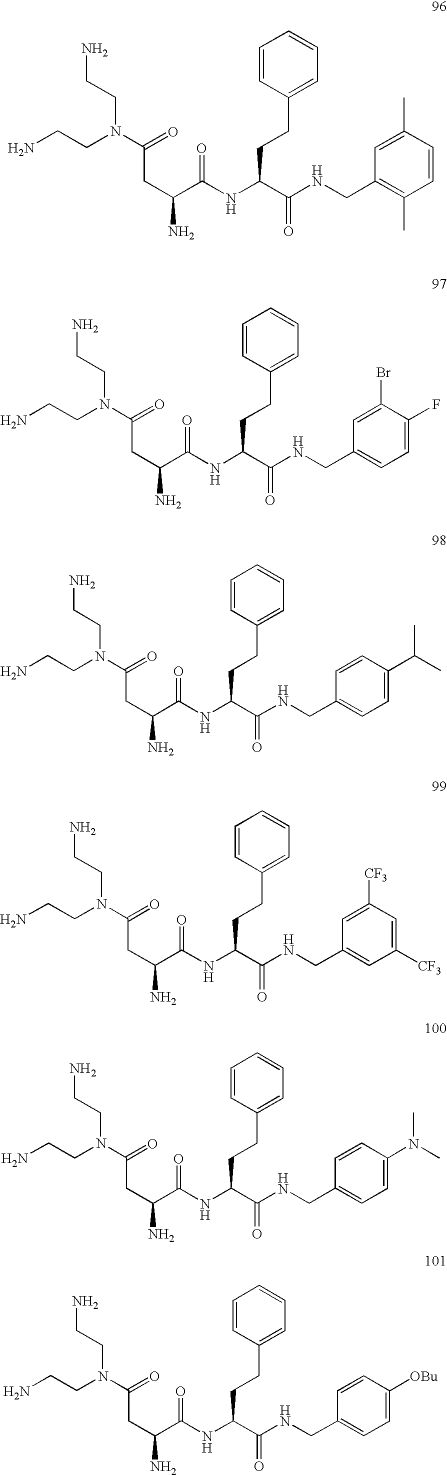

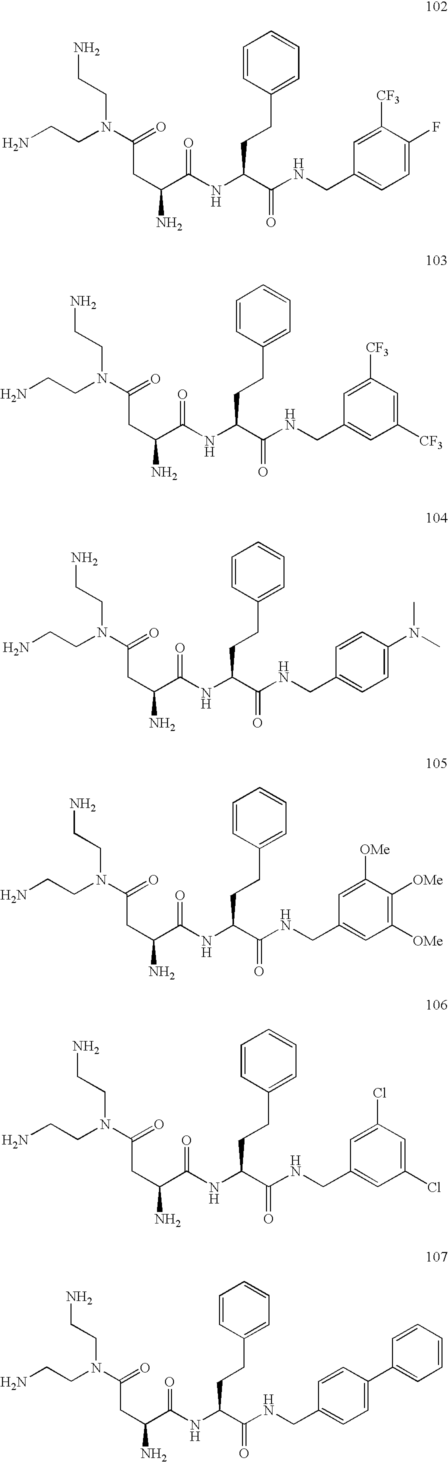

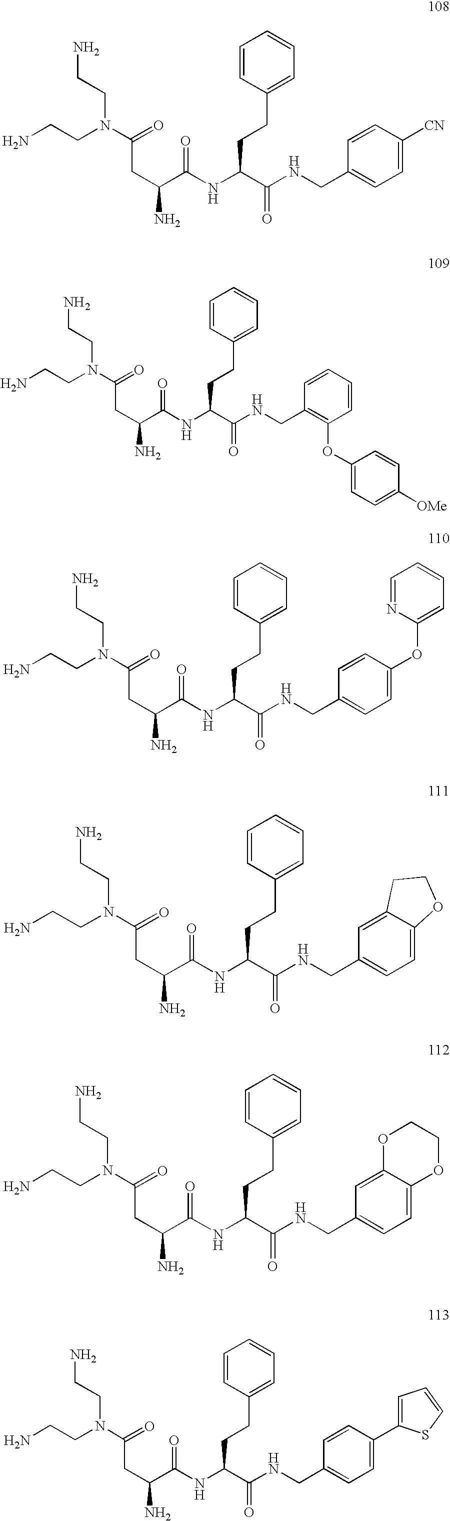

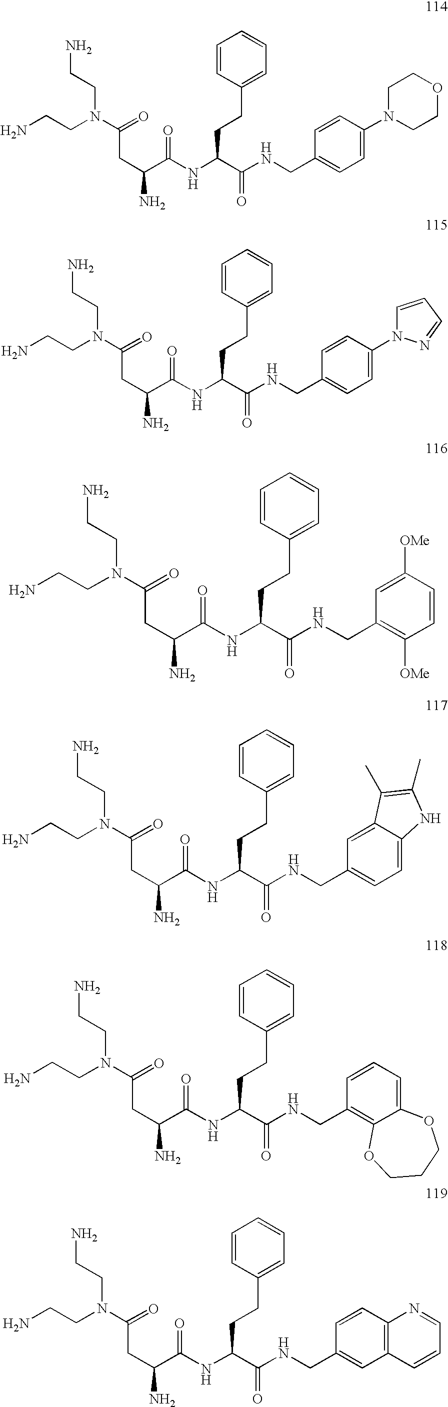

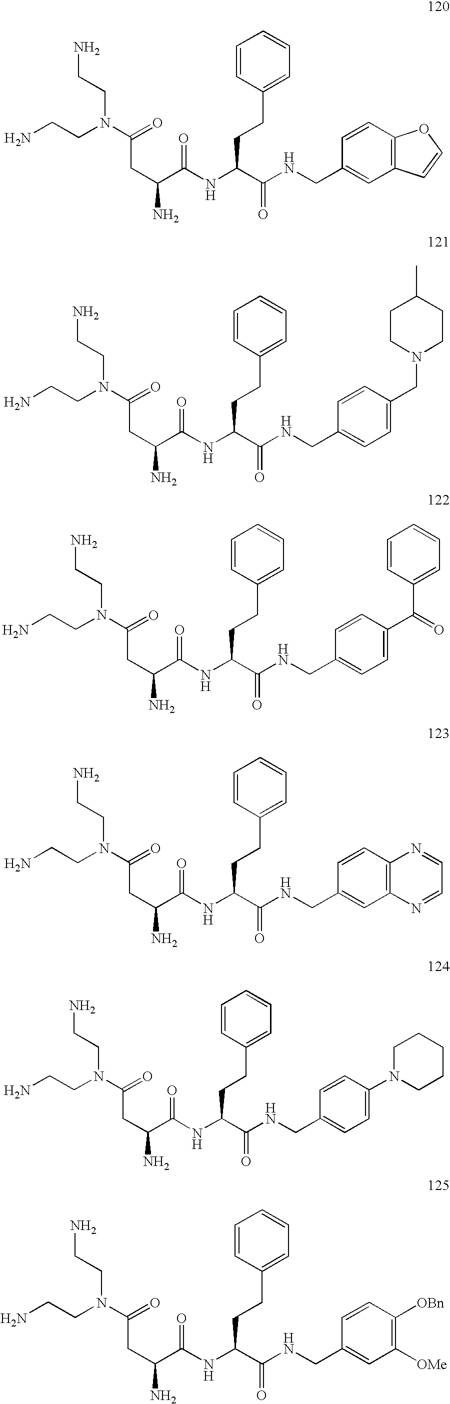

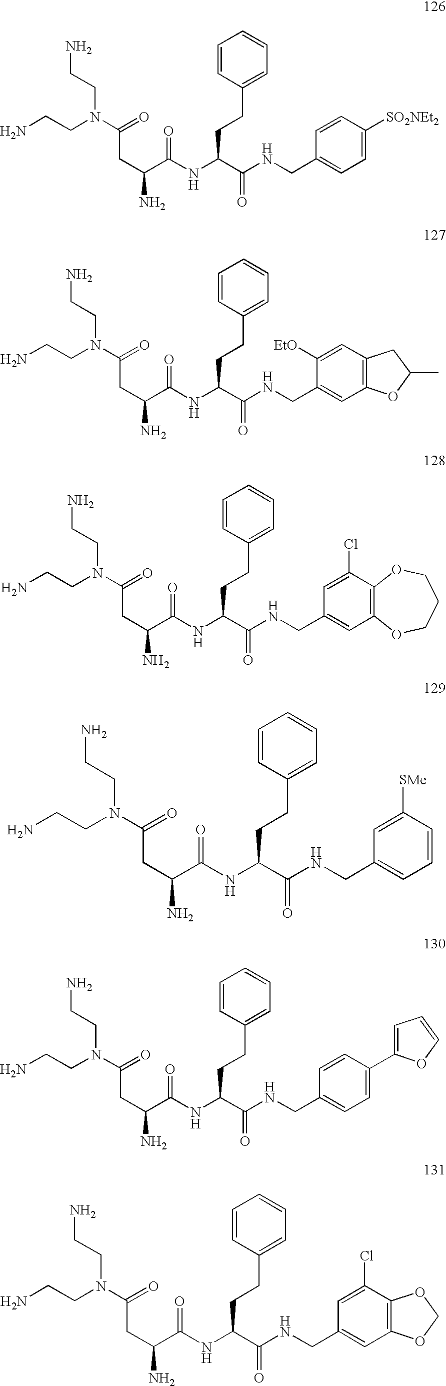

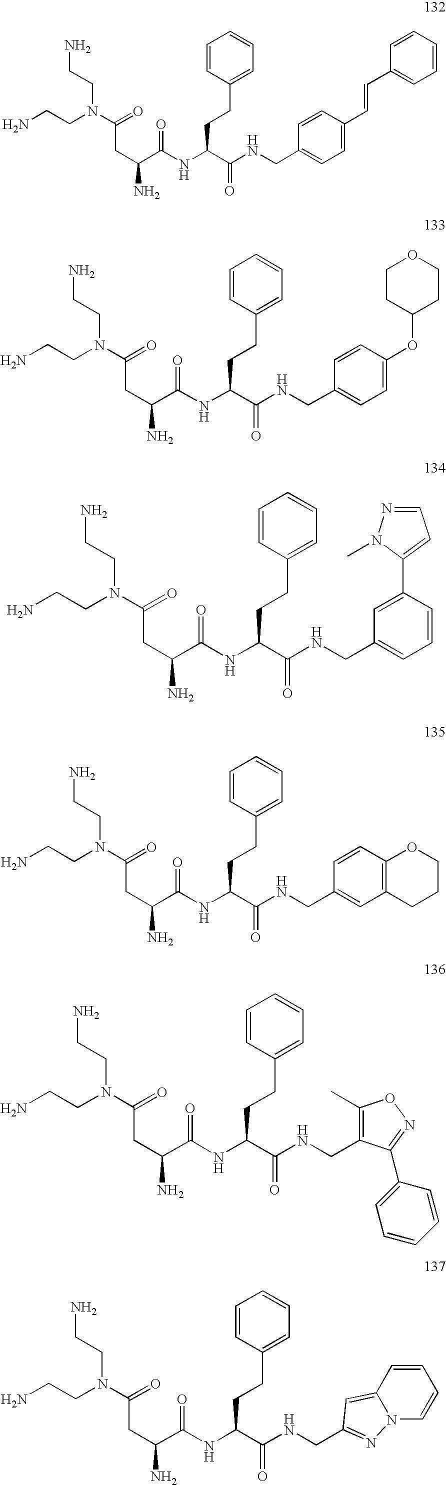

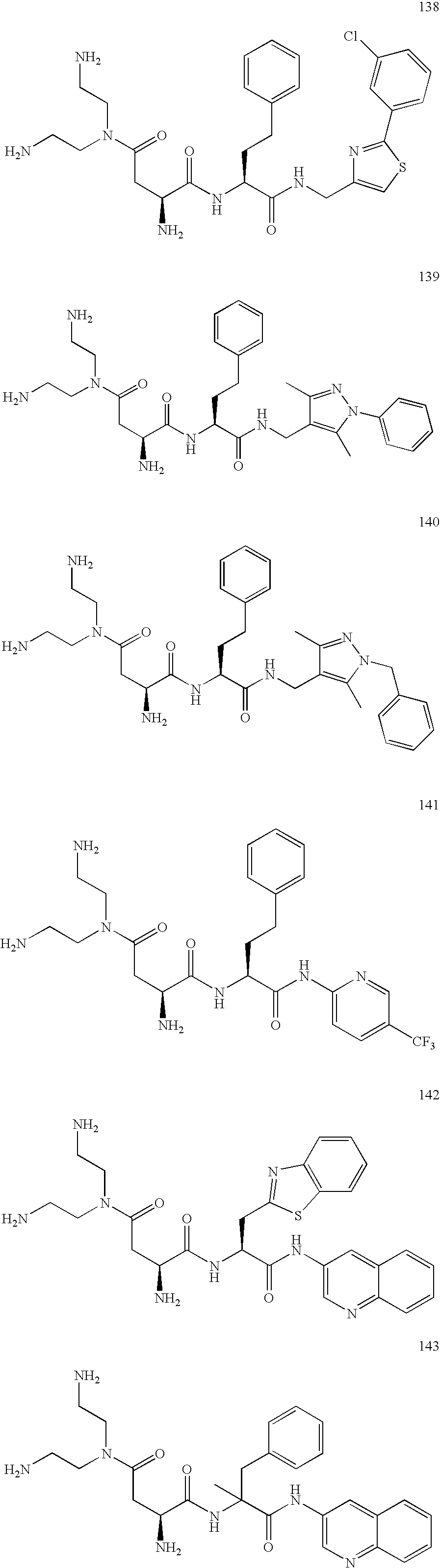

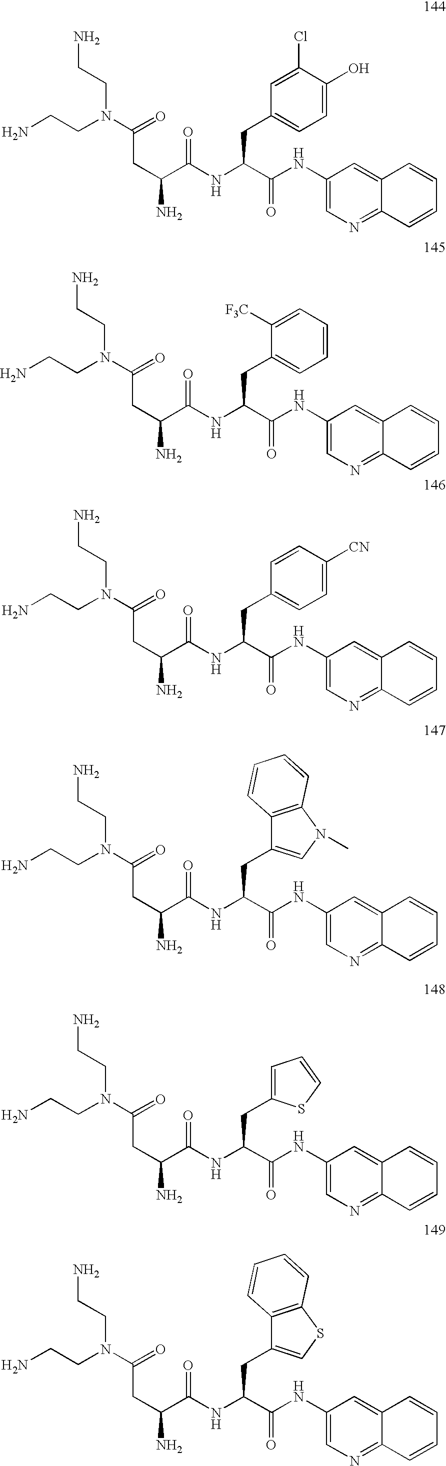

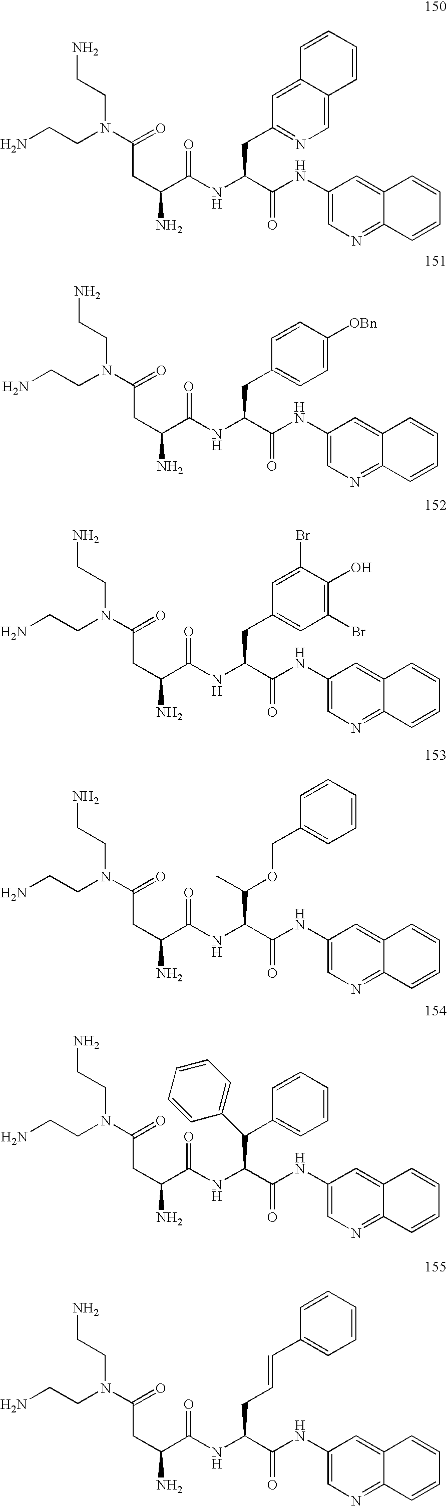

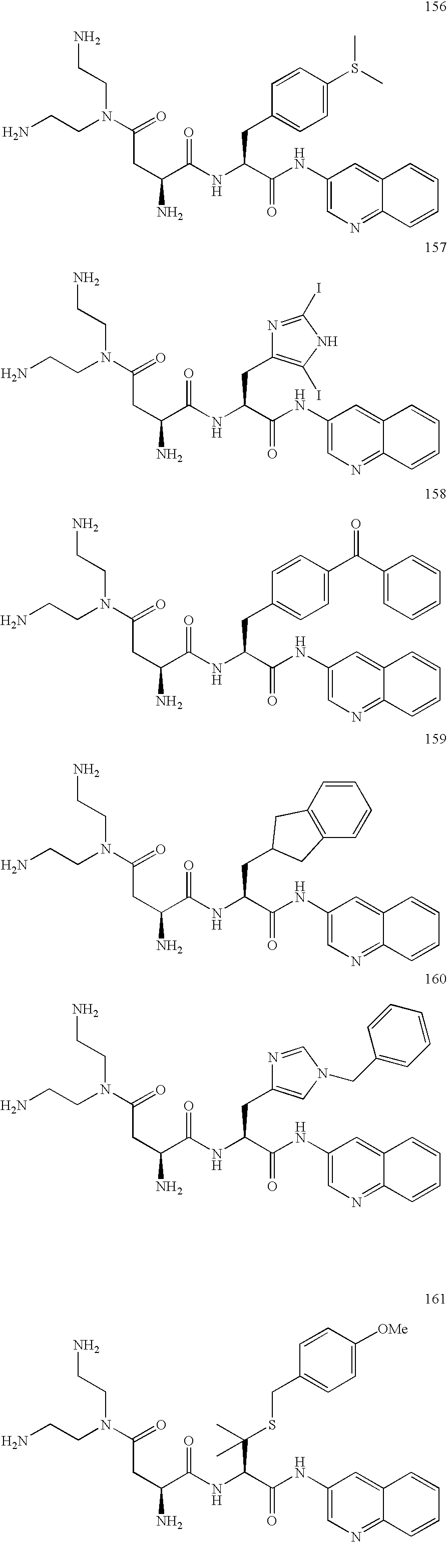

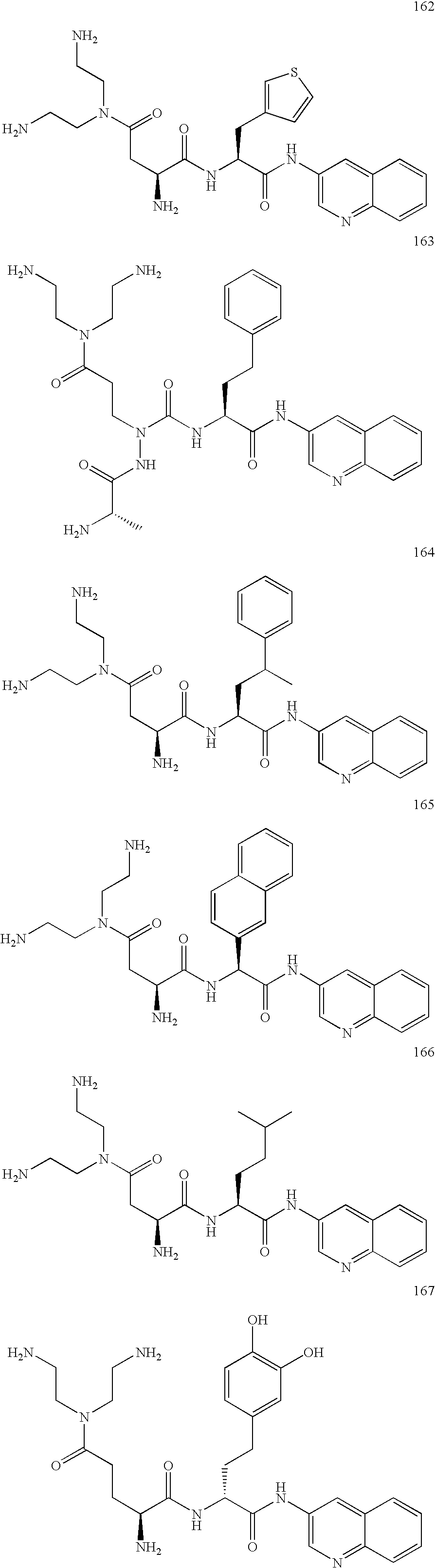

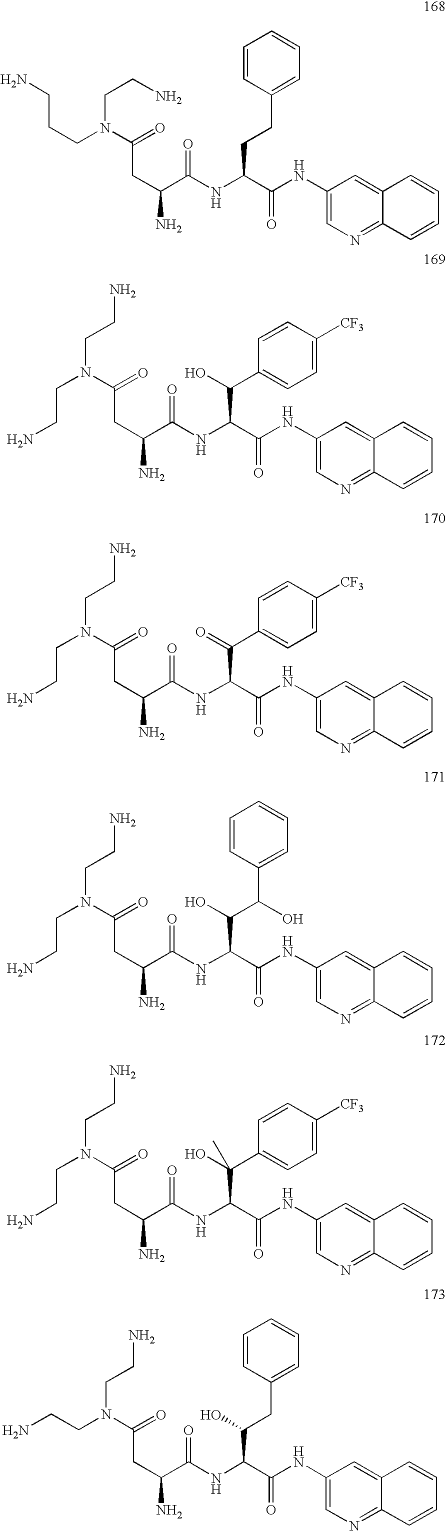

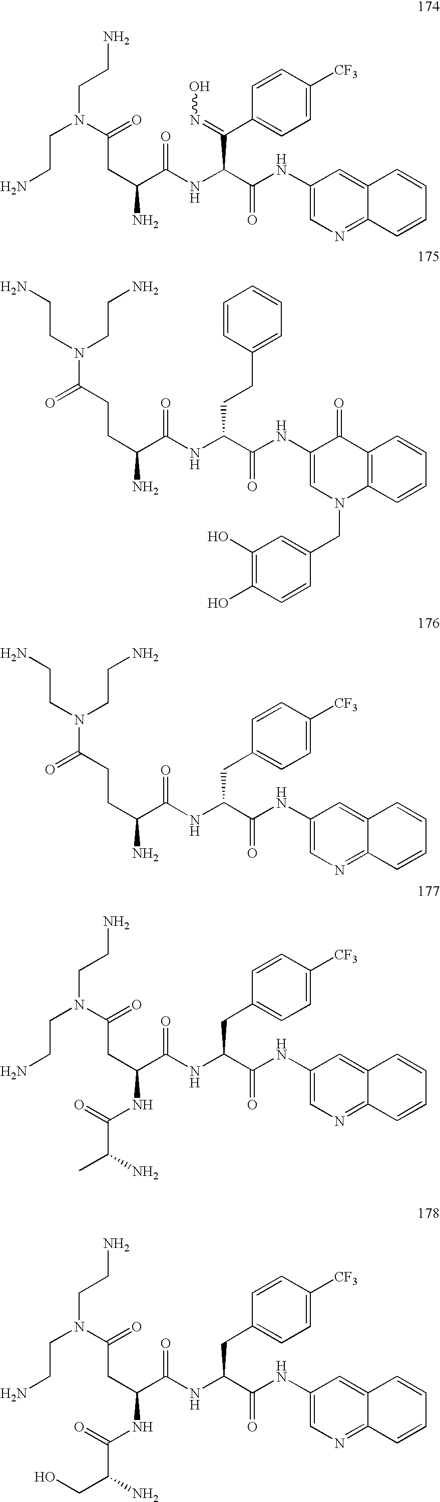

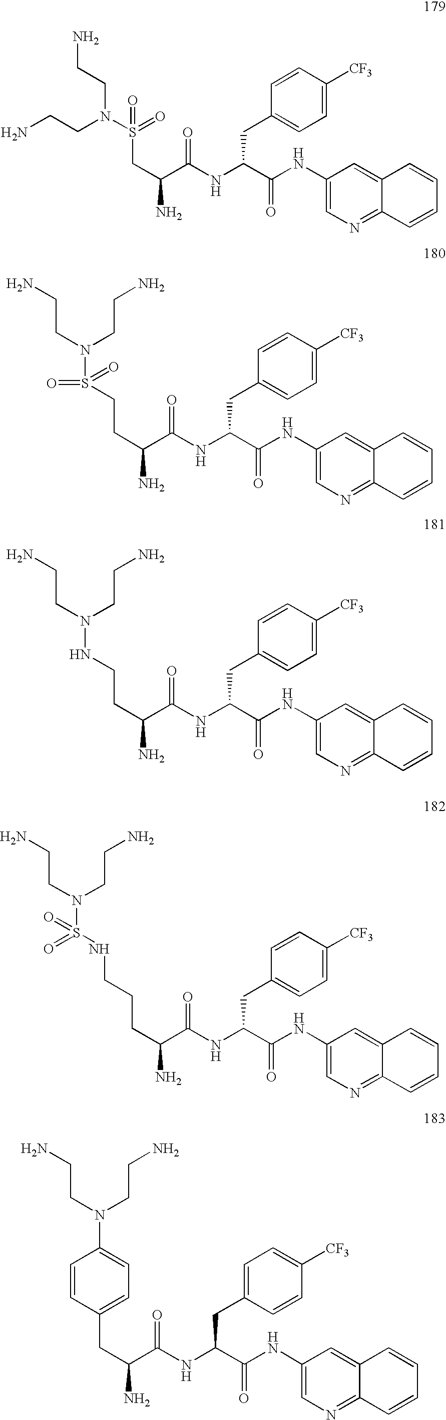

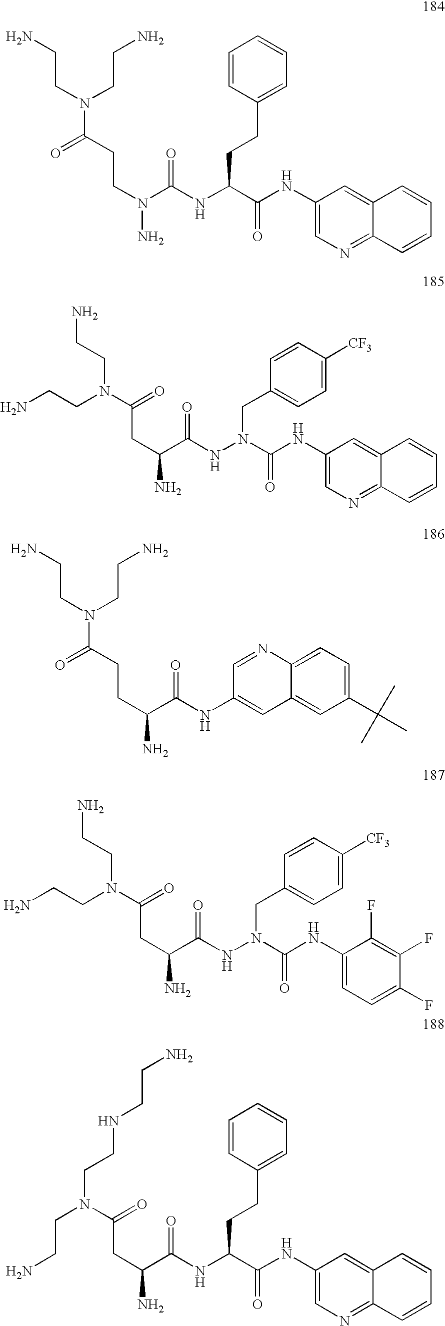

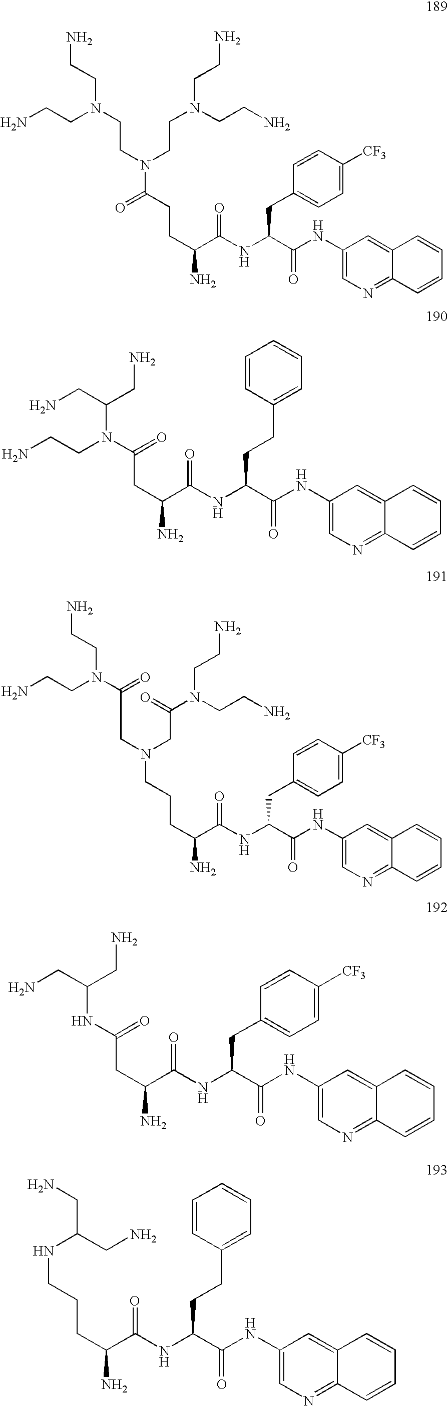

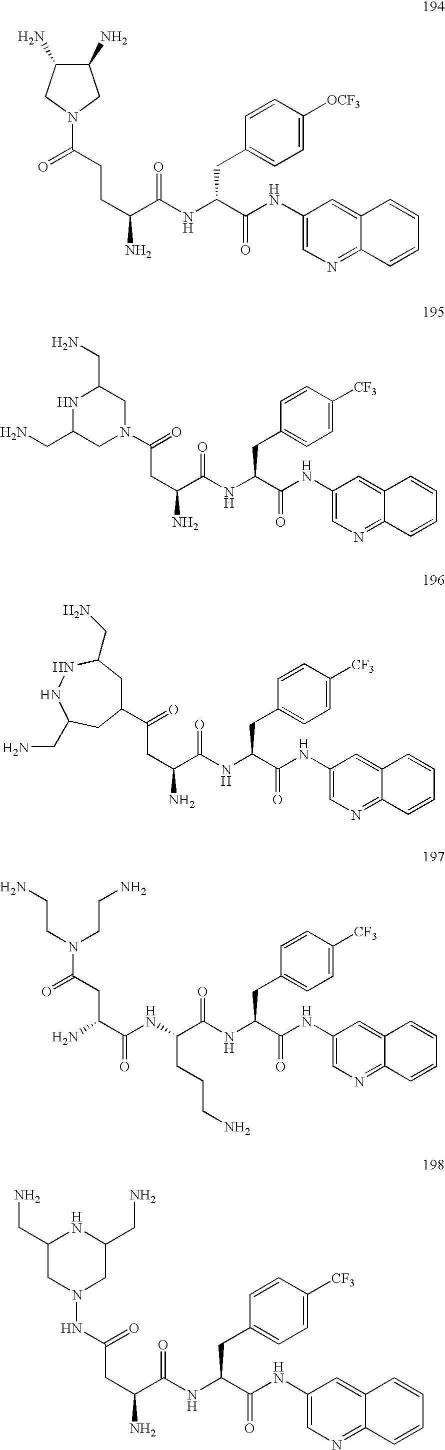

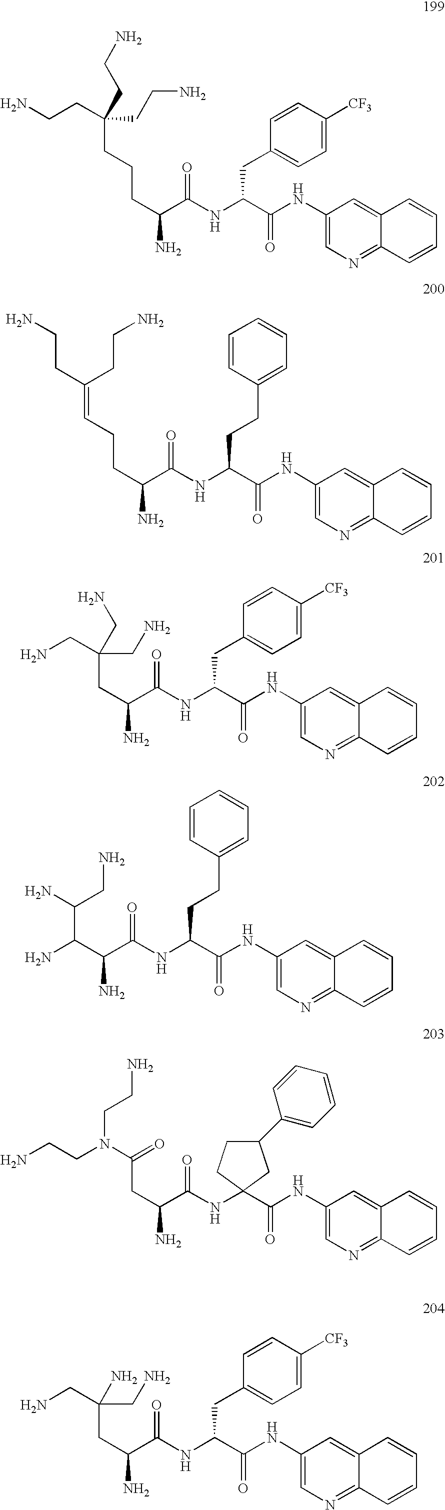

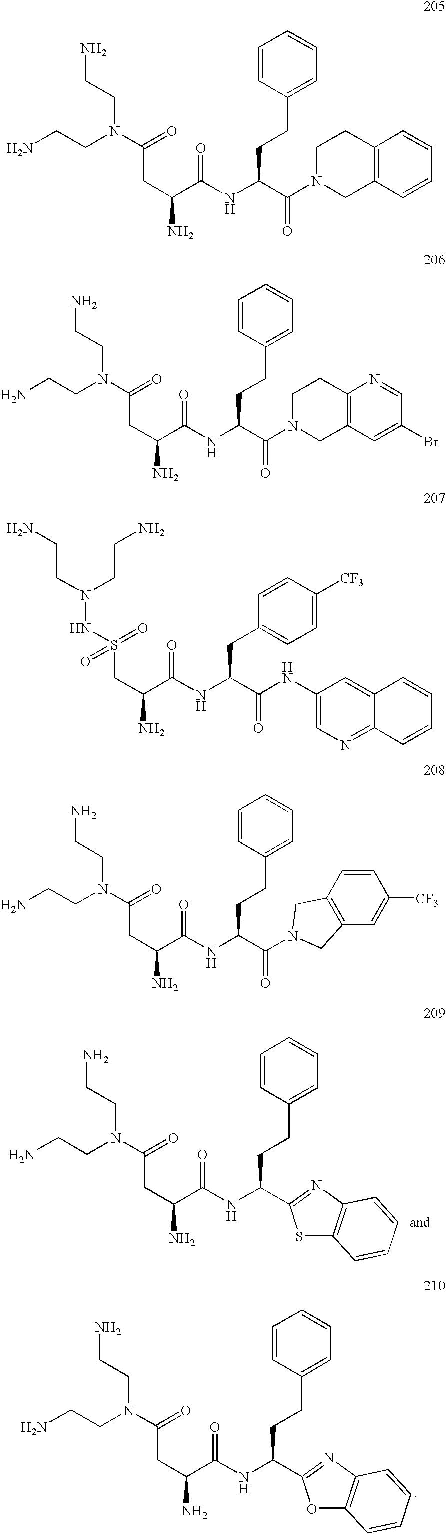

145. The compound of claim 1 having a structure selected from the group consisting of:

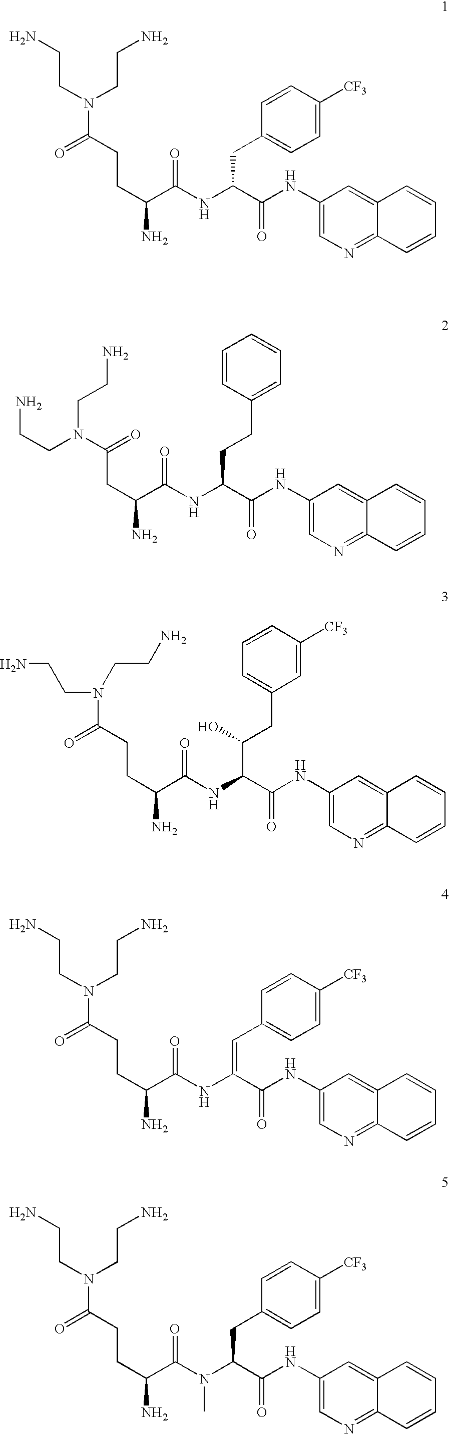

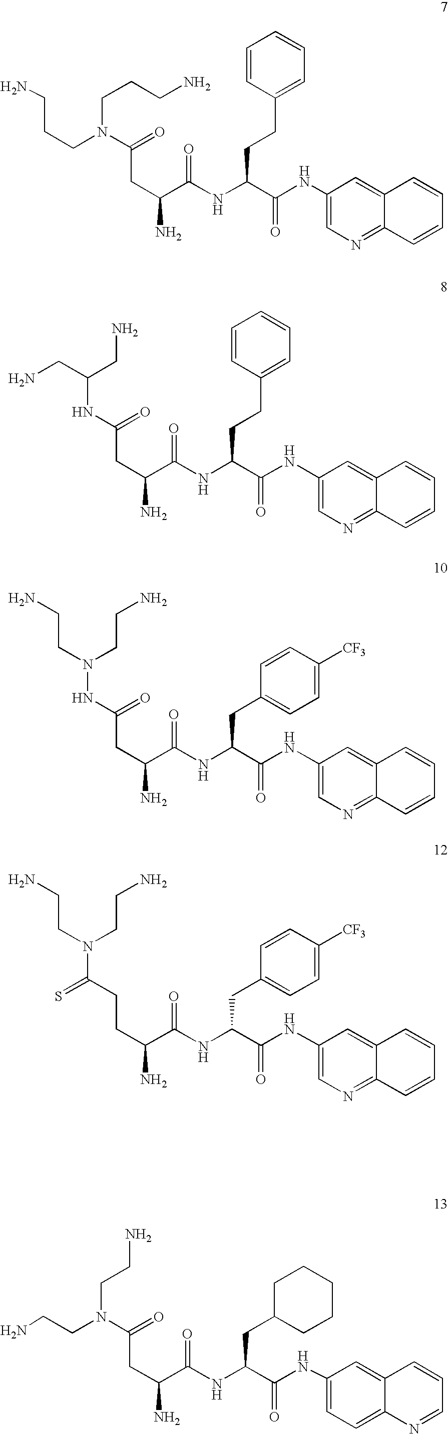

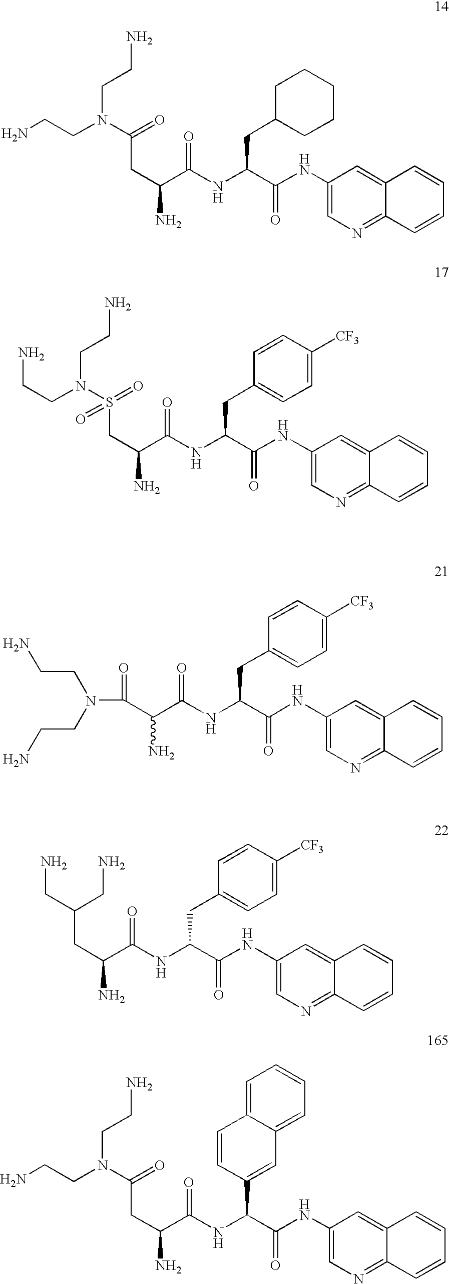

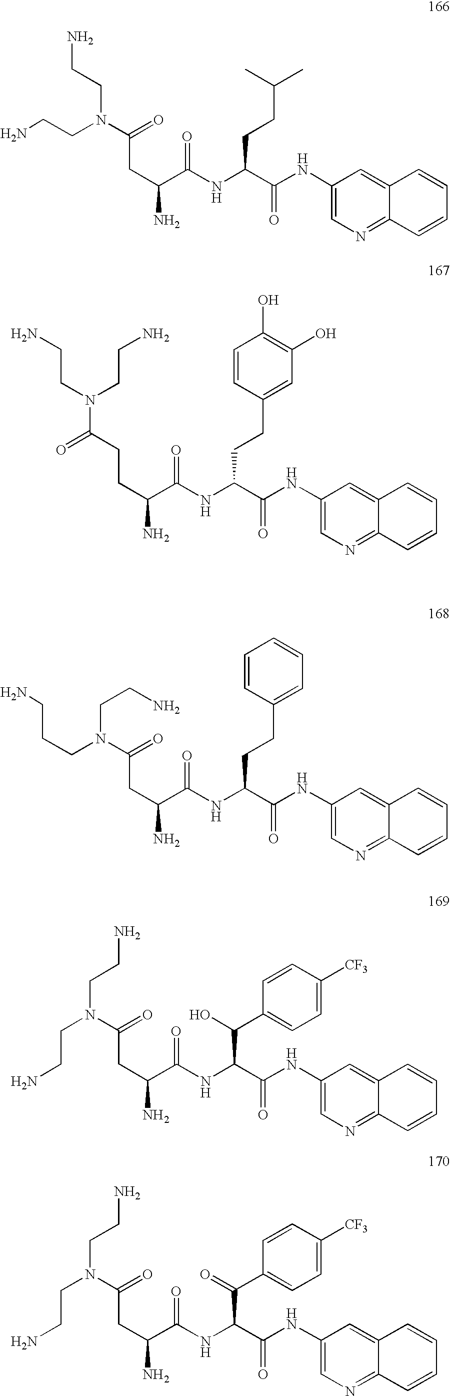

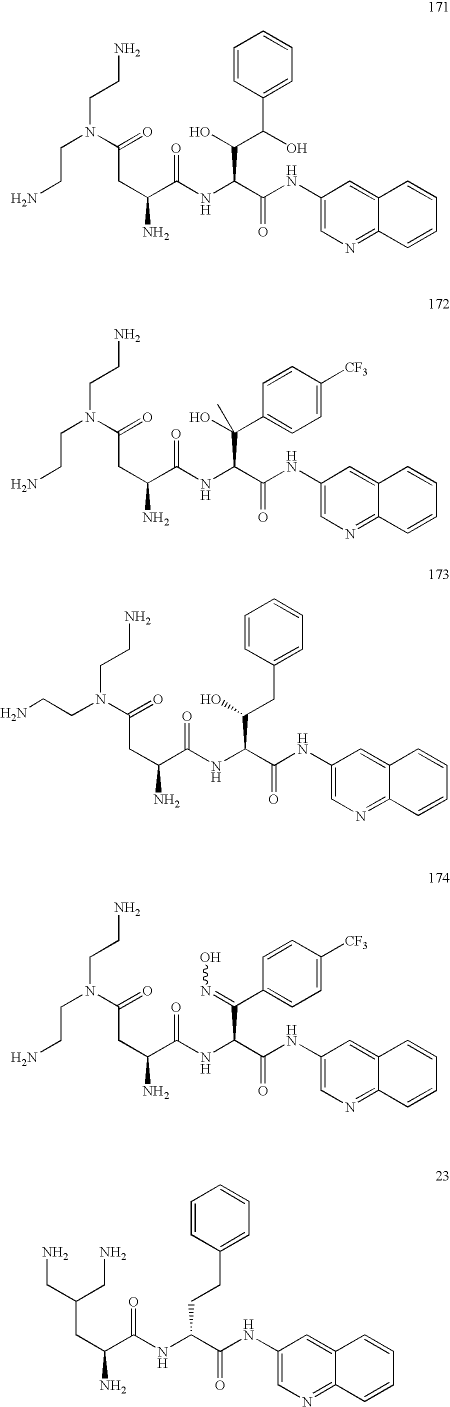

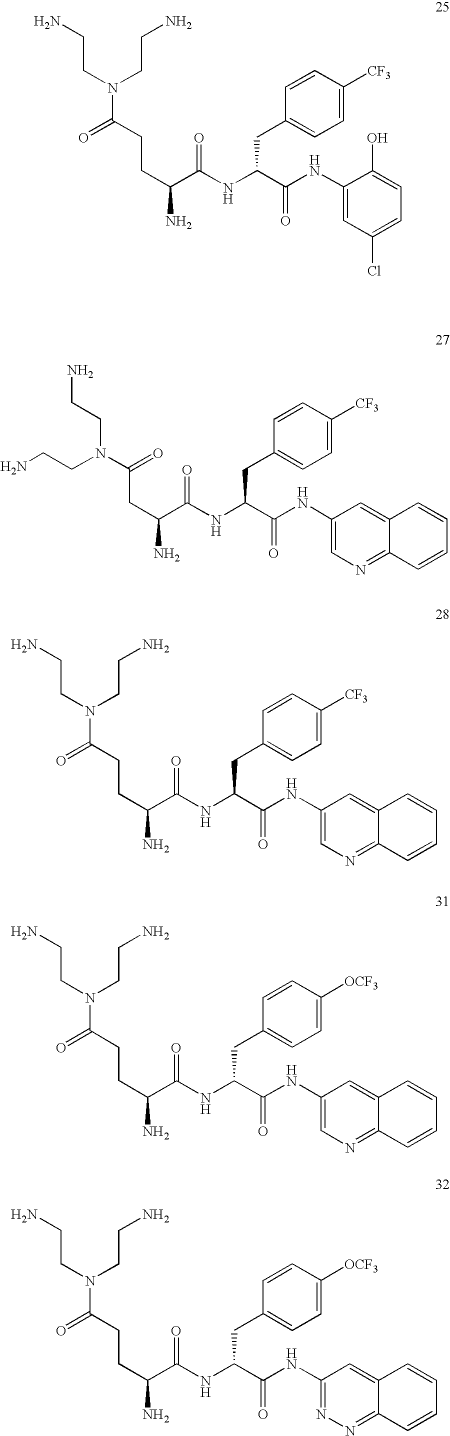

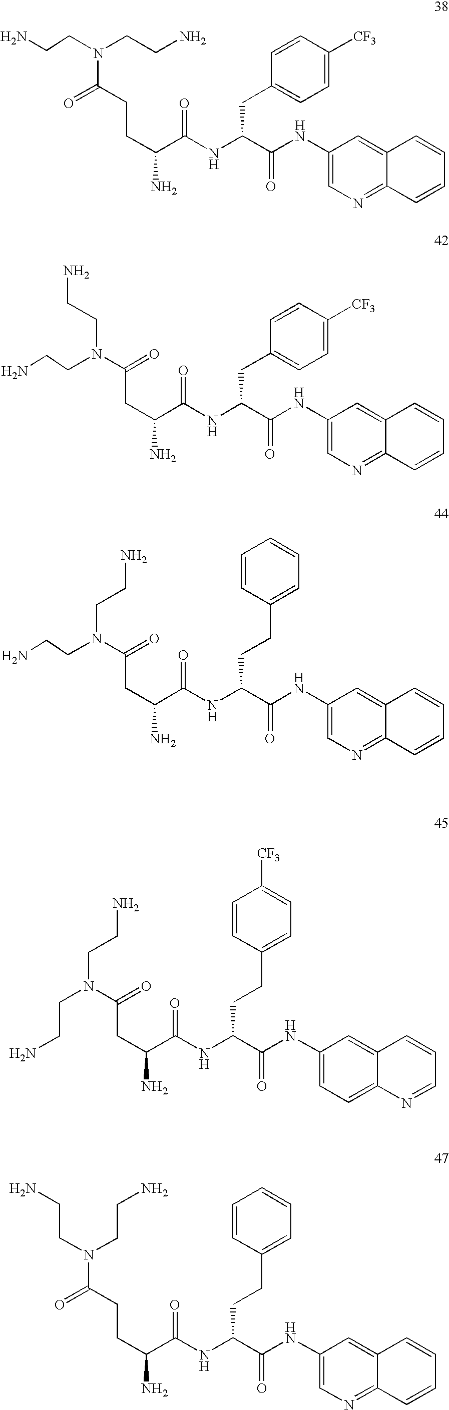

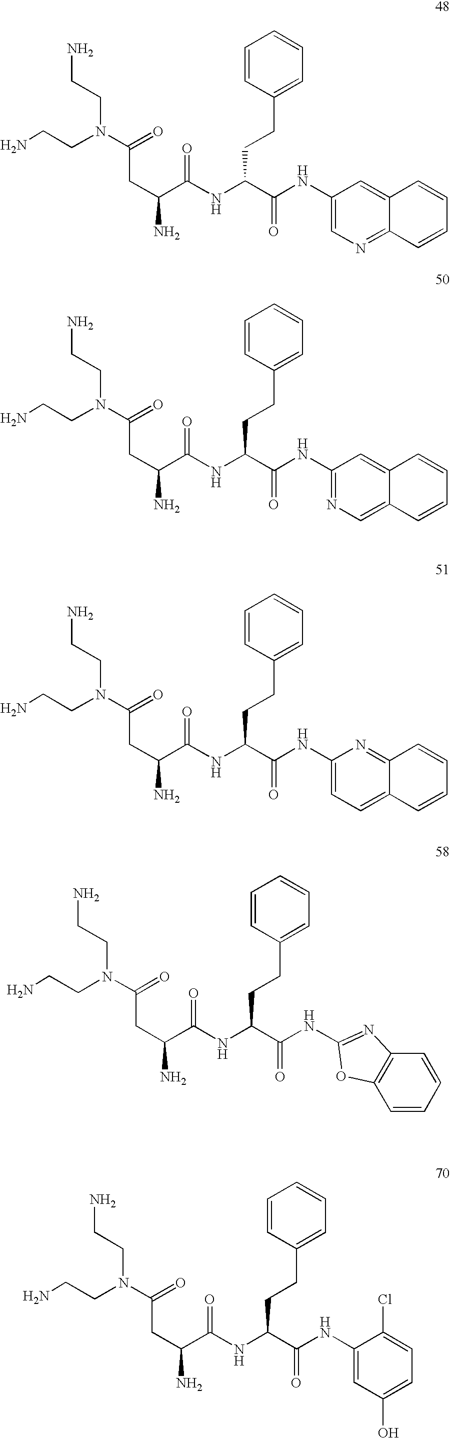

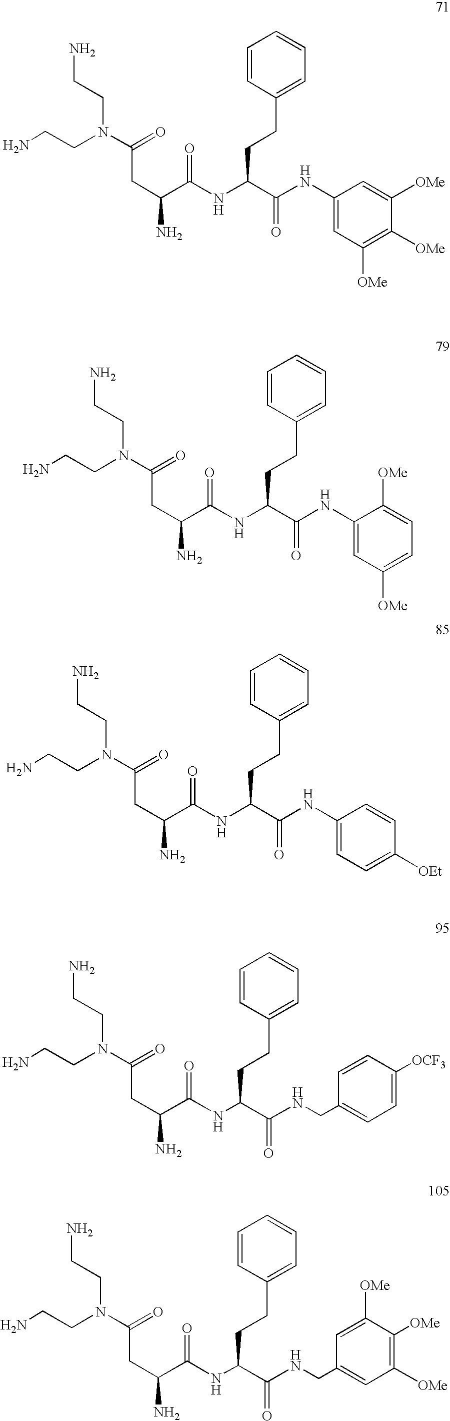

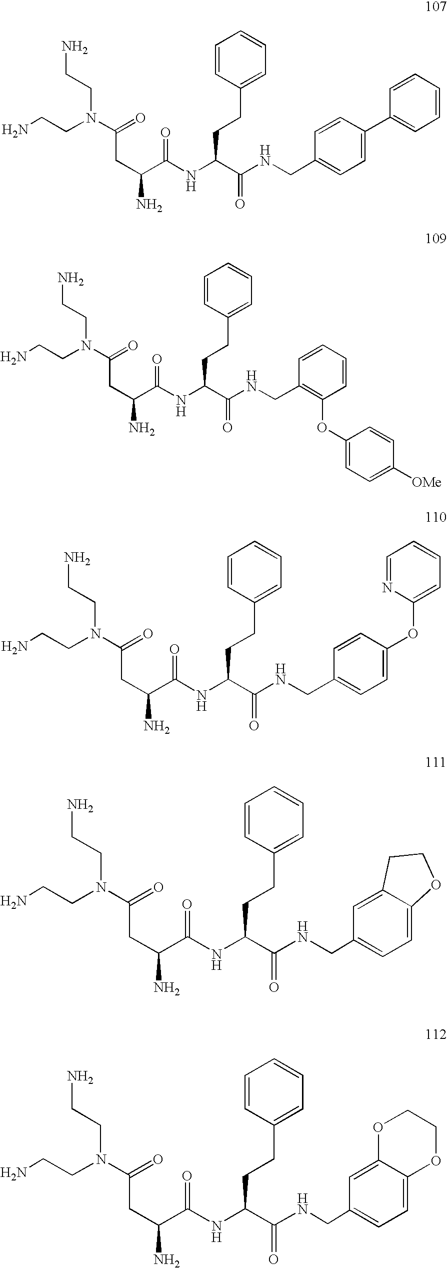

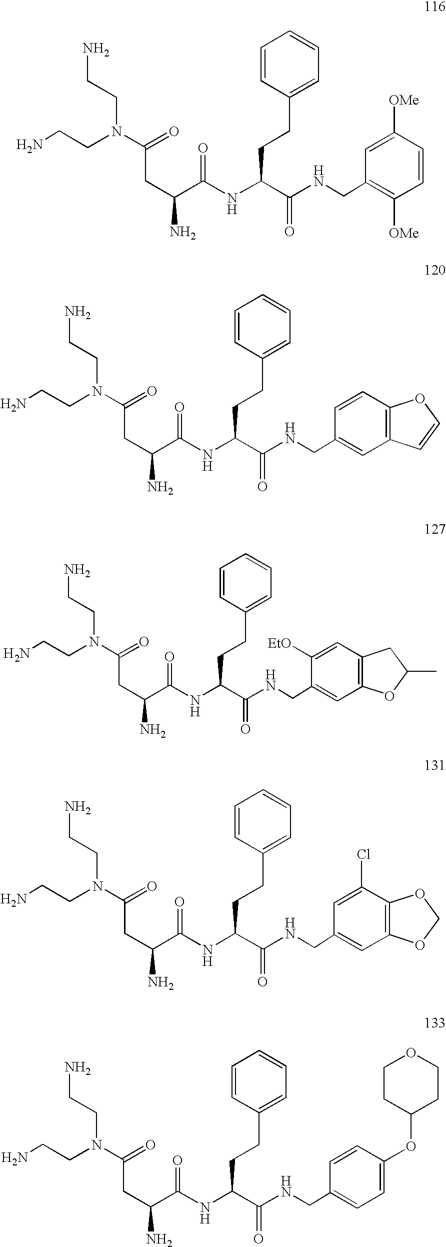

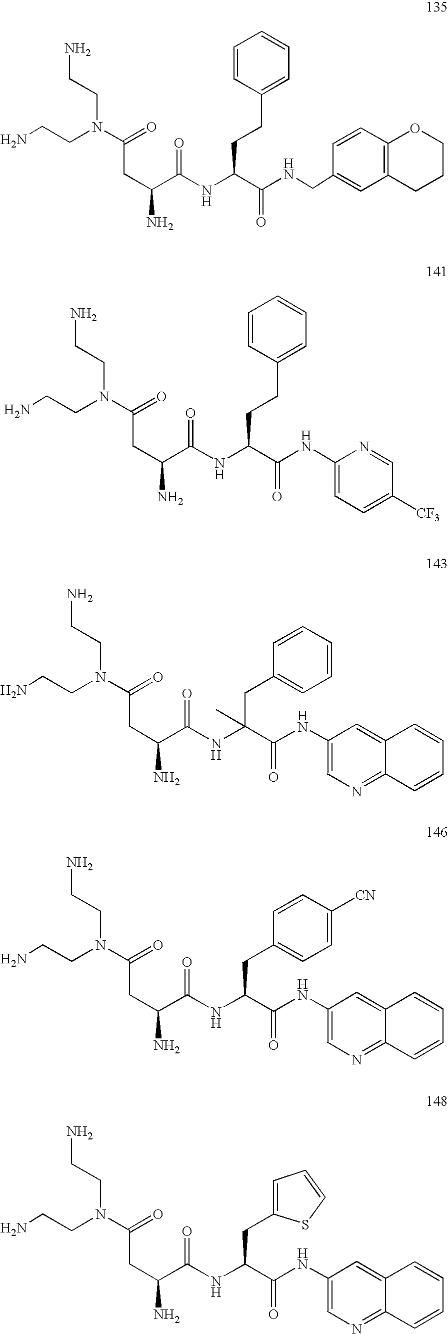

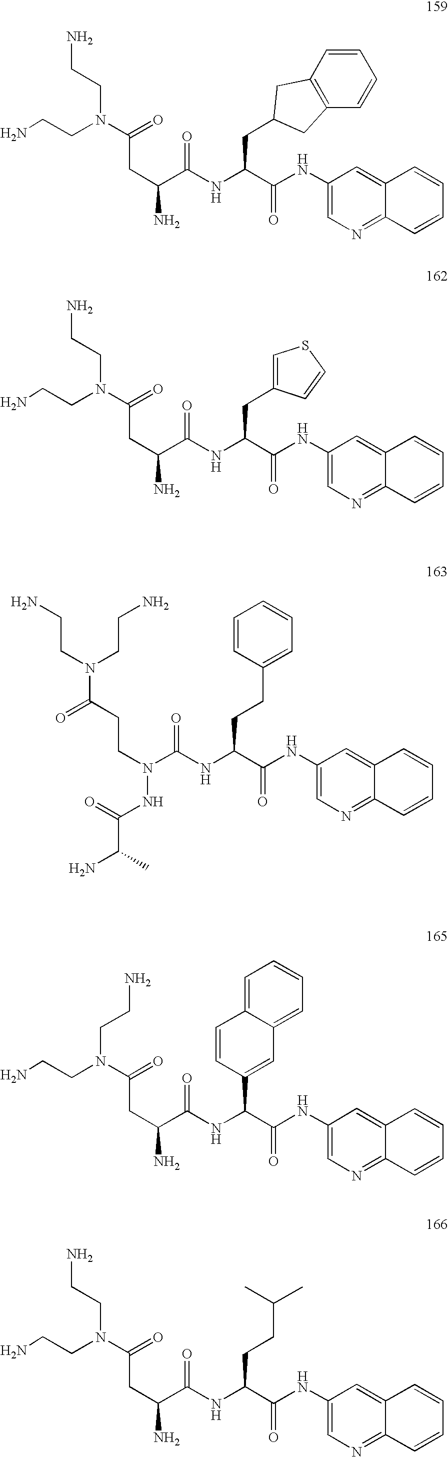

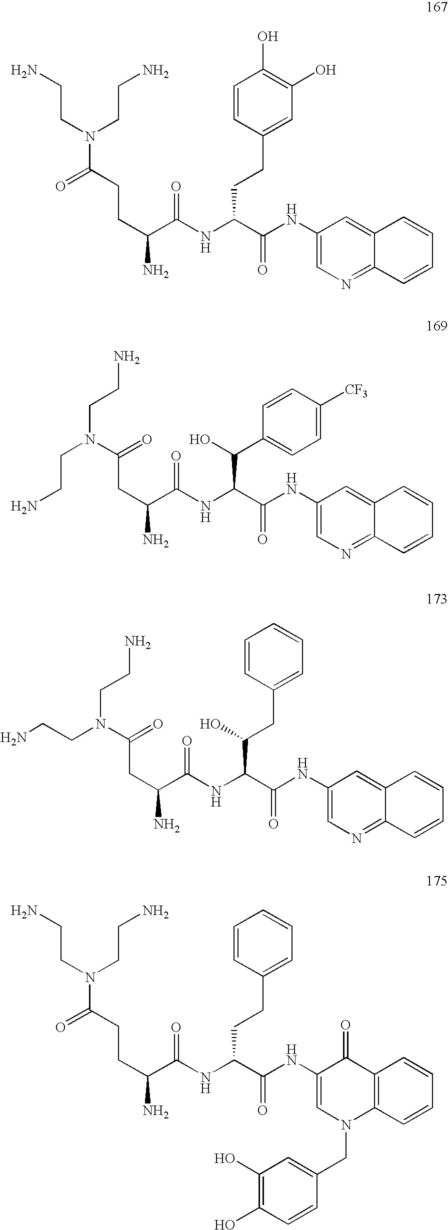

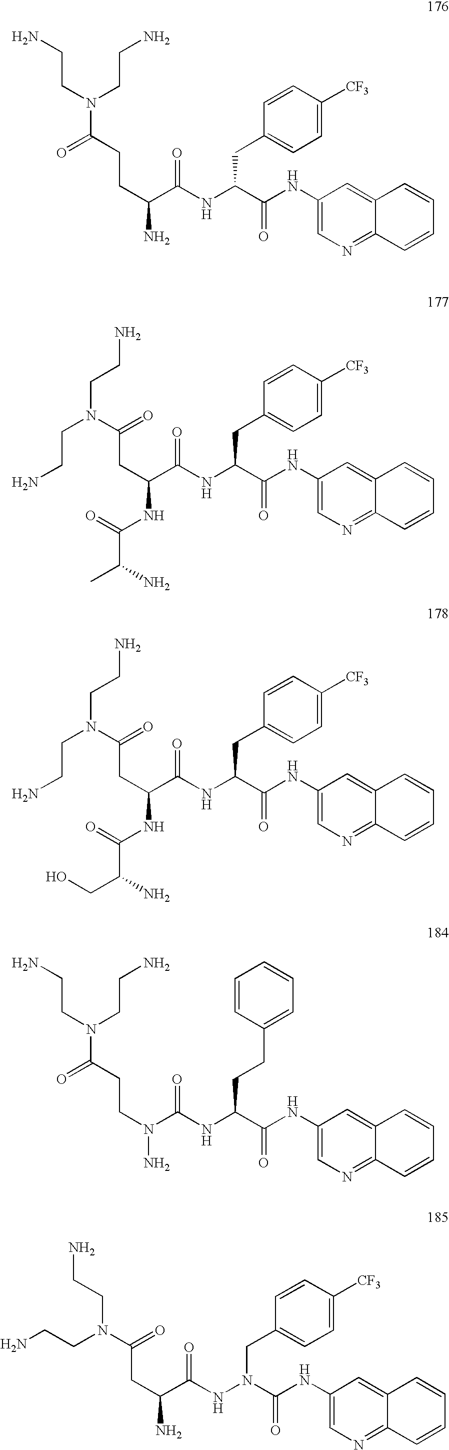

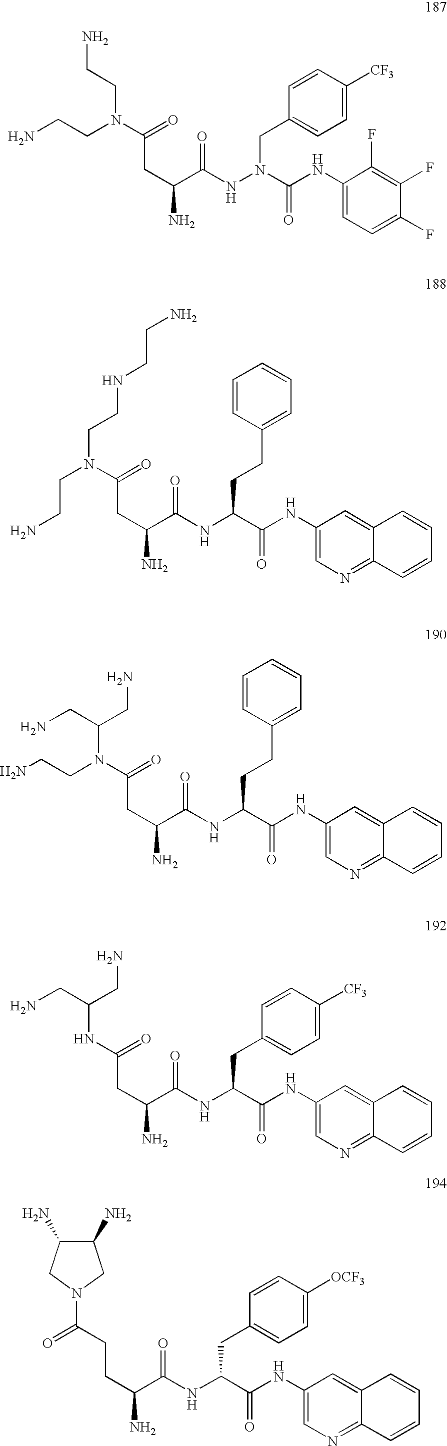

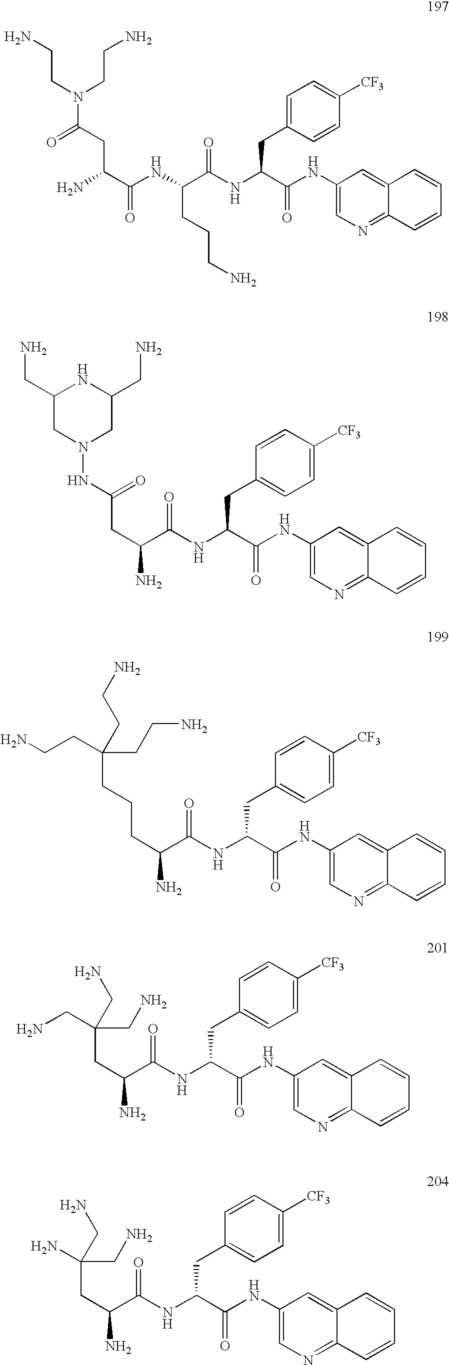

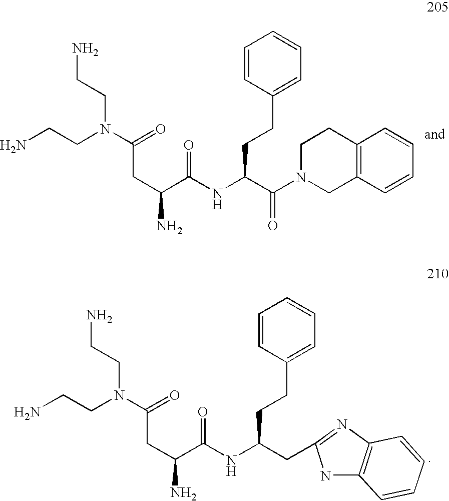

146. The compound of claim 1 having a structure selected from the group consisting of:

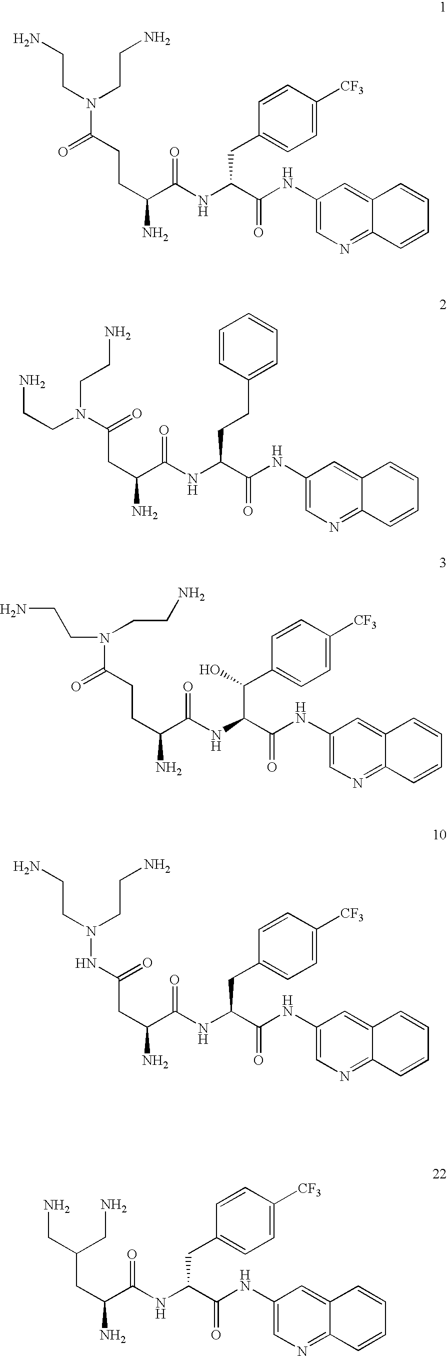

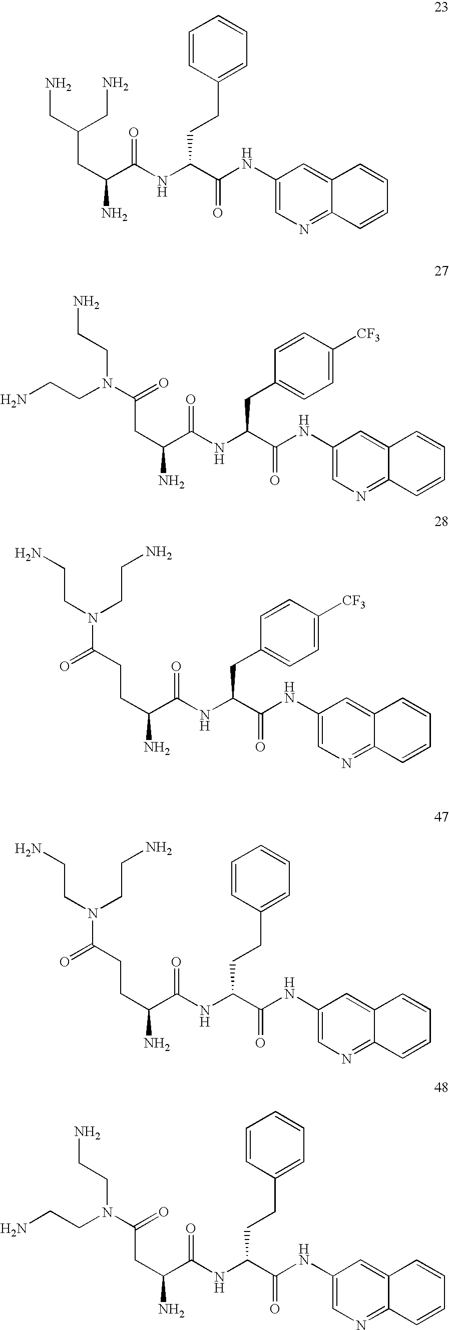

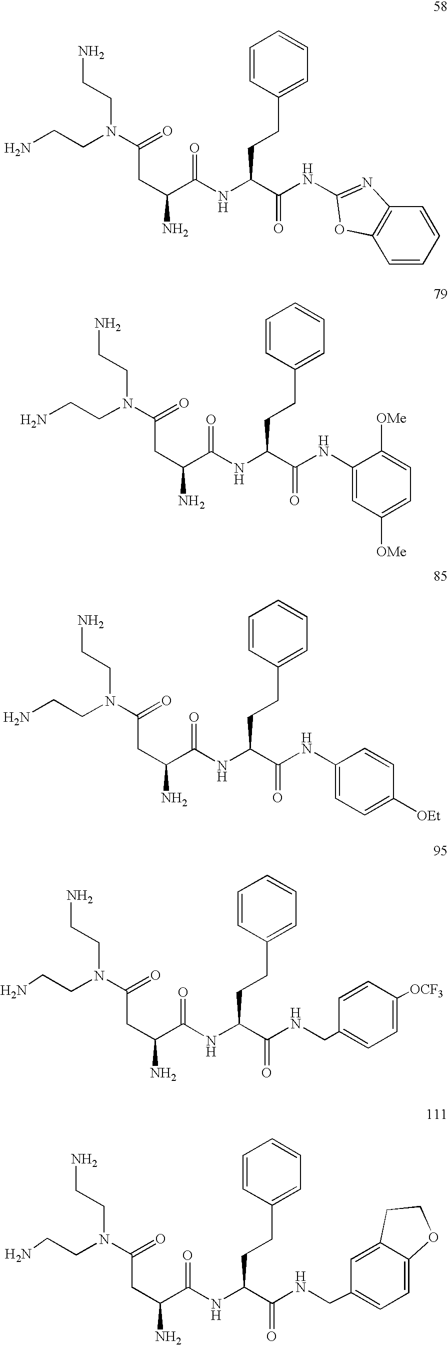

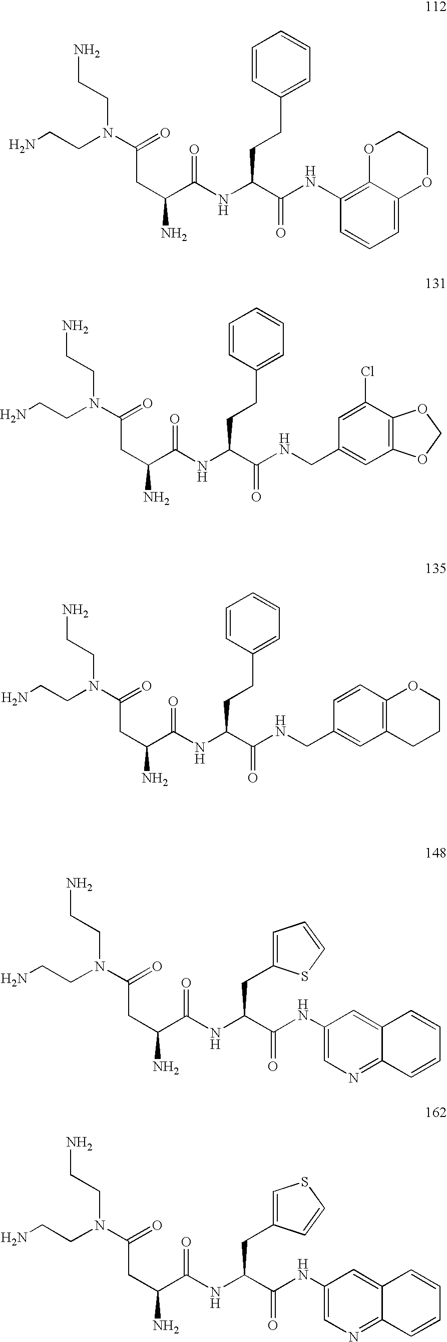

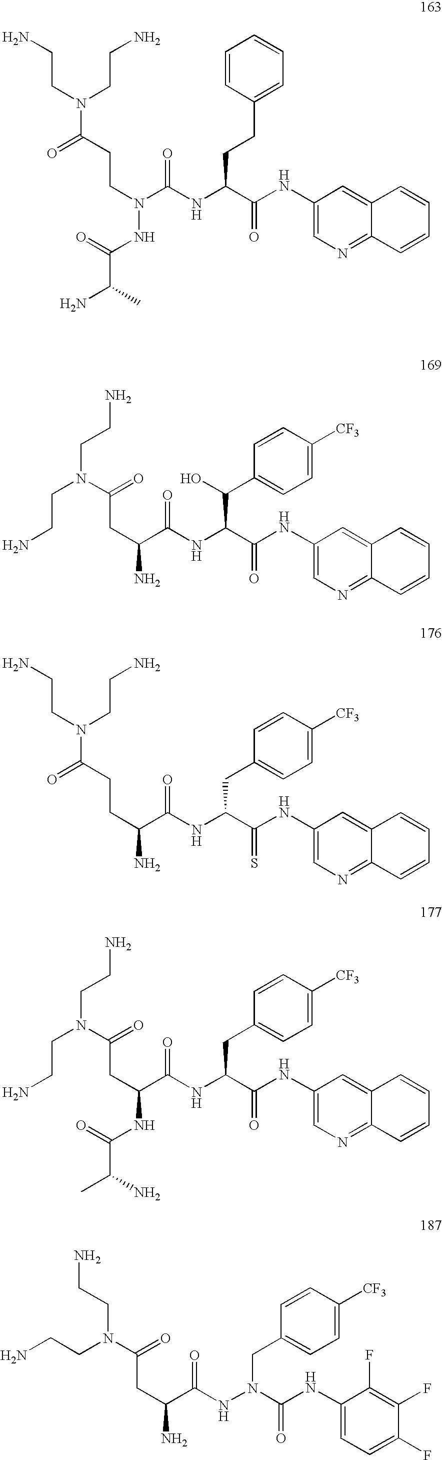

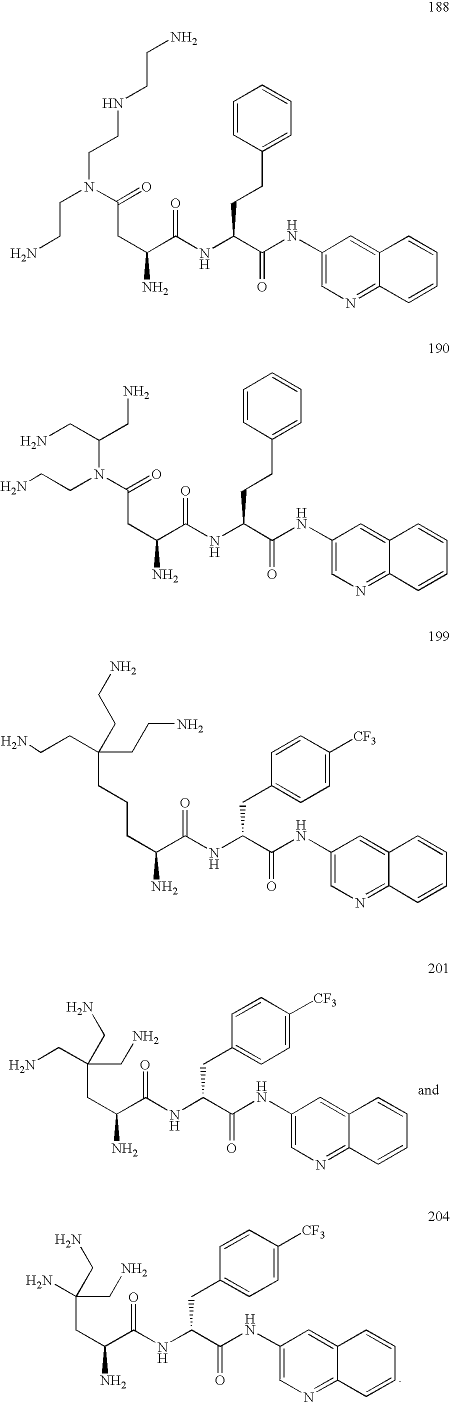

147. A compound having the structure of formula IV: 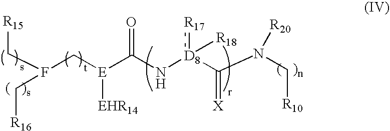
or a pharmaceutically acceptable salt or prodrug thereof, wherein:
D₈ is selected from C and N;
each E is independently CH or N;
F is selected from the group consisting of: 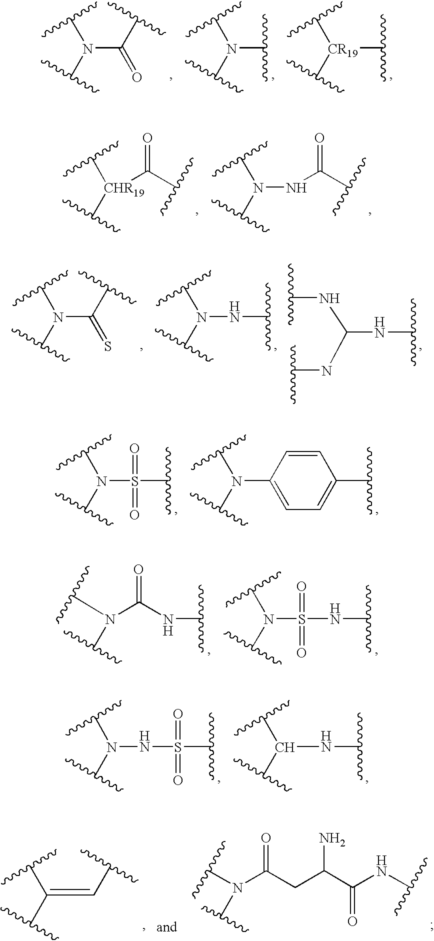
X is O or S;
R₁₀ is selected from carbocyclyl, heterocyclyl, aryl, heteroaryl, —NHC(O)— aryl, and aralkyl, each optionally substituted with up to 3 substituents independently selected from the group consisting of a halide, alkyl, —CF₃, —OCF₃, —NO₂, —CN, —OH, ═O, carbocyclyl, heterocyclyl, aryl optionally substituted with halide or —OH, heteroaryl optionally substituted with alkyl, —O-aryl optionally substituted with —O—C₁-C₆ alkyl, —O-heteroaryl, —O-heterocyclyl, —SO₂NH-heteroaryl, —O—C₁-C₆ alkyl, —SO₂NEt₂, SMe, di(C₁-C₆)alkylamino, —CH₂-heterocyclyl optionally substituted with alkyl, —CH₂-aryl, —C(O)aryl, and —CH═CH-aryl;
R₁₄ is selected from H, —C(O)—CH(Me)(NH₂), —C(O)—CH(CH₂OH)(NH₂), and —(CH₂)ₜNH₂;
R₁₅ and R₁₆ are independently selected from —NH₂, —NHC(═NH)NH₂, —N⁺(CH₃)₃, —NHCH₂CH₂NH₂, —N(CH₂CH₂NH₂)₂, —C(O)N(CH₂CH₂NH₂)₂, —CH(CH₂NH₂)₂, and —CH₂(NH₂)(CH₂NH₂),
or R₁₅ and R₁₆ together with F form a heterocyclyl substituted with at least two substituents independently selected from —(CH₂)ₙNH₂, —(CH₂)ₙNHC(═NH)NH₂—(CH₂)ₙN⁺(CH₃)₃, —(CH₂)ₙNHCH₂CH₂NH₂, —(CH₂)ₙN(CH₂CH₂NH₂)₂, —(CH₂)ₙC(O)N(CH₂CH₂NH₂)₂, and —(CH₂)SCH(CH₂NH₂)₂;
R₁₇ is selected from alkyl, aralkyl, heteroaralkyl, carbocyclyl-alkyl, heterocyclyl-alkyl, aryl, and carbocyclyl, each optionally substituted with up to 3 substituents independently selected from the group consisting of —CF₃, —OH, —OCF₃, halide, —CN, alkyl —O-aralkyl, aryl, —S(CH₃)₂, —C(O)aryl, —S-aralkyl optionally substituted with —OMe, ═O, and ═N—OH;
R₁₈ is H, alkyl, or absent,
or R₁₇ together with R₁₈ form a carbocyclyl optionally substituted with aryl or heteroaryl;
R₁₉ is H, —CH₂NH₂, or —CH₂CH₂NH₂;
R₂₀ is H or alkyl;
each t is independently an integer from 1 to 4;
each s is independently an integer from 0 to 3;
r is 0 or 1; and
n is an integer from 0 to 4.

148. A compound having the Formula V or VI: 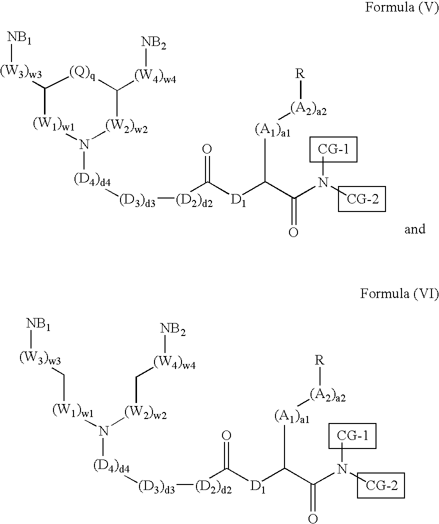
or a pharmaceutically acceptable salt or pro-drug ester thereof wherein;
NB₁ and NB₂ are independently selected from the group consisting of —NH₂, —NHMe, —NHCH(═NH), —NHC(═NH)NH₂, —NH—NH₂, and —NH—NHC(═NH)NH₂; —NHC(═NH)NH—NH₂;
W₁ and W₂ are independently selected from the group consisting of —CH₂—, —C(═O)—, and —(SO₂)—; with the proviso that only one of W₁ and W₂ can be —C(═O)— or —(SO₂)—;
W₃ and W₄ are independently selected from the group consisting of —CH₂—, —CH(═NH)—, and —NH—;
with the proviso that the fragments NB₁—W₃ and NB₂—W₄ do not have more than 2 consecutive heteroatoms in each fragment; and
when there are two consecutive heteroatoms in the NB₁—W₃ or NB₂—W₄ fragments the heteroatom combinations are selected from the group consisting of N—N, N—O, and O—N;
Q is —CH₂—, or —C(═O)—;
D₁ is —NH—, —NMe-, or —O—;
D₂, D₃, D₄, and D₅ are independently selected from the group consisting of —CH(NH₂)—, —CH(OH)—, —CMe(OH)—, —CHMe-, —CEt(OH)—, —CHEt-, —CMe₂-, —CH₂—, —C(CH₂)₂—, —CH₂CH₂—, —C(═O)—, —CH(═NH)—, —NH—, —NMe-, —N(CH₂CH₂NH₂)—, —O—, —S—, and —N(NH₂)—; alternatively any two atoms of D₂, D₃, and D₄ can be additionally connected as to form a four, five or six membered saturated ring selected from the group consisting of C₃N, C₄, C₅, C₄N, C₆, and C₅N;
with the proviso that the combined length of D₄, D₃, and D₂ is not more than 6 atoms; and
when D₄ is —C(═O)— and D₃ is —C(═O)— then D₂ is not —C(═O)— and d2 is equal to 1;
with the proviso that when D₂ is —C(═O)— then D₃ is not —C(═O)—; and
if d3 is equal to 0 then D₄ is not —C(═O)—;
d2, d3, d4, w1, w2, w3, w4 and q independently are 0 or 1; with the proviso that w1+w3>0 and w2+w4>0;
A₁ is —CH₂—, —CH₂CH₂—, or —CH₂CH₂CH₂—;
A₂ is —O— or —S—;
a1 and a2 are independently equal to 0 or 1;
R is C₄-C₈ alkyl, cyclopentyl, cyclohexyl, cycloheptyl, phenyl, naphtyl optionally substituted with up to 3 substituents independently selected from the group consisting of F, Cl, Me, Et, iPr, OH, OMe, CF₃, OCF₃, NH₂, NHMe, NMe₂, NO₂, CN, CO₂Me, CO₂Et, or CO₂iPr;
CG-1 is a carbon-linked capping group; with the proviso that the linking carbon atom is not an α-atom of an α-amino acid;
CG-2 is a hydrogen, C₁-C₄ alkyl or a carbon-linked capping group; with the proviso that the linking carbon atom is not an α-atom of an α-amino acid;
the carbon-linked capping group is selected from the group consisting of: 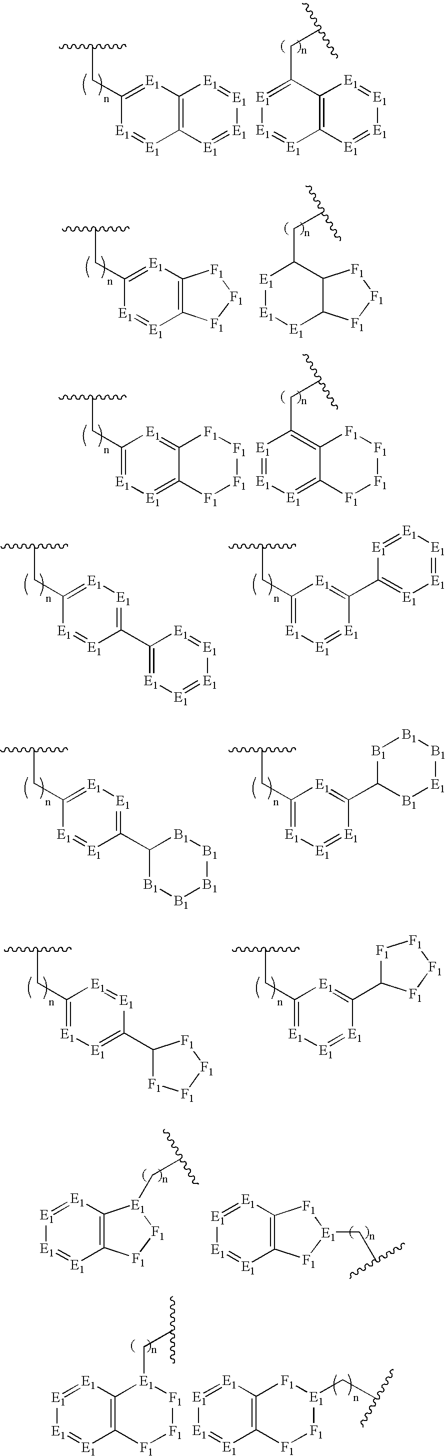 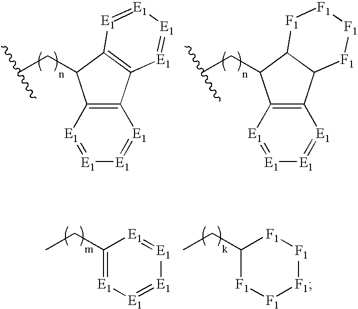
E₁ is CH or N;
F₁ is CH₂, NH, N(R⁶), or O, with the proviso that two consecutive F₁ groups cannot be O;
R⁶ is H or C₁₋₆ alkyl;
n is equal to 0, 1, or 2;
m is equal to 0, 1, 2, 3, or 4; and
k is equal to 0, 1, 2, 3, or 4.

149. The compound of claim 148, wherein D₂ is —CH(NH₂)— and d2 is equal to 1.

150. A method of inhibiting a bacterial efflux pump, comprising administering to a subject infected with a bacteria a compound according to claims 1.

151. A method of treating or preventing a bacterial infection, comprising co-administering to a subject infected with a bacteria or subject to infection with a bacteria, a compound according to claim 1 and another anti-bacterial agent.

152. The method of claim 151 wherein the bacteria is selected from Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

153. The method of claim 151 wherein the bacteria is selected from Pseudomonas aeruginosa, Pseudomonas fluorescens, Stenotrophomonas maltophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Acinetobacter calcoaceticus, Acinetobacter haemolyticus, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Helicobacter pylori, Campylobacter fetus, Campylobacter jejuni, Campylobacter coli, Vibrio cholerae, Vibrio parahaemolyticus, Legionella pneumophila, Listeria monocytogenes, Neisseria gonorrhoeae, Neisseria meningitidis, Moraxella, Bacteroides fragilis, Bacteroides vulgatus, Bacteroides ovalus, Bacteroides thetaiotaomicron, Bacteroides uniformis, Bacteroides eggerthii, or Bacteroides splanchnicus.

154. The method of claim 151 wherein the anti-bacterial agent is selected from quinolones, tetracyclines, glycopeptides, aminoglycosides, β-lactams, rifamycins, macrolides/ketolides, oxazolidinones, coumermycins, and chloramphenicol.

155. A pharmaceutical composition, comprising a compound according to claim 1 and a pharmaceutically acceptable carrier, diluent, or excipient.
